(19) United States
(12) Patent Application Publication
(10) Pub. No.: US 2026/0209772 A1
(43) Pub. Date: Jul. 23, 2026

(54) NOVEL RNA THERAPEUTICS AND USES THEREOF

(71) Applicant: Eli Lilly and Company, Indianapolis, IN (US)

(72) Inventors: Patrick Joseph ANTONELLIS, Norwell, MA (US); Thomas Patrick BEYER, Zionsville, IN (US); Jean Mawuena BRANTTIE, Indianapolis, IN (US); Christine Chih-Tao CHENG, Carmel, IN (US); Marjoke F. DEBETS, Carmel, IN (US); Konstantinos GAVARDINAS, Carmel, IN (US); Gregory Lawrence LACKNER, Indianapolis, IN (US); Jibo WANG, Carmel, IN (US); Takako WILSON, San Diego, CA (US)

(21) Appl. No.: 19/141,216

(22) PCT Filed: Dec. 22, 2023
(86) PCT No.: PCT/US2023/085630

§ 371 Date: Jun. 19, 2025

Related U.S. Application Data
(60) Provisional application No. 63/435,100, filed on Dec. 23, 2022.

(51) Int. Cl. C12N 15/113; A61K 31/712; A61K 31/7125; A61P 3/06; C12N 9/04

(52) U.S. Cl. CPC C12N 15/1137; A61K 31/712; A61K 31/7125; A61P 3/06; C12N 9/0006; C12Y 101/01034; C12N 2310/14; C12N 2310/315; C12N 2310/321; C12N 2310/322; C12N 2310/346; C12N 2310/351; C12N 2310/3521; C12N 2310/3533

(57) ABSTRACT

The present disclosure relates to novel therapeutic compounds, known as RNAi agents, that decrease expression of the HMGCR receptor (expressed by the HMGCR gene), thereby decreasing expression of mRNA and protein expression. Such RNAi agents are useful in the treatment of diseases and disorders involving the regulation of HMGCR expression and function, such as diseases and disorders that are risk factors for ASCVD (such as, dyslipidemia, such as hypercholesteremia).

### BACKGROUND

The present invention relates to novel therapeutic compounds, known as RNAi agents, that decrease expression of the HMGCR (expressed by the HMGCR gene), thereby decreasing expression of HMGCR mRNA and HMGCR protein. Such RNAi agents are useful in the treatment of diseases or disorders involving the regulation of HMGCR expression and function (e.g., diseases or disorders know as risk factors for atherosclerotic cardiovascular disease (ASCVD) such as dyslipidemia).

HMGCR, 3-hydroxy-3-methylglutaryl-CoA reductase or HMGCo reductase, is the rate-limiting enzyme for the synthesis of cholesterol, which plays an important role in cell building and hormone production. HMGCR catalysis produces mevalonate, a precursor for cholesterol biosynthesis. Non-sterol and sterols derived from mevalonate regulate HMGCR via a negative feedback mechanism. In mammalian cells, HMGCR is normally suppressed by cholesterol derived from the internalization and degradation of low-density lipoprotein (LDL) via the LDL receptor.

Plasma cholesterol can become elevated because of genetics but is more often the result of poor diet that is high in fats and/or sugars and a sedentary lifestyle. Cholesterol can deposit in arteries and is an important determinant of atherosclerosis and ischemia. Competitive inhibitors of HMGCR induce the expression of LDL receptors in the liver, which in turn increases the catabolism of plasma LDL and lowers the plasma concentration of cholesterol. However, statins have multiple side effects, including insufficient lowering of cholesterol and/or LDL. Accordingly, more treatments are needed to lower cholesterol and to treat disease or disorders known as risk factors for (ASCVD) (e.g., dyslipidemia).

### SUMMARY OF INVENTION

In one aspect, the present disclosure describes RNAi agents for reducing HMGCR gene expression, wherein the RNAi agent comprises a delivery moiety of Formula I conjugated to R, wherein R is a double stranded RNA (dsRNA) comprising an antisense strand and a sense strand:

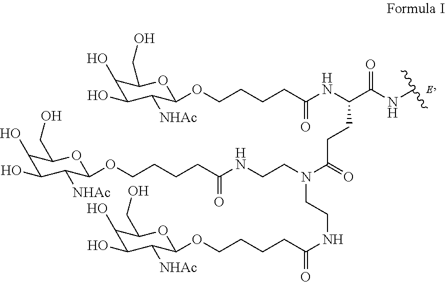

wherein R is conjugated to connection point E of Formula I, optionally via a linker, wherein the sense strand and the antisense strand form a duplex region, and wherein the antisense strand comprises a region of complementarity to a HMGCR mRNA target sequence of SEQ ID NO: 1, and wherein the sense and antisense strand each optionally comprise one or more modified nucleotides and one or more modified internucleotide linkages. In some embodiments, Formula I is conjugated to the sense strand, optionally via a linker. In some embodiments, Formula I is conjugated to the 3′ terminal nucleotide of the sense strand, optionally via a linker.

In some embodiments, the antisense strand is 15 to 50 nucleotides in length. In some embodiments, the sense strand is 15 to 50 nucleotides in length. In some embodiments, the antisense strand is between 18 and 23 nucleotides in length. In some embodiments, the sense strand is between 18 and 21 nucleotides in length. In some embodiments, the antisense strand is 23 nucleotides in length and the sense strand is 21 nucleotides in length.

In some embodiments, the sense strand or the antisense strand comprises a sequence selected from Table 2, 3, 4A, and 4B, as disclosed herein. In some embodiments, the sense strand and the antisense strand comprises a sequence selected from Table 2, 3, 4A, and 4B, as disclosed herein.

In some embodiments, R is conjugated to Formula I via a linker. In some embodiments, the linker comprises a linker of Formula II having connection points A and B or the linker comprises Formula III having connection points C and D, and wherein:

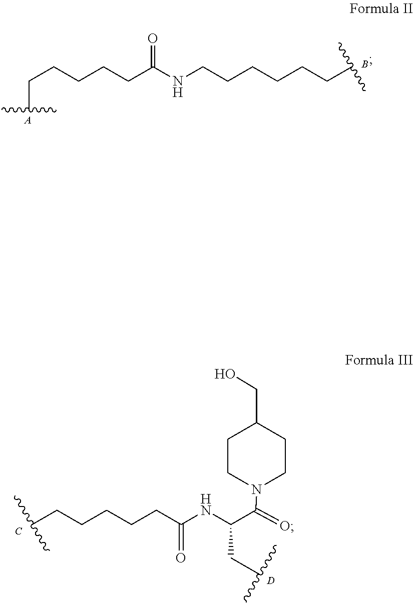

a. Formula I at connection point E is conjugated to Formula II at connection point A and Formula II is conjugated to a phosphate group or a phosphorothioate group at connection point B, and the phosphate group or phosphorothioate group is conjugated to R; or
b. Formula I at connection point E is conjugated to Formula III at connection point C and Formula III is conjugated to a phosphate group or phosphorothioate group at connection point D, and the phosphate group or phosphorothioate group is further conjugated to R.

In another aspect, the present disclosure provides pharmaceutical compositions comprising the RNAi agent described herein and one or more pharmaceutically acceptable excipients.

In another aspect, the present disclosure provides methods of treating a disease or disorder associated with ASCVD. In some embodiments, the disease or disorder is dyslipidemia, primary dysbetalipoproteinemia, hypertriglyceridemia, or atherosclerosis. In some embodiments, the present disclosure provides a method of treating dyslipidemia, comprising administering to a patient an RNAi agent or a pharmaceutical composition thereof as described herein.

In another aspect, the present disclosure provides an RNAi agent for the use in a therapy. In some embodiments, the present disclosure provides an RNAi agent for use in treating a disease or disorder associated with ASCVD. In some embodiments, the present disclosure provides an RNAi agent for use in treating a disease or disorder wherein the disease or disorder is dyslipidemia, primary dysbetalipoproteinemia, hypertriglyceridemia, or atherosclerosis.

In another aspect, the present disclosure provides the use of an RNAi agent for the manufacture of a medicament for use in a therapy. In some embodiments, the present disclosure provides an RNAi agent for the manufacture of a medicament for use in treating a disease or disorder associated with ASCVD. In some embodiments, the present disclosure provides an RNAi agent for the manufacture of a medicament for use in treating a disease or disorder, wherein the disease or disorder is dyslipidemia, primary dysbetalipoproteinemia, hypertriglyceridemia, or atherosclerosis.

### DETAILED DESCRIPTION

HMGCR siRNAs and ASOs have been described, but none have progressed for treatment in patients. Using the HMGCR RNAi agents herein to decrease expression of HMGCR can be employed to treat disease or disorders associated with ASCVD (e.g., dyslipidemia, primary dysbetalipoproteinemia, hypertriglyceridemia, or atherosclerosis). Such siRNAs may exhibit one or more of, e.g., as compared to other liver targeted siRNAs such as HMGCR siRNAs comprising a different delivery ligand, a different sequence, a differently modified sequence, or as compared to treatment with a vehicle control: improved knockdown in the liver; improved tissue exposure, improved exposure in liver hepatocytes; an improved durable response; an improved pharmacokinetic profile; fewer off target effects; and/or an improved toxicity profile. Other embodiments of the HMGCR RNAi agents herein may include one or more of fewer side effects as compared to statins or other standard of care; an improved toxicity profile; an improved safety profile; improved tolerability or compliance; and/or improved liver function tests. Still other siRNAs herein may have other benefits, e.g., in combination with any of the preceding or as a stand-alone benefit, including improved and/or simplified synthesis, synthetic processes with fewer degradation products; or any combination thereof.

The RNAi agents herein comprise a sense strand and an antisense strand, wherein each is an oligonucleotide. In some embodiments, the RNAi agents described herein also comprise a delivery moiety. As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acid polymers such as deoxyribonucleic acid (DNA) and ribonucleic acid (RNA).

As used herein, “oligonucleotide” means a short nucleic acid compound (e.g., less than about 100 nucleotides in length). An oligonucleotide may be single-stranded (ss) or double stranded (ds). An oligonucleotide may or may not have duplex regions. As a set of non-limiting examples, an oligonucleotide may be, but is not limited to, a small interfering RNA (siRNA), microRNA (miRNA), short hairpin RNA (shRNA), Dicer substrate interfering RNA (DsiRNA), or antisense oligonucleotide (ASO).

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

The term “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Clustal W2.0 or Clustal X2.0 or Megalign (DNASTAR) software. In one embodiment herein, sequence identity is calculated use Clustal W2.0 or Clustal X2.0. In another embodiment, sequence identity is calculated using Clustal W2.0. In another embodiment, sequence identity is calculated using Clustal X2.0. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. In some embodiments, percent sequence identity is the percent of nucleotide residues that are identical between two strands using the PID3 calculation, which is the number of identical nucleotide residues divided by the total number of nucleotides of the shortest of the two sequences, multiplied by 100. See, e.g., Raghava, G., Barton, G. J. Quantification of the variation in percentage identity for protein sequence alignments. BMC Bioinformatics 7, 415 (2006).

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

As used herein, “region of complementarity” means a nucleotide sequence of a nucleic acid (e.g., a double stranded oligonucleotide) that is sufficiently complementary to an antiparallel nucleotide sequence to permit hybridization between the two sequences of nucleotides under appropriate hybridization conditions (e.g., in a phosphate buffer, in a cell, etc.). In some embodiments, an oligonucleotide herein includes a targeting sequence having a region of complementary to a mRNA target sequence.

As used herein, “duplex,” in reference to nucleic acids or oligonucleotides, such as a sense strand or an antisense strand means a structure formed through hydrogen bonds of complementary base pairing of two antiparallel sequences of nucleotides under suitable conditions to promote such a structure. A duplex may form despite not having full complementarity between the two strands, or when an abasic nucleotide is present.

RNA interference is a specialized cellular process that utilizes RISC for degrading RNA in a sequence dependent manner. As used herein, “RNAi agent” means an agent comprising either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA. In some embodiments, the RNAi agent described herein also comprises a delivery moiety.

As used herein, a bond illustrated as

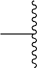

indicates a connection point as described therein. For example, if a generic variable, e.g., X, is stated to be attached at the connection point E as shown below, this is intended to show X is bonded to the atom of the connection point (see the scheme below).

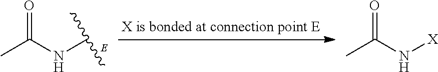

As used herein, an “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

As used herein, “treatment” or “treating” refers to all processes wherein there may be a slowing, controlling, delaying, or stopping of the progression of the disorders or disease disclosed herein, or ameliorating disorder or disease symptoms, and need not indicate a total elimination of all disorder or disease symptoms. Treatment includes administration of an RNAi agent or pharmaceutical composition thereof for treatment of a disease or condition in a mammal including a human.

As used herein, the term “disease or disorder associated with ASCVD” refers to any disease or disorder that is a risk factor for ASCVD.

Provided herein are RNAi agents for reducing HMGCR gene expression, wherein the RNAi agent comprises a delivery moiety of Formula I conjugated to R, wherein R is a double stranded RNA (dsRNA) comprising an antisense strand and a sense strand:

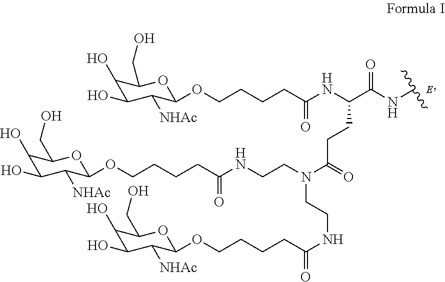

wherein R is conjugated to connection point E of Formula I, optionally via a linker, wherein the sense strand and the antisense strand form a duplex region, and wherein the antisense strand comprises a region of complementarity to a HMGCR mRNA target sequence of SEQ ID NO: 1, and wherein the sense and antisense strand each optionally comprise one or more modified nucleotides and one or more modified internucleotide linkages.

Also provided here are RNAi agents for reducing HMGCR gene expression, wherein the RNAi agent comprises a delivery moiety of Formula Ia conjugated to R, wherein R comprises an antisense strand and a sense strand:

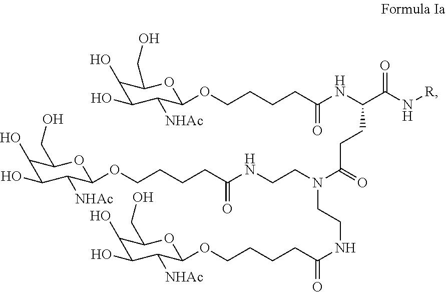

wherein R is conjugated to Formula Ia via a linker, wherein the sense strand and the antisense strand form a duplex region, and wherein the antisense strand comprises a region of complementarity to a HMGCR mRNA target sequence of SEQ ID NO: 1, and wherein the sense and antisense strand each optionally comprise one or more modified nucleotides and one or more modified internucleotide linkages.

Disclosed herein are RNAi agents for reducing HMGCR gene expression, wherein the RNAi agents comprise a sense strand and an antisense strand, wherein the sense strand and the antisense strand form a duplex region, and wherein the antisense strand comprises a region of complementarity of at least 15 nucleotides to the sequence as set forth in SEQ ID NO: 1, and wherein the sense strand and/or the antisense strand each optionally comprise one or more modified nucleotides and/or modified internucleotide linkages. In further embodiments, the antisense strand comprises at least 15 nucleotides of a sequence in Table 2. In further embodiments, the antisense strand comprises at least 18 nucleotides of a sequence in Table 2. In further embodiments, the RNAi agent reduces expression of the HMGCR gene in a liver cell, as compared to a control. In further embodiments, the RNAi agent reduces HMGCR gene expression by about 50% or greater in a cell expressing HMGCR, as compared to a control. In further embodiments, the RNAi agent reduces HMGCR gene expression by reducing the level of HMGCR mRNA transcript, the level of HMGCR protein, or both.

In further embodiments, the antisense strand is 15 to 50 nucleotides in length, and/or the sense strand is 15 to 50 nucleotides in length. In further embodiments, the sense and/or sense strand is independently 15 to 30 nucleotides in length. In further embodiments, the antisense strand is between 18 and 23 nucleotides in length. In further embodiments, the sense strand is between 18 and 21 nucleotides in length.

In further embodiments, the RNAi agent comprises an antisense strand that comprises at least 15 contiguous nucleotides of a sequence selected from the group consisting of SEQ ID NOs: 2 to 387. In still further embodiments, the antisense strand comprises at least 18 contiguous nucleotides of a sequence selected from the group consisting of SEQ ID NOs: 2 to 387.

In other further embodiments, the antisense strand comprises at least 18 contiguous nucleotides of a sequence selected from the group consisting of 18 contiguous nucleotides of a sequence selected from the group consisting of SEQ ID NOs:2 to 387.

In further embodiments, the antisense strand of the RNAi agent is 23 nucleotides in length. In still further embodiments, the sense strand is 21 nucleotides in length. In another embodiment, the antisense strand comprises a sequence selected from the group consisting of 774 to 1159, or a sequence having at least 90% sequence identity thereto. In another embodiment, the sense and antisense strand comprise a sequence selected from the sequences set forth in Table 3.

The sense strand and the antisense strand of the RNAi agents disclosed herein do not require full complementarity. Accordingly, in the RNAi agents disclosed herein, the duplex region between the sense strand and the antisense strand comprises 0, 1, 2, or 3 mismatches between the sense strand and the antisense strand. In further embodiments, the duplex region between the sense strand and the antisense strand consists of 0, 1, 2, or 3 mismatches between the sense strand and the antisense strand.

In further embodiments, the sense strand comprises a sequence selected from the group consisting of SEQ ID NO: 388 to 773.

In further embodiments, the sense strand and the antisense strand each independently comprise one or more modified nucleotides, such as 2′ fluoro modified nucleotides or 2′-O-methyl modified nucleotides. In still further embodiments of the RNAi agents disclosed herein, each nucleotide of the sense strand and each nucleotide of the antisense strand is a modified nucleotide. In further embodiments, each nucleotide is a 2′ fluoro modified nucleotide or a 2′-O-methyl modified nucleotide.

In further embodiments of the RNAi agents disclosed herein, the antisense strand is 23 nucleotides in length, each nucleotide of the antisense strand is a modified nucleotide, and 2′ fluoro modified nucleotides are present at
a. Positions 2, 3, 7, 14, and 16 from the 5′ end of the antisense strand; or
b. Positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.
In further embodiments, the nucleotides that are not 2′ fluoro modified nucleotides are 2′-O-methyl modified nucleotides.

In further embodiments of the RNAi agents disclosed herein, the sense strand and antisense strand each independently comprise one or more modified internucleotide linkages, and each modified internucleotide linkage is a phosphorothioate linkage. In further embodiments, the sense strand and antisense strand each independently comprise four phosphorothioate linkages. In still further embodiments, the two terminal nucleotides at each of the 5′ and 3′ ends of each of the sense and antisense strand are phosphorothioate linkages.

In other embodiments, the 5′ nucleotide of the antisense strand comprises a phosphate group or a phosphate analog. As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

In further embodiments of the RNAi agents disclosed herein, the antisense strand comprises a sequence selected from the group consisting of SEQ ID NOs: 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, or a sequence having at least 90% sequence identity thereto, wherein the 5′ terminal nucleotide of the antisense strand comprises a 5′ a vinyl phosphonate, a phosphate, or a hydroxyl group. In other embodiments, the phosphate group listed at the 5′ end of the recited SEQ ID NO: is removed and replaced with an OH. In other embodiments, the phosphate group listed at the 5′ end of the recited SEQ ID NO: is replaced with a 5′ vinylphosphonate.

In further embodiments of the RNAi agents disclosed herein, the antisense strand comprises a sequence selected from the group consisting of SEQ ID Nos: 1932-2317 or a sequence having at least 90% sequence identity thereto.

In further embodiments of the RNAi agents disclosed herein, the antisense strand comprises a sequence selected from the group consisting of SEQ ID Nos: 1932-2317 or a sequence having at least 95% sequence identity thereto.

In further embodiments the sense strand comprises a sequence selected from the group consisting of SEQ ID NOs: 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, or a sequence having at least 90% sequence identity thereto.

In still further embodiments of the RNAi agents disclosed herein, the sense strand and antisense strand are a pair of oligonucleotide sequences selected from Table 4A, 4B, or a sequence that is at least 80, 81, 82, 83, 84, 85, 86, 87, 88, 89, 90, 91, 92, 93, 94, 95, 96, 97, 98, or 99 percent identical to the sequence in Table 4A or 4B. In further embodiments, 1, 2, or 3 mismatches are introduced into the sense strand of the pair in Table 4A or 4B. In further embodiments, 1, 2, or both terminal nucleotides of 5′ end of the antisense strand are changed.

In some embodiments of the RNAi agents herein, the antisense strand comprises a first nucleic acid sequence that has at least 90% sequence identity to an antisense sequence corresponding to a Duplex NO: in Table 4A or 4B, and the sense strand comprises a second nucleic acid sequence that has at least 90% sequence identity to a sense sequence corresponding to the same Duplex No: in Table 4A or 4B. For example, in one embodiment, the antisense strand comprises a first nucleic acid sequence that has at least 90% sequence identity to an antisense sequence corresponding to a Duplex NO: 387 in Table 4A, that is, a first nucleic acid sequence that has at least 90% sequence identity to SEQ ID NO: 1161, and the sense strand comprises a second nucleic acid sequence that has at least 90% sequence identity to a sense sequence corresponding to Duplex No: 387 in Table 4A, that is, SEQ ID NO: 1160. In further embodiments, the 5′ phosphate of the antisense strand is further modified/replaced, and is a 5′ vinylphophonate or an OH group.

A duplex, (e.g., a “Duplex No.:”), as shown herein, (see, e.g., Tables 4A and 4B), corresponds to a specific sense and antisense strand.

In further embodiments, the 5′ terminal nucleotide of the antisense strand is substituted such that the final sequence contains a vinylphosphonate, a phosphate group, or an OH group. For example, for antisense sequences of SEQ ID NOs 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, or a sequence having at least 90% sequence identity thereto, the 5′ phosphate group is replaced with an OH group.

In further embodiments of the RNAi agents disclosed herein, the antisense strand comprises a sequence selected from the group consisting of SEQ ID Nos: 1932-2317 or a sequence having at least 90% sequence identity thereto.

In other embodiments disclosed herein are RNAi agents having a delivery moiety of Formula I conjugated to R:

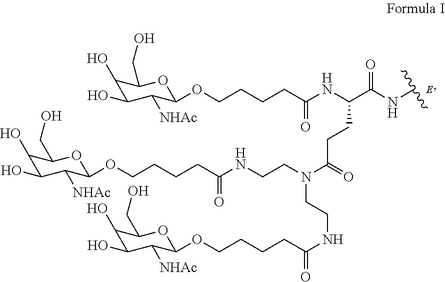 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

wherein R comprises a sense strand and an antisense strand, wherein the antisense strand comprises at least 15 contiguous nucleotides that have complementarity to HMGCR mRNA target sequence of SEQ ID NO:1, and wherein the sense strand and the antisense strand form a region of complementarity of at least 15 nucleotides, and wherein the sense strand and antisense strand are each independently 18 to 23 nucleotides in length, and optionally wherein the sense strand and antisense strand each independently comprise one or more modified nucleotides, and optionally wherein the sense strand and the antisense strand each independently comprise one or more modified internucleotide linkages, and wherein R is conjugated to Formula I via a linker. In further embodiments, the sense or the antisense strand is selected from Table 2, 3, 4A or 4B disclosed herein. In other embodiments, the antisense or antisense strand of the RNAi agent has an antisense strand sequence and/or a sense strand sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity to the corresponding sequence selected from a Table 2, 3, 4A or 4B herein.

In other embodiments, the RNAi agent disclosed herein comprises a linker. In further embodiments, R is conjugated to Formula I via a linker. In other further embodiments R is conjugated to Formula I via a linker. In further embodiments, the linker comprises a linker of Formula II having connection points A and B or the linker comprises Formula III having connection points C and D, and wherein:

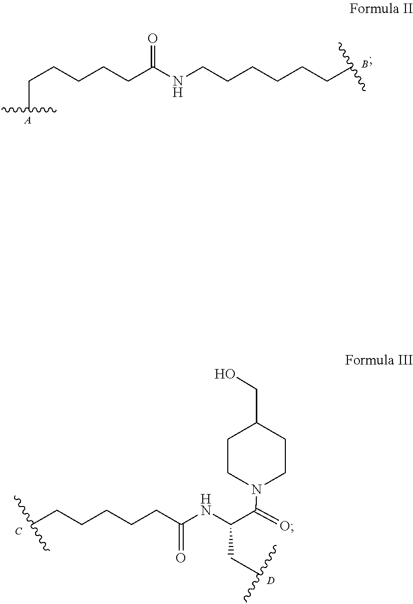

a. the RNAi agent comprises Formula I conjugated to Formula II at connection point A and Formula II is conjugated to a phosphate group at connection point B, and the phosphate group is conjugated to R; or
b. the RNAi agent comprises Formula I conjugated to Formula III at connection point C and Formula III is conjugated to a phosphate group at connection point D, and the phosphate group is further conjugated to R.

In other embodiments wherein the RNAi agent comprises a linker, R is conjugated to Formula I via a linker, and the linker is a linker comprising Formula III having connection points C and D:

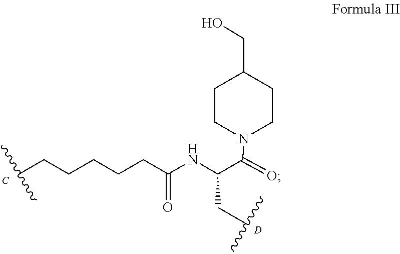

and wherein the RNAi agent comprises Formula I conjugated to Formula III at connection point C and Formula III is conjugated to a phosphate group at connection point D, and the phosphate group is further conjugated to R.

In still other embodiments, the RNAi agent is capable of decreasing expression of the HMGCR gene in a liver cell. In other embodiments, the RNAi agents disclosed herein are for use in therapy. In some embodiments, the use is for the treatment of a disease or disorder associated with ASCVD. In further embodiments, the use is for the treatment of dyslipidemia, primary dysbetalipoproteinemia, hypertriglyceridemia, or atherosclerosis. In some embodiments, the use is for the treatment of dyslipidemia. In some embodiments, the use is for the treatment of primary dysbetalipoproteinemia. In some embodiments, the use is for the treatment of hypertriglyceridemia. In some embodiments, the use is for the treatment of atherosclerosis. In further embodiments, the dyslipidemia is hypercholesteremia. In other embodiments, the use is to reduce the risk of one or more of myocardial infarction (MI), stroke, revascularization procedures, and angina. In other embodiments, the use to reduce such risk is in adult patients without cardiovascular heart disease (CHD), but with one or more risk factors for any one or more of the recited health events. In other embodiments, the use is to reduce the risk of MI and/or stroke, e.g., in adult patients with type 2 diabetes without CHD, but with one or more risk factors. In other embodiments, the use is to reduce the risk of one or more of non-fatal MI, fatal and non-fatal stroke, revascularization procedures, hospitalization for CHF, and angina, in adult patients with CHD. In other embodiments, the use is to reduce one or more of elevated total-C, LDL-C, apo B, and TG levels, and/or to increase HDLC in adult patients with primary hyperlipidemia (heterozygous familial and nonfamilial) and mixed dyslipidemia. In other embodiments, the use is to reduce elevated triglycerides (TG) in adult patients with hypertriglyceridemia and primary dysbetalipoproteinemia. In other embodiments, the use is to reduce total-C and LDL-C in patients with homozygous familial hypercholesterolemia (HoFH). In still other embodiments, the use is to reduce one or more of elevated total-C, LDL-C, and apo B levels in pediatric patients, 10 years to 17 years of age, with heterozygous familial hypercholesterolemia (HeFH), e.g. after failing an adequate trial of diet therapy. In other embodiments, the use is for treatment of any of the preceding, after statin use failed to control one or more symptoms, e.g. failed to reduce one or more of elevated total-C, LDL-C, apo B, and/or failed to increase HDLC. In other embodiments, the use is for the treatment of any of the preceding, in patients that are statin intolerant; in further embodiments, the use is for lowering LDL-C in patients that are statin intolerant. In further embodiments, the use is for any of the preceding uses, after alteration of diet failed to control one or more symptoms. In other further embodiments, the use is for any of the preceding uses, as an adjunct therapy to diet.

The RNAi agents may be formulated into pharmaceutical compositions. Accordingly, disclosed herein are pharmaceutical compositions comprising the RNAi agent disclosed herein, and one or more pharmaceutically acceptable excipients. Pharmaceutical compositions can be prepared by methods well known in the art (e.g., Remington: The Science and Practice of Pharmacy, 23rd edition (2020), A. Loyd et al., Academic Press).

In other embodiment are uses of the RNAi agents herein for the manufacture of a medicament for the treatment of dyslipidemia or any of the uses recited in the preceding paragraph.

In other embodiments are methods of treating dyslipidemia, in patients in need thereof, comprising administering an RNAi agent disclosed herein, or a pharmaceutical composition thereof. In other embodiments are methods of treating dyslipidemia, or any of the uses recited above, in patients in need thereof, or a pharmaceutical composition thereof. In other embodiments are methods of treating a patient unable to achieve lipid levels following statin and/or diet therapy, comprising administering to the patient an RNAi agent disclosed herein, or a pharmaceutical composition thereof.

The RNAi agent can be administered to the patient intravenously or subcutaneously.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In other embodiments are methods of decreasing HMGCR expression in a cell, comprising contacting the cell with an RNAi agent disclosed herein, and incubating the cell for a time sufficient for decreasing the level of HMGCR mRNA by at least 50% as compared to an untreated or control treated cell.

Certain abbreviations are defined as follows: “1,2-DCE” refers to 1,2-dichloroethane; “DCM” refers to dichloromethane; “DIEA” refers to N,N-diisopropylethylamine; “DMF” refers to N,N-dimethylformamide; “DMAP” refers to 4-dimethylaminopyridine; “DMTCl” refers to 4,4′-dimethoxytrityl chloride; “DPP4” refers to dipeptidyl peptidase; “EDC” refers to 1-ethyl-3-(3-dimethylaminopropyl)carbodiimide; “EtOAc” refers to ethyl acetate; “GalNAc” refers to N-acetylgalactosamine; “HATU” refers to 1-[bis(dimethylamino)methylene]-1H-1,2,3-triazolo[4,5-b]pyridinium 3-oxid hexafluorophosphate; “HBTU” refers to O-(benzotriazol-1-yl)-N,N,N′,N′-tetramethyluronium hexafluorophosphate; “HOBt” refers to 1-hydroxybenzotriazole hydrate; “HPRT” refers to hypoxanthine-guanine phosphoribosyltransferase; “IPA” refers to isopropanol and isopropyl alcohol; “LDHA” refers to lactate dehydrogenase-A; “MeCN” refers to acetonitrile; “MeOH” refers to methanol and methyl alcohol; “MWCO” refers to molecular weight cut-off; “NHS” refers to N-hydroxysuccinimide; “OD” refers to optical density; “PBS” refers to phosphate-buffered saline; “PhSiH₃” refers to phenylsilane; “PTS” refers to portable endotoxin testing system; “siRNA” refers to small interfering ribonucleic acid; “TEA” refers to triethylamine; “TFA” refers to trifluoroacetic acid; “THF” refers to tetrahydrofuran; “TLC” refers to thin line chromatography; and “TMP” refers to 2,2,6,6-tetramethylpiperidine.

A delivery moiety comprising Formula I may be made by the following non-limiting synthetic steps and schemes.

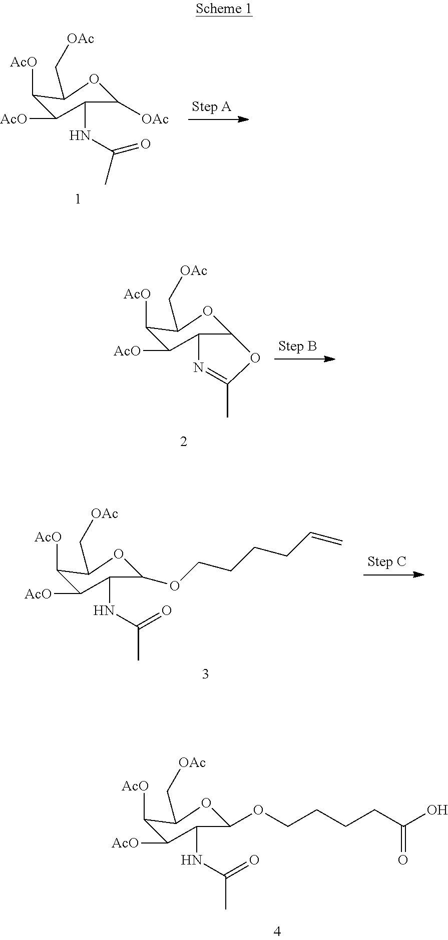 [SMILES: CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O ; C=CCCCCOC1OC(COC(C)=O)C(OC(C)=O)C(OC(C)=O)C1NC(C)=O ; CC(=O)NC1C(OCCCCC(=O)O)OC(COC(C)=O)C(OC(C)=O)C1OC(C)=O]

Scheme 1, step A, depicts the cyclization of compound (1) using trimethyl trifluoromethanesulfonate in a solvent such as 1,2-DCE to give compound (2). Step B shows the addition of hex-5-en-1-ol to compound (2) using trimethylsilyl trifluoromethanesulfonate in a solvent such as 1,2-DCE to give compound (3). The oxidation of compound (3) using an appropriate oxidizing agent such as sodium periodate with a catalyst such as ruthenium(III) chloride to give compound (4) is shown in step C.

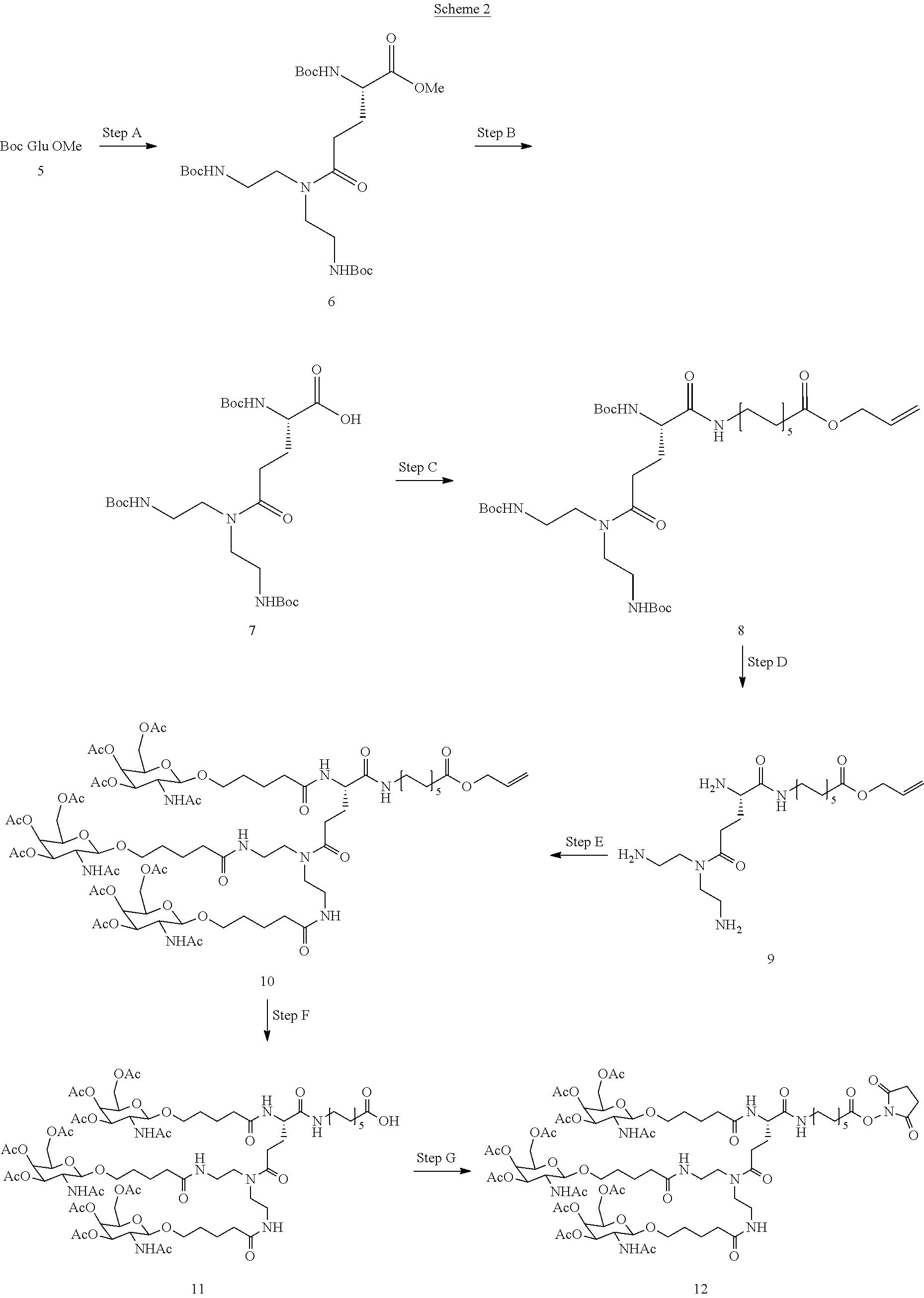

Scheme 2, step A, shows an amide coupling between compound (5) and tert-butyl N-[2-[2-(tert-butoxycarbonylamino)ethylamino]ethyl]carbamate using HBTU and HOBt with an appropriate base such as DIEA in a solvent such as DMF to give compound (6). Step B depicts a basic hydrolysis of compound (6) using a base such as aqueous NaOH in a THF and MeOH solvent system to give compound (7). Step C shows an amide coupling between compound (7) and allyl 11-aminoundecanoate hydrochloride using HATU with an appropriate base such as DIEA in a solvent such as DMF to give compound (8). Step D shows the acidic deprotection of compound (8) with TFA in a solvent such as DCM to give compound (9). The amide coupling between compound (9) and compound (4) using EDC and HOBt in a solvent such as DCM to give compound (10) is shown in step E. Step F shows the deprotection of compound (10) with tetrakis(triphenylphosphine)palladium and PhSiH₃ in a solvent such as DCM to give compound (11). Step F depicts the coupling of compound (11) with NHS using EDC in a solvent such as DCM to give compound (12).

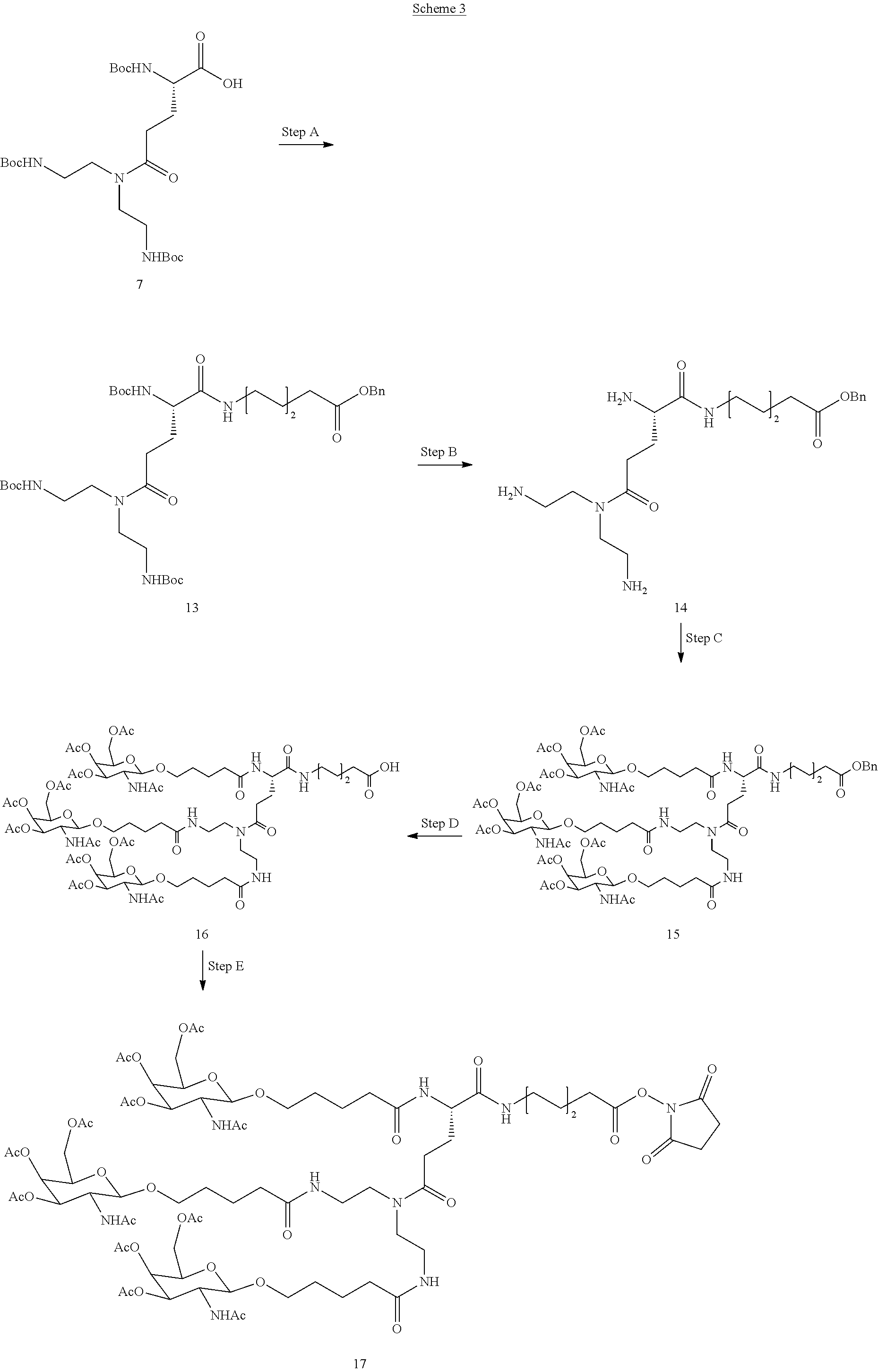

Scheme 3, steps A-C are essentially analogous to those of scheme 2, steps C-E beginning with compound (7) to give compounds (13), (14), and (15). Step D depicts the hydrogenation of compound (15) using palladium on carbon in a solvent such as MeOH to give compound (16). Step E is essentially analogous to the preparation of scheme 2, step G to give compound (17).

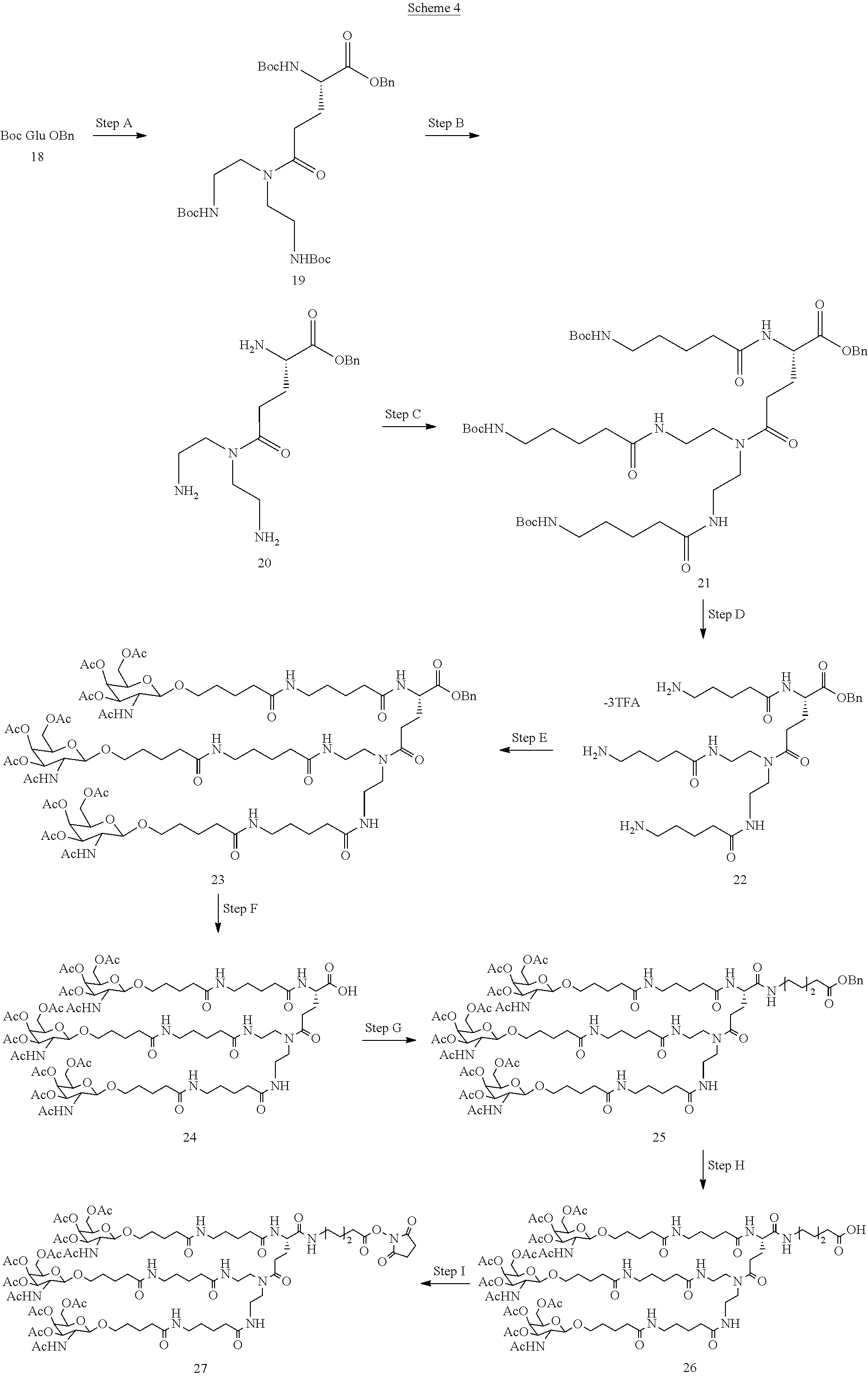

Scheme 4, steps A-I, are composed of a series of amide couplings and deprotections using methods essentially analogous to those found in schemes 2 and 3 beginning with compound (18) to give compound (27).

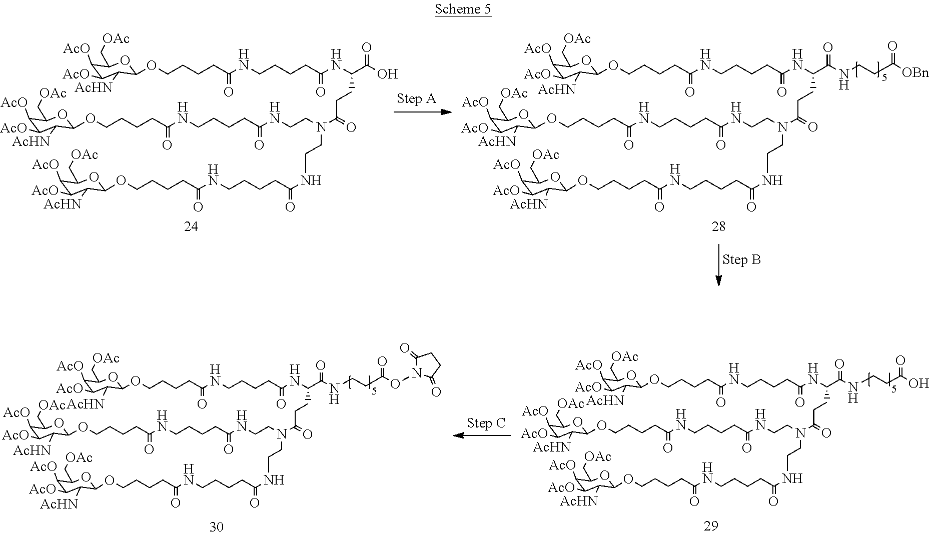

Scheme 5, steps A-C depict methods essentially analogous to those found in scheme 4, steps G-I beginning with compound (24) to give compound (30).

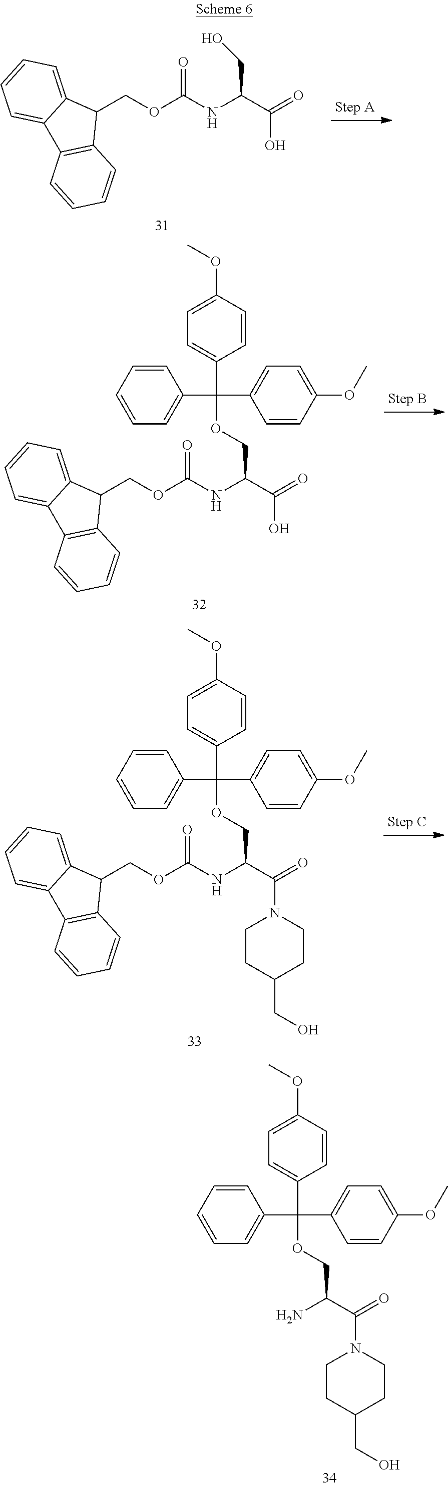 [SMILES: O=C(N[C@@H](CO)C(=O)O)OCC1c2ccccc2-c2ccccc21 ; COc1ccc(C(OC[C@H](NC(=O)OCC2c3ccccc3-c3ccccc32)C(=O)O)(c2ccccc2)c2ccc(OC)cc2)cc1 ; COc1ccc(C(OC[C@H](NC(=O)OCC2c3ccccc3-c3ccccc32)C(=O)N2CCC(CO)CC2)(c2ccccc2)c2ccc(OC)cc2)cc1 ; COc1ccc(C(OC[C@H](N)C(=O)N2CCC(CO)CC2)(c2ccccc2)c2ccc(OC)cc2)cc1]

Scheme 6, step A depicts the protection of compound (31) using DMTCl with a suitable base such as DIEA in a solvent such as DCM to give compound (32). Step B shows an amide coupling between compound (32) and piperidin-4-yl methanol using HBTU and HOBt with TMP in a solvent such as DCM to give compound (33). The deprotection of compound (33) with 20% piperidine in DMF to give compound (34) is shown in step C.

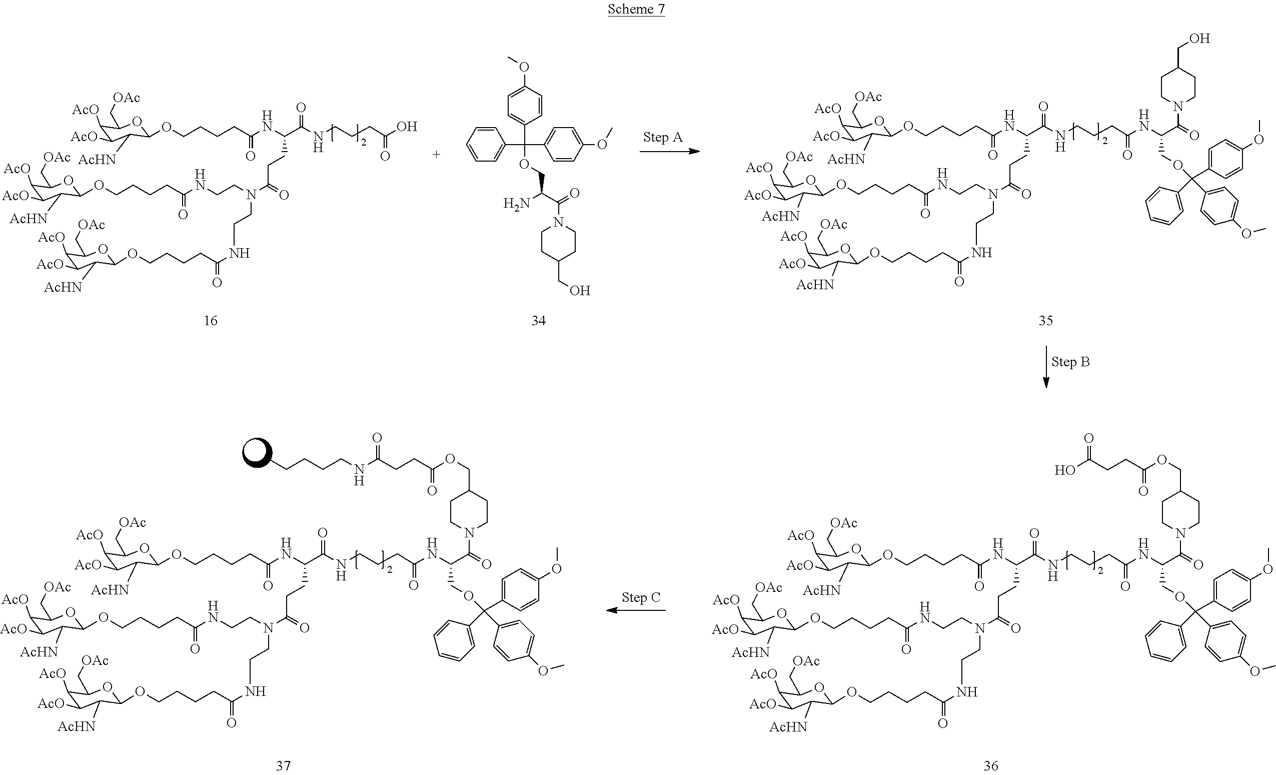

Scheme 7, step A is essentially analogous to scheme 2, step A to give compound (35) from the coupling of compounds (16) and (34). Step B shows the formation of compound (36) by adding succinic anhydride to compound (35) in an appropriate solvent such as DCM with a base system of TEA and DMAP. Step C depicts the loading of compound (36) onto resin with 2-(1H-benzotriazol-1-yl)-1,1,3,3-tetramethyluronium hexafluorophosphate and a base such as DIEA in a solvent system such as MeCN and DCM to give compound (37).

### Preparation 1

### (6,7-Diacetoxy-2-methyl-5,6,7,7a-tetrahydro-3aH-pyrano[3,2-d]oxazol-5-yl)methyl acetate

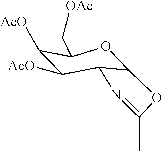 [SMILES: CC(=O)OCC1OC2OC(C)=NC2C(OC(C)=O)C1OC(C)=O]

To a solution of (5-acetamido-3,4,6-triacetoxy-tetrahydropyran-2-yl)methyl acetate (9.00 g, 23.1 mmol) in 1,2-DCE (46 mL) is added trimethylsilyl trifluoromethanesulfonate (6.5 mL, 35 mmol). The mixture is heated to 50° C. and stirred for 18 hours. After this time, the mixture is diluted with DCM (200 mL), washed with saturated NaHCO₃ (200 mL), and saturated aqueous sodium chloride solution (200 mL), dried over sodium sulfate, filtered, and concentrated in vacuo. The resulting residue is purified by silica gel flash chromatography eluting with 0-10% MeOH/DCM to give the title compound (6.434 g, 84%). ES/MS m/z 330 (M+H).

### Preparation 2

### (5-Acetamido-3,4-diacetoxy-6-hex-5-enoxy-tetrahydropyran-2-yl)methyl acetate

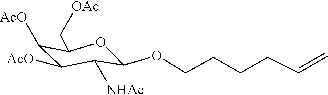 [SMILES: C=CCCCCOC1OC(COC(C)=O)C(OC(C)=O)C(OC(C)=O)C1NC(C)=O]

To a solution of (6,7-diacetoxy-2-methyl-5,6,7,7a-tetrahydro-3aH-pyrano[3,2-d]oxazol-5-yl)methyl acetate (30.43 g, 92.42 mmol) in 1,2-DCE (231 mL) is added hex-5-en-1-ol (22.2 mL, 185 mmol) followed by activated powdered 4 Å molecular sieves (15.6 g). The suspension is stirred at ambient temperature for 30 minutes and trimethylsilyl trifluoromethanesulfonate (19 mL, 101.9 mmol) is then added. The mixture is stirred at ambient temperature for 18 hours. After this time, the solution is filtered through diatomaceous earth and concentrated in vacuo. The resulting residue is purified by silica gel flash chromatography eluting with 30-100% EtOAc/hexanes to give the title compound (34.76 g, 86%). ES/MS m/z 430.4 (M+H).

### Preparation 3

### 5-[3-Acetamido-4,5-diacetoxy-6-(acetoxymethyl)tetrahydropyran-2-yl]oxypentanoic acid

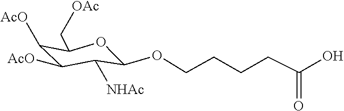 [SMILES: CC(=O)NC1C(OCCCCC(=O)O)OC(COC(C)=O)C(OC(C)=O)C1OC(C)=O]

A solution of (5-acetamido-3,4-diacetoxy-6-hex-5-enoxy-tetrahydropyran-2-yl)methyl acetate (34.76 g, 80.93 mmol) in MeCN (174 mL) and DCM (174 mL) is cooled to 0° C. A solution of sodium periodate (22.4 g, 104.7 mmol) is added and stirring is continued at 0° C. for 10 minutes. After this time, ruthenium(III) chloride (270 mg, 1.3 mmol) is added and the mixture is stirred while warming to ambient temperature. After stirring for 2 hours, additional sodium periodate (66 g, 308.4 mmol) is added and stirring is continued for 18 hours. After this time, the mixture is extracted with 3:1 CH₃Cl:IPA (2×500 mL), washed with saturated aqueous sodium chloride solution (1 L), dried over sodium sulfate, filtered, and concentrated in vacuo. The resulting residue is purified by silica gel flash chromatography eluting with 0-40% MeOH/DCM to give the title compound (29.75 g, 82%). ES/MS m/z 448.4 (M+H).

### Preparation 4

### Benzyl 6-aminohexanoate hydrochloride

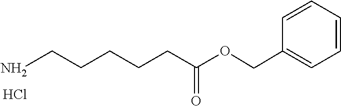 [SMILES: Cl ; NCCCCCC(=O)OCc1ccccc1]

To a suspension of 6-aminohexanoic acid (5.00 g, 38.1 mmol) in THF (38 mL) is added benzyl alcohol (47 mL, 453.7 mmol) and the mixture is cooled to 0° C. Thionyl chloride (8.6 mL, 120 mmol) is added dropwise and the mixture is stirred for 18 hours while warming to ambient temperature. After this time, ether (166 mL) is added and the reaction vessel is transferred to a freezer at −20° C. for 1 hour. After this time, the solid precipitate is collected by filtration to give the title compound (8.57 g, 81%). ES/MS m/z 222 (M+H).

### Preparation 5

### Benzyl 11-aminoundecanoate hydrochloride

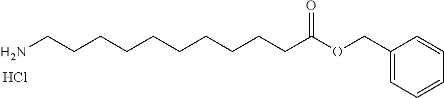 [SMILES: Cl ; NCCCCCCCCCCC(=O)OCc1ccccc1]

The title compound is prepared from 11-aminoundecanoic acid in a manner essentially analogous to the method of preparation 4. ES/MS m/z 292.2 (M+H).

### Preparation 6

### Allyl 11-aminoundecanoate hydrochloride

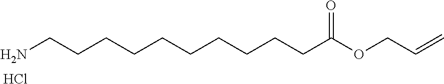 [SMILES: C=CCOC(=O)CCCCCCCCCCN ; Cl]

A vessel is charged with 11-aminoundecanoic acid (9.00 g, 44.7 mmol) in allyl alcohol (42 mL) and the mixture is cooled to 0° C. Thionyl chloride (6.5 mL, 89.4 mmol) is added and the mixture is stirred for 18 hours while warming to ambient temperature. After this time, the mixture is concentrated in vacuo and ether (200 mL) is added to the residue to obtain a white suspension. The mixture is stirred at ambient temperature for 10 minutes and the solid precipitate is collected by filtration to obtain the product (12.0 g, 97%). ES/MS m/z 242.2 (M+H).

### Preparation 7

### (2S)-3-[Bis(4-methoxyphenyl)-phenyl-methoxy]-2-(9H-fluoren-9ylmethoxycarbonylamino) propanoic acid

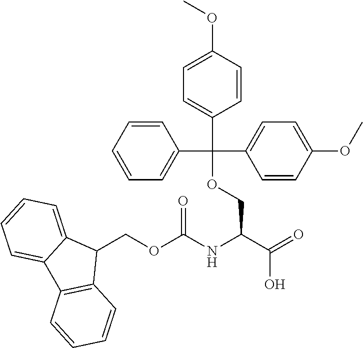 [SMILES: COc1ccc(C(OC[C@H](NC(=O)OCC2c3ccccc3-c3ccccc32)C(=O)O)(c2ccccc2)c2ccc(OC)cc2)cc1]

To a stirring solution of (2S)-2-(9H-fluoren-9-ylmethoxycarbonylamino)-3-hydroxy-propanoic acid (40 g, 0.122 mol) in dry DCM (400 mL) is added DIEA (64 mL, 0.366 mol) at 0° C. under inert atmosphere. To this, a solution of DMTCl (49.6 g, 0.146 mol) in DCM (200 mL) is added slowly. The resulting reaction mixture is brought to ambient temperature and stirred for 16 hours. After this time, the reaction mixture is diluted with water (12.5 vol) and extracted with DCM (25 vol). The organic layer is dried over anhydrous sodium sulphate, filtered, and concentrated in vacuo. The crude obtained is washed with 10% EtOAc/hexane (12.5 vol) and dried under vacuum to give the title compound as a pale brown solid (62 g, crude). This material was taken to next step without any further purification.

TLC: 5% MeOH/CH₂Cl₂ (R_{f}: 0.5) UV, 254 nM.

### Preparation 8

### 9H-Fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate

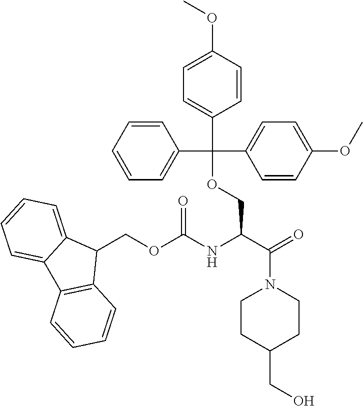 [SMILES: COc1ccc(C(OC[C@H](NC(=O)OCC2c3ccccc3-c3ccccc32)C(=O)N2CCC(CO)CC2)(c2ccccc2)c2ccc(OC)cc2)cc1]

To a stirring solution of (2S)-3-[bis(4-methoxyphenyl)-phenyl-methoxy]-2-(9H-fluoren-9ylmethoxycarbonylamino) propanoic acid (62 g, 0.103 mol) in DCM (750 mL) are added slowly HBTU (78.3 g, 0.206 mol), HOBt (27.9 g, 0.206 mol), and piperidin-4-yl methanol (15.4 g, 0.134 mol) followed by TMP (15 mL, 0.113 mol) at 0° C. under inert atmosphere. The resulting reaction mixture is brought to ambient temperature and stirred for 4 hours. After this time, the reaction mixture is diluted with water (8 vol) and extracted with DCM (15 vol). The organic layer is dried over anhydrous sodium sulphate, filtered, and concentrated in vacuo. The resulting residue is purified by silica gel flash chromatography eluting with 20-40% EtOAc/hexane and 1% MeOH/DCM to give the title compound (40 g, 52% over two steps). ¹H NMR (DMSO-d₆) δ 7.88 (br d, J=7.5 Hz, 2H), 7.79-7.59 (m, 3H), 7.45-7.12 (m, 13H), 6.92-6.76 (m, 4H), 4.79-4.44 (m, 2H), 4.32 (br d, J=11.4 Hz, 2H), 4.20 (br s, 2H), 3.71 (s, 6H), 3.21 (br s, 4H), 2.99-2.79 (m, 1H), 2.69 (br s, 2H), 1.81-1.43 (m, 3H), 1.08-0.73 (m, 2H).

### Preparation 9

### (2S)-2-Amino-3-[bis(4-methoxyphenyl)-phenyl-methoxy]-1-[4-(hydroxymethyl)-1-piperidyl]propan-1-one

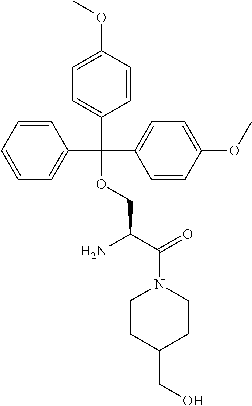 [SMILES: COc1ccc(C(OC[C@H](N)C(=O)N2CCC(CO)CC2)(c2ccccc2)c2ccc(OC)cc2)cc1]

A solution of 20% piperidine in DMF (400 mL) is added slowly to 9H-fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate (40 g, 0.055 mol) at 0° C. under inert atmosphere. The resulting reaction mixture is stirred at ambient temperature for 1 hour. After this time, the mixture is diluted with water (15 vol) and extracted with EtOAc (30 vol). The organic layer is dried over anhydrous sodium sulphate, filtered, and concentrated in vacuo. The resulting residue is purified by silica gel flash chromatography eluting with 1-8% MeOH/DCM to give the title compound as an off white solid (13 g, 47%). ES/MS m/z 1009.5 (2M+H).

### Preparation 10

### Methyl (2S)-5-[bis[2-(tert-butoxycarbonylamino)ethyl]amino]-2-(tert-butoxycarbonylamino)-5-oxo-pentanoate

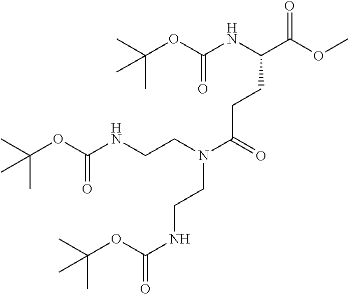 [SMILES: COC(=O)[C@H](CCC(=O)N(CCNC(=O)OC(C)(C)C)CCNC(=O)OC(C)(C)C)NC(=O)OC(C)(C)C]

To a flask containing (S)-4-((tert-butoxycarbonyl)amino)-5-methoxy-5-oxopentanoic acid (7.00 g, 26.8 mmol) and HOBt (4.16 g, 30.8 mmol) are added DMF (179 mL) and (2-(1H-benzotriazol-1-yl)-1,1,3,3-tetramethyluronium hexafluorophosphate (11.7 g, 30.9 mmol). DIEA (14 mL, 80.3 mmol) is added and the mixture is stirred at ambient temperature for 5 minutes. After this time, tert-butyl N-[2-[2-(tert-butoxycarbonylamino)ethylamino]ethyl]carbamate (8.94 g, 29.5 mmol) is added in one portion and stirring is continued at ambient temperature. After stirring for 18 hours, the mixture is diluted with EtOAc (400 mL), washed with water (2×400 mL) and saturated aqueous sodium chloride solution (400 mL), dried over sodium sulfate, filtered, and concentrated in vacuo. The resulting residue is purified by silica gel flash chromatography eluting with 40-100% EtOAc/hexanes to give the title compound (13.01 g, 89%). ES/MS m/z 547.40 (M+H).

### Preparation 11

### (2S)-5-[Bis[2-(tert-butoxycarbonylamino)ethyl]amino]-2-(tert-butoxycarbonylamino)-5-oxo-pentanoic acid

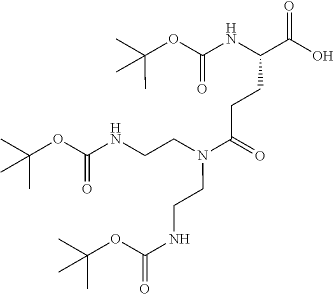 [SMILES: CC(C)(C)OC(=O)NCCN(CCNC(=O)OC(C)(C)C)C(=O)CC[C@H](NC(=O)OC(C)(C)C)C(=O)O]

A flask is charged with methyl (2S)-5-[bis[2-(tert-butoxycarbonylamino)ethyl]amino]-2-(tert-butoxycarbonylamino)-5-oxo-pentanoate (13.01 g, 23.8 mmol), THF (120 mL), and MeOH (120 mL). 1N NaOH (71 mL, 71 mmol) is added and the mixture is stirred at ambient temperature. After 1 hour, the mixture is concentrated in vacuo and redissolved in water (300 mL). 5N HCl (12 mL) is added to bring the pH to 4. The mixture is extracted with DCM (3×300 mL) and the combined organic layers are washed with saturated aqueous sodium chloride solution (1 L), dried over sodium sulfate, filtered, and concentrated to give the title compound (12.41 g, 98%). ES/MS m/z 531.60 (M−H).

### Preparation 12

### Allyl 11-[[(2S)-5-[bis[2-(tert-butoxycarbonylamino)ethyl]amino]-2-(tert-butoxycarbonylamino)-5-oxo-pentanoyl]amino]undecanoate

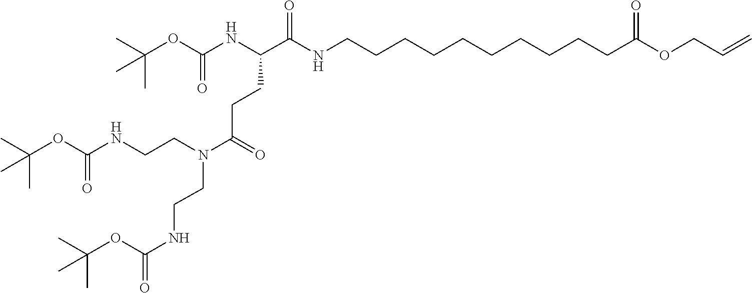 [SMILES: C=CCOC(=O)CCCCCCCCCCNC(=O)[C@H](CCC(=O)N(CCNC(=O)OC(C)(C)C)CCNC(=O)OC(C)(C)C)NC(=O)OC(C)(C)C]

To a flask containing (2S)-5-[bis[2-(tert-butoxycarbonylamino)ethyl]amino]-2-(tert-butoxycarbonylamino)-5-oxo-pentanoic acid (500 mg, 0.94 mmol) and allyl 11-aminoundecanoate hydrochloride (313 mg, 1.13 mmol) is added DMF (6.25 mL) and (1-[bis(dimethylamino)methylene]-1H-1,2,3-triazolo[4,5-b]pyridinium 3-oxide hexafluorophosphate (428 mg, 1.12 mmol). Following addition of DIEA (0.5 mL, 3 mmol) the mixture is stirred at ambient temperature for 18 hours. After this time, the mixture is diluted with EtOAc (200 mL), washed with water (3×200 mL) and saturated aqueous sodium chloride solution (200 mL), dried over sodium sulfate, filtered, and concentrated in vacuo. The resulting residue is purified by silica gel flash chromatography eluting with 40-100% EtOAc/hexanes to give the title compound (687 mg, 97%). ¹H NMR (DMSO-d6) δ 7.78-7.64 (m, 1H), 6.98-6.7 (m, 2H), 5.96-5.84 (m, 1H), 5.31-5.25 (m, 1H), 5.23-5.17 (m, 1H), 4.56-4.50 (m, 2H), 3.88-3.67 (m, 1H), 3.30-3.19 (m, 4H), 3.11-2.91 (m, 6H), 2.35-2.12 (m, 4H), 1.88-1.65 (m, 2H), 1.58-1.47 (m, 2H), 1.46-1.30 (m, 30H), 1.30-1.18 (m, 12H).

### Preparation 13

### Allyl (S)-11-(2-amino-5-(bis(2-aminoethyl)amino)-5-oxopentanamido)undecanoate

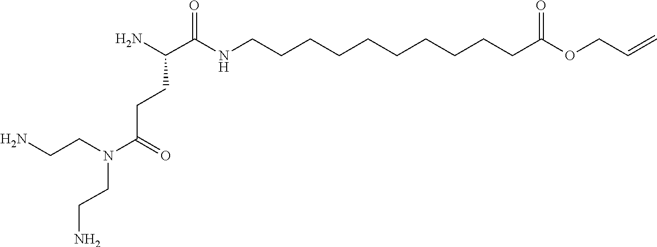 [SMILES: C=CCOC(=O)CCCCCCCCCCNC(=O)[C@@H](N)CCC(=O)N(CCN)CCN]

To a solution of allyl 11-[[(2S)-5-[bis[2-(tert-butoxycarbonylamino)ethyl]amino]-2-(tert-butoxycarbonylamino)-5-oxo-pentanoyl]amino]undecanoate (687 mg, 0.91 mmol) in DCM (15 mL) is added TFA (15 mL). The mixture is stirred at ambient temperature. After 1.5 hours, the mixture is concentrated in vacuo. The residue is taken up in MeOH and applied to an ion exchange cartridge. The cartridge is eluted with MeOH (150 mL) followed by 7N NH₃/MeOH (150 mL). The basic fraction is concentrated in vacuo to give the title compound (410 mg, 99%). ES/MS m/z 456.4 (M+H).

### Preparation 14

### Allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

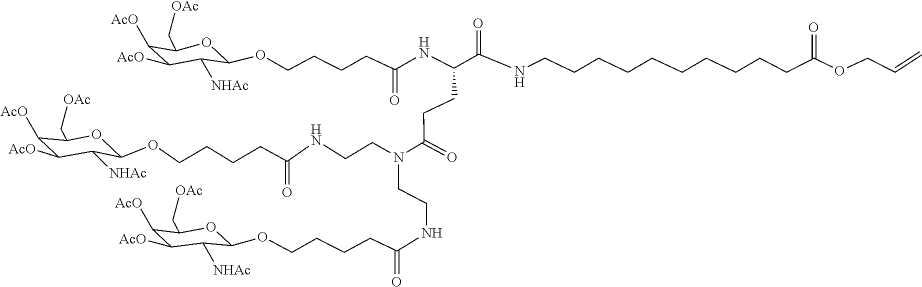

A flask is charged with 5-[3-acetamido-4,5-diacetoxy-6-(acetoxymethyl)tetrahydropyran-2-yl]oxypentanoic acid (489 mg, 1.09 mmol) and allyl (S)-11-(2-amino-5-(bis(2-aminoethyl)amino)-5-oxopentanamido)undecanoate (150 mg, 0.33 mmol). DCM (3.35 mL) is added followed by 1-hydroxybenzotriazole monohydrate (164 mg, 1.07 mmol) and 1-(3-dimethylaminopropyl)-3-ethylcarbodiimide hydrochloride (206 mg, 1.07 mmol). The mixture is stirred at ambient temperature for 18 hours. After this time, the solution is diluted with EtOAc (100 mL), washed with saturated NaHCO₃ (2×100 mL), saturated aqueous NH₄Cl (100 mL), and saturated aqueous sodium chloride solution (100 mL). The organic layer is dried over sodium sulfate, filtered, and concentrated in vacuo. The resulting residue is purified by silica gel flash chromatography eluting with 0-10% MeOH/DCM to give the title compound (424 mg, 74%). ES/MS m/z 872.80 (M+2H)/2.

### Preparation 15

### 11-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic acid

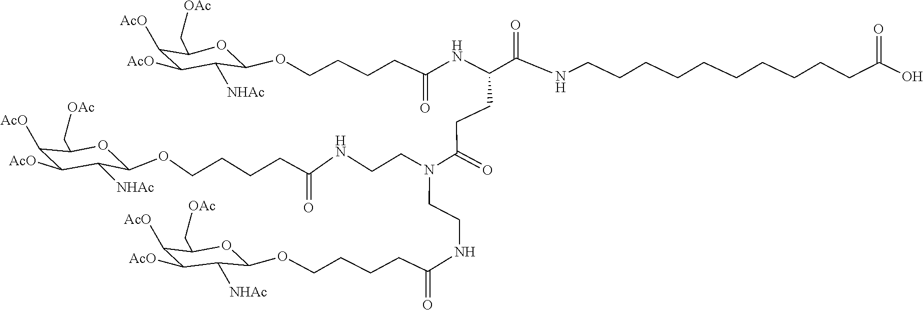

To a solution of allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate (354 mg, 0.20 mmol) in DCM (2 mL) is added tetrakis(triphenylphosphine)palladium (29 mg, 0.02 mmol) followed by PhSiH₃ (51 uL, 0.41 mmol). The mixture is stirred at ambient temperature for 2 hours, after which it is diluted with saturated aqueous NaHCO₃ (100 mL). 1N NaOH (15 mL) is added to bring the pH to about 10. The aqueous solution is washed with DCM (3×100 mL) and then acidified with concentrated HCl (5 mL) and then aqueous 5N HCl (15 mL). The aqueous layer is extracted with DCM (100 mL) and the organic layer is dried over sodium sulfate, filtered, and concentrated in vacuo. The resulting residue is purified by silica gel flash chromatography eluting with 0-20% MeOH/DCM to give the title compound (151 mg, 44%). ES/MS m/z 852.60 (M+2H)/2.

### Preparation 16

### (2,5-Dioxopyrrolidin-1-yl) 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

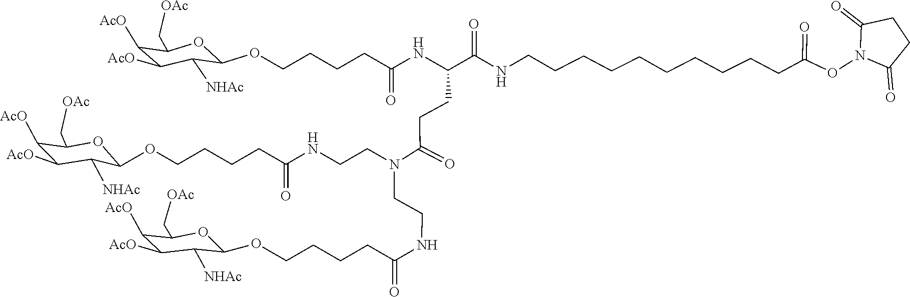 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

To a reaction vial are added 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic acid (50 mg, 0.03 mmol), N-hydroxysuccinimide (5 mg, 0.04 mmol), and 1-(3-dimethylaminopropyl)-3-ethylcarbodiimide hydrochloride (8 mg, 0.04 mmol). DCM (0.3 mL) is added and the mixture is stirred at ambient temperature. After 18 hours, the mixture is loaded directly onto a silica gel cartridge and the crude mixture is purified by silica gel flash chromatography eluting with 0-10% MeOH/DCM to give the title compound (49 mg, 93%). ES/MS m/z 901.40 (M+2H)/2.

### Preparation 17

### Benzyl 6-[[(2S)-5-[bis[2-(tert-butoxycarbonylamino)ethyl]amino]-2-(tert-butoxycarbonylamino)-5-oxo-pentanoyl]amino]hexanoate

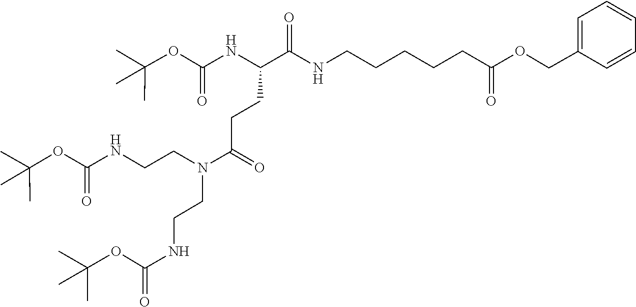 [SMILES: CC(C)(C)OC(=O)NCCN(CCNC(=O)OC(C)(C)C)C(=O)CC[C@H](NC(=O)OC(C)(C)C)C(=O)NCCCCCC(=O)OCc1ccccc1]

The title compound is prepared from (2S)-5-[bis[2-(tert-butoxycarbonylamino)ethyl]amino]-2-(tert-butoxycarbonylamino)-5-oxo-pentanoic acid and benzyl 6-aminohexanoate hydrochloride in a manner essentially analogous to the method of preparation 10. ES/MS m/z 736.40 (M+H).

### Preparation 18

### Benzyl 6-[[(2S)-2-amino-5-[bis(2-aminoethyl)amino]-5-oxo-pentanoyl]amino]hexanoate tris(trifluoroacetic acid)

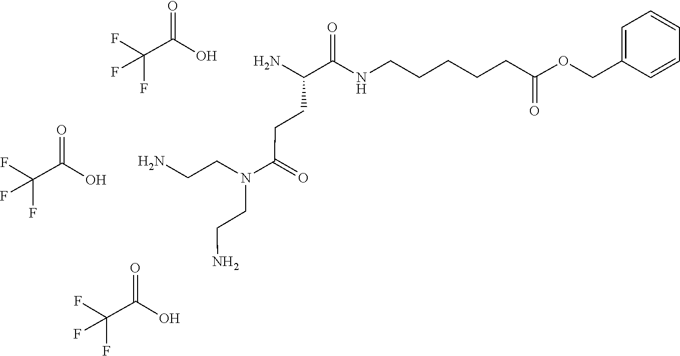 [SMILES: O=C(O)C(F)(F)F ; O=C(O)C(F)(F)F ; NCCN(CCN)C(=O)CC[C@H](N)C(=O)NCCCCCC(=O)OCc1ccccc1 ; O=C(O)C(F)(F)F]

To a solution of benzyl 6-[[(2S)-5-[bis[2-(tert-butoxycarbonylamino)ethyl]amino]-2-(tert-butoxycarbonylamino)-5-oxo-pentanoyl]amino]hexanoate (15.47 g, 21.02 mmol) in DCM (105 mL) is added TFA (16 mL, 210.2 mmol). The mixture is stirred at ambient temperature for 24 hours. After this time, additional TFA (16 mL, 210.2 mmol) is added and stirring is continued for an additional 2 hours. After this time, the mixture is concentrated in vacuo. The resulting residue is azeotroped with toluene (2×30 mL). The resulting oil is further dried in a vacuum oven at 40° C. for 4 hours to give the title compound (28.08 g, 58% purity accounting for residual toluene, 99+%). ES/MS m/z 436.40 (M+H). The compound is dissolved in 70 mL DMF to make a 0.3M solution that is used in the next step.

### Preparation 19

### Benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

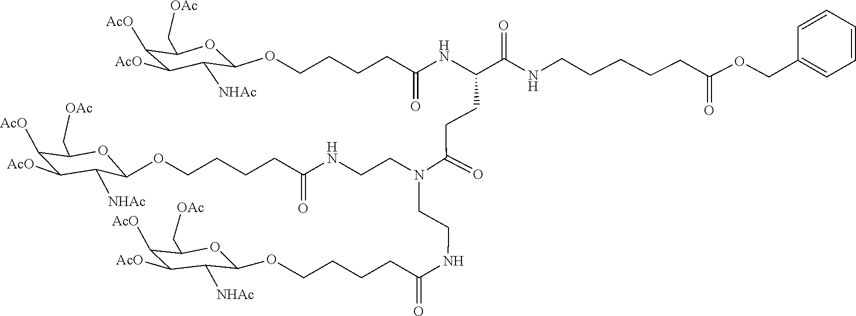 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl 6-[[(2S)-2-amino-5-[bis(2-aminoethyl)amino]-5-oxo-pentanoyl]amino]hexanoate tris trifluoroacetic acid and in a manner essentially analogous to the method of preparation 10. ES/MS m/z 862 (M+2H)/2.

### Preparation 20

### 6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid

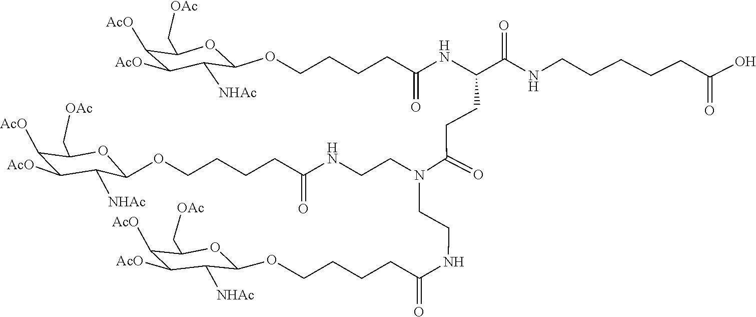 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

Palladium on carbon (1.90 g, 0.89 mmol, 5 mass %, 50% wet) is placed in a round-bottom flask and the vessel is evacuated and backfilled with nitrogen three times. A solution of benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate (15.41 g, 8.94 mmol) in MeOH (178 mL) is added via syringe. The flask is evacuated and backfilled with 1 atm hydrogen and the mixture is stirred at ambient temperature under 1 atm hydrogen for 18 hours. After this time, the mixture is filtered through diatomaceous earth and the filtrate is concentrated in vacuo to give the title compound (13.85 g, 95%). ES/MS m/z 817.2 (M+2H)/2.

### Preparation 21

### (2,5-Dioxopyrrolidin-1-yl) 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

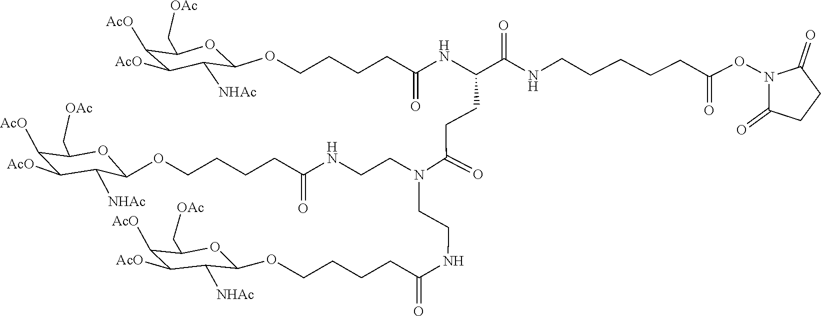 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid in a manner essentially analogous to the method of preparation 16. ES/MS m/z 866.20 (M+2H)/2.

### Preparation 22

### Benzyl (2S)-5-[bis[2-(tert-butoxycarbonylamino)ethyl]amino]-2-(tert-butoxycarbonylamino)-5-oxo-pentanoate

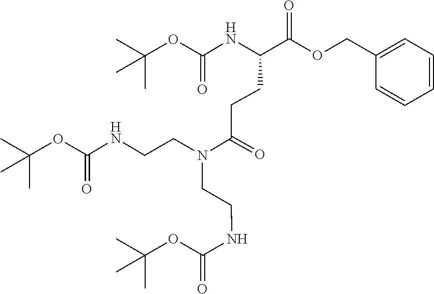 [SMILES: CC(C)(C)OC(=O)NCCN(CCNC(=O)OC(C)(C)C)C(=O)CC[C@H](NC(=O)OC(C)(C)C)C(=O)OCc1ccccc1]

The title compound is prepared from tert-butyl N-[2-[2-(tert-butoxycarbonylamino)ethylamino]ethyl]carbamate and (4S)-5-benzyloxy-4-(tert-butoxycarbonylamino)-5-oxo-pentanoic acid in a manner essentially analogous to the method of preparation 12. ES/MS m/z 623.6 (M+H).

### Preparation 23

### Benzyl (2S)-2-amino-5-[bis(2-aminoethyl)amino]-5-oxo-pentanoate tris(trifluoroacetic acid) salt

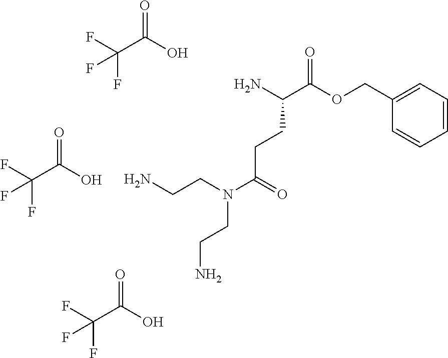 [SMILES: O=C(O)C(F)(F)F ; O=C(O)C(F)(F)F ; NCCN(CCN)C(=O)CC[C@H](N)C(=O)OCc1ccccc1 ; O=C(O)C(F)(F)F]

The title compound is prepared from benzyl (2S)-5-[bis[2-(tert-butoxycarbonylamino)ethyl]amino]-2-(tert-butoxycarbonylamino)-5-oxo-pentanoate in a manner essentially analogous to the method of preparation 18. ES/MS m/z 323.2 (M+H).

### Preparation 24

### Benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate

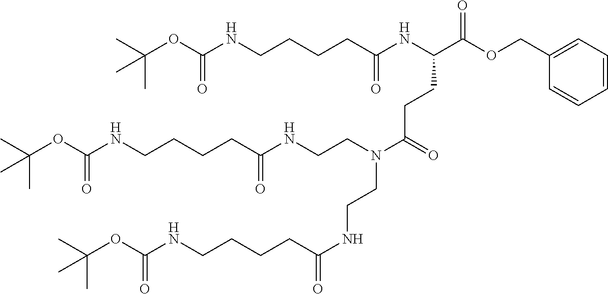 [SMILES: CC(C)(C)OC(=O)NCCCCC(=O)NCCN(CCNC(=O)CCCCNC(=O)OC(C)(C)C)C(=O)CC[C@H](NC(=O)CCCCNC(=O)OC(C)(C)C)C(=O)OCc1ccccc1]

The title compound is prepared from 5-(tert-butoxycarbonylamino)pentanoic acid and benzyl (2S)-2-amino-5-[bis(2-aminoethyl)amino]-5-oxo-pentanoate tris(trifluoroacetic acid) salt in a manner essentially analogous to the method of preparation 10. ES/MS m/z 920.6 (M+H).

### Preparation 25

### Benzyl (2S)-2-(5-aminopentanoylamino)-5-[bis[2-(5-aminopentanoylamino)ethyl]amino]-5-oxo-pentanoate tris(trifluoroacetic acid) salt

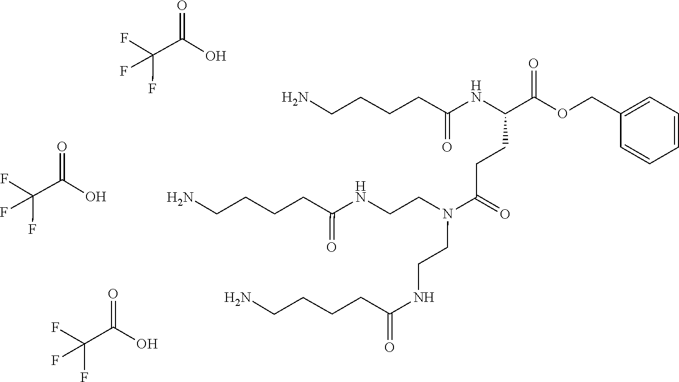 [SMILES: O=C(O)C(F)(F)F ; O=C(O)C(F)(F)F ; O=C(O)C(F)(F)F ; NCCCCC(=O)NCCN(CCNC(=O)CCCCN)C(=O)CC[C@H](NC(=O)CCCCN)C(=O)OCc1ccccc1]

The title compound is prepared from benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate in a manner essentially analogous to the method of preparation 18. ES/MS m/z 620.4 (M+H).

### Preparation 26

### Benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate

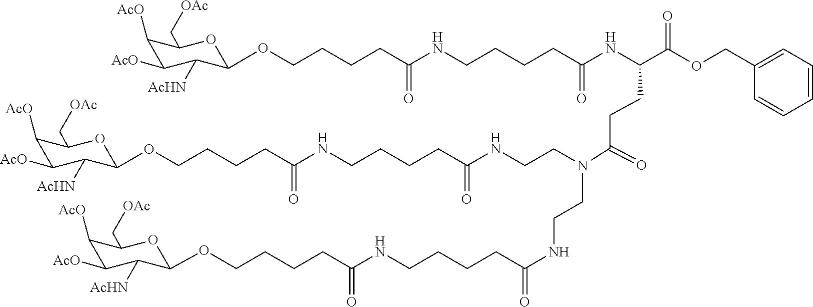 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl (2S)-2-(5-aminopentanoylamino)-5-[bis[2-(5-aminopentanoylamino)ethyl]amino]-5-oxo-pentanoate tris(trifluoroacetic acid) salt and in a manner essentially analogous to the method of preparation 10. ES/MS m/z 954.80 (M+2H)/2.

### Preparation 27

### (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid

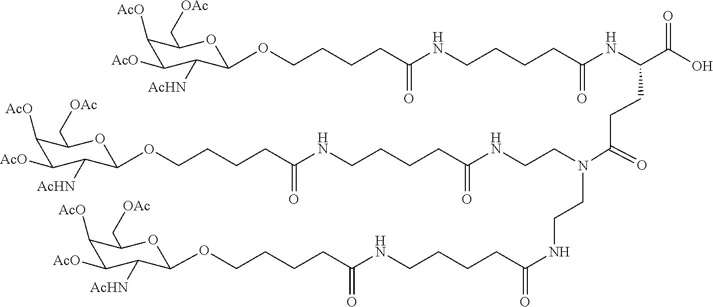 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)O)OC(COC(C)=O)C(OC(C)=O)C1OC(C)=O]

A round-bottom flask is charged with palladium on carbon (467 mg, 0.22 mmol, 5 mass %, 50% wet) and the flask is evacuated and backfilled with nitrogen three times. A solution of benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate (4.19 g, 2.20 mmol) in MeOH (44 mL) is added via syringe followed by three drops of acetic acid. The flask is evacuated and backfilled with 1 atm hydrogen and the mixture is stirred at ambient temperature under 1 atm hydrogen. After 2 hours, the mixture is filtered through diatomaceous earth and the filtrate is concentrated in vacuo to give the title compound (3.99 g, 99+%). ES/MS m/z 909.6 (M+2H)/2.

### Preparation 28

### Benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

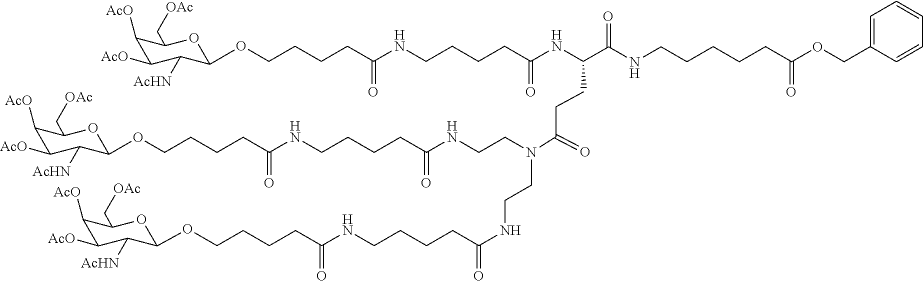 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 6-aminohexanoate hydrochloride and in a manner essentially analogous to the method of preparation 10. ES/MS m/z 1011.6 (M+2H)/2.

### Preparation 29

### 6-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic acid

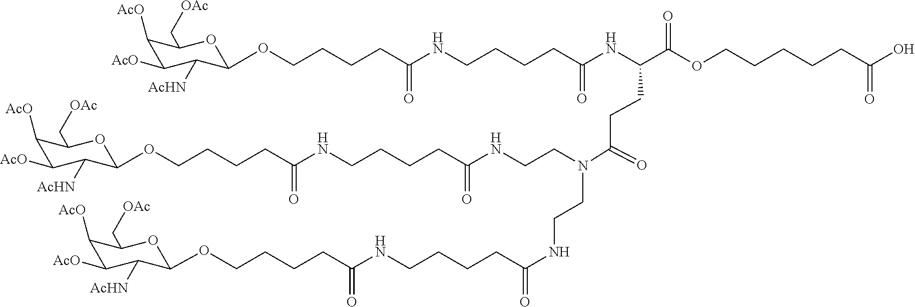 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)OCCCCCC(=O)O)OC(COC(C)=O)C(OC(C)=O)C1OC(C)=O]

A round-bottom flask is charged with palladium on carbon (24 mg, 0.01 mmol, 5% by mass, 50% wet) and the flask is evacuated and backfilled with nitrogen. A solution of benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate (222 mg, 0.11 mmol) in MeOH (2.2 mL) is added via syringe followed by three drops of acetic acid. The flask is evacuated and backfilled with 1 atm hydrogen and the mixture is stirred under 1 atm hydrogen at ambient temperature. After 5 hours, the flask is purged with nitrogen and the mixture is filtered through diatomaceous earth. The filtrate is concentrated in vacuo to give the title compound (180 mg, 85%). ES/MS m/z 966.2 (M+2H)/2.

### Preparation 30

### (2,5-Dioxopyrrolidin-1-yl) 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

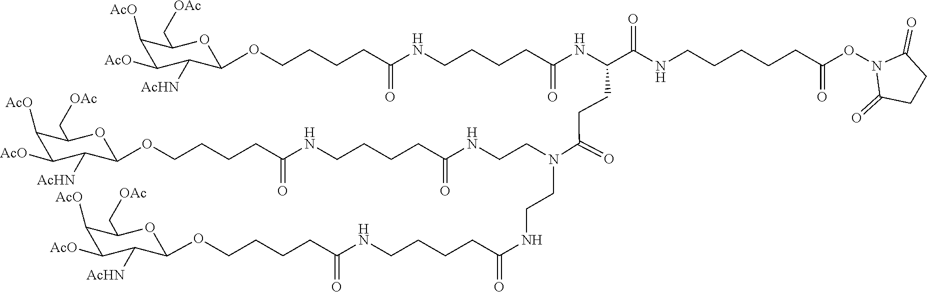 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic acid in a manner essentially analogous to the method of preparation 16. ES/MS m/z 1014.6 (M+2H)/2.

### Preparation 31

### Benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

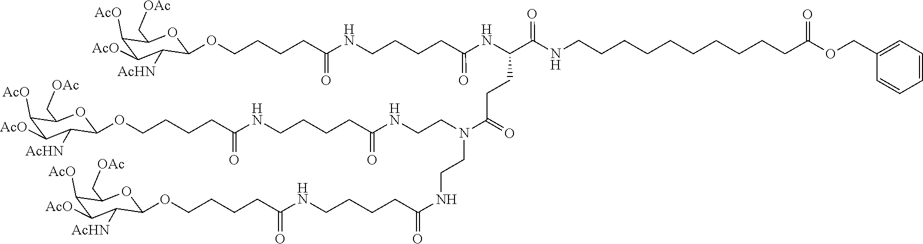 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 11-aminoudecanoate hydrochloride in a manner essentially analogous to the method of preparation 10. ES/MS m/z 1046.6 (M+2H)/2.

### Preparation 32

### 11-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic acid

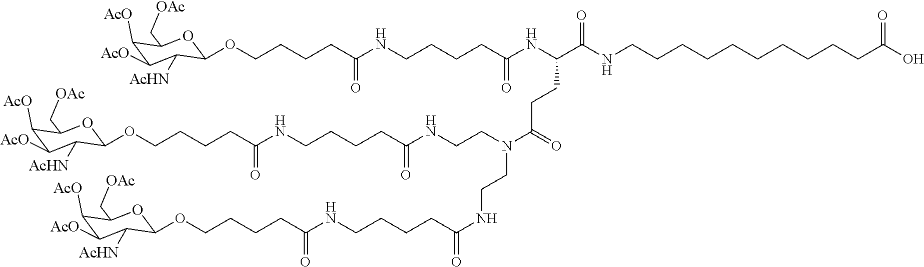 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)O)OC(COC(C)=O)C(OC(C)=O)C1OC(C)=O]

To a round-bottom flask is added palladium on carbon (35 mg, 0.02 mmol, 5 mass %, 50% wet) and the flask is evacuated and backfilled with nitrogen three times. A solution of benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate (285 mg, 80% purity, 0.11 mmol) is added via syringe. The vessel is evacuated and backfilled with 1 atm hydrogen and the mixture is then stirred at ambient temperature under 1 atm hydrogen. After stirring for 3 hours, the flask is purged with nitrogen and the mixture is filtered through diatomaceous earth. The filtrate is concentrated to give the title compound (213 mg, 79% purity, 77%). ES/MS m/z 1001.20 (M+2H)/2.

### Preparation 33

### (2,5-Dioxopyrrolidin-1-yl) 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

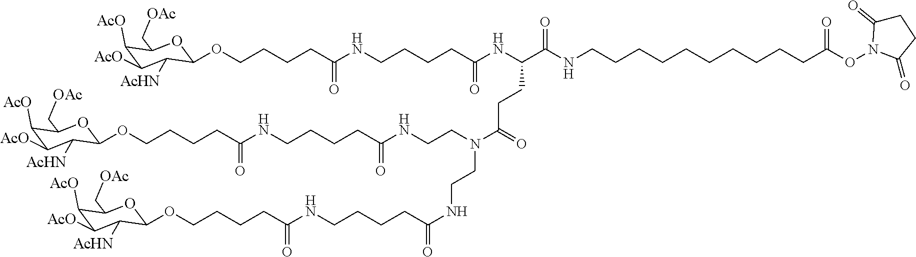 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

The title compound is prepared from 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic acid in a manner essentially analogous to the method of preparation 16. ES/MS m/z 1050 (M+2H)/2

### Preparation 34

### [5-Acetamido-6-[5-[2-[[(4S)-4-[5-[3-acetamido-4,5-diacetoxy-6-(acetoxymethyl)tetrahydropyran-2-yl]oxypentanoylamino]-5-[[6-[[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl acetate

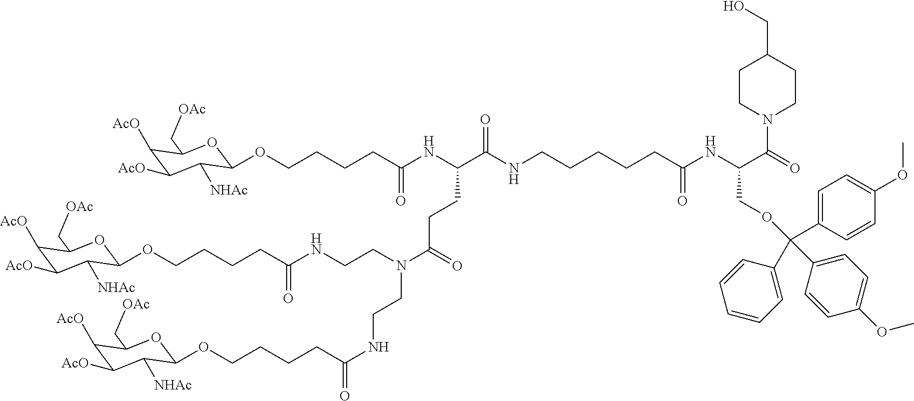 [SMILES: COc1ccc(C(OC[C@H](NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(CO)CC2)(c2ccccc2)c2ccc(OC)cc2)cc1]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid and (2S)-2-amino-3-[bis(4-methoxyphenyl)-phenyl-methoxy]-1-[4-(hydroxymethyl)-1-piperidyl]propan-1-one in a manner essentially analogous to the method of preparation 10. ES/MS m/z 1059.2 (M-2H)/2.

### Preparation 35

### 4-[[1-[(2S)-2-[6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid

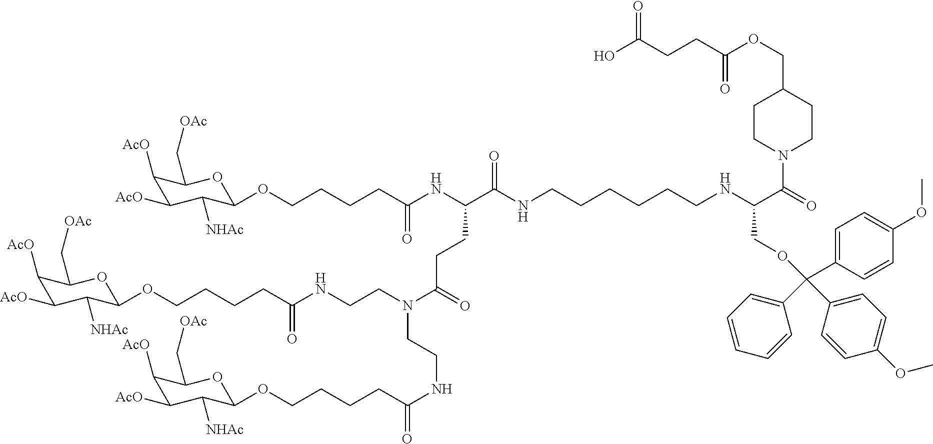 [SMILES: COc1ccc(C(OC[C@H](NCCCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(COC(=O)CCC(=O)O)CC2)(c2ccccc2)c2ccc(OC)cc2)cc1]

To a solution of [5-acetamido-6-[5-[2-[[(4S)-4-[5-[3-acetamido-4,5-diacetoxy-6-(acetoxymethyl)tetrahydropyran-2-yl]oxypentanoylamino]-5-[[6-[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl acetate (1.194 g, 0.56 mmol) in DCM (11 mL) is added succinic anhydride (113 mg, 1.13 mmol), TEA (0.4 mL, 3 mmol) and DMAP (213 mg, 1.69 mmol). The mixture is stirred at ambient temperature for 1 hour. After this time, the mixture is diluted with saturated NH₄Cl (200 mL) and extracted with DCM (3×200 mL) and 3:1 CHCl₃:IPA (200 mL). The organic layers are combined, dried over sodium sulfate, filtered, and concentrated in vacuo. The resulting residue is purified by silica gel flash chromatography eluting with 0-40% MeOH/DCM and the resulting product is dried in a vacuum oven at 40° C. for 3 hours to give the title compound (1.081 g, 86%). ES/MS m/z 1109.60 (M-2H)/2.

### Preparation 36

Resin loading

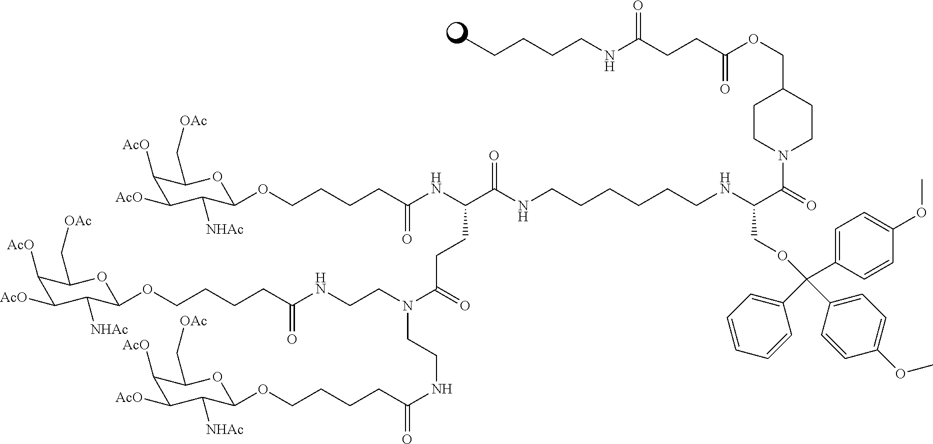 [SMILES: CCCCCNC(=O)CCC(=O)OCC1CCN(C(=O)[C@H](COC(c2ccccc2)(c2ccc(OC)cc2)c2ccc(OC)cc2)NCCCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)CC1]

A solution of 4-[[1-[(2S)-2-[6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid (1.00 g, 0.61 mmol) in MeCN (6 mL) and DCM (1 mL) is transferred to a resin loading cartridge. To the vessel are added 2-(1H-benzotriazol-1-yl)-1,1,3,3-tetramethyluronium hexafluorophosphate (386 mg, 0.97 mmol) and DIEA (0.25 mL, 0.48 mmol) and the cartridge is shaken at ambient temperature for 5 minutes. After this time, 1000 Å LCAA controlled-pore glass resin (5.39 g, 90 μmol/g loading, purchased from ChemGenes) is added and the mixture is shaken at ambient temperature for 18 hours. After this time, the cartridge is drained by suction and the resin is washed by shaking with DCM (10 mL) for 10 minutes. The cartridge is drained and the washing and draining procedure is repeated with 10% MeOH/DCM (10 mL) and Et₂O (10 mL). After draining, a solution of acetic anhydride (6.4 mL), pyridine (20 mL) and TEA (0.22 mL) is added and the cartridge is shaken for 2 hours. After this time, the cartridge is drained and the washing and draining procedure above is repeated using DCM (10 mL), 10% MeOH/DCM (10 mL) and diethyl ether (10 mL). After draining, the resin is dried under vacuum for 30 minutes. The resin loading is determined using a standard trityl assay. The resin loading was calculated to be 34.7 μmol/g.

### Preparation 37

### Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

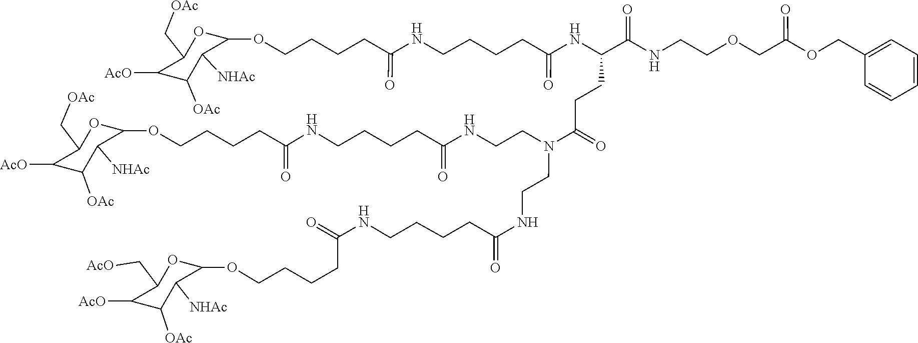 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)OCc2ccccc2)OC(COC(C)=O)C(OC(C)=O)C1OC(C)=O]

The title compound is prepared from (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-(2-aminoethoxy)acetate hydrochloride in a manner essentially analogous to the method of preparation 10. ES/MS m/z 1005.2 (M+2H/2).

### Preparation 38

### 2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoylamino]ethoxy]acetic acid

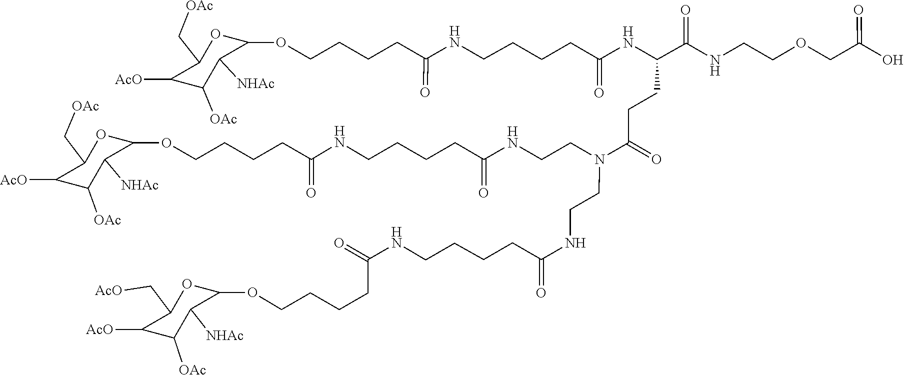 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)O)OC(COC(C)=O)C(OC(C)=O)C1OC(C)=O]

Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate (0.120 mmol, 240 mg) is combined with 5% Pd/C (1.17 mmol, 124 mg) in MeOH (12.0 ml). The mixture is hydrogenated on a Parr shaker (ambient temperature, 10 psi) for 48 minutes, filtered through diatomaceous earth, and concentrated in vacuo to give the title compound as a gray solid (187 mg, 82%). ES/MS m/z 960.0 (M+2H/2).

### Preparation 39

### (2,3,5,6-Tetrafluorophenyl) 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

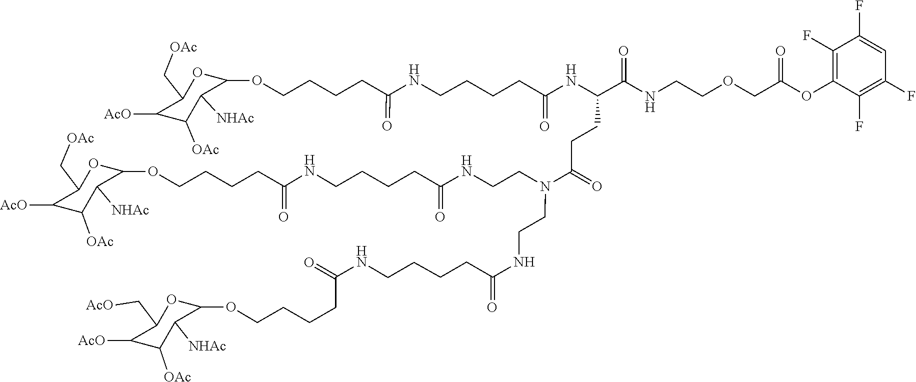 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)Oc2c(F)c(F)cc(F)c2F)OC(COC(C)=O)C(OC(C)=O)C1OC(C)=O]

To 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetic acid (0.096 mmol, 184 mg) and DIEA (0.765 mmol, 140 μL) in DCM (3.0 ml) is added (2,3,5,6-tetrafluorophenyl) 2,2,2-trifluoroacetate (0.383 mmol, 100 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (197 mg, 99%). ES/MS m/z 1034.0 (M+2H/2).

### Preparation 40

### Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

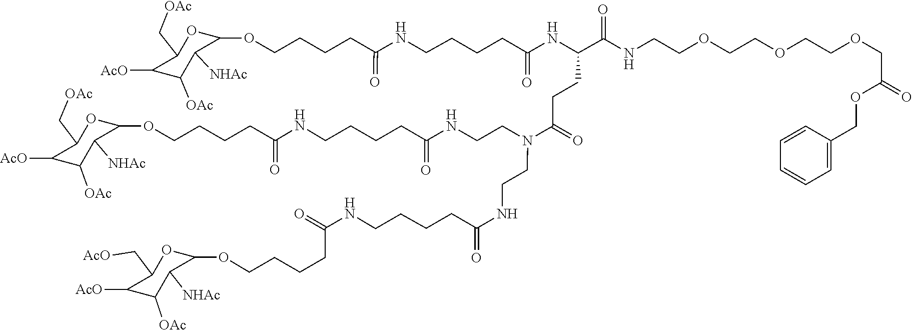 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-[2-[2-(2-aminoethoxy)ethoxy]ethoxy]acetate hydrochloride in a manner essentially analogous to the method of preparation 10. ES/MS m/z 1049.0 (M+2H/2).

### Preparation 41

### 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic acid

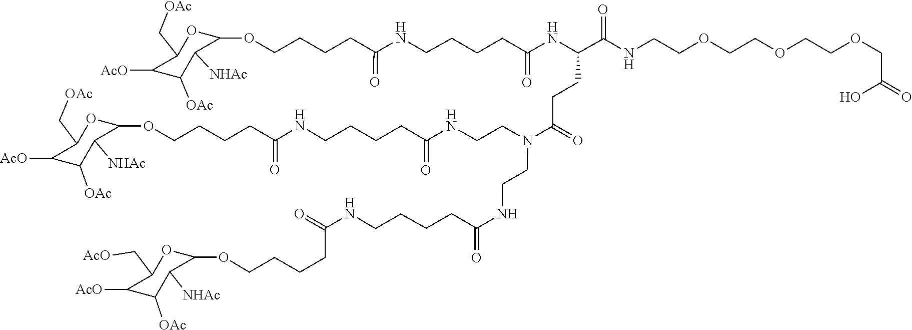 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)O)OC(COC(C)=O)C(OC(C)=O)C1OC(C)=O]

Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate (0.118 mmol, 247 mg) is combined with 5% Pd/C (1.17 mmol, 124 mg) in MeOH (12.0 mL). The mixture is hydrogenated on a Parr shaker (ambient temperature, 10 psi) for 1 hour, filtered through diatomaceous earth, and concentrated in vacuo to give the title compound as a gray solid (227 mg, 96%). ES/MS m/z 1004.0 (M+2H/2).

### Preparation 42

### (2,3,5,6-Tetrafluorophenyl) 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

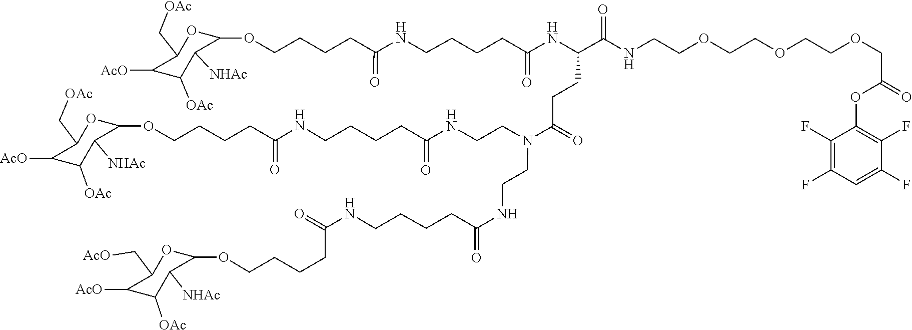 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)Oc2c(F)c(F)cc(F)c2F)OC(COC(C)=O)C(OC(C)=O)C1OC(C)=O]

To 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic acid (0.111 mmol, 222 mg) and DIEA (0.883 mmol, 154 μL) in DCM (3.0 ml) is added (2,3,5,6-tetrafluorophenyl) 2,2,2-trifluoroacetate (0.443 mmol, 116 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (174 mg, 73%). ES/MS m/z 1078.2 (M+2H/2).

### Example 1: Conjugation Protocol

For the synthesis of GalNAc-conjugated sense strands, a sense strand with a 3′ C6-NH₂ functional group was first synthesized using standard phosphoramidite chemistry. A stock solution of GalNAc ligand-NHS ester (10 mmol/L in acetonitrile; 1 eq) was prepared. Borate buffer (10% v/v; 20×) was added to oligonucleotide C6-NH₂ sense strand in an Eppendorf tube, then GalNAc ligand (5 eq) was added. The mixture was shaken at ambient temperature for 16 hours. After this time, the mixture was transferred to a 15 mL falcon tube, ammonium hydroxide (28 mass %) was added, and the mixture was shaken at ambient temperature for 2 hours. The ammonia was then removed in vacuo. The residue was purified by ion-exchange chromatography. Conditions: Solvent A: 15% MeCN/20 mM NaH₂PO₄, Solvent B: 15% MeCN/20 mM NaH₂PO₄, 1M NaBr; 35-55% B over 5 CV at 8 mL/min, column temperature 60° C. The desired fractions were pooled and desalted by spin-filtration using an Eppendorf centrifuge or desalting column. After desalting, the material was recovered and OD and volume were measured to obtain concentration.

Alternatively, conjugation to the 5′ position of the sense strand through immobilizing the GalNAc ligand on microporous polystyrene resin or controlled pore glass and synthesized using established solid phase oligonucleotide synthesis methods with 5′-CE β-cyanoethyl) phosphoramidites.

Alternatively, the GalNAc ligand was converted to a suitable phosphoramidite and delivered to the 5′ position of the sense strand using standard phosphoramidite chemistry.

### Example 2: Annealing

To generate the siRNA duplexes of a sense and antisense strand, the following procedures were performed. To a falcon tube containing oligonucleotide sense strand-GalNAc conjugate, the corresponding antisense oligonucleotide (1 eq) was added and vortexed for 10 seconds before spin-filtering through 100K MWCO Amicon filter unit to remove particulates. The filtrate was recovered and concentrated in vacuo on a Genevac evaporator. The residue was reconstituted in 1×PBS, filtered through 0.2μ filter, and OD and volume were measured to obtain concentration.

An endotoxin test was performed using a Limulus amebocyte lysate on an Endosafe®-nexgen PTS instrument.

TABLE 1
Exemplary molecules synthesized utilizing the aforementioned conjugation and
annealing protocols.

| Molecule Identifier | Ligand attached to 3′ of sense strand |
|---|---|
| 1 | 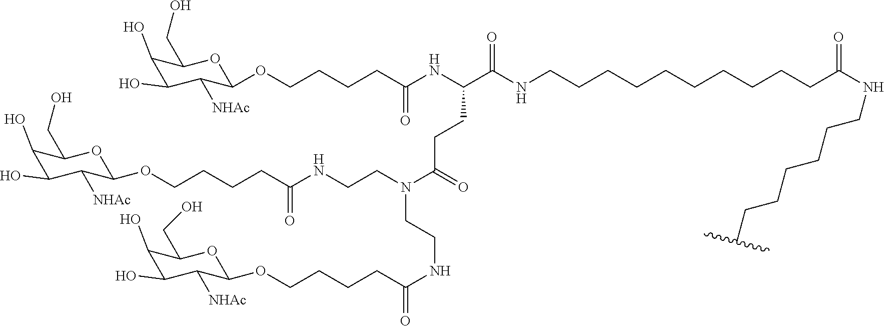 [SMILES: *CCCCCCNC(=O)CCCCCCCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 2 | 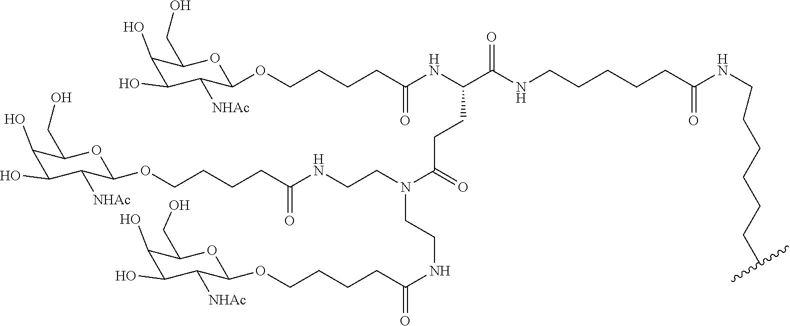 [SMILES: *CCCCCCNC(=O)CCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 3 | 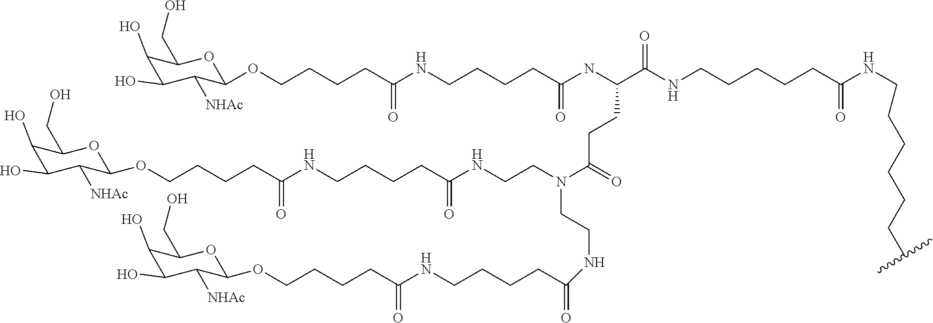 [SMILES: *CCCCCCNC(=O)CCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 4 | 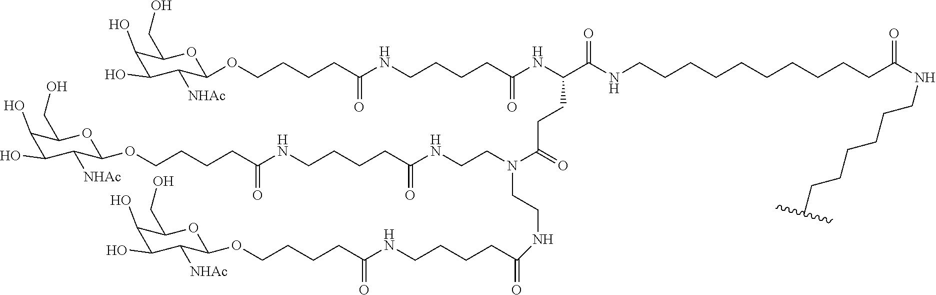 [SMILES: *CCCCCCNC(=O)CCCCCCCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 5 | 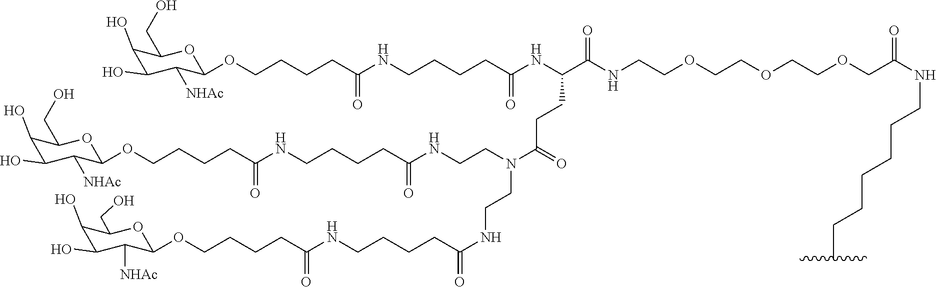 [SMILES: *CCCCCCNC(=O)COCCOCCOCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 6 | 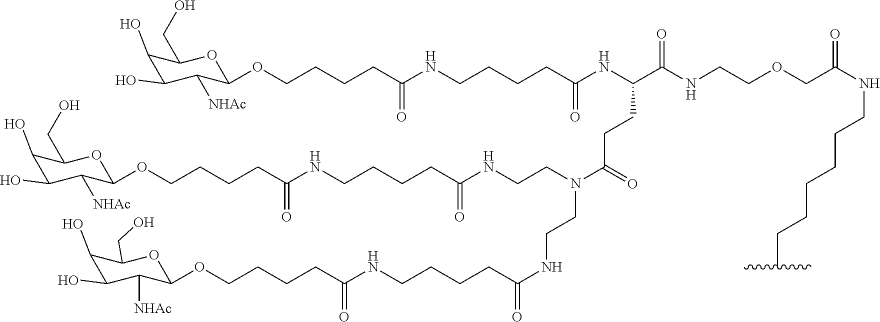 [SMILES: *CCCCCCNC(=O)COCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |

### Example 3

### General procedure for oligo synthesis using GalNAc-functionalized CPG

Oligo synthesis was conducted on a MerMade™ 12 instrument using phosphoramidite chemistry. Sense strands were synthesized from the prefunctionalized GalNAc solid support and antisense strands were synthesized using standard support preloaded with the first nucleotide of the oligo sequence. Oligos were cleaved and deprotected using concentrated ammonium hydroxide solution (28% by mass) and purified by ion exchange chromatography using conditions described above. Desalting, annealing, and endotoxin testing were conducted.

The sequence of antisense oligonucleotides were designed using 15 to 50 nucleotides of the following HMGCR transcript (SEQ ID NO: 1), where T nucleotides were replaced by U nucleotides, and where one or more nucleotides and one or more internucleotide linkages were optionally further modified as described herein.

Homo sapiens HMGCR Cell Death Receptor (HMGCR) transcript,
SEQ ID NO: 1
ccttccgctc cgcgactgcg ttaactggag ccaggctgag cgtcggcgcc ggggttcggt
ggcctctagt gagatctgga ggatccaagg attctgtagc tacaatgttg tcaagacttt
ttcgaatgca tggcctcttt gtggcctccc atccctggga agtcatagtg gggacagtga
cactgaccat ctgcatgatg tccatgaaca tgtttactgg taacaataag atctgtggtt
ggaattatga atgtccaaag tttgaagagg atgttttgag cagtgacatt ataattctga
caataacacg atgcatagcc atcctgtata tttacttcca gttccagaat ttacgtcaac
ttggatcaaa atatattttg ggtattgctg gccttttcac aattttctca agttttgtat
tcagtacagt tgtcattcac ttcttagaca aagaattgac aggcttgaat gaagctttgc
cctttttcct acttttgatt gacctttcca gagcaagcac attagcaaag tttgccctca
gttccaactc acaggatgaa gtaagggaaa atattgctcg tggaatggca attttaggtc
ctacgtttac cctcgatgct cttgttgaat gtcttgtgat tggagttggt accatgtcag
gggtacgtca gcttgaaatt atgtgctgct ttggctgcat gtcagttctt gccaactact
tcgtgttcat gactttcttc ccagcttgtg tgtccttggt attagagctt tctcgggaaa
gccgcgaggg tcgtccaatt tggcagctca gccattttgc ccgagtttta gaagaagaag
aaaataagcc gaatcctgta actcagaggg tcaagatgat tatgtctcta ggcttggttc
ttgttcatgc tcacagtcgc tggatagctg atccttctcc tcaaaacagt acagcagata
cttctaaggt ttcattagga ctggatgaaa atgtgtccaa gagaattgaa ccaagtgttt
ccctctggca gttttatctc tctaaaatga tcagcatgga tattgaacaa gttattaccc
taagtttagc tctccttctg gctgtcaagt acatcttctt tgaacaaaca gagacagaat
ctacactctc attaaaaaac cctatcacat ctcctgtagt gacacaaaag aaagtcccag
acaattgttg tagacgtgaa cctatgctgg tcagaaataa ccagaaatgt gattcagtag
aggaagagac agggataaac cgagaaagaa aagttgaggt tataaaaccc ttagtggctg
aaacagatac cccaaacaga gctacatttg tggttggtaa ctcctcctta ctcgatactt
catcagtact ggtgacacag gaacctgaaa ttgaacttcc cagggaacct cggcctaatg
aagaatgtct acagatactt gggaatgcag agaaaggtgc aaaattcctt agtgatgctg
agatcatcca gttagtcaat gctaagcata tcccagccta caagttggaa actctgatgg
aaactcatga gcgtggtgta tctattcgcc gacagttact ttccaagaag ctttcagaac
cttcttctct ccagtaccta ccttacaggg attataatta ctccttggtg atgggagctt
gttgtgagaa tgttattgga tatatgccca tccctgttgg agtggcagga cccctttgct
tagatgaaaa agaatttcag gttccaatgg caacaacaga aggttgtctt gtggccagca
ccaatagagg ctgcagagca ataggtcttg gtggaggtgc cagcagccga gtccttgcag
atgggatgac tcgtggccca gttgtgcgtc ttccacgtgc ttgtgactct gcagaagtga
aagcctggct cgaaacatct gaagggttcg cagtgataaa ggaggcattt gacagcacta
gcagatttgc acgtctacag aaacttcata caagtatagc tggacgcaac ctttatatcc
gtttccagtc caggtcaggg gatgccatgg ggatgaacat gatttcaaag ggtacagaga
aagcactttc aaaacttcac gagtatttcc ctgaaatgca gattctagcc gttagtggta
actattgtac tgacaagaaa cctgctgcta taaattggat agagggaaga ggaaaatctg
ttgtttgtga agctgtcatt ccagccaagg ttgtcagaga agtattaaag actaccacag
aggctatgat tgaggtcaac attaacaaga atttagtggg ctctgccatg gctgggagca
taggaggcta caacgcccat gcagcaaaca ttgtcaccgc catctacatt gcctgtggac
aggatgcagc acagaatgtt ggtagttcaa actgtattac tttaatggaa gcaagtggtc
ccacaaatga agatttatat atcagctgca ccatgccatc tatagagata ggaacggtgg
gtggtgggac caacctacta cctcagcaag cctgtttgca gatgctaggt gttcaaggag
catgcaaaga taatcctggg gaaaatgccc ggcagcttgc ccgaattgtg tgtgggaccg
taatggctgg ggaattgtca cttatggcag cattggcagc aggacatctt gtcaaaagtc
acatgattca caacaggtcg aagatcaatt tacaagacct ccaaggagct tgcaccaaga
agacagcctg aatagcccga cagttctgaa ctggaacatg ggcattgggt tctaaaggac
taacataaaa tctgtgaatt aaaaaagctc aatgcattgt cttgtggagg atgaatagat
gtgatcactg agacagccac ttggtttttg gctctttcag agaggtctca ggttctttcc
atgcagactc ctcagatctg aacacagttt agtgctttac atgctgtgct ctttgaagag
atttcaacaa gaatattgta tgttaaagca tcagagatgg taatctacag ctcacctctg
aaggcaaata taagctggga aaaaagtttt gatgaaattc ttgaagttca tggtgatcag
tgcaattgac cttctccctc actcctgcca gttgaaaatg gatttttaaa ttatactgta
gctgatgaaa ctcctgattt tgtagttaat ttattaagtc tgggatgtag aacttcaaga
agtaagagct aagttctaag ttcatgtttg taaattaata cttcatttgg tgctggtcta
ttttgatttt ggggggtaat cagcattatt cttcagaagg ggacctgttt tcttcaaggg
aagaaacact cttattccca aactacagaa taatgtgtta aacatgctaa atagttctat
caggaaaaca aatcactgta tttatctccg caggctattt gttcagagag gccttttgtt
taaatataaa tgtttaaata taaatgtttg tctggattgg ctataacatg tctttcagca
ttaggctttt aagaaacaca gggttttgta ttctttacta aagatatcag agctcttaat
gttgcttaga tgagggtgac tgtcaagtac aagcaagact gggaccttag aaatcattgt
agaaacacag ttttgaaaga aaaataccat gtctctaagc caactttaat tgcttaaaag
acatttttat ttagttgaaa aatctagttt tttttgtaaa ctgtatcaaa tctgtatatg
ttgtaataaa acttatgcta gtttattgga agtgttcaag aaataaaaat caacttgtgt
actgataaaa tactctagcc tgggccagag aagataatgt tctttaatgt tgtccaggaa
accctggctt gcttgccgag cctaatgaaa gggaaagtca gctttcagag ccagtgaagg
agccacgtga atggccctag aactgtgcct agttcctgtg gccaggaggt tggtgactga
aacattcaca cagggctctt tgatggaccc acgaacgctc ttagctttct cagggggtca
gcagagttat tgaatcttaa ttttttttaa tgtacaagtt ttgtataaat aataaagaac
tccttatttt gtattacatc taatgcttca agtgttgctc ttggaaagct gatgatgtct
cttgtagaag atggactctg aaaaacattc caggaaacca tggcagcatg gagagcctct
tagtgattgt gtctgcattg ttattgtgga agatttacct tttctgttgt acgtaaagct
taaattgctt ttgttgtgac tttttagcca gtgacttttt ctgagctttt catggaagtg
gcagtgaaaa atatgttgag tgttcatttt agtgactgta attaatatct tgctggatta
atgttttgta caattactaa attgtataca ttttgttata gaatactttt ttctagtttc
agtaaataat gaaaaggaag ttaataccaa

Exemplary antisense strand sequences of 18 nucleotides in length are shown in Table 2 below, which may be optionally further modified and synthesized and incorporated into the RNAi agents, as described herein.

TABLE 2
Antisense 18 mers of HMGCR RNAi agents

| SEQ ID: | Antisense 18 Mer |
|---|---|
| SEQ ID: 2 | AGUCUUGACAACAUUGUA |
| SEQ ID: 3 | AAGUCUUGACAACAUUGU |
| SEQ ID: 4 | AACAUGUUCAUGGACAUC |
| SEQ ID: 5 | CUGGAACUGGAAGUAAAU |
| SEQ ID: 6 | UUCUGGAACUGGAAGUAA |
| SEQ ID: 7 | AAAAAGGGCAAAGCUUCA |
| SEQ ID: 8 | GGGCAAACUUUGCUAAUG |
| SEQ ID: 9 | AGGGCAAACUUUGCUAAU |
| SEQ ID: 10 | GAGGGCAAACUUUGCUAA |
| SEQ ID: 11 | ACUGAGGGCAAACUUUGC |
| SEQ ID: 12 | GAACUGAGGGCAAACUUU |
| SEQ ID: 13 | CCUUACUUCAUCCUGUGA |
| SEQ ID: 14 | UUCCCUUACUUCAUCCUG |
| SEQ ID: 15 | UUUUCCCUUACUUCAUCC |
| SEQ ID: 16 | AUUUUCCCUUACUUCAUC |
| SEQ ID: 17 | CCAAAUUGGACGACCCUC |
| SEQ ID: 18 | GCCAAAUUGGACGACCCU |
| SEQ ID: 19 | UGCCAAAUUGGACGACCC |
| SEQ ID: 20 | GAGCUGCCAAAUUGGACG |
| SEQ ID: 21 | UGAGCUGCCAAAUUGGAC |
| SEQ ID: 22 | CUGAGCUGCCAAAUUGGA |
| SEQ ID: 23 | GGCUGAGCUGCCAAAUUG |
| SEQ ID: 24 | AAUGGCUGAGCUGCCAAA |
| SEQ ID: 25 | CAAAAUGGCUGAGCUGCC |
| SEQ ID: 26 | GCAAAAUGGCUGAGCUGC |
| SEQ ID: 27 | CUUCUUCUUCUAAAACUC |
| SEQ ID: 28 | CAUAAUCAUCUUGACCCU |
| SEQ ID: 29 | AGACAUAAUCAUCUUGAC |
| SEQ ID: 30 | GGAUCAGCUAUCCAGCGA |
| SEQ ID: 31 | AGGAUCAGCUAUCCAGCG |
| SEQ ID: 32 | AAGGAUCAGCUAUCCAGC |
| SEQ ID: 33 | GUUUUAUAACCUCAACUU |
| SEQ ID: 34 | GGUUUUAUAACCUCAACU |
| SEQ ID: 35 | CAGGGAUGGGCAUAUAUC |
| SEQ ID: 36 | ACAGGGAUGGGCAUAUAU |
| SEQ ID: 37 | CAACAGGGAUGGGCAUAU |
| SEQ ID: 38 | CCAACAGGGAUGGGCAUA |
| SEQ ID: 39 | UCCAACAGGGAUGGGCAU |
| SEQ ID: 40 | CUCCAACAGGGAUGGGCA |
| SEQ ID: 41 | CGGCUGCUGGCACCUCCA |
| SEQ ID: 42 | GUCAUCCCAUCUGCAAGG |
| SEQ ID: 43 | AGUCAUCCCAUCUGCAAG |
| SEQ ID: 44 | CGAGUCAUCCCAUCUGCA |
| SEQ ID: 45 | ACAAGCACGUGGAAGACG |
| SEQ ID: 46 | CACAAGCACGUGGAAGAC |
| SEQ ID: 47 | GUCACAAGCACGUGGAAG |
| SEQ ID: 48 | UGCAGAGUCACAAGCACG |
| SEQ ID: 49 | CUUCUGCAGAGUCACAAG |
| SEQ ID: 50 | GGACUGGAAACGGAUAUA |
| SEQ ID: 51 | UGGACUGGAAACGGAUAU |
| SEQ ID: 52 | CCUGGACUGGAAACGGAU |
| SEQ ID: 53 | UAUGCUCCCAGCCAUGGC |
| SEQ ID: 54 | CUAUGCUCCCAGCCAUGG |
| SEQ ID: 55 | CCUAUGCUCCCAGCCAUG |
| SEQ ID: 56 | CAUUCUGUGCUGCAUCCU |
| SEQ ID: 57 | AGUAAUACAGUUUGAACU |
| SEQ ID: 58 | UAAAGUAAUACAGUUUGA |
| SEQ ID: 59 | UUAAAGUAAUACAGUUUG |
| SEQ ID: 60 | AUUAAAGUAAUACAGUUU |
| SEQ ID: 61 | UCCAUUAAAGUAAUACAG |
| SEQ ID: 62 | UUCCAUUAAAGUAAUACA |
| SEQ ID: 63 | CUUCCAUUAAAGUAAUAC |
| SEQ ID: 64 | GCUUCCAUUAAAGUAAUA |
| SEQ ID: 65 | AUGGUGCAGCUGAUAUAU |
| SEQ ID: 66 | CAUGGUGCAGCUGAUAUA |
| SEQ ID: 67 | GCAUGGUGCAGCUGAUAU |
| SEQ ID: 68 | GGCAUGGUGCAGCUGAUA |
| SEQ ID: 69 | UGGCAUGGUGCAGCUGAU |
| SEQ ID: 70 | GAUGGCAUGGUGCAGCUG |
| SEQ ID: 71 | GAACACCUAGCAUCUGCA |
| SEQ ID: 72 | UUGAACACCUAGCAUCUG |
| SEQ ID: 73 | CUUGAACACCUAGCAUCU |
| SEQ ID: 74 | CCUUGAACACCUAGCAUC |
| SEQ ID: 75 | UCCUUGAACACCUAGCAU |
| SEQ ID: 76 | AUGCUCCUUGAACACCUA |
| SEQ ID: 77 | GCAUGCUCCUUGAACACC |
| SEQ ID: 78 | UGCAUGCUCCUUGAACAC |
| SEQ ID: 79 | UUGCAUGCUCCUUGAACA |
| SEQ ID: 80 | CUUUGCAUGCUCCUUGAA |
| SEQ ID: 81 | UCUUUGCAUGCUCCUUGA |
| SEQ ID: 82 | AAUUCGGGCAAGCUGCCG |
| SEQ ID: 83 | ACAAUUCGGGCAAGCUGC |
| SEQ ID: 84 | AAGAUGUCCUGCUGCCAA |
| SEQ ID: 85 | CAAGAUGUCCUGCUGCCA |
| SEQ ID: 86 | GACAAGAUGUCCUGCUGC |
| SEQ ID: 87 | AAGGCCUCUCUGAACAAA |
| SEQ ID: 88 | AGCUCUGAUAUCUUUAGU |
| SEQ ID: 89 | UUACAACAUAUACAGAUU |
| SEQ ID: 90 | AUUACAACAUAUACAGAU |
| SEQ ID: 91 | CUAGAGUAUUUUAUCAGU |
| SEQ ID: 92 | AGCCAGGGUUUCCUGGAC |
| SEQ ID: 93 | GCAAGCCAGGGUUUCCUG |
| SEQ ID: 94 | UCAGAGUCCAUCUUCUAC |
| SEQ ID: 95 | UUCAGAGUCCAUCUUCUA |
| SEQ ID: 96 | AGUAAUUGUACAAAACAU |
| SEQ ID: 97 | AUUCUAUAACAAAAUGUA |
| SEQ ID: 98 | GUAUUCUAUAACAAAAUG |
| SEQ ID: 99 | AGUAUUCUAUAACAAAAU |
| SEQ ID: 100 | AAAAAGUCUUGACAACAU |
| SEQ ID: 101 | GAAAAAGUCUUGACAACA |
| SEQ ID: 102 | CGAAAAAGUCUUGACAAC |
| SEQ ID: 103 | GUUCAUGGACAUCAUGCA |
| SEQ ID: 104 | UGUUCAUGGACAUCAUGC |
| SEQ ID: 105 | AAUUCCAACCACAGAUCU |
| SEQ ID: 106 | UAAUUCCAACCACAGAUC |
| SEQ ID: 107 | AUAAUUCCAACCACAGAU |
| SEQ ID: 108 | GAACUGGAAGUAAAUAUA |
| SEQ ID: 109 | GGAACUGGAAGUAAAUAU |
| SEQ ID: 110 | UGGAACUGGAAGUAAAUA |
| SEQ ID: 111 | AUUCUGGAACUGGAAGUA |
| SEQ ID: 112 | GGCCAGCAAUACCCAAAA |
| SEQ ID: 113 | AAUGACAACUGUACUGAA |
| SEQ ID: 114 | GAAUGACAACUGUACUGA |
| SEQ ID: 115 | AGCCUGUCAAUUCUUUGU |
| SEQ ID: 116 | AAGCCUGUCAAUUCUUUG |
| SEQ ID: 117 | GGGCAAAGCUUCAUUCAA |
| SEQ ID: 118 | AGGGCAAAGCUUCAUUCA |
| SEQ ID: 119 | AAGGGCAAAGCUUCAUUC |
| SEQ ID: 120 | GAAAAAGGGCAAAGCUUC |
| SEQ ID: 121 | GGAACUGAGGGCAAACUU |
| SEQ ID: 122 | UUGGAACUGAGGGCAAAC |
| SEQ ID: 123 | UUACUUCAUCCUGUGAGU |
| SEQ ID: 124 | CUUACUUCAUCCUGUGAG |
| SEQ ID: 125 | GCAAUAUUUUCCCUUACU |
| SEQ ID: 126 | AGCAAUAUUUUCCCUUAC |
| SEQ ID: 127 | CGAGCAAUAUUUUCCCUU |
| SEQ ID: 128 | CACGAGCAAUAUUUUCCC |
| SEQ ID: 129 | CCACGAGCAAUAUUUUCC |
| SEQ ID: 130 | UCCACGAGCAAUAUUUUC |
| SEQ ID: 131 | UUCCACGAGCAAUAUUUU |
| SEQ ID: 132 | CCAUUCCACGAGCAAUAU |
| SEQ ID: 133 | GCCAUUCCACGAGCAAUA |
| SEQ ID: 134 | UGCCAUUCCACGAGCAAU |
| SEQ ID: 135 | UUGCCAUUCCACGAGCAA |
| SEQ ID: 136 | AUUGCCAUUCCACGAGCA |
| SEQ ID: 137 | AAUUGCCAUUCCACGAGC |
| SEQ ID: 138 | UCCAAUCACAAGACAUUC |
| SEQ ID: 139 | CUCCAAUCACAAGACAUU |
| SEQ ID: 140 | UACCAACUCCAAUCACAA |
| SEQ ID: 141 | GUACCAACUCCAAUCACA |
| SEQ ID: 142 | GGUACCAACUCCAAUCAC |
| SEQ ID: 143 | AUGGUACCAACUCCAAUC |
| SEQ ID: 144 | ACAUGGUACCAACUCCAA |
| SEQ ID: 145 | GACAUGGUACCAACUCCA |
| SEQ ID: 146 | CUGACAUGGUACCAACUC |
| SEQ ID: 147 | CCUGACAUGGUACCAACU |
| SEQ ID: 148 | UGCAGCCAAAGCAGCACA |
| SEQ ID: 149 | GAACACGAAGUAGUUGGC |
| SEQ ID: 150 | UGAACACGAAGUAGUUGG |
| SEQ ID: 151 | AUGAACACGAAGUAGUUG |
| SEQ ID: 152 | CAUGAACACGAAGUAGUU |
| SEQ ID: 153 | GUCAUGAACACGAAGUAG |
| SEQ ID: 154 | UUUCCCGAGAAAGCUCUA |
| SEQ ID: 155 | CUUUCCCGAGAAAGCUCU |
| SEQ ID: 156 | GCUUUCCCGAGAAAGCUC |
| SEQ ID: 157 | CGGCUUUCCCGAGAAAGC |
| SEQ ID: 158 | ACGACCCUCGCGGCUUUC |
| SEQ ID: 159 | GACGACCCUCGCGGCUUU |
| SEQ ID: 160 | GGACGACCCUCGCGGCUU |
| SEQ ID: 161 | UGGACGACCCUCGCGGCU |
| SEQ ID: 162 | UUGGACGACCCUCGCGGC |
| SEQ ID: 163 | AUUGGACGACCCUCGCGG |
| SEQ ID: 164 | AAUUGGACGACCCUCGCG |
| SEQ ID: 165 | AAAUUGGACGACCCUCGC |
| SEQ ID: 166 | CAAAUUGGACGACCCUCG |
| SEQ ID: 167 | UUCUUCUUCUAAAACUCG |
| SEQ ID: 168 | AAUCAUCUUGACCCUCUG |
| SEQ ID: 169 | GAGACAUAAUCAUCUUGA |
| SEQ ID: 170 | AGAGACAUAAUCAUCUUG |
| SEQ ID: 171 | CUAGAGACAUAAUCAUCU |
| SEQ ID: 172 | CCUAGAGACAUAAUCAUC |
| SEQ ID: 173 | GCCUAGAGACAUAAUCAU |
| SEQ ID: 174 | AGCCUAGAGACAUAAUCA |
| SEQ ID: 175 | AAGCCUAGAGACAUAAUC |
| SEQ ID: 176 | CAAGCCUAGAGACAUAAU |
| SEQ ID: 177 | UAUCCAGCGACUGUGAGC |
| SEQ ID: 178 | CUAUCCAGCGACUGUGAG |
| SEQ ID: 179 | GCUAUCCAGCGACUGUGA |
| SEQ ID: 180 | AGCUAUCCAGCGACUGUG |
| SEQ ID: 181 | CAGCUAUCCAGCGACUGU |
| SEQ ID: 182 | UCAGCUAUCCAGCGACUG |
| SEQ ID: 183 | GGAAACACUUGGUUCAAU |
| SEQ ID: 184 | CAGAGGGAAACACUUGGU |
| SEQ ID: 185 | AAUAUCCAUGCUGAUCAU |
| SEQ ID: 186 | UCAAUAUCCAUGCUGAUC |
| SEQ ID: 187 | ACUUGACAGCCAGAAGGA |
| SEQ ID: 188 | UACUUGACAGCCAGAAGG |
| SEQ ID: 189 | GUGUAGAUUCUGUCUCUG |
| SEQ ID: 190 | AGUGUAGAUUCUGUCUCU |
| SEQ ID: 191 | AGGUUCACGUCUACAACA |
| SEQ ID: 192 | GGGUUUUAUAACCUCAAC |
| SEQ ID: 193 | AAGGGUUUUAUAACCUCA |
| SEQ ID: 194 | UAAGGGUUUUAUAACCUC |
| SEQ ID: 195 | CUAAGGGUUUUAUAACCU |
| SEQ ID: 196 | AAUUUUGCACCUUUCUCU |
| SEQ ID: 197 | GAAUUUUGCACCUUUCUC |
| SEQ ID: 198 | GGAAUUUUGCACCUUUCU |
| SEQ ID: 199 | AGGAAUUUUGCACCUUUC |
| SEQ ID: 200 | AAGGAAUUUUGCACCUUU |
| SEQ ID: 201 | UAAGGAAUUUUGCACCUU |
| SEQ ID: 202 | CUAAGGAAUUUUGCACCU |
| SEQ ID: 203 | ACUAAGGAAUUUUGCACC |
| SEQ ID: 204 | CACUAAGGAAUUUUGCAC |
| SEQ ID: 205 | CAUCACUAAGGAAUUUUG |
| SEQ ID: 206 | GCAUCACUAAGGAAUUUU |
| SEQ ID: 207 | CUCAGCAUCACUAAGGAA |
| SEQ ID: 208 | GAUCUCAGCAUCACUAAG |
| SEQ ID: 209 | UGAUCUCAGCAUCACUAA |
| SEQ ID: 210 | GACUAACUGGAUGAUCUC |
| SEQ ID: 211 | UGACUAACUGGAUGAUCU |
| SEQ ID: 212 | UUGACUAACUGGAUGAUC |
| SEQ ID: 213 | GCAUUGACUAACUGGAUG |
| SEQ ID: 214 | GGGAUAUGCUUAGCAUUG |
| SEQ ID: 215 | GUAGGCUGGGAUAUGCUU |
| SEQ ID: 216 | UUGUAGGCUGGGAUAUGC |
| SEQ ID: 217 | AGUUUCCAUCAGAGUUUC |
| SEQ ID: 218 | GAGUUUCCAUCAGAGUUU |
| SEQ ID: 219 | UGAGUUUCCAUCAGAGUU |
| SEQ ID: 220 | AUGAGUUUCCAUCAGAGU |
| SEQ ID: 221 | CACGCUCAUGAGUUUCCA |
| SEQ ID: 222 | CCACGCUCAUGAGUUUCC |
| SEQ ID: 223 | ACCACGCUCAUGAGUUUC |
| SEQ ID: 224 | CACCACGCUCAUGAGUUU |
| SEQ ID: 225 | ACACCACGCUCAUGAGUU |
| SEQ ID: 226 | GCGAAUAGAUACACCACG |
| SEQ ID: 227 | AUUAUAAUCCCUGUAAGG |
| SEQ ID: 228 | AAUUAUAAUCCCUGUAAG |
| SEQ ID: 229 | CAAGGAGUAAUUAUAAUC |
| SEQ ID: 230 | CCAAGGAGUAAUUAUAAU |
| SEQ ID: 231 | ACCAAGGAGUAAUUAUAA |
| SEQ ID: 232 | CACCAAGGAGUAAUUAUA |
| SEQ ID: 233 | CCCAUCACCAAGGAGUAA |
| SEQ ID: 234 | UCCCAUCACCAAGGAGUA |
| SEQ ID: 235 | ACAAGCUCCCAUCACCAA |
| SEQ ID: 236 | ACAACAAGCUCCCAUCAC |
| SEQ ID: 237 | GGAUGGGCAUAUAUCCAA |
| SEQ ID: 238 | GGGAUGGGCAUAUAUCCA |
| SEQ ID: 239 | GGCCACAAGACAACCUUC |
| SEQ ID: 240 | UGCUGGCCACAAGACAAC |
| SEQ ID: 241 | UGGUGCUGGCCACAAGAC |
| SEQ ID: 242 | UUGGUGCUGGCCACAAGA |
| SEQ ID: 243 | UGGCACCUCCACCAAGAC |
| SEQ ID: 244 | CUGGCACCUCCACCAAGA |
| SEQ ID: 245 | UCGGCUGCUGGCACCUCC |
| SEQ ID: 246 | AAGGACUCGGCUGCUGGC |
| SEQ ID: 247 | CUGCAAGGACUCGGCUGC |
| SEQ ID: 248 | UCUGCAAGGACUCGGCUG |
| SEQ ID: 249 | AUCUGCAAGGACUCGGCU |
| SEQ ID: 250 | CAUCUGCAAGGACUCGGC |
| SEQ ID: 251 | CCCAUCUGCAAGGACUCG |
| SEQ ID: 252 | AUCCCAUCUGCAAGGACU |
| SEQ ID: 253 | ACGAGUCAUCCCAUCUGC |
| SEQ ID: 254 | CACGAGUCAUCCCAUCUG |
| SEQ ID: 255 | CCACGAGUCAUCCCAUCU |
| SEQ ID: 256 | GCCACGAGUCAUCCCAUC |
| SEQ ID: 257 | GGCCACGAGUCAUCCCAU |
| SEQ ID: 258 | GGGCCACGAGUCAUCCCA |
| SEQ ID: 259 | UGGGCCACGAGUCAUCCC |
| SEQ ID: 260 | CUGGGCCACGAGUCAUCC |
| SEQ ID: 261 | ACUGGGCCACGAGUCAUC |
| SEQ ID: 262 | AAGACGCACAACUGGGCC |
| SEQ ID: 263 | GAAGACGCACAACUGGGC |
| SEQ ID: 264 | UGGAAGACGCACAACUGG |
| SEQ ID: 265 | GUGGAAGACGCACAACUG |
| SEQ ID: 266 | CGUGGAAGACGCACAACU |
| SEQ ID: 267 | ACGUGGAAGACGCACAAC |
| SEQ ID: 268 | CACGUGGAAGACGCACAA |
| SEQ ID: 269 | UCACUUCUGCAGAGUCAC |
| SEQ ID: 270 | UAUCACUGCGAACCCUUC |
| SEQ ID: 271 | UUAUCACUGCGAACCCUU |
| SEQ ID: 272 | UUUAUCACUGCGAACCCU |
| SEQ ID: 273 | CCUUUAUCACUGCGAACC |
| SEQ ID: 274 | UCCUUUAUCACUGCGAAC |
| SEQ ID: 275 | GCCUCCUUUAUCACUGCG |
| SEQ ID: 276 | AAAUGCCUCCUUUAUCAC |
| SEQ ID: 277 | UAGACGUGCAAAUCUGCU |
| SEQ ID: 278 | GUAGACGUGCAAAUCUGC |
| SEQ ID: 279 | UGUAGACGUGCAAAUCUG |
| SEQ ID: 280 | CUGUAGACGUGCAAAUCU |
| SEQ ID: 281 | UCUGUAGACGUGCAAAUC |
| SEQ ID: 282 | UUUCUGUAGACGUGCAAA |
| SEQ ID: 283 | GUUUCUGUAGACGUGCAA |
| SEQ ID: 284 | GAUAUAAAGGUUGCGUCC |
| SEQ ID: 285 | GGAUAUAAAGGUUGCGUC |
| SEQ ID: 286 | CGGAUAUAAAGGUUGCGU |
| SEQ ID: 287 | ACGGAUAUAAAGGUUGCG |
| SEQ ID: 288 | AAACGGAUAUAAAGGUUG |
| SEQ ID: 289 | GAAACGGAUAUAAAGGUU |
| SEQ ID: 290 | GGAAACGGAUAUAAAGGU |
| SEQ ID: 291 | ACUGGAAACGGAUAUAAA |
| SEQ ID: 292 | GACUGGAAACGGAUAUAA |
| SEQ ID: 293 | ACCUGGACUGGAAACGGA |
| SEQ ID: 294 | AAUGACAGCUUCACAAAC |
| SEQ ID: 295 | UGGAAUGACAGCUUCACA |
| SEQ ID: 296 | CUGGAAUGACAGCUUCAC |
| SEQ ID: 297 | UUGGCUGGAAUGACAGCU |
| SEQ ID: 298 | ACCUUGGCUGGAAUGACA |
| SEQ ID: 299 | AACCUUGGCUGGAAUGAC |
| SEQ ID: 300 | CAACCUUGGCUGGAAUGA |
| SEQ ID: 301 | ACAACCUUGGCUGGAAUG |
| SEQ ID: 302 | UAGCCUCCUAUGCUCCCA |
| SEQ ID: 303 | GUAGCCUCCUAUGCUCCC |
| SEQ ID: 304 | UUGUAGCCUCCUAUGCUC |
| SEQ ID: 305 | GUUGUAGCCUCCUAUGCU |
| SEQ ID: 306 | GCGUUGUAGCCUCCUAUG |
| SEQ ID: 307 | GGCGUUGUAGCCUCCUAU |
| SEQ ID: 308 | GGGCGUUGUAGCCUCCUA |
| SEQ ID: 309 | UGGGCGUUGUAGCCUCCU |
| SEQ ID: 310 | UGCAUGGGCGUUGUAGCC |
| SEQ ID: 311 | GCUGCAUGGGCGUUGUAG |
| SEQ ID: 312 | UGCUGCAUGGGCGUUGUA |
| SEQ ID: 313 | UUGCUGCAUGGGCGUUGU |
| SEQ ID: 314 | GUUUGCUGCAUGGGCGUU |
| SEQ ID: 315 | UGUUUGCUGCAUGGGCGU |
| SEQ ID: 316 | AUGUUUGCUGCAUGGGCG |
| SEQ ID: 317 | GACAAUGUUUGCUGCAUG |
| SEQ ID: 318 | GUGCUGCAUCCUGUCCAC |
| SEQ ID: 319 | CAACAUUCUGUGCUGCAU |
| SEQ ID: 320 | AAUACAGUUUGAACUACC |
| SEQ ID: 321 | UAAUACAGUUUGAACUAC |
| SEQ ID: 322 | GUAAUACAGUUUGAACUA |
| SEQ ID: 323 | UGCUUCCAUUAAAGUAAU |
| SEQ ID: 324 | UUGCUUCCAUUAAAGUAA |
| SEQ ID: 325 | ACUUGCUUCCAUUAAAGU |
| SEQ ID: 326 | CACUUGCUUCCAUUAAAG |
| SEQ ID: 327 | UAAAUCUUCAUUUGUGGG |
| SEQ ID: 328 | GCAGCUGAUAUAUAAAUC |
| SEQ ID: 329 | AGAUGGCAUGGUGCAGCU |
| SEQ ID: 330 | UAGAUGGCAUGGUGCAGC |
| SEQ ID: 331 | AUAGAUGGCAUGGUGCAG |
| SEQ ID: 332 | UAUAGAUGGCAUGGUGCA |
| SEQ ID: 333 | CUAUAGAUGGCAUGGUGC |
| SEQ ID: 334 | UCUAUAGAUGGCAUGGUG |
| SEQ ID: 335 | CUCUAUAGAUGGCAUGGU |
| SEQ ID: 336 | UCUCUAUAGAUGGCAUGG |
| SEQ ID: 337 | CCUAUCUCUAUAGAUGGC |
| SEQ ID: 338 | CACCGUUCCUAUCUCUAU |
| SEQ ID: 339 | CCACCGUUCCUAUCUCUA |
| SEQ ID: 340 | CCCACCGUUCCUAUCUCU |
| SEQ ID: 341 | ACAGGCUUGCUGAGGUAG |
| SEQ ID: 342 | AACACCUAGCAUCUGCAA |
| SEQ ID: 343 | AUCUUUGCAUGCUCCUUG |
| SEQ ID: 344 | UAUCUUUGCAUGCUCCUU |
| SEQ ID: 345 | UUAUCUUUGCAUGCUCCU |
| SEQ ID: 346 | AUUAUCUUUGCAUGCUCC |
| SEQ ID: 347 | GAUUAUCUUUGCAUGCUC |
| SEQ ID: 348 | GGAUUAUCUUUGCAUGCU |
| SEQ ID: 349 | UUCGGGCAAGCUGCCGGG |
| SEQ ID: 350 | CACAAUUCGGGCAAGCUG |
| SEQ ID: 351 | ACACAAUUCGGGCAAGCU |
| SEQ ID: 352 | CACACAAUUCGGGCAAGC |
| SEQ ID: 353 | GUCCUGCUGCCAAUGCUG |
| SEQ ID: 354 | UGUCCUGCUGCCAAUGCU |
| SEQ ID: 355 | AUGUCCUGCUGCCAAUGC |
| SEQ ID: 356 | UUUGACAAGAUGUCCUGC |
| SEQ ID: 357 | UUUUGACAAGAUGUCCUG |
| SEQ ID: 358 | ACUUUUGACAAGAUGUCC |
| SEQ ID: 359 | GACUUUUGACAAGAUGUC |
| SEQ ID: 360 | UGACUUUUGACAAGAUGU |
| SEQ ID: 361 | AGUUCUACAUCCCAGACU |
| SEQ ID: 362 | GGCCUCUCUGAACAAAUA |
| SEQ ID: 363 | AGGCCUCUCUGAACAAAU |
| SEQ ID: 364 | UAUUUCUUGAACACUUCC |
| SEQ ID: 365 | UUAUUUCUUGAACACUUC |
| SEQ ID: 366 | UAGAGUAUUUUAUCAGUA |
| SEQ ID: 367 | CAAGCAAGCCAGGGUUUC |
| SEQ ID: 368 | UGGCCACAGGAACUAGGC |
| SEQ ID: 369 | AACCUCCUGGCCACAGGA |
| SEQ ID: 370 | CACCAACCUCCUGGCCAC |
| SEQ ID: 371 | UCAGUCACCAACCUCCUG |
| SEQ ID: 372 | AUUAAGAUUCAAUAACUC |
| SEQ ID: 373 | AAUUAAGAUUCAAUAACU |
| SEQ ID: 374 | AAAUUAAGAUUCAAUAAC |
| SEQ ID: 375 | CAAAACUUGUACAUUAAA |
| SEQ ID: 376 | UACAAAACUUGUACAUUA |
| SEQ ID: 377 | UAAGGAGUUCUUUAUUAU |
| SEQ ID: 378 | AUAAGGAGUUCUUUAUUA |
| SEQ ID: 379 | AAUAAGGAGUUCUUUAUU |
| SEQ ID: 380 | AAAUAAGGAGUUCUUUAU |
| SEQ ID: 381 | AAAAUAAGGAGUUCUUUA |
| SEQ ID: 382 | ACACUUGAAGCAUUAGAU |
| SEQ ID: 383 | ACUUCCAUGAAAAGCUCA |
| SEQ ID: 384 | CCACUUCCAUGAAAAGCU |
| SEQ ID: 385 | UGCCACUUCCAUGAAAAG |
| SEQ ID: 386 | UUCACUGCCACUUCCAUG |
| SEQ ID: 387 | GUAAUUGUACAAAACAUU |

TABLE 3
Exemplary full-length sense and antisense strands of HMGCR RNAi agents

| | | | | | Start |
|---|---|---|---|---|---|
| | | | | | Position on |
| | | | | | human HMGCR |
| | SEQ ID | | SEQ ID | | transcript |
| Duplex NO: | ID NO: | Sense Strand 5′ to 3′ | ID NO: | Antisense Strand 5′ to 3′ | NM_000859.3 |
| Duplex | SEQ ID | GCUACAAUGUUGUCAAGACUA | SEQ ID | UAGUCUUGACAACAUUGUAGCUA | 99 |
| NO: 1 | NO: 388 | | NO: 774 | | |
| Duplex | SEQ ID | CUACAAUGUUGUCAAGACUUA | SEQ ID | UAAGUCUUGACAACAUUGUAGCU | 100 |
| NO: 2 | NO: 389 | | NO: 775 | | |
| Duplex | SEQ ID | AUGAUGUCCAUGAACAUGUUA | SEQ ID | UAACAUGUUCAUGGACAUCAUGC | 195 |
| NO: 3 | NO: 390 | | NO: 776 | | |
| Duplex | SEQ ID | AUAUUUACUUCCAGUUCCAGA | SEQ ID | UCUGGAACUGGAAGUAAAUAUAC | 328 |
| NO: 4 | NO: 391 | | NO: 777 | | |
| Duplex | SEQ ID | AUUUACUUCCAGUUCCAGAAA | SEQ ID | UUUCUGGAACUGGAAGUAAAUAU | 330 |
| NO: 5 | NO: 392 | | NO: 778 | | |
| Duplex | SEQ ID | AAUGAAGCUUUGCCCUUUUUA | SEQ ID | UAAAAAGGGCAAAGCUUCAUUCA | 468 |
| NO: 6 | NO: 393 | | NO: 779 | | |
| Duplex | SEQ ID | CACAUUAGCAAAGUUUGCCCA | SEQ ID | UGGGCAAACUUUGCUAAUGUGCU | 518 |
| NO: 7 | NO: 394 | | NO: 780 | | |
| Duplex | SEQ ID | ACAUUAGCAAAGUUUGCCCUA | SEQ ID | UAGGGCAAACUUUGCUAAUGUGC | 519 |
| NO: 8 | NO: 395 | | NO: 781 | | |
| Duplex | SEQ ID | CAUUAGCAAAGUUUGCCCUCA | SEQ ID | UGAGGGCAAACUUUGCUAAUGUG | 520 |
| NO: 9 | NO: 396 | | NO: 782 | | |
| Duplex | SEQ ID | UAGCAAAGUUUGCCCUCAGUA | SEQ ID | UACUGAGGGCAAACUUUGCUAAU | 523 |
| NO: 10 | NO: 397 | | NO: 783 | | |
| Duplex | SEQ ID | GCAAAGUUUGCCCUCAGUUCA | SEQ ID | UGAACUGAGGGCAAACUUUGCUA | 525 |
| NO: 11 | NO: 398 | | NO: 784 | | |
| Duplex | SEQ ID | ACUCACAGGAUGAAGUAAGGA | SEQ ID | UCCUUACUUCAUCCUGUGAGUUG | 547 |
| NO: 12 | NO: 399 | | NO: 785 | | |
| Duplex | SEQ ID | CACAGGAUGAAGUAAGGGAAA | SEQ ID | UUUCCCUUACUUCAUCCUGUGAG | 550 |
| NO: 13 | NO: 400 | | NO: 786 | | |
| Duplex | SEQ ID | CAGGAUGAAGUAAGGGAAAAA | SEQ ID | UUUUUCCCUUACUUCAUCCUGUG | 552 |
| NO: 14 | NO: 401 | | NO: 787 | | |
| Duplex | SEQ ID | AGGAUGAAGUAAGGGAAAAUA | SEQ ID | UAUUUUCCCUUACUUCAUCCUGU | 553 |
| NO: 15 | NO: 402 | | NO: 788 | | |
| Duplex | SEQ ID | GCGAGGGUCGUCCAAUUUGGA | SEQ ID | UCCAAAUUGGACGACCCUCGCGG | 784 |
| NO: 16 | NO: 403 | | NO: 789 | | |
| Duplex | SEQ ID | CGAGGGUCGUCCAAUUUGGCA | SEQ ID | UGCCAAAUUGGACGACCCUCGCG | 785 |
| NO: 17 | NO: 404 | | NO: 790 | | |
| Duplex | SEQ ID | GAGGGUCGUCCAAUUUGGCAA | SEQ ID | UUGCCAAAUUGGACGACCCUCGC | 786 |
| NO: 18 | NO: 405 | | NO: 791 | | |
| Duplex | SEQ ID | GUCGUCCAAUUUGGCAGCUCA | SEQ ID | UGAGCUGCCAAAUUGGACGACCC | 790 |
| NO: 19 | NO: 406 | | NO: 792 | | |
| Duplex | SEQ ID | UCGUCCAAUUUGGCAGCUCAA | SEQ ID | UUGAGCUGCCAAAUUGGACGACC | 791 |
| NO: 20 | NO: 407 | | NO: 793 | | |
| Duplex | SEQ ID | CGUCCAAUUUGGCAGCUCAGA | SEQ ID | UCUGAGCUGCCAAAUUGGACGAC | 792 |
| NO: 21 | NO: 408 | | NO: 794 | | |
| Duplex | SEQ ID | UCCAAUUUGGCAGCUCAGCCA | SEQ ID | UGGCUGAGCUGCCAAAUUGGACG | 794 |
| NO: 22 | NO: 409 | | NO: 795 | | |
| Duplex | SEQ ID | AAUUUGGCAGCUCAGCCAUUA | SEQ ID | UAAUGGCUGAGCUGCCAAAUUGG | 797 |
| NO: 23 | NO: 410 | | NO: 796 | | |
| Duplex | SEQ ID | UUGGCAGCUCAGCCAUUUUGA | SEQ ID | UCAAAAUGGCUGAGCUGCCAAAU | 800 |
| NO: 24 | NO: 411 | | NO: 797 | | |
| Duplex | SEQ ID | UGGCAGCUCAGCCAUUUUGCA | SEQ ID | UGCAAAAUGGCUGAGCUGCCAAA | 801 |
| NO: 25 | NO: 412 | | NO: 798 | | |
| Duplex | SEQ ID | CCGAGUUUUAGAAGAAGAAGA | SEQ ID | UCUUCUUCUUCUAAAACUCGGGC | 821 |
| NO: 26 | NO: 413 | | NO: 799 | | |
| Duplex | SEQ ID | AGAGGGUCAAGAUGAUUAUGA | SEQ ID | UCAUAAUCAUCUUGACCCUCUGA | 865 |
| NO: 27 | NO: 414 | | NO: 800 | | |
| Duplex | SEQ ID | GGGUCAAGAUGAUUAUGUCUA | SEQ ID | UAGACAUAAUCAUCUUGACCCUC | 868 |
| NO: 28 | NO: 415 | | NO: 801 | | |
| Duplex | SEQ ID | AGUCGCUGGAUAGCUGAUCCA | SEQ ID | UGGAUCAGCUAUCCAGCGACUGU | 915 |
| NO: 29 | NO: 416 | | NO: 802 | | |
| Duplex | SEQ ID | GUCGCUGGAUAGCUGAUCCUA | SEQ ID | UAGGAUCAGCUAUCCAGCGACUG | 916 |
| NO: 30 | NO: 417 | | NO: 803 | | |
| Duplex | SEQ ID | UCGCUGGAUAGCUGAUCCUUA | SEQ ID | UAAGGAUCAGCUAUCCAGCGACU | 917 |
| NO: 31 | NO: 418 | | NO: 804 | | |
| Duplex | SEQ ID | AAAAGUUGAGGUUAUAAAACA | SEQ ID | UGUUUUAUAACCUCAACUUUUCU | 1289 |
| NO: 32 | NO: 419 | | NO: 805 | | |
| Duplex | SEQ ID | AAAGUUGAGGUUAUAAAACCA | SEQ ID | UGGUUUUAUAACCUCAACUUUUC | 1290 |
| NO: 33 | NO: 420 | | NO: 806 | | |
| Duplex | SEQ ID | UGGAUAUAUGCCCAUCCCUGA | SEQ ID | UCAGGGAUGGGCAUAUAUCCAAU | 1697 |
| NO: 34 | NO: 421 | | NO: 807 | | |
| Duplex | SEQ ID | GGAUAUAUGCCCAUCCCUGUA | SEQ ID | UACAGGGAUGGGCAUAUAUCCAA | 1698 |
| NO: 35 | NO: 422 | | NO: 808 | | |
| Duplex | SEQ ID | AUAUAUGCCCAUCCCUGUUGA | SEQ ID | UCAACAGGGAUGGGCAUAUAUCC | 1700 |
| NO: 36 | NO: 423 | | NO: 809 | | |
| Duplex | SEQ ID | UAUAUGCCCAUCCCUGUUGGA | SEQ ID | UCCAACAGGGAUGGGCAUAUAUC | 1701 |
| NO: 37 | NO: 424 | | NO: 810 | | |
| Duplex | SEQ ID | AUAUGCCCAUCCCUGUUGGAA | SEQ ID | UUCCAACAGGGAUGGGCAUAUAU | 1702 |
| NO: 38 | NO: 425 | | NO: 811 | | |
| Duplex | SEQ ID | UAUGCCCAUCCCUGUUGGAGA | SEQ ID | UCUCCAACAGGGAUGGGCAUAUA | 1703 |
| NO: 39 | NO: 426 | | NO: 812 | | |
| Duplex | SEQ ID | GGUGGAGGUGCCAGCAGCCGA | SEQ ID | UCGGCUGCUGGCACCUCCACCAA | 1830 |
| NO: 40 | NO: 427 | | NO: 813 | | |
| Duplex | SEQ ID | GUCCUUGCAGAUGGGAUGACA | SEQ ID | UGUCAUCCCAUCUGCAAGGACUC | 1851 |
| NO: 41 | NO: 428 | | NO: 814 | | |
| Duplex | SEQ ID | UCCUUGCAGAUGGGAUGACUA | SEQ ID | UAGUCAUCCCAUCUGCAAGGACU | 1852 |
| NO: 42 | NO: 429 | | NO: 815 | | |
| Duplex | SEQ ID | CUUGCAGAUGGGAUGACUCGA | SEQ ID | UCGAGUCAUCCCAUCUGCAAGGA | 1854 |
| NO: 43 | NO: 430 | | NO: 816 | | |
| Duplex | SEQ ID | UGCGUCUUCCACGUGCUUGUA | SEQ ID | UACAAGCACGUGGAAGACGCACA | 1885 |
| NO: 44 | NO: 431 | | NO: 817 | | |
| Duplex | SEQ ID | GCGUCUUCCACGUGCUUGUGA | SEQ ID | UCACAAGCACGUGGAAGACGCAC | 1886 |
| NO: 45 | NO: 432 | | NO: 818 | | |
| Duplex | SEQ ID | GUCUUCCACGUGCUUGUGACA | SEQ ID | UGUCACAAGCACGUGGAAGACGC | 1888 |
| NO: 46 | NO: 433 | | NO: 819 | | |
| Duplex | SEQ ID | CACGUGCUUGUGACUCUGCAA | SEQ ID | UUGCAGAGUCACAAGCACGUGGA | 1894 |
| NO: 47 | NO: 434 | | NO: 821 | | |
| Duplex | SEQ ID | UGCUUGUGACUCUGCAGAAGA | SEQ ID | UCUUCUGCAGAGUCACAAGCACG | 1898 |
| NO: 48 | NO: 435 | | NO: 821 | | |
| Duplex | SEQ ID | UUUAUAUCCGUUUCCAGUCCA | SEQ ID | UGGACUGGAAACGGAUAUAAAGG | 2032 |
| NO: 49 | NO: 436 | | NO: 822 | | |
| Duplex | SEQ ID | UUAUAUCCGUUUCCAGUCCAA | SEQ ID | UUGGACUGGAAACGGAUAUAAAG | 2033 |
| NO: 50 | NO: 437 | | NO: 823 | | |
| Duplex | SEQ ID | AUAUCCGUUUCCAGUCCAGGA | SEQ ID | UCCUGGACUGGAAACGGAUAUAA | 2035 |
| NO: 51 | NO: 438 | | NO: 824 | | |
| Duplex | SEQ ID | CUGCCAUGGCUGGGAGCAUAA | SEQ ID | UUAUGCUCCCAGCCAUGGCAGAG | 2323 |
| NO: 52 | NO: 439 | | NO: 825 | | |
| Duplex | SEQ ID | UGCCAUGGCUGGGAGCAUAGA | SEQ ID | UCUAUGCUCCCAGCCAUGGCAGA | 2324 |
| NO: 53 | NO: 440 | | NO: 826 | | |
| Duplex | SEQ ID | GCCAUGGCUGGGAGCAUAGGA | SEQ ID | UCCUAUGCUCCCAGCCAUGGCAG | 2325 |
| NO: 54 | NO: 441 | | NO: 827 | | |
| Duplex | SEQ ID | ACAGGAUGCAGCACAGAAUGA | SEQ ID | UCAUUCUGUGCUGCAUCCUGUUC | 2399 |
| NO: 55 | NO: 442 | | NO: 828 | | |
| Duplex | SEQ ID | GUAGUUCAAACUGUAUUACUA | SEQ ID | UAGUAAUACAGUUUGAACUACCA | 2422 |
| NO: 56 | NO: 443 | | NO: 829 | | |
| Duplex | SEQ ID | GUUCAAACUGUAUUACUUUAA | SEQ ID | UUAAAGUAAUACAGUUUGAACUA | 2425 |
| NO: 57 | NO: 444 | | NO: 830 | | |
| Duplex | SEQ ID | UUCAAACUGUAUUACUUUAAA | SEQ ID | UUUAAAGUAAUACAGUUUGAACU | 2426 |
| NO: 58 | NO: 445 | | NO: 831 | | |
| Duplex | SEQ ID | UCAAACUGUAUUACUUUAAUA | SEQ ID | UAUUAAAGUAAUACAGUUUGAAC | 2427 |
| NO: 59 | NO: 446 | | NO: 832 | | |
| Duplex | SEQ ID | AACUGUAUUACUUUAAUGGAA | SEQ ID | UUCCAUUAAAGUAAUACAGUUUG | 2430 |
| NO: 60 | NO: 447 | | NO: 833 | | |
| Duplex | SEQ ID | ACUGUAUUACUUUAAUGGAAA | SEQ ID | UUUCCAUUAAAGUAAUACAGUUU | 2431 |
| NO: 61 | NO: 448 | | NO: 834 | | |
| Duplex | SEQ ID | CUGUAUUACUUUAAUGGAAGA | SEQ ID | UCUUCCAUUAAAGUAAUACAGUU | 2432 |
| NO: 62 | NO: 449 | | NO: 835 | | |
| Duplex | SEQ ID | UGUAUUACUUUAAUGGAAGCA | SEQ ID | UGCUUCCAUUAAAGUAAUACAGU | 2433 |
| NO: 63 | NO: 450 | | NO: 836 | | |
| Duplex | SEQ ID | UUAUAUAUCAGCUGCACCAUA | SEQ ID | UAUGGUGCAGCUGAUAUAUAAAU | 2475 |
| NO: 64 | NO: 451 | | NO: 837 | | |
| Duplex | SEQ ID | UAUAUAUCAGCUGCACCAUGA | SEQ ID | UCAUGGUGCAGCUGAUAUAUAAA | 2476 |
| NO: 65 | NO: 452 | | NO: 838 | | |
| Duplex | SEQ ID | AUAUAUCAGCUGCACCAUGCA | SEQ ID | UGCAUGGUGCAGCUGAUAUAUAA | 2477 |
| NO: 66 | NO: 453 | | NO: 839 | | |
| Duplex | SEQ ID | UAUAUCAGCUGCACCAUGCCA | SEQ ID | UGGCAUGGUGCAGCUGAUAUAUA | 2478 |
| NO: 67 | NO: 454 | | NO: 840 | | |
| Duplex | SEQ ID | AUAUCAGCUGCACCAUGCCAA | SEQ ID | UUGGCAUGGUGCAGCUGAUAUAU | 2479 |
| NO: 68 | NO: 455 | | NO: 841 | | |
| Duplex | SEQ ID | AUCAGCUGCACCAUGCCAUCA | SEQ ID | UGAUGGCAUGGUGCAGCUGAUAU | 2481 |
| NO: 69 | NO: 456 | | NO: 842 | | |
| Duplex | SEQ ID | UUUGCAGAUGCUAGGUGUUCA | SEQ ID | UGAACACCUAGCAUCUGCAAACA | 2555 |
| NO: 70 | NO: 457 | | NO: 843 | | |
| Duplex | SEQ ID | UGCAGAUGCUAGGUGUUCAAA | SEQ ID | UUUGAACACCUAGCAUCUGCAAA | 2557 |
| NO: 71 | NO: 458 | | NO: 844 | | |
| Duplex | SEQ ID | GCAGAUGCUAGGUGUUCAAGA | SEQ ID | UCUUGAACACCUAGCAUCUGCAA | 2558 |
| NO: 72 | NO: 459 | | NO: 845 | | |
| Duplex | SEQ ID | CAGAUGCUAGGUGUUCAAGGA | SEQ ID | UCCUUGAACACCUAGCAUCUGCA | 2559 |
| NO: 73 | NO: 460 | | NO: 846 | | |
| Duplex | SEQ ID | AGAUGCUAGGUGUUCAAGGAA | SEQ ID | UUCCUUGAACACCUAGCAUCUGC | 2560 |
| NO: 74 | NO: 461 | | NO: 847 | | |
| Duplex | SEQ ID | GCUAGGUGUUCAAGGAGCAUA | SEQ ID | UAUGCUCCUUGAACACCUAGCAU | 2564 |
| NO: 75 | NO: 462 | | NO: 848 | | |
| Duplex | SEQ ID | UAGGUGUUCAAGGAGCAUGCA | SEQ ID | UGCAUGCUCCUUGAACACCUAGC | 2566 |
| NO: 76 | NO: 463 | | NO: 849 | | |
| Duplex | SEQ ID | AGGUGUUCAAGGAGCAUGCAA | SEQ ID | UUGCAUGCUCCUUGAACACCUAG | 2567 |
| NO: 77 | NO: 464 | | NO: 850 | | |
| Duplex | SEQ ID | GGUGUUCAAGGAGCAUGCAAA | SEQ ID | UUUGCAUGCUCCUUGAACACCUA | 2568 |
| NO: 78 | NO: 465 | | NO: 851 | | |
| Duplex | SEQ ID | UGUUCAAGGAGCAUGCAAAGA | SEQ ID | UCUUUGCAUGCUCCUUGAACACC | 2570 |
| NO: 79 | NO: 466 | | NO: 852 | | |
| Duplex | SEQ ID | GUUCAAGGAGCAUGCAAAGAA | SEQ ID | UUCUUUGCAUGCUCCUUGAACAC | 2571 |
| NO: 80 | NO: 467 | | NO: 853 | | |
| Duplex | SEQ ID | CCCGGCAGCUUGCCCGAAUUA | SEQ ID | UAAUUCGGGCAAGCUGCCGGGCA | 2608 |
| NO: 81 | NO: 468 | | NO: 854 | | |
| Duplex | SEQ ID | CGGCAGCUUGCCCGAAUUGUA | SEQ ID | UACAAUUCGGGCAAGCUGCCGGG | 2610 |
| NO: 82 | NO: 469 | | NO: 855 | | |
| Duplex | SEQ ID | CAUUGGCAGCAGGACAUCUUA | SEQ ID | UAAGAUGUCCUGCUGCCAAUGCU | 2671 |
| NO: 83 | NO: 470 | | NO: 856 | | |
| Duplex | SEQ ID | AUUGGCAGCAGGACAUCUUGA | SEQ ID | UCAAGAUGUCCUGCUGCCAAUGC | 2672 |
| NO: 84 | NO: 471 | | NO: 857 | | |
| Duplex | SEQ ID | UGGCAGCAGGACAUCUUGUCA | SEQ ID | UGACAAGAUGUCCUGCUGCCAAU | 2674 |
| NO: 85 | NO: 472 | | NO: 858 | | |
| Duplex | SEQ ID | UAUUUGUUCAGAGAGGCCUUA | SEQ ID | UAAGGCCUCUCUGAACAAAUAGC | 3456 |
| NO: 86 | NO: 473 | | NO: 859 | | |
| Duplex | SEQ ID | UUACUAAAGAUAUCAGAGCUA | SEQ ID | UAGCUCUGAUAUCUUUAGUAAAG | 3575 |
| NO: 87 | NO: 474 | | NO: 860 | | |
| Duplex | SEQ ID | CAAAUCUGUAUAUGUUGUAAA | SEQ ID | UUUACAACAUAUACAGAUUUGAU | 3767 |
| NO: 88 | NO: 475 | | NO: 861 | | |
| Duplex | SEQ ID | AAAUCUGUAUAUGUUGUAAUA | SEQ ID | UAUUACAACAUAUACAGAUUUGA | 3768 |
| NO: 89 | NO: 476 | | NO: 862 | | |
| Duplex | SEQ ID | GUACUGAUAAAAUACUCUAGA | SEQ ID | UCUAGAGUAUUUUAUCAGUACAC | 3839 |
| NO: 90 | NO: 477 | | NO: 863 | | |
| Duplex | SEQ ID | UUGUCCAGGAAACCCUGGCUA | SEQ ID | UAGCCAGGGUUUCCUGGACAACA | 3890 |
| NO: 91 | NO: 478 | | NO: 864 | | |
| Duplex | SEQ ID | UCCAGGAAACCCUGGCUUGCA | SEQ ID | UGCAAGCCAGGGUUUCCUGGACA | 3893 |
| NO: 92 | NO: 479 | | NO: 865 | | |
| Duplex | SEQ ID | UUGUAGAAGAUGGACUCUGAA | SEQ ID | UUCAGAGUCCAUCUUCUACAAGA | 4202 |
| NO: 93 | NO: 480 | | NO: 866 | | |
| Duplex | SEQ ID | UGUAGAAGAUGGACUCUGAAA | SEQ ID | UUUCAGAGUCCAUCUUCUACAAG | 4203 |
| NO: 94 | NO: 481 | | NO: 867 | | |
| Duplex | SEQ ID | UAAUGUUUUGUACAAUUACUA | SEQ ID | UAGUAAUUGUACAAAACAUUAAU | 4439 |
| NO: 95 | NO: 482 | | NO: 868 | | |
| Duplex | SEQ ID | UAUACAUUUUGUUAUAGAAUA | SEQ ID | UAUUCUAUAACAAAAUGUAUACA | 4465 |
| NO: 96 | NO: 483 | | NO: 869 | | |
| Duplex | SEQ ID | UACAUUUUGUUAUAGAAUACA | SEQ ID | UGUAUUCUAUAACAAAAUGUAUA | 4467 |
| NO: 97 | NO: 484 | | NO: 870 | | |
| Duplex | SEQ ID | ACAUUUUGUUAUAGAAUACUA | SEQ ID | UAGUAUUCUAUAACAAAAUGUAU | 4468 |
| NO: 98 | NO: 485 | | NO: 871 | | |
| Duplex | SEQ ID | CAAUGUUGUCAAGACUUUUUA | SEQ ID | UAAAAAGUCUUGACAACAUUGUA | 103 |
| NO: 99 | NO: 486 | | NO: 872 | | |
| Duplex | SEQ ID | AAUGUUGUCAAGACUUUUUCA | SEQ ID | UGAAAAAGUCUUGACAACAUUGU | 104 |
| NO: 100 | NO: 487 | | NO: 873 | | |
| Duplex | SEQ ID | AUGUUGUCAAGACUUUUUCGA | SEQ ID | UCGAAAAAGUCUUGACAACAUUG | 105 |
| NO: 101 | NO: 488 | | NO: 874 | | |
| Duplex | SEQ ID | UCUGCAUGAUGUCCAUGAACA | SEQ ID | UGUUCAUGGACAUCAUGCAGAUG | 190 |
| NO: 102 | NO: 489 | | NO: 875 | | |
| Duplex | SEQ ID | CUGCAUGAUGUCCAUGAACAA | SEQ ID | UUGUUCAUGGACAUCAUGCAGAU | 191 |
| NO: 103 | NO: 490 | | NO: 876 | | |
| Duples | SEQ ID | UAAGAUCUGUGGUUGGAAUUA | SEQ ID | UAAUUCCAACCACAGAUCUUAUU | 227 |
| NO: 104 | NO: 491 | | NO: 877 | | |
| Duplex | SEQ ID | AAGAUCUGUGGUUGGAAUUAA | SEQ ID | UUAAUUCCAACCACAGAUCUUAU | 228 |
| NO: 105 | NO: 492 | | NO: 878 | | |
| Duplex | SEQ ID | AGAUCUGUGGUUGGAAUUAUA | SEQ ID | UAUAAUUCCAACCACAGAUCUUA | 229 |
| NO: 106 | NO: 493 | | NO: 879 | | |
| Duplex | SEQ ID | UGUAUAUUUACUUCCAGUUCA | SEQ ID | UGAACUGGAAGUAAAUAUACAGG | 325 |
| NO: 107 | NO: 494 | | NO: 880 | | |
| Duplex | SEQ ID | GUAUAUUUACUUCCAGUUCCA | SEQ ID | UGGAACUGGAAGUAAAUAUACAG | 326 |
| NO: 108 | NO: 495 | | NO: 881 | | |
| Duplex | SEQ ID | UAUAUUUACUUCCAGUUCCAA | SEQ ID | UUGGAACUGGAAGUAAAUAUACA | 327 |
| NO: 109 | NO: 496 | | NO: 882 | | |
| Duplex | SEQ ID | UUUACUUCCAGUUCCAGAAUA | SEQ ID | UAUUCUGGAACUGGAAGUAAAUA | 331 |
| NO: 110 | NO: 497 | | NO: 883 | | |
| Duplex | SEQ ID | UAUUUUGGGUAUUGCUGGCCA | SEQ ID | UGGCCAGCAAUACCCAAAAUAUA | 374 |
| NO: 111 | NO: 498 | | NO: 884 | | |
| Duplex | SEQ ID | UAUUCAGUACAGUUGUCAUUA | SEQ ID | UAAUGACAACUGUACUGAAUACA | 418 |
| NO: 112 | NO: 499 | | NO: 885 | | |
| Duplex | SEQ ID | AUUCAGUACAGUUGUCAUUCA | SEQ ID | UGAAUGACAACUGUACUGAAUAC | 419 |
| NO: 113 | NO: 500 | | NO: 886 | | |
| Duplex | SEQ ID | AGACAAAGAAUUGACAGGCUA | SEQ ID | UAGCCUGUCAAUUCUUUGUCUAA | 446 |
| NO: 114 | NO: 501 | | NO: 887 | | |
| Duplex | SEQ ID | GACAAAGAAUUGACAGGCUUA | SEQ ID | UAAGCCUGUCAAUUCUUUGUCUA | 447 |
| NO: 115 | NO: 502 | | NO: 888 | | |
| Duplex | SEQ ID | GCUUGAAUGAAGCUUUGCCCA | SEQ ID | UGGGCAAAGCUUCAUUCAAGCCU | 463 |
| NO: 116 | NO: 503 | | NO: 889 | | |
| Duplex | SEQ ID | CUUGAAUGAAGCUUUGCCCUA | SEQ ID | UAGGGCAAAGCUUCAUUCAAGCC | 464 |
| NO: 117 | NO: 504 | | NO: 890 | | |
| Duplex | SEQ ID | UUGAAUGAAGCUUUGCCCUUA | SEQ ID | UAAGGGCAAAGCUUCAUUCAAGC | 465 |
| NO: 118 | NO: 505 | | NO: 891 | | |
| Duplex | SEQ ID | AUGAAGCUUUGCCCUUUUUCA | SEQ ID | UGAAAAAGGGCAAAGCUUCAUUC | 469 |
| NO: 119 | NO: 506 | | NO: 892 | | |
| Duplex | SEQ ID | CAAAGUUUGCCCUCAGUUCCA | SEQ ID | UGGAACUGAGGGCAAACUUUGCU | 526 |
| NO: 120 | NO: 507 | | NO: 893 | | |
| Duplex | SEQ ID | AAGUUUGCCCUCAGUUCCAAA | SEQ ID | UUUGGAACUGAGGGCAAACUUUG | 528 |
| NO: 121 | NO: 508 | | NO: 894 | | |
| Duplex | SEQ ID | CAACUCACAGGAUGAAGUAAA | SEQ ID | UUUACUUCAUCCUGUGAGUUGGA | 545 |
| NO: 122 | NO: 509 | | NO: 895 | | |
| Duplex | SEQ ID | AACUCACAGGAUGAAGUAAGA | SEQ ID | UCUUACUUCAUCCUGUGAGUUGG | 546 |
| NO: 123 | NO: 510 | | NO: 896 | | |
| Duplex | SEQ ID | GAAGUAAGGGAAAAUAUUGCA | SEQ ID | UGCAAUAUUUUCCCUUACUUCAU | 558 |
| NO: 124 | NO: 511 | | NO: 897 | | |
| Duplex | SEQ ID | AAGUAAGGGAAAAUAUUGCUA | SEQ ID | UAGCAAUAUUUUCCCUUACUUCA | 559 |
| NO: 125 | NO: 512 | | NO: 898 | | |
| Duplex | SEQ ID | GUAAGGGAAAAUAUUGCUCGA | SEQ ID | UCGAGCAAUAUUUUCCCUUACUU | 561 |
| NO: 126 | NO: 513 | | NO: 899 | | |
| Duplex | SEQ ID | AAGGGAAAAUAUUGCUCGUGA | SEQ ID | UCACGAGCAAUAUUUUCCCUUAC | 563 |
| NO: 127 | NO: 514 | | NO: 900 | | |
| Duplex | SEQ ID | AGGGAAAAUAUUGCUCGUGGA | SEQ ID | UCCACGAGCAAUAUUUUCCCUUA | 564 |
| NO: 128 | NO: 515 | | NO: 901 | | |
| Duplex | SEQ ID | GGGAAAAUAUUGCUCGUGGAA | SEQ ID | UUCCACGAGCAAUAUUUUCCCUU | 565 |
| NO: 129 | NO: 516 | | NO: 902 | | |
| Duplex | SEQ ID | GGAAAAUAUUGCUCGUGGAAA | SEQ ID | UUUCCACGAGCAAUAUUUUCCCU | 566 |
| NO: 130 | NO: 517 | | NO: 903 | | |
| Duplex | SEQ ID | AAAUAUUGCUCGUGGAAUGGA | SEQ ID | UCCAUUCCACGAGCAAUAUUUUC | 569 |
| NO: 131 | NO: 518 | | NO: 904 | | |
| Duplex | SEQ ID | AAUAUUGCUCGUGGAAUGGCA | SEQ ID | UGCCAUUCCACGAGCAAUAUUUU | 570 |
| NO: 132 | NO: 516 | | NO: 905 | | |
| Duplex | SEQ ID | AUAUUGCUCGUGGAAUGGCAA | SEQ ID | UUGCCAUUCCACGAGCAAUAUUU | 571 |
| NO: 133 | NO: 520 | | NO: 906 | | |
| Duplex | SEQ ID | UAUUGCUCGUGGAAUGGCAAA | SEQ ID | UUUGCCAUUCCACGAGCAAUAUU | 572 |
| NO: 134 | NO: 521 | | NO: 907 | | |
| Duplex | SEQ ID | AUUGCUCGUGGAAUGGCAAUA | SEQ ID | UAUUGCCAUUCCACGAGCAAUAU | 573 |
| NO: 135 | NO: 522 | | NO: 908 | | |
| Duplex | SEQ ID | UUGCUCGUGGAAUGGCAAUUA | SEQ ID | UAAUUGCCAUUCCACGAGCAAUA | 574 |
| NO: 136 | NO: 523 | | NO: 909 | | |
| Duplex | SEQ ID | UUGAAUGUCUUGUGAUUGGAA | SEQ ID | UUCCAAUCACAAGACAUUCAACA | 625 |
| NO: 137 | NO: 524 | | NO: 910 | | |
| Duplex | SEQ ID | UGAAUGUCUUGUGAUUGGAGA | SEQ ID | UCUCCAAUCACAAGACAUUCAAC | 626 |
| NO: 138 | NO: 525 | | NO: 911 | | |
| Duplex | SEQ ID | UCUUGUGAUUGGAGUUGGUAA | SEQ ID | UUACCAACUCCAAUCACAAGACA | 632 |
| NO: 139 | NO: 526 | | NO: 912 | | |
| Duplex | SEQ ID | CUUGUGAUUGGAGUUGGUACA | SEQ ID | UGUACCAACUCCAAUCACAAGAC | 633 |
| NO: 141 | NO: 527 | | NO: 913 | | |
| Duplex | SEQ ID | UUGUGAUUGGAGUUGGUACCA | SEQ ID | UGGUACCAACUCCAAUCACAAGA | 634 |
| NO: 141 | NO: 528 | | NO: 914 | | |
| Duplex | SEQ ID | GUGAUUGGAGUUGGUACCAUA | SEQ ID | UAUGGUACCAACUCCAAUCACAA | 636 |
| NO: 142 | NO: 529 | | NO: 915 | | |
| Duplex | SEQ ID | GAUUGGAGUUGGUACCAUGUA | SEQ ID | UACAUGGUACCAACUCCAAUCAC | 638 |
| NO: 143 | NO: 530 | | NO: 916 | | |
| Duplex | SEQ ID | AUUGGAGUUGGUACCAUGUCA | SEQ ID | UGACAUGGUACCAACUCCAAUCA | 639 |
| NO: 144 | NO: 531 | | NO: 917 | | |
| Duplex | SEQ ID | UGGAGUUGGUACCAUGUCAGA | SEQ ID | UCUGACAUGGUACCAACUCCAAU | 641 |
| NO: 145 | NO: 532 | | NO: 918 | | |
| Duplex | SEQ ID | GGAGUUGGUACCAUGUCAGGA | SEQ ID | UCCUGACAUGGUACCAACUCCAA | 642 |
| NO: 146 | NO: 533 | | NO: 919 | | |
| Duplex | SEQ ID | UAUGUGCUGCUUUGGCUGCAA | SEQ ID | UUGCAGCCAAAGCAGCACAUAAU | 680 |
| NO: 147 | NO: 534 | | NO: 920 | | |
| Duplex | SEQ ID | UUGCCAACUACUUCGUGUUCA | SEQ ID | UGAACACGAAGUAGUUGGCAAGA | 709 |
| NO: 148 | NO: 535 | | NO: 921 | | |
| Duplex | SEQ ID | UGCCAACUACUUCGUGUUCAA | SEQ ID | UUGAACACGAAGUAGUUGGCAAG | 710 |
| NO: 149 | NO: 536 | | NO: 922 | | |
| Duplex | SEQ ID | GCCAACUACUUCGUGUUCAUA | SEQ ID | UAUGAACACGAAGUAGUUGGCAA | 711 |
| NO: 150 | NO: 537 | | NO: 923 | | |
| Duplex | SEQ ID | CCAAUCACUUCGUGUUCAUGA | SEQ ID | UCAUGAACACGAAGUAGUUGGCA | 712 |
| NO: 151 | NO: 538 | | NO: 924 | | |
| Duplex | SEQ ID | AACUACUUCGUGUUCAUGACA | SEQ ID | UGUCAUGAACACGAAGUAGUUGG | 714 |
| NO: 152 | NO: 539 | | NO: 925 | | |
| Duplex | SEQ ID | AUUAGAGCUUUCUCGGGAAAA | SEQ ID | UUUUCCCGAGAAAGCUCUAAUAC | 761 |
| NO: 153 | NO: 540 | | NO: 926 | | |
| Duplex | SEQ ID | UUAGAGCUUUCUCGGGAAAGA | SEQ ID | UCUUUCCCGAGAAAGCUCUAAUA | 762 |
| NO: 154 | NO: 541 | | NO: 927 | | |
| Duplex | SEQ ID | UAGAGCUUUCUCGGGAAAGCA | SEQ ID | UGCUUUCCCGAGAAAGCUCUAAU | 763 |
| NO: 155 | NO: 542 | | NO: 928 | | |
| Duplex | SEQ ID | GAGCUUUCUCGGGAAAGCCGA | SEQ ID | UCGGCUUUCCCGAGAAAGCUCUA | 765 |
| NO: 156 | NO: 543 | | NO: 929 | | |
| Duplex | SEQ ID | GGGAAAGCCGCGAGGGUCGUA | SEQ ID | UACGACCCUCGCGGCUUUCCCGA | 775 |
| NO: 157 | NO: 544 | | NO: 930 | | |
| Duplex | SEQ ID | GGAAAGCCGCGAGGGUCGUCA | SEQ ID | UGACGACCCUCGCGGCUUUCCCG | 776 |
| NO: 158 | NO: 545 | | NO: 931 | | |
| Duplex | SEQ ID | GAAAGCCGCGAGGGUCGUCCA | SEQ ID | UGGACGACCCUCGCGGCUUUCCC | 777 |
| NO: 159 | NO: 546 | | NO: 932 | | |
| Duplex | SEQ ID | AAAGCCGCGAGGGUCGUCCAA | SEQ ID | UUGGACGACCCUCGCGGCUUUCC | 778 |
| NO: 160 | NO: 547 | | NO: 933 | | |
| Duplex | SEQ ID | AAGCCGCGAGGGUCGUCCAAA | SEQ ID | UUUGGACGACCCUCGCGGCUUUC | 779 |
| NO: 161 | NO: 548 | | NO: 934 | | |
| Duplex | SEQ ID | AGCCGCGAGGGUCGUCCAAUA | SEQ ID | UAUUGGACGACCCUCGCGGCUUU | 780 |
| NO: 162 | NO: 549 | | NO: 935 | | |
| Duplex | SEQ ID | GCCGCGAGGGUCGUCCAAUUA | SEQ ID | UAAUUGGACGACCCUCGCGGCUU | 781 |
| NO: 163 | NO: 550 | | NO: 936 | | |
| Duplex | SEQ ID | CCGCGAGGGUCGUCCAAUUUA | SEQ ID | UAAAUUGGACGACCCUCGCGGCU | 782 |
| NO: 164 | NO: 551 | | NO: 937 | | |
| Duplex | SEQ ID | CGCGAGGGUCGUCCAAUUUGA | SEQ ID | UCAAAUUGGACGACCCUCGCGGC | 783 |
| NO: 165 | NO: 552 | | NO: 938 | | |
| Duplex | SEQ ID | CCCGAGUUUUAGAAGAAGAAA | SEQ ID | UUUCUUCUUCUAAAACUCGGGCA | 820 |
| NO: 166 | NO: 553 | | NO: 939 | | |
| Duplex | SEQ ID | CUCAGAGGGUCAAGAUGAUUA | SEQ ID | UAAUCAUCUUGACCCUCUGAGUU | 862 |
| NO: 167 | NO: 554 | | NO: 940 | | |
| Duplex | SEQ ID | GGUCAAGAUGAUUAUGUCUCA | SEQ ID | UGAGACAUAAUCAUCUUGACCCU | 869 |
| NO: 168 | NO: 555 | | NO: 941 | | |
| Duplex | SEQ ID | GUCAAGAUGAUUAUGUCUCUA | SEQ ID | UAGAGACAUAAUCAUCUUGACCC | 870 |
| NO: 169 | NO: 556 | | NO: 942 | | |
| Duplex | SEQ ID | CAAGAUGAUUAUGUCUCUAGA | SEQ ID | UCUAGAGACAUAAUCAUCUUGAC | 872 |
| NO: 170 | NO: 557 | | NO: 943 | | |
| Duplex | SEQ ID | AAGAUGAUUAUGUCUCUAGGA | SEQ ID | UCCUAGAGACAUAAUCAUCUUGA | 873 |
| NO: 171 | NO: 558 | | NO: 944 | | |
| Duplex | SEQ ID | AGAUGAUUAUGUCUCUAGGCA | SEQ ID | UGCCUAGAGACAUAAUCAUCUUG | 874 |
| NO: 172 | NO: 559 | | NO: 945 | | |
| Duplex | SEQ ID | GAUGAUUAUGUCUCUAGGCUA | SEQ ID | UAGCCUAGAGACAUAAUCAUCUU | 875 |
| NO: 173 | NO: 560 | | NO: 946 | | |
| Duplex | SEQ ID | AUGAUUAUGUCUCUAGGCUUA | SEQ ID | UAAGCCUAGAGACAUAAUCAUCU | 876 |
| NO: 174 | NO: 561 | | NO: 947 | | |
| Duplex | SEQ ID | AGAUUAUGUCUCUAGGCUUGA | SEQ ID | UCAAGCCUAGAGACAUAAUCAUC | 877 |
| NO: 175 | NO: 562 | | NO: 948 | | |
| Duplex | SEQ ID | AUGCUCACAGUCGCUGGAUAA | SEQ ID | UUAUCCAGCGACUGUGAGCAUGA | 907 |
| NO: 176 | NO: 563 | | NO: 949 | | |
| Duplex | SEQ ID | UGCUCACAGUCGCUGGAUAGA | SEQ ID | UCUAUCCAGCGACUGUGAGCAUG | 908 |
| NO: 177 | NO: 564 | | NO: 950 | | |
| Duplex | SEQ ID | GCUCACAGUCGCUGGAUAGCA | SEQ ID | UGCUAUCCAGCGACUGUGAGCAU | 909 |
| NO: 178 | NO: 565 | | NO: 951 | | |
| Duplex | SEQ ID | CUCACAGUCGCUGGAUAGCUA | SEQ ID | UAGCUAUCCAGCGACUGUGAGCA | 910 |
| NO: 179 | NO: 566 | | NO: 952 | | |
| Duplex | SEQ ID | UCACAGUCGCUGGAUAGCUGA | SEQ ID | UCAGCUAUCCAGCGACUGUGAGC | 911 |
| NO: 180 | NO: 567 | | NO: 953 | | |
| Duplex | SEQ ID | CACAGUCGCUGGAUAGCUGAA | SEQ ID | UUCAGCUAUCCAGCGACUGUGAG | 912 |
| NO: 181 | NO: 568 | | NO: 954 | | |
| Duplex | SEQ ID | GAAUUGAACCAAGUGUUUCCA | SEQ ID | UGGAAACACUUGGUUCAAUUCUC | 1003 |
| NO: 182 | NO: 569 | | NO: 955 | | |
| Duplex | SEQ ID | GAACCAAGUGUUUCCCUCUGA | SEQ ID | UCAGAGGGAAACACUUGGUUCAA | 1008 |
| NO: 183 | NO: 570 | | NO: 956 | | |
| Duplex | SEQ ID | AAAUGAUCAGCAUGGAUAUUA | SEQ ID | UAAUAUCCAUGCUGAUCAUUUUA | 1045 |
| NO: 184 | NO: 571 | | NO: 957 | | |
| Duplex | SEQ ID | AUGAUCAGCAUGGAUAUUGAA | SEQ ID | UUCAAUAUCCAUGCUGAUCAUUU | 1047 |
| NO: 185 | NO: 572 | | NO: 958 | | |
| Duplex | SEQ ID | UCUCCUUCUGGCUGUCAAGUA | SEQ ID | UACUUGACAGCCAGAAGGAGAGC | 1091 |
| NO: 186 | NO: 573 | | NO: 959 | | |
| Duplex | SEQ ID | CUCCUUCUGGCUGUCAAGUAA | SEQ ID | UUACUUGACAGCCAGAAGGAGAG | 1092 |
| NO: 187 | NO: 574 | | NO: 960 | | |
| Duplex | SEQ ID | AACAGAGACAGAAUCUACACA | SEQ ID | UGUGUAGAUUCUGUCUCUGUUUG | 1127 |
| NO: 188 | NO: 575 | | NO: 961 | | |
| Duplex | SEQ ID | ACAGAGACAGAAUCUACACUA | SEQ ID | UAGUGUAGAUUCUGUCUCUGUUU | 1128 |
| NO: 189 | NO: 576 | | NO: 962 | | |
| Duplex | SEQ ID | AUUGUUGUAGACGUGAACCUA | SEQ ID | UAGGUUCACGUCUACAACAAUUG | 1204 |
| NO: 190 | NO: 577 | | NO: 963 | | |
| Duplex | SEQ ID | AAGUUGAGGUUAUAAAACCCA | SEQ ID | UGGGUUUUAUAACCUCAACUUUU | 1291 |
| NO: 191 | NO: 578 | | NO: 964 | | |
| Duplex | SEQ ID | GUUGAGGUUAUAAAACCCUUA | SEQ ID | UAAGGGUUUUAUAACCUCAACUU | 1293 |
| NO: 192 | NO: 579 | | NO: 965 | | |
| Duplex | SEQ ID | UUGAGGUUAUAAAACCCUUAA | SEQ ID | UUAAGGGUUUUAUAACCUCAACU | 1294 |
| NO: 193 | NO: 580 | | NO: 966 | | |
| Duplex | SEQ ID | UGAGGUUAUAAAACCCUUAGA | SEQ ID | UCUAAGGGUUUUAUAACCUCAAC | 1295 |
| NO: 194 | NO: 581 | | NO: 967 | | |
| Duplex | SEQ ID | GCAGAGAAAGGUGCAAAAUUA | SEQ ID | UAAUUUUGCACCUUUCUCUGCAU | 1467 |
| NO: 195 | NO: 582 | | NO: 968 | | |
| Duplex | SEQ ID | CAGAGAAAGGUGCAAAAUUCA | SEQ ID | UGAAUUUUGCACCUUUCUCUGCA | 1468 |
| NO: 196 | NO: 583 | | NO: 969 | | |
| Duplex | SEQ ID | AGAGAAAGGUGCAAAAUUCCA | SEQ ID | UGGAAUUUUGCACCUUUCUCUGC | 1469 |
| NO: 197 | NO: 584 | | NO: 970 | | |
| Duplex | SEQ ID | GAGAAAGGUGCAAAAUUCCUA | SEQ ID | UAGGAAUUUUGCACCUUUCUCUG | 1470 |
| NO: 198 | NO: 585 | | NO: 971 | | |
| Duplex | SEQ ID | AGAAAGGUGCAAAAUUCCUUA | SEQ ID | UAAGGAAUUUUGCACCUUUCUCU | 1471 |
| NO: 199 | NO: 586 | | NO: 972 | | |
| Duplex | SEQ ID | GAAAGGUGCAAAAUUCCUUAA | SEQ ID | UUAAGGAAUUUUGCACCUUUCUC | 1472 |
| NO: 200 | NO: 587 | | NO: 973 | | |
| Duplex | SEQ ID | AAAGGUGCAAAAUUCCUUAGA | SEQ ID | UCUAAGGAAUUUUGCACCUUUCU | 1473 |
| NO: 201 | NO: 588 | | NO: 974 | | |
| Duplex | SEQ ID | AAGGUGCAAAAUUCCUUAGUA | SEQ ID | UACUAAGGAAUUUUGCACCUUUC | 1474 |
| NO: 202 | NO: 589 | | NO: 975 | | |
| Duplex | SEQ ID | AGGUGCAAAAUUCCUUAGUGA | SEQ ID | UCACUAAGGAAUUUUGCACCUUU | 1475 |
| NO: 203 | NO: 590 | | NO: 976 | | |
| Duplex | SEQ ID | AGCAAAAUUCCUUAGUGAUGA | SEQ ID | UCAUCACUAAGGAAUUUUGCACC | 1478 |
| NO: 204 | NO: 591 | | NO: 977 | | |
| Duplex | SEQ ID | GCAAAAUUCCUUAGUGAUGCA | SEQ ID | UGCAUCACUAAGGAAUUUUGCAC | 1479 |
| NO: 205 | NO: 592 | | NO: 978 | | |
| Duplex | SEQ ID | AAUUCCUUAGUGAUGCUGAGA | SEQ ID | UCUCAGCAUCACUAAGGAAUUUU | 1483 |
| NO: 206 | NO: 593 | | NO: 979 | | |
| Duplex | SEQ ID | UCCUUAGUGAUGCUGAGAUCA | SEQ ID | UGAUCUCAGCAUCACUAAGGAAU | 1486 |
| NO: 207 | NO: 594 | | NO: 980 | | |
| Duplex | SEQ ID | CCUUAGUGAUGCUGAGAUCAA | SEQ ID | UUGAUCUCAGCAUCACUAAGGAA | 1487 |
| NO: 208 | NO: 595 | | NO: 981 | | |
| Duplex | SEQ ID | CUGAGAUCAUCCAGUUAGUCA | SEQ ID | UGACUAACUGGAUGAUCUCAGCA | 1498 |
| NO: 209 | NO: 596 | | NO: 982 | | |
| Duplex | SEQ ID | UGAGAUCAUCCAGUUAGUCAA | SEQ ID | UUGACUAACUGGAUGAUCUCAGC | 1499 |
| NO: 210 | NO: 597 | | NO: 983 | | |
| Duplex | SEQ ID | GAGAUCAUCCAGUUAGUCAAA | SEQ ID | UUUGACUAACUGGAUGAUCUCAG | 1500 |
| NO: 211 | NO: 598 | | NO: 984 | | |
| Duplex | SEQ ID | AUCAUCCAGUUAGUCAAUGCA | SEQ ID | UGCAUUGACUAACUGGAUGAUCU | 1503 |
| NO: 212 | NO: 599 | | NO: 985 | | |
| Duplex | SEQ ID | GUCAAUGCUAAGCAUAUCCCA | SEQ ID | UGGGAUAUGCUUAGCAUUGACUA | 1515 |
| NO: 213 | NO: 600 | | NO: 986 | | |
| Duplex | SEQ ID | CUAAGCAUAUCCCAGCCUACA | SEQ ID | UGUAGGCUGGGAUAUGCUUAGCA | 1522 |
| NO: 214 | NO: 601 | | NO: 987 | | |
| Duplex | SEQ ID | AAGCAUAUCCCAGCCUACAAA | SEQ ID | UUUGUAGGCUGGGAUAUGCUUAG | 1524 |
| NO: 215 | NO: 602 | | NO: 988 | | |
| Duplex | SEQ ID | UGGAAACUCUGAUGGAAACUA | SEQ ID | UAGUUUCCAUCAGAGUUUCCAAC | 1546 |
| NO: 216 | NO: 603 | | NO: 989 | | |
| Duplex | SEQ ID | GGAAACUCUGAUGGAAACUCA | SEQ ID | UGAGUUUCCAUCAGAGUUUCCAA | 1547 |
| NO: 217 | NO: 604 | | NO: 990 | | |
| Duplex | SEQ ID | GAAACUCUGAUGGAAACUCAA | SEQ ID | UUGAGUUUCCAUCAGAGUUUCCA | 1548 |
| NO: 218 | NO: 605 | | NO: 991 | | |
| Duplex | SEQ ID | AAACUCUGAUGGAAACUCAUA | SEQ ID | UAUGAGUUUCCAUCAGAGUUUCC | 1549 |
| NO: 219 | NO: 606 | | NO: 992 | | |
| Duplex | SEQ ID | GAUGGAAACUCAUGAGCGUGA | SEQ ID | UCACGCUCAUGAGUUUCCAUCAG | 1556 |
| NO: 220 | NO: 607 | | NO: 993 | | |
| Duplex | SEQ ID | AUGGAAACUCAUGAGCGUGGA | SEQ ID | UCCACGCUCAUGAGUUUCCAUCA | 1557 |
| NO: 221 | NO: 608 | | NO: 994 | | |
| Duplex | SEQ ID | UGGAAACUCAUGAGCGUGGUA | SEQ ID | UACCACGCUCAUGAGUUUCCAUC | 1558 |
| NO: 222 | NO: 609 | | NO: 995 | | |
| Duplex | SEQ ID | GGAAACUCAUGAGCGUGGUGA | SEQ ID | UCACCACGCUCAUGAGUUUCCAU | 1559 |
| NO: 223 | NO: 610 | | NO: 996 | | |
| Duplex | SEQ ID | GAAACUCAUGAGCGUGGUGUA | SEQ ID | UACACCACGCUCAUGAGUUUCCA | 1560 |
| NO: 224 | NO: 611 | | NO: 997 | | |
| Duplex | SEQ ID | AGCGUGGUGUAUCUAUUCGCA | SEQ ID | UGCGAAUAGAUACACCACGCUCA | 1570 |
| NO: 225 | NO: 612 | | NO: 998 | | |
| Duplex | SEQ ID | UACCUUACAGGGAUUAUAAUA | SEQ ID | UAUUAUAAUCCCUGUAAGGUAGG | 1639 |
| NO: 226 | NO: 613 | | NO: 999 | | |
| Duplex | SEQ ID | ACCUUACAGGGAUUAUAAUUA | SEQ ID | UAAUUAUAAUCCCUGUAAGGUAG | 1640 |
| NO: 227 | NO: 614 | | NO: 1000 | | |
| Duplex | SEQ ID | GGGAUUAUAAUUACUCCUUGA | SEQ ID | UCAAGGAGUAAUUAUAAUCCCUG | 1648 |
| NO: 228 | NO: 615 | | NO: 1001 | | |
| Duplex | SEQ ID | GGAUUAUAAUUACUCCUUGGA | SEQ ID | UCCAAGGAGUAAUUAUAAUCCCU | 6149 |
| NO: 229 | NO: 616 | | NO: 1002 | | |
| Duplex | SEQ ID | GAUUAUAAUUACUCCUUGGUA | SEQ ID | UACCAAGGAGUAAUUAUAAUCCC | 1650 |
| NO: 230 | NO: 617 | | NO: 1003 | | |
| Duplex | SEQ ID | AUUAUAAUUACUCCUUGGUGA | SEQ ID | UCACCAAGGAGUAAUUAUAAUCC | 1651 |
| NO: 231 | NO: 618 | | NO: 1004 | | |
| Duplex | SEQ ID | AAUUACUCCUUGGUGAUGGGA | SEQ ID | UCCCAUCACCAAGGAGUAAUUAU | 1656 |
| NO: 232 | NO: 619 | | NO: 1005 | | |
| Duplex | SEQ ID | AUUACUCCUUGGUGAUGGGAA | SEQ ID | UUCCCAUCACCAAGGAGUAAUUA | 1657 |
| NO: 233 | NO: 620 | | NO: 1006 | | |
| Duplex | SEQ ID | CCUUGGUGAUGGGAGCUUGUA | SEQ ID | UACAAGCUCCCAUCACCAAGGAG | 1663 |
| NO: 234 | NO: 621 | | NO: 1007 | | |
| Duplex | SEQ ID | UGGUGAUGGGAGCUUGUUGUA | SEQ ID | UACAACAAGCUCCCAUCACCAAG | 1666 |
| NO: 235 | NO: 622 | | NO: 1008 | | |
| Duplex | SEQ ID | UAUUGGAUAUAUGCCCAUCCA | SEQ ID | UGGAUGGGCAUAUAUCCAAUAAC | 1694 |
| NO: 236 | NO: 623 | | NO: 1009 | | |
| Duplex | SEQ ID | AUUGGAUAUAUGCCCAUCCCA | SEQ ID | UGGGAUGGGCAUAUAUCCAAUAA | 1695 |
| NO: 237 | NO: 624 | | NO: 1010 | | |
| Duplex | SEQ ID | CAGAAGGUUGUCUUGUGGCCA | SEQ ID | UGGCCACAAGACAACCUUCUGUU | 1777 |
| NO: 238 | NO: 625 | | NO: 1011 | | |
| Duplex | SEQ ID | AGGUUGUCUUGUGGCCAGCAA | SEQ ID | UUGCUGGCCACAAGACAACCUUC | 1781 |
| NO: 239 | NO: 626 | | NO: 1012 | | |
| Duplex | SEQ ID | UUGUCUUGUGGCCAGCACCAA | SEQ ID | UUGGUGCUGGCCACAAGACAACC | 1784 |
| NO: 240 | NO: 627 | | NO: 1013 | | |
| Duplex | SEQ ID | UGUCUUGUGGCCAGCACCAAA | SEQ ID | UUUGGUGCUGGCCACAAGACAAC | 1785 |
| NO: 241 | NO: 628 | | NO: 1014 | | |
| Duplex | SEQ ID | AGGUCUUGGUGGAGGUGCCAA | SEQ ID | UUGGCACCUCCACCAAGACCUAU | 1823 |
| NO: 242 | NO: 629 | | NO: 1015 | | |
| Duplex | SEQ ID | GGUCUUGGUGGAGGUGCCAGA | SEQ ID | UCUGGCACCUCCACCAAGACCUA | 1824 |
| NO: 243 | NO: 630 | | NO: 1016 | | |
| Duplex | SEQ ID | GUGGAGGUGCCAGCAGCCGAA | SEQ ID | UUCGGCUGCUGGCACCUCCACCA | 1831 |
| NO: 244 | NO: 631 | | NO: 1017 | | |
| Duplex | SEQ ID | GUGCCAGCAGCCGAGUCCUUA | SEQ ID | UAAGGACUCGGCUGCUGGCACCU | 1837 |
| NO: 245 | NO: 632 | | NO: 1018 | | |
| Duplex | SEQ ID | CAGCAGCCGAGUCCUUGCAGA | SEQ ID | UCUGCAAGGACUCGGCUGCUGGC | 1841 |
| NO: 246 | NO: 633 | | NO: 1019 | | |
| Duplex | SEQ ID | AGCAGCCGAGUCCUUGCAGAA | SEQ ID | UUCUGCAAGGACUCGGCUGCUGG | 1842 |
| NO: 247 | NO: 634 | | NO: 1020 | | |
| Duplex | SEQ ID | GCAGCCGAGUCCUUGCAGAUA | SEQ ID | UAUCUGCAAGGACUCGGCUGCUG | 1843 |
| NO: 248 | NO: 635 | | NO: 1021 | | |
| Duplex | SEQ ID | CAGCCGAGUCCUUGCAGAUGA | SEQ ID | UCAUCUGCAAGGACUCGGCUGCU | 1844 |
| NO: 249 | NO: 636 | | NO: 1022 | | |
| Duplex | SEQ ID | GCCGAGUCCUUGCAGAUGGGA | SEQ ID | UCCCAUCUGCAAGGACUCGGCUG | 1846 |
| NO: 250 | NO: 637 | | NO: 1023 | | |
| Duplex | SEQ ID | CGAGUCCUUGCAGAUGGGAUA | SEQ ID | UAUCCCAUCUGCAAGGACUCGGC | 1848 |
| NO: 251 | NO: 638 | | NO: 1024 | | |
| Duplex | SEQ ID | UUGCAGAUGGGAUGACUCGUA | SEQ ID | UACGAGUCAUCCCAUCUGCAAGG | 1855 |
| NO: 252 | NO: 639 | | NO: 1025 | | |
| Duplex | SEQ ID | UGCAGAUGGGAUGACUCGUGA | SEQ ID | UCACGAGUCAUCCCAUCUGCAAG | 1856 |
| NO: 253 | NO: 640 | | NO: 1026 | | |
| Duplex | SEQ ID | GCAGAUGGGAUGACUCGUGGA | SEQ ID | UCCACGAGUCAUCCCAUCUGCAA | 1857 |
| NO: 254 | NO: 641 | | NO: 1027 | | |
| Duplex | SEQ ID | CAGAUGGGAUGACUCGUGGCA | SEQ ID | UGCCACGAGUCAUCCCAUCUGCA | 1858 |
| NO: 255 | NO: 642 | | NO: 1028 | | |
| Duplex | SEQ ID | AGAUGGGAUGACUCGUGGCCA | SEQ ID | UGGCCACGAGUCAUCCCAUCUGC | 1859 |
| NO: 256 | NO: 643 | | NO: 1029 | | |
| Duplex | SEQ ID | GAUGGGAUGACUCGUGGCCCA | SEQ ID | UGGGCCACGAGUCAUCCCAUCUG | 1860 |
| NO: 257 | NO: 644 | | NO: 1030 | | |
| Duplex | SEQ ID | AUGGGAUGACUCGUGGCCCAA | SEQ ID | UUGGGCCACGAGUCAUCCCAUCU | 1861 |
| NO: 258 | NO: 645 | | NO: 1031 | | |
| Duplex | SEQ ID | UGGGAUGACUCGUGGCCCAGA | SEQ ID | UCUGGGCCACGAGUCAUCCCAUC | 1862 |
| NO: 259 | NO: 646 | | NO: 1032 | | |
| Duplex | SEQ ID | GGGAUGACUCGUGGCCCAGUA | SEQ ID | UACUGGGCCACGAGUCAUCCCAU | 1863 |
| NO: 260 | NO: 647 | | NO: 1033 | | |
| Duplex | SEQ ID | GUGGCCCAGUUGUGCGUCUUA | SEQ ID | UAAGACGCACAACUGGGCCACGA | 1873 |
| NO: 261 | NO: 648 | | NO: 1034 | | |
| Duplex | SEQ ID | UGGCCCAGUUGUGCGUCUUCA | SEQ ID | UGAAGACGCACAACUGGGCCACG | 1874 |
| NO: 262 | NO: 649 | | NO: 1035 | | |
| Duplex | SEQ ID | GCCCAGUUGUGCGUCUUCCAA | SEQ ID | UUGGAAGACGCACAACUGGGCCA | 1876 |
| NO: 263 | NO: 650 | | NO: 1036 | | |
| Duplex | SEQ ID | CCCAGUUGUGCGUCUUCCACA | SEQ ID | UGUGGAAGACGCACAACUGGGCC | 1877 |
| NO: 264 | NO: 651 | | NO: 1037 | | |
| Duplex | SEQ ID | CCAGUUGUGCGUCUUCCACGA | SEQ ID | UCGUGGAAGACGCACAACUGGGC | 1878 |
| NO: 265 | NO: 652 | | NO: 1038 | | |
| Duplex | SEQ ID | CAGUUGUGCGUCUUCCACGUA | SEQ ID | UACGUGGAAGACGCACAACUGGG | 1879 |
| NO: 266 | NO: 653 | | NO: 1039 | | |
| Duplex | SEQ ID | AGUUGUGCGUCUUCCACGUGA | SEQ ID | UCACGUGGAAGACGCACAACUGG | 1880 |
| NO: 267 | NO: 654 | | NO: 1040 | | |
| Duplex | SEQ ID | UUGUGACUCUGCAGAAGUGAA | SEQ ID | UUCACUUCUGCAGAGUCACAAGC | 1901 |
| NO: 268 | NO: 655 | | NO: 1041 | | |
| Duplex | SEQ ID | CUGAAGGGUUCGCAGUGAUAA | SEQ ID | UUAUCACUGCGAACCCUUCAGAU | 1939 |
| NO: 269 | NO: 656 | | NO: 1042 | | |
| Duplex | SEQ ID | UGAAGGGUUCGCAGUGAUAAA | SEQ ID | UUUAUCACUGCGAACCCUUCAGA | 1940 |
| NO: 270 | NO: 657 | | NO: 1043 | | |
| Duplex | SEQ ID | GAAGGGUUCGCAGUGAUAAAA | SEQ ID | UUUUAUCACUGCGAACCCUUCAG | 1941 |
| NO: 271 | NO: 658 | | NO: 1044 | | |
| Duplex | SEQ ID | AGGGUUCGCAGUGAUAAAGGA | SEQ ID | UCCUUUAUCACUGCGAACCCUUC | 1943 |
| NO: 272 | NO: 659 | | NO: 1045 | | |
| Duplex | SEQ ID | GGGUUCGCAGUGAUAAAGGAA | SEQ ID | UUCCUUUAUCACUGCGAACCCUU | 1944 |
| NO: 273 | NO: 660 | | NO: 1046 | | |
| Duplex | SEQ ID | UUCGCAGUGAUAAAGGAGGCA | SEQ ID | UGCCUCCUUUAUCACUGCGAACC | 1947 |
| NO: 274 | NO: 661 | | NO: 1047 | | |
| Duplex | SEQ ID | CAGUGAUAAAGGAGGCAUUUA | SEQ ID | UAAAUGCCUCCUUUAUCACUGCG | 1951 |
| NO: 275 | NO: 662 | | NO: 1048 | | |
| Duplex | SEQ ID | CUAGCAGAUUUGCACGUCUAA | SEQ ID | UUAGACGUGCAAAUCUGCUAGUG | 1978 |
| NO: 276 | NO: 663 | | NO: 1049 | | |
| Duplex | SEQ ID | UAGCAGAUUUGCACGUCUACA | SEQ ID | UGUAGACGUGCAAAUCUGCUAGU | 1979 |
| NO: 277 | NO: 664 | | NO: 1050 | | |
| Duplex | SEQ ID | AGCAGAUUUGCACGUCUACAA | SEQ ID | UUGUAGACGUGCAAAUCUGCUAG | 1980 |
| NO: 278 | NO: 665 | | NO: 1051 | | |
| Duplex | SEQ ID | GCAGAUUUGCACGUCUACAGA | SEQ ID | UCUGUAGACGUGCAAAUCUGCUA | 1981 |
| NO: 279 | NO: 666 | | NO: 1052 | | |
| Duplex | SEQ ID | CAGAUUUGCACGUCUACAGAA | SEQ ID | UUCUGUAGACGUGCAAAUCUGCU | 1982 |
| NO: 280 | NO: 667 | | NO: 1053 | | |
| Duplex | SEQ ID | GAUUUGCACGUCUACAGAAAA | SEQ ID | UUUUCUGUAGACGUGCAAAUCUG | 1984 |
| NO: 281 | NO: 668 | | NO: 1054 | | |
| Duplex | SEQ ID | AUUUGCACGUCUACAGAAACA | SEQ ID | UGUUUCUGUAGACGUGCAAAUCU | 1985 |
| NO: 282 | NO: 669 | | NO: 1055 | | |
| Duplex | SEQ ID | CUGGACGCAACCUUUAUAUCA | SEQ ID | UGAUAUAAAGGUUGCGUCCAGCU | 2020 |
| NO: 283 | NO: 670 | | NO: 1056 | | |
| Duplex | SEQ ID | UGGACGCAACCUUUAUAUCCA | SEQ ID | UGGAUAUAAAGGUUGCGUCCAGC | 2021 |
| NO: 284 | NO: 671 | | NO: 1057 | | |
| Duplex | SEQ ID | GGACGCAACCUUUAUAUCCGA | SEQ ID | UCGGAUAUAAAGGUUGCGUCCAG | 2022 |
| NO: 285 | NO: 672 | | NO: 1058 | | |
| Duplex | SEQ ID | GACGCAACCUUUAUAUCCGUA | SEQ ID | UACGGAUAUAAAGGUUGCGUCCA | 2023 |
| NO: 286 | NO: 673 | | NO: 1059 | | |
| Duplex | SEQ ID | CGCAACCUUUAUAUCCGUUUA | SEQ ID | UAAACGGAUAUAAAGGUUGCGUC | 2025 |
| NO: 287 | NO: 674 | | NO: 1060 | | |
| Duplex | SEQ ID | GCAACCUUUAUAUCCGUUUCA | SEQ ID | UGAAACGGAUAUAAAGGUUGCGU | 2026 |
| NO: 288 | NO: 675 | | NO: 1061 | | |
| Duplex | SEQ ID | CAACCUUUAUAUCCGUUUCCA | SEQ ID | UGGAAACGGAUAUAAAGGUUGCG | 2027 |
| NO: 289 | NO: 676 | | NO: 1062 | | |
| Duplex | SEQ ID | CCUUUAUAUCCGUUUCCAGUA | SEQ ID | UACUGGAAACGGAUAUAAAGGUU | 2030 |
| NO: 290 | NO: 677 | | NO: 1063 | | |
| Duplex | SEQ ID | CUUUAUAUCCGUUUCCAGUCA | SEQ ID | UGACUGGAAACGGAUAUAAAGGU | 2031 |
| NO: 291 | NO: 678 | | NO: 1064 | | |
| Duplex | SEQ ID | UAUCCGUUUCCAGUCCAGGUA | SEQ ID | UACCUGGACUGGAAACGGAUAUA | 2036 |
| NO: 292 | NO: 679 | | NO: 1065 | | |
| Duplex | SEQ ID | UUGUUUGUGAAGCUGUCAUUA | SEQ ID | UAAUGACAGCUUCACAAACAACA | 2221 |
| NO: 293 | NO: 680 | | NO: 1066 | | |
| Duplex | SEQ ID | UUUGUGAAGCUGUCAUUCCAA | SEQ ID | UUGGAAUGACAGCUUCACAAACA | 2224 |
| NO: 294 | NO: 681 | | NO: 1067 | | |
| Duplex | SEQ ID | UUGUGAAGCUGUCAUUCCAGA | SEQ ID | UCUGGAAUGACAGCUUCACAAAC | 2225 |
| NO: 295 | NO: 682 | | NO: 1068 | | |
| Duplex | SEQ ID | GAAGCUGUCAUUCCAGCCAAA | SEQ ID | UUUGGCUGGAAUGACAGCUUCAC | 2229 |
| NO: 296 | NO: 683 | | NO: 1069 | | |
| Duplex | SEQ ID | GCUGUCAUUCCAGCCAAGGUA | SEQ ID | UACCUUGGCUGGAAUGACAGCUU | 2232 |
| NO: 297 | NO: 684 | | NO: 1070 | | |
| Duplex | SEQ ID | CUGUCAUUCCAGCCAAGGUUA | SEQ ID | UAACCUUGGCUGGAAUGACAGCU | 2233 |
| NO: 298 | NO: 685 | | NO: 1071 | | |
| Duplex | SEQ ID | UGUCAUUCCAGCCAAGGUUGA | SEQ ID | UCAACCUUGGCUGGAAUGACAGC | 2234 |
| NO: 299 | NO: 686 | | NO: 1072 | | |
| Duplex | SEQ ID | GUCAUUCCAGCCAAGGUUGUA | SEQ ID | UACAACCUUGGCUGGAAUGACAG | 2235 |
| NO: 300 | NO: 687 | | NO: 1073 | | |
| Duplex | SEQ ID | GCUGGGAGCAUAGGAGGCUAA | SEQ ID | UUAGCCUCCUAUGCUCCCAGCCA | 2331 |
| NO: 301 | NO: 688 | | NO: 1074 | | |
| Duplex | SEQ ID | CUGGGAGCAUAGGAGGCUACA | SEQ ID | UGUAGCCUCCUAUGCUCCCAGCC | 2332 |
| NO: 302 | NO: 689 | | NO: 1075 | | |
| Duplex | SEQ ID | GGGAGCAUAGGAGGCUACAAA | SEQ ID | UUUGUAGCCUCCUAUGCUCCCAG | 2334 |
| NO: 303 | NO: 690 | | NO: 1076 | | |
| Duplex | SEQ ID | GGAGCAUAGGAGGCUACAACA | SEQ ID | UGUUGUAGCCUCCUAUGCUCCCA | 2335 |
| NO: 304 | NO: 691 | | NO: 1077 | | |
| Duplex | SEQ ID | AGCAUAGGAGGCUACAACGCA | SEQ ID | UGCGUUGUAGCCUCCUAUGCUCC | 2337 |
| NO: 305 | NO: 692 | | NO: 1078 | | |
| Duplex | SEQ ID | GCAUAGGAGGCUACAACGCCA | SEQ ID | UGGCGUUGUAGCCUCCUAUGCUC | 2338 |
| NO: 306 | NO: 693 | | NO: 1079 | | |
| Duplex | SEQ ID | CAUAGGAGGCUACAACGCCCA | SEQ ID | UGGGCGUUGUAGCCUCCUAUGCU | 2339 |
| NO: 307 | NO: 694 | | NO: 1080 | | |
| Duplex | SEQ ID | AUAGGAGGCUACAACGCCCAA | SEQ ID | UUGGGCGUUGUAGCCUCCUAUGC | 2340 |
| NO: 308 | NO: 695 | | NO: 1081 | | |
| Duplex | SEQ ID | GAGGCUACAACGCCCAUGCAA | SEQ ID | UUGCAUGGGCGUUGUAGCCUCCU | 2344 |
| NO: 309 | NO: 696 | | NO: 1082 | | |
| Duplex | SEQ ID | GGCUACAACGCCCAUGCAGCA | SEQ ID | UGCUGCAUGGGCGUUGUAGCCUC | 2346 |
| NO: 310 | NO: 697 | | NO: 1083 | | |
| Duplex | SEQ ID | GCUACAACGCCCAUGCAGCAA | SEQ ID | UUGCUGCAUGGGCGUUGUAGCCU | 2347 |
| NO: 311 | NO: 698 | | NO: 1084 | | |
| Duplex | SEQ ID | CUACAACGCCCAUGCAGCAAA | SEQ ID | UUUGCUGCAUGGGCGUUGUAGCC | 2348 |
| NO: 312 | NO: 699 | | NO: 1085 | | |
| Duplex | SEQ ID | ACAACGCCCAUGCAGCAAACA | SEQ ID | UGUUUGCUGCAUGGGCGUUGUAG | 2350 |
| NO: 313 | NO: 700 | | NO: 1086 | | |
| Duplex | SEQ ID | CAACGCCCAUGCAGCAAACAA | SEQ ID | UUGUUUGCUGCAUGGGCGUUGUA | 2351 |
| NO: 314 | NO: 701 | | NO: 1087 | | |
| Duplex | SEQ ID | AACGCCCAUGCAGCAAACAUA | SEQ ID | UAUGUUUGCUGCAUGGGCGUUGU | 2352 |
| NO: 315 | NO: 702 | | NO: 1088 | | |
| Duplex | SEQ ID | CCCAUGCAGCAAACAUUGUCA | SEQ ID | UGACAAUGUUUGCUGCAUGGGCG | 2356 |
| NO: 316 | NO: 703 | | NO: 1089 | | |
| Duplex | SEQ ID | CUGUGGACAGGAUGCAGCACA | SEQ ID | UGUGCUGCAUCCUGUCCACAGGC | 2393 |
| NO: 317 | NO: 704 | | NO: 1090 | | |
| Duplex | SEQ ID | GGAUGCAGCACAGAAUGUUGA | SEQ ID | UCAACAUUCUGUGCUGCAUCCUG | 2402 |
| NO: 318 | NO: 705 | | NO: 1091 | | |
| Duplex | SEQ ID | UUGGUAGUUCAAACUGUAUUA | SEQ ID | UAAUACAGUUUGAACUACCAACA | 2419 |
| NO: 319 | NO: 706 | | NO: 1092 | | |
| Duplex | SEQ ID | UGGUAGUUCAAACUGUAUUAA | SEQ ID | UUAAUACAGUUUGAACUACCAAC | 2420 |
| NO: 320 | NO: 707 | | NO: 1093 | | |
| Duplex | SEQ ID | GGUAGUUCAAACUGUAUUACA | SEQ ID | UGUAAUACAGUUUGAACUACCAA | 2421 |
| NO: 321 | NO: 708 | | NO: 1094 | | |
| Duplex | SEQ ID | GUAUUACUUUAAUGGAAGCAA | SEQ ID | UUGCUUCCAUUAAAUGAAUACAG | 2434 |
| NO: 322 | NO: 709 | | NO: 1095 | | |
| Duplex | SEQ ID | UAUUACUUUAAUGGAAGCAAA | SEQ ID | UUUGCUUCCAUUAAAGUAAUACA | 2435 |
| NO: 323 | NO: 710 | | NO: 1096 | | |
| Duplex | SEQ ID | UUACUUUAAUGGAAGCAAGUA | SEQ ID | UACUUGCUUCCAUUAAAGUAAUA | 2437 |
| NO: 324 | NO: 711 | | NO: 1097 | | |
| Duplex | SEQ ID | UACUUUAAUGGAAGCAAGUGA | SEQ ID | UCACUUGCUUCCAUUAAAGUAAU | 2438 |
| NO: 325 | NO: 712 | | NO: 1098 | | |
| Duplex | SEQ ID | GUCCCACAAAUGAAGAUUUAA | SEQ ID | UUAAAUCUUCAUUUGUGGGACCA | 2458 |
| NO: 326 | NO: 713 | | NO: 1099 | | |
| Duplex | SEQ ID | AAGAUUUAUAUAUCAGCUGCA | SEQ ID | UGCAGCUGAUAUAUAAAUCUUCA | 2470 |
| NO: 327 | NO: 714 | | NO: 1100 | | |
| Duplex | SEQ ID | UCAGCUGCACCAUGCCAUCUA | SEQ ID | UAGAUGGCAUGGUGCAGCUGAUA | 2482 |
| NO: 328 | NO: 715 | | NO: 1101 | | |
| Duplex | SEQ ID | CAGCUGCACCAUGCCAUCUAA | SEQ ID | UUAGAUGGCAUGGUGCAGCUGAU | 2483 |
| NO: 329 | NO: 716 | | NO: 1102 | | |
| Duplex | SEQ ID | AGCUGCACCAUGCCAUCUAUA | SEQ ID | UAUAGAUGGCAUGGUGCAGCUGA | 2484 |
| NO: 330 | NO: 717 | | NO: 1103 | | |
| Duplex | SEQ ID | GCUGCACCAUGCCAUCUAUAA | SEQ ID | UUAUAGAUGGCAUGGUGCAGCUG | 2485 |
| NO: 331 | NO: 718 | | NO: 1104 | | |
| Duplex | SEQ ID | CUGCACCAUGCCAUCUAUAGA | SEQ ID | UCUAUAGAUGGCAUGGUGCAGCU | 2486 |
| NO: 332 | NO: 719 | | NO: 1105 | | |
| Duplex | SEQ ID | UGCACCAUGCCAUCUAUAGAA | SEQ ID | UUCUAUAGAUGGCAUGGUGCAGC | 2487 |
| NO: 333 | NO: 720 | | NO: 1106 | | |
| Duplex | SEQ ID | GCACCAUGCCAUCUAUAGAGA | SEQ ID | UCUCUAUAGAUGGCAUGGUGCAG | 2488 |
| NO: 334 | NO: 721 | | NO: 1107 | | |
| Duplex | SEQ ID | CACCAUGCCAUCUAUAGAGAA | SEQ ID | UUCUCUAUAGAUGGCAUGGUGCA | 2489 |
| NO: 335 | NO: 722 | | NO: 1108 | | |
| Duplex | SEQ ID | AUGCCAUCUAUAGAGAUAGGA | SEQ ID | UCCUAUCUCUAUAGAUGGCAUGG | 2493 |
| NO: 336 | NO: 723 | | NO: 1109 | | |
| Duplex | SEQ ID | CUAUAGAGAUAGGAACGGUGA | SEQ ID | UCACCGUUCCUAUCUCUAUAGAU | 2500 |
| NO: 337 | NO: 724 | | NO: 1110 | | |
| Duplex | SEQ ID | UAUAGAGAUAGGAACGGUGGA | SEQ ID | UCCACCGUUCCUAUCUCUAUAGA | 2501 |
| NO: 338 | NO: 725 | | NO: 1111 | | |
| Duplex | SEQ ID | AUAGAGAUAGGAACGGUGGGA | SEQ ID | UCCCACCGUUCCUAUCUCUAUAG | 2502 |
| NO: 339 | NO: 726 | | NO: 1112 | | |
| Duplex | SEQ ID | UACUACCUCAGCAAGCCUGUA | SEQ ID | UACAGGCUUGCUGAGGUAGUAGG | 2536 |
| NO: 340 | NO: 727 | | NO: 1113 | | |
| Duplex | SEQ ID | GUUUGCAGAUGCUAGGUGUUA | SEQ ID | UAACACCUAGCAUCUGCAAACAG | 2554 |
| NO: 341 | NO: 728 | | NO: 1114 | | |
| Duplex | SEQ ID | UUCAAGGAGCAUGCAAAGAUA | SEQ ID | UAUCUUUGCAUGCUCCUUGAACA | 2572 |
| NO: 342 | NO: 729 | | NO: 1115 | | |
| Duplex | SEQ ID | UCAAGGAGCAUGCAAAGAUAA | SEQ ID | UUAUCUUUGCAUGCUCCUUGAAC | 2573 |
| NO: 343 | NO: 730 | | NO: 1116 | | |
| Duplex | SEQ ID | CAAGGAGCAUGCAAAGAUAAA | SEQ ID | UUUAUCUUUGCAUGCUCCUUGAA | 2574 |
| NO: 344 | NO: 731 | | NO: 1117 | | |
| Duplex | SEQ ID | AAGGAGCAUGCAAAGAUAAUA | SEQ ID | UAUUAUCUUUGCAUGCUCCUUGA | 2575 |
| NO: 345 | NO: 732 | | NO: 1118 | | |
| Duplex | SEQ ID | AGGAGCAUGCAAAGAUAAUCA | SEQ ID | UGAUUAUCUUUGCAUGCUCCUUG | 2576 |
| NO: 346 | NO: 733 | | NO: 1119 | | |
| Duplex | SEQ ID | GGAGCAUGCAAAGAUAAUCCA | SEQ ID | UGGAUUAUCUUUGCAUGCUCCUU | 2577 |
| NO: 347 | NO: 734 | | NO: 1120 | | |
| Duplex | SEQ ID | UGCCCGGCAGCUUGCCCGAAA | SEQ ID | UUUCGGGCAAGCUGCCGGGCAUU | 2606 |
| NO: 348 | NO: 735 | | NO: 1121 | | |
| Duplex | SEQ ID | GGCAGCUUGCCCGAAUUGUGA | SEQ ID | UCACAAUUCGGGCAAGCUGCCGG | 2611 |
| NO: 349 | NO: 736 | | NO: 1122 | | |
| Duplex | SEQ ID | GCAGCUUGCCCGAAUUGUGUA | SEQ ID | UACACAAUUCGGGCAAGCUGCCG | 2612 |
| NO: 350 | NO: 737 | | NO: 1123 | | |
| Duplex | SEQ ID | CAGCUUGCCCGAAUUGUGUGA | SEQ ID | UCACACAAUUCGGGCAAGCUGCC | 2613 |
| NO: 351 | NO: 738 | | NO: 1124 | | |
| Duplex | SEQ ID | GGCAGCAUUGGCAGCAGGACA | SEQ ID | UGUCCUGCUGCCAAUGCUGCCAU | 2666 |
| NO: 352 | NO: 739 | | NO: 1125 | | |
| Duplex | SEQ ID | GCAGCAUUGGCAGCAGGACAA | SEQ ID | UUGUCCUGCUGCCAAUGCUGCCA | 2667 |
| NO: 353 | NO: 740 | | NO: 1126 | | |
| Duplex | SEQ ID | CAGCAUUGGCAGCAGGACAUA | SEQ ID | UAUGUCCUGCUGCCAAUGCUGCC | 2668 |
| NO: 354 | NO: 741 | | NO: 1127 | | |
| Duplex | SEQ ID | CAGCAGGACAUCUUGUCAAAA | SEQ ID | UUUUGACAAGAUGUCCUGCUGCC | 2677 |
| NO: 355 | NO: 742 | | NO: 1128 | | |
| Duplex | SEQ ID | AGCAGGACAUCUUGUCAAAAA | SEQ ID | UUUUUGACAAGAUGUCCUGCUGC | 2678 |
| NO: 356 | NO: 743 | | NO: 1129 | | |
| Duplex | SEQ ID | CAGGACAUCUUGUCAAAAGUA | SEQ ID | UACUUUUGACAAGAUGUCCUGCU | 2680 |
| NO: 357 | NO: 744 | | NO: 1130 | | |
| Duplex | SEQ ID | AGGACAUCUUGUCAAAAGUCA | SEQ ID | UGACUUUUGACAAGAUGUCCUGC | 2681 |
| NO: 358 | NO: 745 | | NO: 1131 | | |
| Duplex | SEQ ID | GGACAUCUUGUCAAAAGUCAA | SEQ ID | UUGACUUUUGACAAGAUGUCCUG | 2682 |
| NO: 359 | NO: 746 | | NO: 1132 | | |
| Duplex | SEQ ID | UAAGUCUGGGAUGUAGAACUA | SEQ ID | UAGUUCUACAUCCCAGACUUAAU | 3215 |
| NO: 360 | NO: 747 | | NO: 1133 | | |
| Duplex | SEQ ID | GCUAUUUGUUCAGAGAGGCCA | SEQ ID | UGGCCUCUCUGAACAAAUAGCCU | 3454 |
| NO: 361 | NO: 748 | | NO: 1134 | | |
| Duplex | SEQ ID | CUAUUUGUUCAGAGAGGCCUA | SEQ ID | UAGGCCUCUCUGAACAAAUAGCC | 3455 |
| NO: 362 | NO: 749 | | NO: 1135 | | |
| Duplex | SEQ ID | UUGGAAGUGUUCAAGAAAUAA | SEQ ID | UUAUUUCUUGAACACUUCCAAUA | 3806 |
| NO: 363 | NO: 750 | | NO: 1136 | | |
| Duplex | SEQ ID | UGGAAGUGUUCAAGAAAUAAA | SEQ ID | UUUAUUUCUUGAACACUUCCAAU | 3807 |
| NO: 364 | NO: 751 | | NO: 1137 | | |
| Duplex | SEQ ID | UGUACUGAUAAAAUACUCUAA | SEQ ID | UUAGAGUAUUUUAUCAGUACACA | 3838 |
| NO: 365 | NO: 752 | | NO: 1138 | | |
| Duplex | SEQ ID | AGGAAACCCUGGCUUGCUUGA | SEQ ID | UCAAGCAAGCCAGGGUUUCCUGG | 3896 |
| NO: 366 | NO: 753 | | NO: 1139 | | |
| Duplex | SEQ ID | GUGCCUAGUUCCUGUGGCCAA | SEQ ID | UUGGCCACAGGAACUAGGCACAG | 3985 |
| NO: 367 | NO: 754 | | NO: 1140 | | |
| Duplex | SEQ ID | GUUCCUGUGGCCAGGAGGUUA | SEQ ID | UAACCUCCUGGCCACAGGAACUA | 3992 |
| NO: 368 | NO: 755 | | NO: 1141 | | |
| Duplex | SEQ ID | CUGUGGCCAGGAGGUUGGUGA | SEQ ID | UCACCAACCUCCUGGCCACAGGA | 3996 |
| NO: 369 | NO: 756 | | NO: 1142 | | |
| Duplex | SEQ ID | GCCAGGAGGUUGGUGACUGAA | SEQ ID | UUCAGUCACCAACCUCCUGGCCA | 4001 |
| NO: 370 | NO: 757 | | NO: 1143 | | |
| Duplex | SEQ ID | CAGAGUUAUUGAAUCUUAAUA | SEQ ID | UAUUAAGAUUCAAUAACUCUGCU | 4082 |
| NO: 371 | NO: 758 | | NO: 1144 | | |
| Duplex | SEQ ID | AGAGUUAUUGAAUCUUAAUUA | SEQ ID | UAAUUAAGAUUCAAUAACUCUGC | 4083 |
| NO: 372 | NO: 759 | | NO: 1145 | | |
| Duplex | SEQ ID | GAGUUAUUGAAUCUUAAUUUA | SEQ ID | UAAAUUAAGAUUCAAUAACUCUG | 4084 |
| NO: 373 | NO: 760 | | NO: 1146 | | |
| Duplex | SEQ ID | UUUUUAAUGUACAAGUUUUGA | SEQ ID | UCAAAACUUGUACAUUAAAAAAA | 4104 |
| NO: 374 | NO: 761 | | NO: 1147 | | |
| Duplex | SEQ ID | UUUAAUGUACAAGUUUUGUAA | SEQ ID | UUACAAAACUUGUACAUUAAAAA | 4106 |
| NO: 375 | NO: 762 | | NO: 1148 | | |
| Duplex | SEQ ID | AAAUAAUAAAGAACUCCUUAA | SEQ ID | UUAAGGAGUUCUUUAUUAUUUAU | 4127 |
| NO: 376 | NO: 763 | | NO: 1149 | | |
| Duplex | SEQ ID | AAUAAUAAAGAACUCCUUAUA | SEQ ID | UAUAAGGAGUUCUUUAUUAUUUA | 4128 |
| NO: 377 | NO: 764 | | NO: 1150 | | |
| Duplex | SEQ ID | AUAAUAAAGAACUCCUUAUUA | SEQ ID | UAAUAAGGAGUUCUUUAUUAUUU | 4129 |
| NO: 378 | NO: 765 | | NO: 1151 | | |
| Duplex | SEQ ID | UAAUAAAGAACUCCUUAUUUA | SEQ ID | UAAAUAAGGAGUUCUUUAUUAUU | 4130 |
| NO: 379 | NO: 766 | | NO: 1152 | | |
| Duplex | SEQ ID | AAUAAAGAACUCCUUAUUUUA | SEQ ID | UAAAAUAAGGAGUUCUUUAUUAU | 4131 |
| NO: 380 | NO: 767 | | NO: 1153 | | |
| Duplex | SEQ ID | ACAUCUAAUGCUUCAAGUGUA | SEQ ID | UACACUUGAAGCAUUAGAUGUAA | 4156 |
| NO: 381 | NO: 768 | | NO: 1154 | | |
| Duplex | SEQ ID | UCUGAGCUUUUCAUGGAAGUA | SEQ ID | UACUUCCAUGAAAAGCUCAGAAA | 4360 |
| NO: 382 | NO: 769 | | NO: 1155 | | |
| Duplex | SEQ ID | UGAGCUUUUCAUGGAAGUGGA | SEQ ID | UCCACUUCCAUGAAAAGCUCAGA | 4362 |
| NO: 383 | NO: 770 | | NO: 1156 | | |
| Duplex | SEQ ID | AGCUUUUCAUGGAAGUGGCAA | SEQ ID | UUGCCACUUCCAUGAAAAGCUCA | 4364 |
| NO: 384 | NO: 771 | | NO: 1157 | | |
| Duplex | SEQ ID | UUCAUGGAAGUGGCAGUGAAA | SEQ ID | UUUCACUGCCACUUCCAUGAAAA | 4369 |
| NO: 385 | NO: 772 | | NO: 1158 | | |
| Duplex | SEQ ID | UUAAUGUUUUGUACAAUUACA | SEQ ID | UGUAAUUGUACAAAACAUUAAUC | 4438 |
| NO: 386 | NO: 773 | | NO: 1159 | | |

TABLE 4A
HMGCR-GalNAc RNAi agents, modified sense and antisense strands

| Duplex NO: | SEQ ID NO: | Modified Sequence |
|---|---|---|
| Duplex NO: 387 | SEQ ID NO: 1160 | mG*mC*mUmAmCmAmAmUfGfUfUmGmUmCmAmAmGmAmC*mU*mA |
| | SEQ ID NO: 1161 | PmU*fA*mGmUfCmUfUmGmAmCmAmAmCfAmUfUmGmUmAmGmC*mU*mA |
| Duplex NO: 388 | SEQ ID NO: 1162 | mC*mU*mAmCmAmAmUmGfUfUfGmUmCmAmAmGmAmCmU*mU*mA |
| | SEQ ID NO: 1163 | PmU*fA*mAmGfUmCfUmUmGmAmCmAmAfCmAfUmUmGmUmAmG*mC*mU |
| Duplex NO: 389 | SEQ ID NO: 1164 | mA*mU*mGmAmUmGmUmCfCfAfUmGmAmAmCmAmUmGmU*mU*mA |
| | SEQ ID NO: 1165 | PmU*fA*mAmCfAmUfGmUmUmCmAmUmGfGmAfCmAmUmCmAmU*mG*mC |
| Duplex NO: 390 | SEQ ID NO: 1166 | mA*mU*mAmUmUmUmAmCfUfUfCmCmAmGmUmUmCmCmA*mG*mA |
| | SEQ ID NO: 1167 | PmU*fC*mUmGfGmAfAmCmUmGmGmAmAfGmUfAmAmAmUmAmU*mA*mC |
| Duplex NO: 391 | SEQ ID NO: 1168 | mA*mU*mUmUmAmCmUmUfCfCfAmGmUmUmCmCmAmGmA*mA*mA |
| | SEQ ID NO: 1169 | PmU*fU*mUmCfUmGfGmAmAmCmUmGmGfAmAfGmUmAmAmAmU*mA*mU |
| Duplex NO: 392 | SEQ ID NO: 1170 | mA*mA*mUmGmAmAmGmCfUfUfUmGmCmCmCmUmUmUmU*mU*mA |
| | SEQ ID NO: 1171 | PmU*fA*mAmAfAmAfGmGmGmCmAmAmAfGmCfUmUmCmAmUmU*mC*mA |
| Duplex NO: 393 | SEQ ID NO: 1172 | mC*mA*mCmAmUmUmAmGfCfAfAmAmGmUmUmUmGmCmC*mC*mA |
| | SEQ ID NO: 1173 | PmU*fG*mGmGfCmAfAmAmCmUmUmUmGfCmUfAmAmUmGmUmG*mC*mU |
| Duplex NO: 394 | SEQ ID NO: 1174 | mA*mC*mAmUmUmAmGmCfAfAfAmGmUmUmUmGmCmCmC*mU*mA |
| | SEQ ID NO: 1175 | PmU*fA*mGmGfGmCfAmAmAmCmUmUmUfGmCfUmAmAmUmGmU*mG*mC |
| Duplex NO: 395 | SEQ ID NO: 1176 | mC*mA*mUmUmAmGmCmAfAfAfGmUmUmUmGmCmCmCmU*mC*mA |
| | SEQ ID NO: 1177 | PmU*fG*mAmGfGmGfCmAmAmAmCmUmUfUmGfCmUmAmAmUmG*mU*mG |
| Duplex NO: 396 | SEQ ID NO: 1178 | mU*mA*mGmCmAmAmAmGfUfUfUmGmCmCmCmUmCmAmG*mU*mA |
| | SEQ ID NO: 1179 | PmU*fA*mCmUfGmAfGmGmGmCmAmAmAfCmUfUmUmGmCmUmA*mA*mU |
| Duplex NO: 397 | SEQ ID NO: 1180 | mG*mC*mAmAmAmGmUmUfUfGfCmCmCmUmCmAmGmUmU*mC*mA |
| | SEQ ID NO: 1181 | PmU*fG*mAmAfCmUfGmAmGmGmGmCmAfAmAfCmUmUmUmGmC*mU*mA |
| Duplex NO: 398 | SEQ ID NO: 1182 | mA*mC*mUmCmAmCmAmGfGfAfUmGmAmAmGmUmAmAmG*mG*mA |
| | SEQ ID NO: 1183 | PmU*fC*mCmUfUmAfCmUmUmCmAmUmCfCmUfGmUmGmAmGmU*mU*mG |
| Duplex NO: 399 | SEQ ID NO: 1184 | mC*mA*mCmAmGmGmAmUfGfAfAmGmUmAmAmGmGmGmA*mA*mA |
| | SEQ ID NO: 1185 | PmU*fU*mUmCfCmCfUmUmAmCmUmUmCfAmUfCmCmUmGmUmG*mA*mG |
| Duplex NO: 400 | SEQ ID NO: 1186 | mC*mA*mGmGmAmUmGmAfAfGfUmAmAmGmGmGmAmAmA*mA*mA |
| | SEQ ID NO: 1187 | PmU*fU*mUmUfUmCfCmCmUmUmAmCmUfUmCfAmUmCmCmUmG*mU*mG |
| Duplex NO: 401 | SEQ ID NO: 1188 | mA*mG*mGmAmUmGmAmAfGfUfAmAmGmGmGmAmAmAmA*mU*mA |
| | SEQ ID NO: 1189 | PmU*fA*mUmUfUmUfCmCmCmUmUmAmCfUmUfCmAmUmCmCmU*mG*mU |
| Duplex NO: 402 | SEQ ID NO: 1190 | mG*mC*mGmAmGmGmGmUfCfGfUmCmCmAmAmUmUmUmG*mG*mA |
| | SEQ ID NO: 1191 | PmU*fC*mCmAfAmAfUmUmGmGmAmCmGfAmCfCmCmUmCmGmC*mG*mG |
| Duplex NO: 403 | SEQ ID NO: 1192 | mC*mG*mAmGmGmGmUmCfGfUfCmCmAmAmUmUmUmGmG*mC*mA |
| | SEQ ID NO: 1193 | PmU*fG*mCmCfAmAfAmUmUmGmGmAmCfGmAfCmCmCmUmCmG*mC*mG |
| Duplex NO: 404 | SEQ ID NO: 1194 | mG*mA*mGmGmGmUmCmGfUfCfCmAmAmUmUmUmGmGmC*mA*mA |
| | SEQ ID NO: 1195 | PmU*fU*mGmCfCmAfAmAmUmUmGmGmAfCmGfAmCmCmCmUmC*mG*mC |
| Duplex NO: 405 | SEQ ID NO: 1196 | mG*mU*mCmGmUmCmCmAfAfUfUmUmGmGmCmAmGmCmU*mC*mA |
| | SEQ ID NO: 1197 | PmU*fG*mAmGfCmUfGmCmCmAmAmAmUfUmGfGmAmCmGmAmC*mC*mC |
| Duplex NO: 406 | SEQ ID NO: 1198 | mU*mC*mGmUmCmCmAmAfUfUfUmGmGmCmAmGmCmUmC*mA*mA |
| | SEQ ID NO: 1199 | PmU*fU*mGmAfGmCfUmGmCmCmAmAmAfUmUfGmGmAmCmGmA*mC*mC |
| Duplex NO: 407 | SEQ ID NO: 1200 | mC*mG*mUmCmCmAmAmUfUfUfGmGmCmAmGmCmUmCmA*mG*mA |
| | SEQ ID NO: 1201 | PmU*fC*mUmGfAmGfCmUmGmCmCmAmAfAmUfUmGmGmAmCmG*mA*mC |
| Duplex NO: 408 | SEQ ID NO: 1202 | mU*mC*mCmAmAmUmUmUfGfGfCmAmGmCmUmCmAmGmC*mC*mA |
| | SEQ ID NO: 1203 | PmU*fG*mGmCfUmGfAmGmCmUmGmCmCfAmAfAmUmUmGmGmA*mC*mG |
| Duplex NO: 409 | SEQ ID NO: 1204 | mA*mA*mUmUmUmGmGmCfAfGfCmUmCmAmGmCmCmAmU*mU*mA |
| | SEQ ID NO: 1205 | PmU*fA*mAmUfGmGfCmUmGmAmGmCmUfGmCfCmAmAmAmUmU*mG*mG |
| Duplex NO: 410 | SEQ ID NO: 1206 | mU*mU*mGmGmCmAmGmCfUfCfAmGmCmCmAmUmUmUmU*mG*mA |
| | SEQ ID NO: 1207 | PmU*fC*mAmAfAmAfUmGmGmCmUmGmAfGmCfUmGmCmCmAmA*mA*mU |
| Duplex NO: 411 | SEQ ID NO: 1208 | mU*mG*mGmCmAmGmCmUfCfAfGmCmCmAmUmUmUmUmG*mC*mA |
| | SEQ ID NO: 1209 | PmU*fG*mCmAfAmAfAmUmGmGmCmUmGfAmGfCmUmGmCmCmA*mA*mA |
| Duplex NO: 412 | SEQ ID NO: 1210 | mC*mC*mGmAmGmUmUmUfUfAfGmAmAmGmAmAmGmAmA*mG*mA |
| | SEQ ID NO: 1211 | PmU*fC*mUmUfCmUfUmCmUmUmCmUmAfAmAfAmCmUmCmGmG*mG*mC |
| Duplex NO: 413 | SEQ ID NO: 1212 | mA*mG*mAmGmGmGmUmCfAfAfGmAmUmGmAmUmUmAmU*mG*mA |
| | SEQ ID NO: 1213 | PmU*fC*mAmUfAmAfUmCmAmUmCmUmUfGmAfCmCmCmUmCmU*mG*mA |
| Duplex NO: 414 | SEQ ID NO: 1214 | mG*mG*mGmUmCmAmAmGfAfUfGmAmUmUmAmUmGmUmC*mU*mA |
| | SEQ ID NO: 1215 | PmU*fA*mGmAfCmAfUmAmAmUmCmAmUfCmUfUmGmAmCmCmC*mU*mC |
| Duplex NO: 415 | SEQ ID NO: 1216 | mA*mG*mUmCmGmCmUmGfGfAfUmAmGmCmUmGmAmUmC*mC*mA |
| | SEQ ID NO: 1217 | PmU*fG*mGmAfUmCfAmGmCmUmAmUmCfCmAfGmCmGmAmCmU*mG*mU |
| Duplex NO: 416 | SEQ ID NO: 1218 | mG*mU*mCmGmCmUmGmGfAfUfAmGmCmUmGmAmUmCmC*mU*mA |
| | SEQ ID NO: 1219 | PmU*fA*mGmGfAmUfCmAmGmCmUmAmUfCmCfAmGmCmGmAmC*mU*mG |
| Duplex NO: 417 | SEQ ID NO: 1220 | mU*mC*mGmCmUmGmGmAfUfAfGmCmUmGmAmUmCmCmU*mU*mA |
| | SEQ ID NO: 1221 | PmU*fA*mAmGfGmAfUmCmAmGmCmUmAfUmCfCmAmGmCmGmA*mC*mU |
| Duplex NO: 418 | SEQ ID NO: 1222 | mA*mA*mAmAmGmUmUmGfAfGfGmUmUmAmUmAmAmAmA*mC*mA |
| | SEQ ID NO: 1223 | PmU*fG*mUmUfUmUfAmUmAmAmCmCmUfCmAfAmCmUmUmUmU*mC*mU |
| Duplex NO: 419 | SEQ ID NO: 1224 | mA*mA*mAmGmUmUmGmAfGfGfUmUmAmUmAmAmAmAmC*mC*mA |
| | SEQ ID NO: 1225 | PmU*fG*mGmUfUmUfUmAmUmAmAmCmCfUmCfAmAmCmUmUmU*mU*mC |
| Duplex NO: 420 | SEQ ID NO: 1226 | mU*mG*mGmAmUmAmUmAfUfGfCmCmCmAmUmCmCmCmU*mG*mA |
| | SEQ ID NO: 1227 | PmU*fC*mAmGfGmGfAmUmGmGmGmCmAfUmAfUmAmUmCmCmA*mA*mU |
| Duplex NO: 421 | SEQ ID NO: 1228 | mG*mG*mAmUmAmUmAmUfGfCfCmCmAmUmCmCmCmUmG*mU*mA |
| | SEQ ID NO: 1229 | PmU*fA*mCmAfGmGfGmAmUmGmGmGmCfAmUfAmUmAmUmCmC*mA*mA |
| Duplex NO: 422 | SEQ ID NO: 1230 | mA*mU*mAmUmAmUmGmCfCfCfAmUmCmCmCmUmGmUmU*mG*mA |
| | SEQ ID NO: 1231 | PmU*fC*mAmAfCmAfGmGmGmAmUmGmGfGmCfAmUmAmUmAmU*mC*mC |
| Duplex NO: 423 | SEQ ID NO: 1232 | mU*mA*mUmAmUmGmCmCfCfAfUmCmCmCmUmGmUmUmG*mG*mA |
| | SEQ ID NO: 1233 | PmU*fC*mCmAfAmCfAmGmGmGmAmUmGfGmGfCmAmUmAmUmA*mU*mC |
| Duplex NO: 424 | SEQ ID NO: 1234 | mA*mU*mAmUmGmCmCmCfAfUfCmCmCmUmGmUmUmGmG*mA*mA |
| | SEQ ID NO: 1235 | PmU*fU*mCmCfAmAfCmAmGmGmGmAmUfGmGfGmCmAmUmAmU*mA*mU |
| Duplex NO: 425 | SEQ ID NO: 1236 | mU*mA*mUmGmCmCmCmAfUfCfCmCmUmGmUmUmGmGmA*mG*mA |
| | SEQ ID NO: 1237 | PmU*fC*mUmCfCmAfAmCmAmGmGmGmAfUmGfGmGmCmAmUmA*mU*mA |
| Duplex NO: 426 | SEQ ID NO: 1238 | mG*mG*mUmGmGmAmGmGfUfGfCmCmAmGmCmAmGmCmC*mG*mA |
| | SEQ ID NO: 1239 | PmU*fC*mGmGfCmUfGmCmUmGmGmCmAfCmCfUmCmCmAmCmC*mA*mA |
| Duplex NO: 427 | SEQ ID NO: 1240 | mG*mU*mCmCmUmUmGmCfAfGfAmUmGmGmGmAmUmGmA*mC*mA |
| | SEQ ID NO: 1241 | PmU*fG*mUmCfAmUfCmCmCmAmUmCmUfGmCfAmAmGmGmAmC*mU*mC |
| Duplex NO: 428 | SEQ ID NO: 1242 | mU*mC*mCmUmUmGmCmAfGfAfUmGmGmGmAmUmGmAmC*mU*mA |
| | SEQ ID NO: 1243 | PmU*fA*mGmUfCmAfUmCmCmCmAmUmCfUmGfCmAmAmGmGmA*mC*mU |
| Duplex NO: 429 | SEQ ID NO: 1244 | mC*mU*mUmGmCmAmGmAfUfGfGmGmAmUmGmAmCmUmC*mG*mA |
| | SEQ ID NO: 1245 | PmU*fC*mGmAfGmUfCmAmUmCmCmCmAfUmCfUmGmCmAmAmG*mG*mA |
| Duplex NO: 430 | SEQ ID NO: 1246 | mU*mG*mCmGmUmCmUmUfCfCfAmCmGmUmGmCmUmUmG*mU*mA |
| | SEQ ID NO: 1247 | PmU*fA*mCmAfAmGfCmAmCmGmUmGmGfAmAfGmAmCmGmCmA*mC*mA |
| Duplex NO: 431 | SEQ ID NO: 1248 | mG*mC*mGmUmCmUmUmCfCfAfCmGmUmGmCmUmUmGmU*mG*mA |
| | SEQ ID NO: 1249 | PmU*fC*mAmCfAmAfGmCmAmCmGmUmGfGmAfAmGmAmCmGmC*mA*mC |
| Duplex NO: 432 | SEQ ID NO: 1250 | mG*mU*mCmUmUmCmCmAfCfGfUmGmCmUmUmGmUmGmA*mC*mA |
| | SEQ ID NO: 1251 | PmU*fG*mUmCfAmCfAmAmGmCmAmCmGfUmGfGmAmAmGmAmC*mG*mC |
| Duplex NO: 433 | SEQ ID NO: 1252 | mC*mA*mCmGmUmGmCmUfUfGfUmGmAmCmUmCmUmGmC*mA*mA |
| | SEQ ID NO: 1253 | PmU*fU*mGmCfAmGfAmGmUmCmAmCmAfAmGfCmAmCmGmUmG*mG*mA |
| Duplex NO: 434 | SEQ ID NO: 1254 | mU*mG*mCmUmUmGmUmGfAfCfUmCmUmGmCmAmGmAmA*mG*mA |
| | SEQ ID NO: 1255 | PmU*fC*mUmUfCmUfGmCmAmGmAmGmUfCmAfCmAmAmGmCmA*mC*mG |
| Duplex NO: 435 | SEQ ID NO: 1256 | mU*mU*mUmAmUmAmUmCfCfGfUmUmUmCmCmAmGmUmC*mC*mA |
| | SEQ ID NO: 1257 | PmU*fG*mGmAfCmUfGmGmAmAmAmCmGfGmAfUmAmUmAmAmA*mG*mG |
| Duplex NO: 436 | SEQ ID NO: 1258 | mU*mU*mAmUmAmUmCmCfGfUfUmUmCmCmAmGmUmCmC*mA*mA |
| | SEQ ID NO: 1259 | PmU*fU*mGmGfAmCfUmGmGmAmAmAmCfGmGfAmUmAmUmAmA*mA*mG |
| Duplex NO: 437 | SEQ ID NO: 1260 | mA*mU*mAmUmCmCmGmUfUfUfCmCmAmGmUmCmCmAmG*mG*mA |
| | SEQ ID NO: 1261 | PmU*fC*mCmUfGmGfAmCmUmGmGmAmAfAmCfGmGmAmUmAmU*mA*mA |
| Duplex NO: 438 | SEQ ID NO: 1262 | mC*mU*mGmCmCmAmUmGfGfCfUmGmGmGmAmGmCmAmU*mA*mA |
| | SEQ ID NO: 1263 | PmU*fU*mAmUfGmCfUmCmCmCmAmGmCfCmAfUmGmGmCmAmG*mA*mG |
| Duplex NO: 439 | SEQ ID NO: 1264 | mU*mG*mCmCmAmUmGmGfCfUfGmGmGmAmGmCmAmUmA*mG*mA |
| | SEQ ID NO: 1265 | PmU*fC*mUmAfUmGfCmUmCmCmCmAmGfCmCfAmUmGmGmCmA*mG*mA |
| Duplex NO: 440 | SEQ ID NO: 1266 | mG*mC*mCmAmUmGmGmCfUfGfGmGmAmGmCmAmUmAmG*mG*mA |
| | SEQ ID NO: 1267 | PmU*fC*mCmUfAmUfGmCmUmCmCmCmAfGmCfCmAmUmGmGmC*mA*mG |
| Duplex NO: 441 | SEQ ID NO: 1268 | mA*mC*mAmGmGmAmUmGfCfAfGmCmAmCmAmGmAmAmU*mG*mA |
| | SEQ ID NO: 1269 | PmU*fC*mAmUfUmCfUmGmUmGmCmUmGfCmAfUmCmCmUmGmU*mC*mC |
| Duplex NO: 442 | SEQ ID NO: 1270 | mG*mU*mAmGmUmUmCmAfAfAfCmUmGmUmAmUmUmAmC*mU*mA |
| | SEQ ID NO: 1271 | PmU*fA*mGmUfAmAfUmAmCmAmGmUmUfUmGfAmAmCmUmAmC*mC*mA |
| Duplex NO: 443 | SEQ ID NO: 1272 | mG*mU*mUmCmAmAmAmCfUfGfUmAmUmUmAmCmUmUmU*mA*mA |
| | SEQ ID NO: 1273 | PmU*fU*mAmAfAmGfUmAmAmUmAmCmAfGmUfUmUmGmAmAmC*mU*mA |
| Duplex NO: 444 | SEQ ID NO: 1274 | mU*mU*mCmAmAmAmCmUfGfUfAmUmUmAmCmUmUmUmA*mA*mA |
| | SEQ ID NO: 1275 | PmU*fU*mUmAfAmAfGmUmAmAmUmAmCfAmGfUmUmUmGmAmA*mC*mU |
| Duplex NO: 445 | SEQ ID NO: 1276 | mU*mC*mAmAmAmCmUmGfUfAfUmUmAmCmUmUmUmAmA*mU*mA |
| | SEQ ID NO: 1277 | PmU*fA*mUmUfAmAfAmGmUmAmAmUmAfCmAfGmUmUmUmGmA*mA*mC |
| Duplex NO: 446 | SEQ ID NO: 1278 | mA*mA*mCmUmGmUmAmUfUfAfCmUmUmUmAmAmUmGmG*mA*mA |
| | SEQ ID NO: 1279 | PmU*fU*mCmCfAmUfUmAmAmAmGmUmAfAmUfAmCmAmGmUmU*mU*mG |
| Duplex NO: 447 | SEQ ID NO: 1280 | mA*mC*mUmGmUmAmUmUfAfCfUmUmUmAmAmUmGmGmA*mA*mA |
| | SEQ ID NO: 1281 | PmU*fU*mUmCfCmAfUmUmAmAmAmGmUfAmAfUmAmCmAmGmU*mU*mU |
| Duplex NO: 448 | SEQ ID NO: 1282 | mC*mU*mGmUmAmUmUmAfCfUfUmUmAmAmUmGmGmAmA*mG*mA |
| | SEQ ID NO: 1283 | PmU*fC*mUmUfCmCfAmUmUmAmAmAmGfUmAfAmUmAmCmAmG*mU*mU |
| Duplex NO: 449 | SEQ ID NO: 1284 | mU*mG*mUmAmUmUmAmCfUfUfUmAmAmUmGmGmAmAmG*mC*mA |
| | SEQ ID NO: 1285 | PmU*fG*mCmUfUmCfCmAmUmUmAmAmAfGmUfAmAmUmAmCmA*mG*mU |
| Duplex NO: 450 | SEQ ID NO: 1286 | mU*mU*mAmUmAmUmAmUfCfAfGmCmUmGmCmAmCmCmA*mU*mA |
| | SEQ ID NO: 1287 | PmU*fA*mUmGfGmUfGmCmAmGmCmUmGfAmUfAmUmAmUmAmA*mA*mU |
| Duplex NO: 451 | SEQ ID NO: 1288 | mU*mA*mUmAmUmAmUmCfAfGfCmUmGmCmAmCmCmAmU*mG*mA |
| | SEQ ID NO: 1289 | PmU*fC*mAmUfGmGfUmGmCmAmGmCmUfGmAfUmAmUmAmUmA*mA*mA |
| Duplex NO: 452 | SEQ ID NO: 1290 | mA*mU*mAmUmAmUmCmAfGfCfUmGmCmAmCmCmAmUmG*mC*mA |
| | SEQ ID NO: 1291 | PmU*fG*mCmAfUmGfGmUmGmCmAmGmCfUmGfAmUmAmUmAmU*mA*mA |
| Duplex NO: 453 | SEQ ID NO: 1292 | mU*mA*mUmAmUmCmAmGfCfUfGmCmAmCmCmAmUmGmC*mC*mA |
| | SEQ ID NO: 1293 | PmU*fG*mGmCfAmUfGmGmUmGmCmAmGfCmUfGmAmUmAmUmA*mU*mA |
| Duplex NO: 454 | SEQ ID NO: 1294 | mA*mU*mAmUmCmAmGmCfUfGfCmAmCmCmAmUmGmCmC*mA*mA |
| | SEQ ID NO: 1295 | PmU*fU*mGmGfCmAfUmGmGmUmGmCmAfGmCfUmGmAmUmAmU*mA*mU |
| Duplex NO: 455 | SEQ ID NO: 1296 | mA*mU*mCmAmGmCmUmGfCfAfCmCmAmUmGmCmCmAmU*mC*mA |
| | SEQ ID NO: 1297 | PmU*fG*mAmUfGmGfCmAmUmGmGmUmGfCmAfGmCmUmGmAmU*mA*mU |
| Duplex NO: 456 | SEQ ID NO: 1298 | mU*mU*mUmGmCmAmGmAfUfGfCmUmAmGmGmUmGmUmU*mC*mA |
| | SEQ ID NO: 1299 | PmU*fG*mAmAfCmAfCmCmUmAmGmCmAfUmCfUmGmCmAmAmA*mC*mA |
| Duplex NO: 457 | SEQ ID NO: 1300 | mU*mG*mCmAmGmAmUmGfCfUfAmGmGmUmGmUmUmCmA*mA*mA |
| | SEQ ID NO: 1301 | PmU*fU*mUmGfAmAfCmAmCmCmUmAmGfCmAfUmCmUmGmCmA*mA*mA |
| Duplex NO: 458 | SEQ ID NO: 1302 | mG*mC*mAmGmAmUmGmCfUfAfGmGmUmGmUmUmCmAmA*mG*mA |
| | SEQ ID NO: 1303 | PmU*fC*mUmUfGmAfAmCmAmCmCmUmAfGmCfAmUmCmUmGmC*mA*mA |
| Duplex NO: 459 | SEQ ID NO: 1304 | mC*mA*mGmAmUmGmCmUfAfGfGmUmGmUmUmCmAmAmG*mG*mA |
| | SEQ ID NO: 1305 | PmU*fC*mCmUfUmGfAmAmCmAmCmCmUfAmGfCmAmUmCmUmG*mC*mA |
| Duplex NO: 460 | SEQ ID NO: 1306 | mA*mG*mAmUmGmCmUmAfGfGfUmGmUmUmCmAmAmGmG*mA*mA |
| | SEQ ID NO: 1307 | PmU*fU*mCmCfUmUfGmAmAmCmAmCmCfUmAfGmCmAmUmCmU*mG*mC |
| Duplex NO: 461 | SEQ ID NO: 1308 | mG*mC*mUmAmGmGmUmGfUfUfCmAmAmGmGmAmGmCmA*mU*mA |
| | SEQ ID NO: 1309 | PmU*fA*mUmGfCmUfCmCmUmUmGmAmAfCmAfCmCmUmAmGmC*mA*mU |
| Duplex NO: 462 | SEQ ID NO: 1310 | mU*mA*mGmGmUmGmUmUfCfAfAmGmGmAmGmCmAmUmG*mC*mA |
| | SEQ ID NO: 1311 | PmU*fG*mCmAfUmGfCmUmCmCmUmUmGfAmAfCmAmCmCmUmA*mG*mC |
| Duplex NO: 463 | SEQ ID NO: 1312 | mA*mG*mGmUmGmUmUmCfAfAfGmGmAmGmCmAmUmGmC*mA*mA |
| | SEQ ID NO: 1313 | PmU*fU*mGmCfAmUfGmCmUmCmCmUmUfGmAfAmCmAmCmCmU*mA*mG |
| Duplex NO: 464 | SEQ ID NO: 1314 | mG*mG*mUmGmUmUmCmAfAfGfGmAmGmCmAmUmGmCmA*mA*mA |
| | SEQ ID NO: 1315 | PmU*fU*mUmGfCmAfUmGmCmUmCmCmUfUmGfAmAmCmAmCmC*mU*mA |
| Duplex NO: 465 | SEQ ID NO: 1316 | mU*mG*mUmUmCmAmAmGfGfAfGmCmAmUmGmCmAmAmA*mG*mA |
| | SEQ ID NO: 1317 | PmU*fC*mUmUfUmGfCmAmUmGmCmUmCfCmUfUmGmAmAmCmA*mC*mC |
| Duplex NO: 466 | SEQ ID NO: 1318 | mG*mU*mUmCmAmAmGmGfAfGfCmAmUmGmCmAmAmAmG*mA*mA |
| | SEQ ID NO: 1319 | PmU*fU*mCmUfUmUfGmCmAmUmGmCmUfCmCfUmUmGmAmAmC*mA*mC |
| Duplex NO: 467 | SEQ ID NO: 1320 | mC*mC*mCmGmGmCmAmGfCfUfUmGmCmCmCmGmAmAmU*mU*mA |
| | SEQ ID NO: 1321 | PmU*fA*mAmUfUmCfGmGmGmCmAmAmGfCmUfGmCmCmGmGmG*mC*mA |
| Duplex NO: 468 | SEQ ID NO: 1322 | mC*mG*mGmCmAmGmCmUfUfGfCmCmCmGmAmAmUmUmG*mU*mA |
| | SEQ ID NO: 1323 | PmU*fA*mCmAfAmUfUmCmGmGmGmCmAfAmGfCmUmGmCmCmG*mG*mG |
| Duplex NO: 469 | SEQ ID NO: 1324 | mC*mA*mUmUmGmGmCmAfGfCfAmGmGmAmCmAmUmCmU*mU*mA |
| | SEQ ID NO: 1325 | PmU*fA*mAmGfAmUfGmUmCmCmUmGmCfUmGfCmCmAmAmUmG*mC*mU |
| Duplex NO: 470 | SEQ ID NO: 1326 | mA*mU*mUmGmGmCmAmGfCfAfGmGmAmCmAmUmCmUmU*mG*mA |
| | SEQ ID NO: 1327 | PmU*fC*mAmAfGmAfUmGmUmCmCmUmGfCmUfGmCmCmAmAmU*mG*mC |
| Duplex NO: 471 | SEQ ID NO: 1328 | mU*mG*mGmCmAmGmCmAfGfGfAmCmAmUmCmUmUmGmU*mC*mA |
| | SEQ ID NO: 1329 | PmU*fG*mAmCfAmAfGmAmUmGmUmCmCfUmGfCmUmGmCmCmA*mA*mU |
| Duplex NO: 472 | SEQ ID NO: 1330 | mU*mA*mUmUmUmGmUmUfCfAfGmAmGmAmGmGmCmCmU*mU*mA |
| | SEQ ID NO: 1331 | PmU*fA*mAmGfGmCfCmUmCmUmCmUmGfAmAfCmAmAmAmUmA*mG*mC |
| Duplex NO: 473 | SEQ ID NO: 1332 | mU*mU*mAmCmUmAmAmAfGfAfUmAmUmCmAmGmAmGmC*mU*mA |
| | SEQ ID NO: 1333 | PmU*fA*mGmCfUmCfUmGmAmUmAmUmCfUmUfUmAmGmUmAmA*mA*mG |
| Duplex NO: 474 | SEQ ID NO: 1334 | mC*mA*mAmAmUmCmUmGfUfAfUmAmUmGmUmUmGmUmA*mA*mA |
| | SEQ ID NO: 1335 | PmU*fU*mUmAfCmAfAmCmAmUmAmUmAfCmAfGmAmUmUmUmG*mA*mU |
| Duplex NO: 475 | SEQ ID NO: 1336 | mA*mA*mAmUmCmUmGmUfAfUfAmUmGmUmUmGmUmAmA*mU*mA |
| | SEQ ID NO: 1337 | PmU*fA*mUmUfAmCfAmAmCmAmUmAmUfAmCfAmGmAmUmUmU*mG*mA |
| Duplex NO: 476 | SEQ ID NO: 1338 | mG*mU*mAmCmUmGmAmUfAfAfAmAmUmAmCmUmCmUmA*mG*mA |
| | SEQ ID NO: 1339 | PmU*fC*mUmAfGmAfGmUmAmUmUmUmUfAmUfCmAmGmUmAmC*mA*mC |
| Duplex NO: 477 | SEQ ID NO: 1340 | mU*mU*mGmUmCmCmAmGfGfAfAmAmCmCmCmUmGmGmC*mU*mA |
| | SEQ ID NO: 1341 | PmU*fA*mGmCfCmAfGmGmGmUmUmUmCfCmUfGmGmAmCmAmA*mC*mA |
| Duplex NO: 478 | SEQ ID NO: 1342 | mU*mC*mCmAmGmGmAmAfAfCfCmCmUmGmGmCmUmUmG*mC*mA |
| | SEQ ID NO: 1343 | PmU*fG*mCmAfAmGfCmCmAmGmGmGmUfUmUfCmCmUmGmGmA*mC*mA |
| Duplex NO: 479 | SEQ ID NO: 1344 | mU*mU*mGmUmAmGmAmAfGfAfUmGmGmAmCmUmCmUmG*mA*mA |
| | SEQ ID NO: 1345 | PmU*fU*mCmAfGmAfGmUmCmCmAmUmCfUmUfCmUmAmCmAmA*mG*mA |
| Duplex NO: 480 | SEQ ID NO: 1346 | mU*mG*mUmAmGmAmAmGfAfUfGmGmAmCmUmCmUmGmA*mA*mA |
| | SEQ ID NO: 1347 | PmU*fU*mUmCfAmGfAmGmUmCmCmAmUfCmUfUmCmUmAmCmA*mA*mG |
| Duplex NO: 481 | SEQ ID NO: 1348 | mU*mA*mAmUmGmUmUmUfUfGfUmAmCmAmAmUmUmAmC*mU*mA |
| | SEQ ID NO: 1349 | PmU*fA*mGmUfAmAfUmUmGmUmAmCmAfAmAfAmCmAmUmUmA*mA*mU |
| Duplex NO: 482 | SEQ ID NO: 1350 | mU*mA*mUmAmCmAmUmUfUfUfGmUmUmAmUmAmGmAmA*mU*mA |
| | SEQ ID NO: 1351 | PmU*fA*mUmUfCmUfAmUmAmAmCmAmAfAmAfUmGmUmAmUmA*mC*mA |
| Duplex NO: 483 | SEQ ID NO: 1352 | mU*mA*mCmAmUmUmUmUfGfUfUmAmUmAmGmAmAmUmA*mC*mA |
| | SEQ ID NO: 1353 | PmU*fG*mUmAfUmUfCmUmAmUmAmAmCfAmAfAmAmUmGmUmA*mU*mA |
| Duplex NO: 484 | SEQ ID NO: 1354 | mA*mC*mAmUmUmUmUmGfUfUfAmUmAmGmAmAmUmAmC*mU*mA |
| | SEQ ID NO: 1355 | PmU*fA*mGmUfAmUfUmCmUmAmUmAmAfCmAfAmAmAmUmGmU*mA*mU |
| Duplex NO: 485 | SEQ ID NO: 1356 | mC*mA*mAmUmGmUmUmGfUfCfAmAmGmAmCmUmUmUmU*mU*mA |
| | SEQ ID NO: 1357 | PmU*fA*mAmAfAmAfGmUmCmUmUmGmAfCmAfAmCmAmUmUmG*mU*mA |
| Duplex NO: 486 | SEQ ID NO: 1358 | mA*mA*mUmGmUmUmGmUfCfAfAmGmAmCmUmUmUmUmU*mC*mA |
| | SEQ ID NO: 1359 | PmU*fG*mAmAfAmAfAmGmUmCmUmUmGfAmCfAmAmCmAmUmU*mG*mU |
| Duplex NO: 487 | SEQ ID NO: 1360 | mA*mU*mGmUmUmGmUmCfAfAfGmAmCmUmUmUmUmUmC*mG*mA |
| | SEQ ID NO: 1361 | PmU*fC*mGmAfAmAfAmAmGmUmCmUmUfGmAfCmAmAmCmAmU*mU*mG |
| Duplex NO: 488 | SEQ ID NO: 1362 | mU*mC*mUmGmCmAmUmGfAfUfGmUmCmCmAmUmGmAmA*mC*mA |
| | SEQ ID NO: 1363 | PmU*fG*mUmUfCmAfUmGmGmAmCmAmUfCmAfUmGmCmAmGmA*mU*mG |
| Duplex NO: 489 | SEQ ID NO: 1364 | mC*mU*mGmCmAmUmGmAfUfGfUmCmCmAmUmGmAmAmC*mA*mA |
| | SEQ ID NO: 1365 | PmU*fU*mGmUfUmCfAmUmGmGmAmCmAfUmCfAmUmGmCmAmG*mA*mU |
| Duplex NO: 490 | SEQ ID NO: 1366 | mU*mA*mAmGmAmUmCmUfGfUfGmGmUmUmGmGmAmAmU*mU*mA |
| | SEQ ID NO: 1367 | PmU*fA*mAmUfUmCfCmAmAmCmCmAmCfAmGfAmUmCmUmUmA*mU*mU |
| Duplex NO: 491 | SEQ ID NO: 1368 | mA*mA*mGmAmUmCmUmGfUfGfGmUmUmGmGmAmAmUmU*mA*mA |
| | SEQ ID NO: 1369 | PmU*fU*mAmAfUmUfCmCmAmAmCmCmAfCmAfGmAmUmCmUmU*mA*mU |
| Duplex NO: 492 | SEQ ID NO: 1370 | mA*mG*mAmUmCmUmGmUfGfGfUmUmGmGmAmAmUmUmA*mU*mA |
| | SEQ ID NO: 1371 | PmU*fA*mUmAfAmUfUmCmCmAmAmCmCfAmCfAmGmAmUmCmU*mU*mA |
| Duplex NO: 493 | SEQ ID NO: 1372 | mU*mG*mUmAmUmAmUmUfUfAfCmUmUmCmCmAmGmUmU*mC*mA |
| | SEQ ID NO: 1373 | PmU*fG*mAmAfCmUfGmGmAmAmGmUmAfAmAfUmAmUmAmCmA*mG*mG |
| Duplex NO: 494 | SEQ ID NO: 1374 | mG*mU*mAmUmAmUmUmUfAfCfUmUmCmCmAmGmUmUmC*mC*mA |
| | SEQ ID NO: 1375 | PmU*fG*mGmAfAmCfUmGmGmAmAmGmUfAmAfAmUmAmUmAmC*mA*mG |
| Duplex NO: 495 | SEQ ID NO: 1376 | mU*mA*mUmAmUmUmUmAfCfUfUmCmCmAmGmUmUmCmC*mA*mA |
| | SEQ ID NO: 1377 | PmU*fU*mGmGfAmAfCmUmGmGmAmAmGfUmAfAmAmUmAmUmA*mC*mA |
| Duplex NO: 496 | SEQ ID NO: 1378 | mU*mU*mUmAmCmUmUmCfCfAfGmUmUmCmCmAmGmAmA*mU*mA |
| | SEQ ID NO: 1379 | PmU*fA*mUmUfCmUfGmGmAmAmCmUmGfGmAfAmGmUmAmAmA*mU*mA |
| Duplex NO: 497 | SEQ ID NO: 1380 | mU*mA*mUmUmUmUmGmGfGfUfAmUmUmGmCmUmGmGmC*mC*mA |
| | SEQ ID NO: 1381 | PmU*fG*mGmCfCmAfGmCmAmAmUmAmCfCmCfAmAmAmAmUmA*mU*mA |
| Duplex NO: 498 | SEQ ID NO: 1382 | mU*mA*mUmUmCmAmGmUfAfCfAmGmUmUmGmUmCmAmU*mU*mA |
| | SEQ ID NO: 1383 | PmU*fA*mAmUfGmAfCmAmAmCmUmGmUfAmCfUmGmAmAmUmA*mC*mA |
| Duplex NO: 499 | SEQ ID NO: 1384 | mA*mU*mUmCmAmGmUmAfCfAfGmUmUmGmUmCmAmUmU*mC*mA |
| | SEQ ID NO: 1385 | PmU*fG*mAmAfUmGfAmCmAmAmCmUmGfUmAfCmUmGmAmAmU*mA*mC |
| Duplex NO: 500 | SEQ ID NO: 1386 | mA*mG*mAmCmAmAmAmGfAfAfUmUmGmAmCmAmGmGmC*mU*mA |
| | SEQ ID NO: 1387 | PmU*fA*mGmCfCmUfGmUmCmAmAmUmUfCmUfUmUmGmUmCmU*mA*mA |
| Duplex NO: 501 | SEQ ID NO: 1388 | mG*mA*mCmAmAmAmGmAfAfUfUmGmAmCmAmGmGmCmU*mU*mA |
| | SEQ ID NO: 1389 | PmU*fA*mAmGfCmCfUmGmUmCmAmAmUfUmCfUmUmUmGmUmC*mU*mA |
| Duplex NO: 502 | SEQ ID NO: 1390 | mG*mC*mUmUmGmAmAmUfGfAfAmGmCmUmUmUmGmCmC*mC*mA |
| | SEQ ID NO: 1391 | PmU*fG*mGmGfCmAfAmAmGmCmUmUmCfAmUfUmCmAmAmGmC*mC*mU |
| Duplex NO: 503 | SEQ ID NO: 1392 | mC*mU*mUmGmAmAmUmGfAfAfGmCmUmUmUmGmCmCmC*mU*mA |
| | SEQ ID NO: 1393 | PmU*fA*mGmGfGmCfAmAmAmGmCmUmUfCmAfUmUmCmAmAmG*mC*mC |
| Duplex NO: 504 | SEQ ID NO: 1394 | mU*mU*mGmAmAmUmGmAfAfGfCmUmUmUmGmCmCmCmU*mU*mA |
| | SEQ ID NO: 1395 | PmU*fA*mAmGfGmGfCmAmAmAmGmCmUfUmCfAmUmUmCmAmA*mG*mC |
| Duplex NO: 505 | SEQ ID NO: 1396 | mA*mU*mGmAmAmGmCmUfUfUfGmCmCmCmUmUmUmUmU*mC*mA |
| | SEQ ID NO: 1397 | PmU*fG*mAmAfAmAfAmGmGmGmCmAmAfAmGfCmUmUmCmAmU*mU*mC |
| Duplex NO: 506 | SEQ ID NO: 1398 | mC*mA*mAmAmGmUmUmUfGfCfCmCmUmCmAmGmUmUmC*mC*mA |
| | SEQ ID NO: 1399 | PmU*fG*mGmAfAmCfUmGmAmGmGmGmCfAmAfAmCmUmUmUmG*mC*mU |
| Duplex NO: 507 | SEQ ID NO: 1400 | mA*mA*mGmUmUmUmGmCfCfCfUmCmAmGmUmUmCmCmA*mA*mA |
| | SEQ ID NO: 1401 | PmU*fU*mUmGfGmAfAmCmUmGmAmGmGfGmCfAmAmAmCmUmU*mU*mG |
| Duplex NO: 508 | SEQ ID NO: 1402 | mC*mA*mAmCmUmCmAmCfAfGfGmAmUmGmAmAmGmUmA*mA*mA |
| | SEQ ID NO: 1403 | PmU*fU*mUmAfCmUfUmCmAmUmCmCmUfGmUfGmAmGmUmUmG*mG*mA |
| Duplex NO: 509 | SEQ ID NO: 1404 | mA*mA*mCmUmCmAmCmAfGfGfAmUmGmAmAmGmUmAmA*mG*mA |
| | SEQ ID NO: 1405 | PmU*fC*mUmUfAmCfUmUmCmAmUmCmCfUmGfUmGmAmGmUmU*mG*mG |
| Duplex NO: 510 | SEQ ID NO: 1406 | mG*mA*mAmGmUmAmAmGfGfGfAmAmAmAmUmAmUmUmG*mC*mA |
| | SEQ ID NO: 1407 | PmU*fG*mCmAfAmUfAmUmUmUmUmCmCfCmUfUmAmCmUmUmC*mA*mU |
| Duplex NO: 511 | SEQ ID NO: 1408 | mA*mA*mGmUmAmAmGmGfGfAfAmAmAmUmAmUmUmGmC*mU*mA |
| | SEQ ID NO: 1409 | PmU*fA*mGmCfAmAfUmAmUmUmUmUmCfCmCfUmUmAmCmUmU*mC*mA |
| Duplex NO: 512 | SEQ ID NO: 1410 | mG*mU*mAmAmGmGmGmAfAfAfAmUmAmUmUmGmCmUmC*mG*mA |
| | SEQ ID NO: 1411 | PmU*fC*mGmAfGmCfAmAmUmAmUmUmUfUmCfCmCmUmUmAmC*mU*mU |
| Duplex NO: 513 | SEQ ID NO: 1412 | mA*mA*mGmGmGmAmAmAfAfUfAmUmUmGmCmUmCmGmU*mG*mA |
| | SEQ ID NO: 1413 | PmU*fC*mAmCfGmAfGmCmAmAmUmAmUfUmUfUmCmCmCmUmU*mA*mC |
| Duplex NO: 514 | SEQ ID NO: 1414 | mA*mG*mGmGmAmAmAmAfUfAfUmUmGmCmUmCmGmUmG*mG*mA |
| | SEQ ID NO: 1415 | PmU*fC*mCmAfCmGfAmGmCmAmAmUmAfUmUfUmUmCmCmCmU*mU*mA |
| Duplex NO: 515 | SEQ ID NO: 1416 | mG*mG*mGmAmAmAmAmUfAfUfUmGmCmUmCmGmUmGmG*mA*mA |
| | SEQ ID NO: 1417 | PmU*fU*mCmCfAmCfGmAmGmCmAmAmUfAmUfUmUmUmCmCmC*mU*mU |
| Duplex NO: 516 | SEQ ID NO: 1418 | mG*mG*mAmAmAmAmUmAfUfUfGmCmUmCmGmUmGmGmA*mA*mA |
| | SEQ ID NO: 1419 | PmU*fU*mUmCfCmAfCmGmAmGmCmAmAfUmAfUmUmUmUmCmC*mC*mU |
| Duplex NO: 517 | SEQ ID NO: 1420 | mA*mA*mAmUmAmUmUmGfCfUfCmGmUmGmGmAmAmUmG*mG*mA |
| | SEQ ID NO: 1421 | PmU*fC*mCmAfUmUfCmCmAmCmGmAmGfCmAfAmUmAmUmUmU*mU*mC |
| Duplex NO: 518 | SEQ ID NO: 1422 | mA*mA*mUmAmUmUmGmCfUfCfGmUmGmGmAmAmUmGmG*mC*mA |
| | SEQ ID NO: 1423 | PmU*fG*mCmCfAmUfUmCmCmAmCmGmAfGmCfAmAmUmAmUmU*mU*mU |
| Duplex NO: 519 | SEQ ID NO: 1424 | mA*mU*mAmUmUmGmCmUfCfGfUmGmGmAmAmUmGmGmC*mA*mA |
| | SEQ ID NO: 1425 | PmU*fU*mGmCfCmAfUmUmCmCmAmCmGfAmGfCmAmAmUmAmU*mU*mU |
| Duplex NO: 520 | SEQ ID NO: 1426 | mU*mA*mUmUmGmCmUmCfGfUfGmGmAmAmUmGmGmCmA*mA*mA |
| | SEQ ID NO: 1427 | PmU*fU*mUmGfCmCfAmUmUmCmCmAmCfGmAfGmCmAmAmUmA*mU*mU |
| Duplex NO: 521 | SEQ ID NO: 1428 | mA*mU*mUmGmCmUmCmGfUfGfGmAmAmUmGmGmCmAmA*mU*mA |
| | SEQ ID NO: 1429 | PmU*fA*mUmUfGmCfCmAmUmUmCmCmAfCmGfAmGmCmAmAmU*mA*mU |
| Duplex NO: 522 | SEQ ID NO: 1430 | mU*mU*mGmCmUmCmGmUfGfGfAmAmUmGmGmCmAmAmU*mU*mA |
| | SEQ ID NO: 1431 | PmU*fA*mAmUfUmGfCmCmAmUmUmCmCfAmCfGmAmGmCmAmA*mU*mA |
| Duplex NO: 523 | SEQ ID NO: 1432 | mU*mU*mGmAmAmUmGmUfCfUfUmGmUmGmAmUmUmGmG*mA*mA |
| | SEQ ID NO: 1433 | PmU*fU*mCmCfAmAfUmCmAmCmAmAmGfAmCfAmUmUmCmAmA*mC*mA |
| Duplex NO: 524 | SEQ ID NO: 1434 | mU*mG*mAmAmUmGmUmCfUfUfGmUmGmAmUmUmGmGmA*mG*mA |
| | SEQ ID NO: 1435 | PmU*fC*mUmCfCmAfAmUmCmAmCmAmAfGmAfCmAmUmUmCmA*mA*mC |
| Duplex NO: 525 | SEQ ID NO: 1436 | mU*mC*mUmUmGmUmGmAfUfUfGmGmAmGmUmUmGmGmU*mA*mA |
| | SEQ ID NO: 1437 | PmU*fU*mAmCfCmAfAmCmUmCmCmAmAfUmCfAmCmAmAmGmA*mC*mA |
| Duplex NO: 526 | SEQ ID NO: 1438 | mC*mU*mUmGmUmGmAmUfUfGfGmAmGmUmUmGmGmUmA*mC*mA |
| | SEQ ID NO: 1439 | PmU*fG*mUmAfCmCfAmAmCmUmCmCmAfAmUfCmAmCmAmAmG*mA*mC |
| Duplex NO: 527 | SEQ ID NO: 1440 | mU*mU*mGmUmGmAmUmUfGfGfAmGmUmUmGmGmUmAmC*mC*mA |
| | SEQ ID NO: 1441 | PmU*fG*mGmUfAmCfCmAmAmCmUmCmCfAmAfUmCmAmCmAmA*mG*mA |
| Duplex NO: 528 | SEQ ID NO: 1442 | mG*mU*mGmAmUmUmGmGfAfGfUmUmGmGmUmAmCmCmA*mU*mA |
| | SEQ ID NO: 1443 | PmU*fA*mUmGfGmUfAmCmCmAmAmCmUfCmCfAmAmUmCmAmC*mA*mA |
| Duplex NO: 529 | SEQ ID NO: 1444 | mG*mA*mUmUmGmGmAmGfUfUfGmGmUmAmCmCmAmUmG*mU*mA |
| | SEQ ID NO: 1445 | PmU*fA*mCmAfUmGfGmUmAmCmCmAmAfCmUfCmCmAmAmUmC*mA*mC |
| Duplex NO: 530 | SEQ ID NO: 1446 | mA*mU*mUmGmGmAmGmUfUfGfGmUmAmCmCmAmUmGmU*mC*mA |
| | SEQ ID NO: 1447 | PmU*fG*mAmCfAmUfGmGmUmAmCmCmAfAmCfUmCmCmAmAmU*mC*mA |
| Duplex NO: 531 | SEQ ID NO: 1448 | mU*mG*mGmAmGmUmUmGfGfUfAmCmCmAmUmGmUmCmA*mG*mA |
| | SEQ ID NO: 1449 | PmU*fC*mUmGfAmCfAmUmGmGmUmAmCfCmAfAmCmUmCmCmA*mA*mU |
| Duplex NO: 532 | SEQ ID NO: 1450 | mG*mG*mAmGmUmUmGmGfUfAfCmCmAmUmGmUmCmAmG*mG*mA |
| | SEQ ID NO: 1451 | PmU*fC*mCmUfGmAfCmAmUmGmGmUmAfCmCfAmAmCmUmCmC*mA*mA |
| Duplex NO: 533 | SEQ ID NO: 1452 | mU*mA*mUmGmUmGmCmUfGfCfUmUmUmGmGmCmUmGmC*mA*mA |
| | SEQ ID NO: 1453 | PmU*fU*mGmCfAmGfCmCmAmAmAmGmCfAmGfCmAmCmAmUmA*mA*mU |
| Duplex NO: 534 | SEQ ID NO: 1454 | mU*mU*mGmCmCmAmAmCfUfAfCmUmUmCmGmUmGmUmU*mC*mA |
| | SEQ ID NO: 1455 | PmU*fG*mAmAfCmAfCmGmAmAmGmUmAfGmUfUmGmGmCmAmA*mG*mA |
| Duplex NO: 535 | SEQ ID NO: 1456 | mU*mG*mCmCmAmAmCmUfAfCfUmUmCmGmUmGmUmUmC*mA*mA |
| | SEQ ID NO: 1457 | PmU*fU*mGmAfAmCfAmCmGmAmAmGmUfAmGfUmUmGmGmCmA*mA*mG |
| Duplex NO: 536 | SEQ ID NO: 1458 | mG*mC*mCmAmAmCmUmAfCfUfUmCmGmUmGmUmUmCmA*mU*mA |
| | SEQ ID NO: 1459 | PmU*fA*mUmGfAmAfCmAmCmGmAmAmGfUmAfGmUmUmGmGmC*mA*mA |
| Duplex NO: 537 | SEQ ID NO: 1460 | mC*mC*mAmAmCmUmAmCfUfUfCmGmUmGmUmUmCmAmU*mG*mA |
| | SEQ ID NO: 1461 | PmU*fC*mAmUfGmAfAmCmAmCmGmAmAfGmUfAmGmUmUmGmG*mC*mA |
| Duplex NO: 538 | SEQ ID NO: 1462 | mA*mA*mCmUmAmCmUmUfCfGfUmGmUmUmCmAmUmGmA*mC*mA |
| | SEQ ID NO: 1463 | PmU*fG*mUmCfAmUfGmAmAmCmAmCmGfAmAfGmUmAmGmUmU*mG*mG |
| Duplex NO: 539 | SEQ ID NO: 1464 | mA*mU*mUmAmGmAmGmCfUfUfUmCmUmCmGmGmGmAmA*mA*mA |
| | SEQ ID NO: 1465 | PmU*fU*mUmUfCmCfCmGmAmGmAmAmAfGmCfUmCmUmAmAmU*mA*mC |
| Duplex NO: 540 | SEQ ID NO: 1466 | mU*mU*mAmGmAmGmCmUfUfUfCmUmCmGmGmGmAmAmA*mG*mA |
| | SEQ ID NO: 1467 | PmU*fC*mUmUfUmCfCmCmGmAmGmAmAfAmGfCmUmCmUmAmA*mU*mA |
| Duplex NO: 541 | SEQ ID NO: 1468 | mU*mA*mGmAmGmCmUmUfUfCfUmCmGmGmGmAmAmAmG*mC*mA |
| | SEQ ID NO: 1469 | PmU*fG*mCmUfUmUfCmCmCmGmAmGmAfAmAfGmCmUmCmUmA*mA*mU |
| Duplex NO: 542 | SEQ ID NO: 1470 | mG*mA*mGmCmUmUmUmCfUfCfGmGmGmAmAmAmGmCmC*mG*mA |
| | SEQ ID NO: 1471 | PmU*fC*mGmGfCmUfUmUmCmCmCmGmAfGmAfAmAmGmCmUmC*mU*mA |
| Duplex NO: 543 | SEQ ID NO: 1472 | mG*mG*mGmAmAmAmGmCfCfGfCmGmAmGmGmGmUmCmG*mU*mA |
| | SEQ ID NO: 1473 | PmU*fA*mCmGfAmCfCmCmUmCmGmCmGfGmCfUmUmUmCmCmC*mG*mA |
| Duplex NO: 544 | SEQ ID NO: 1474 | mG*mG*mAmAmAmGmCmCfGfCfGmAmGmGmGmUmCmGmU*mC*mA |
| | SEQ ID NO: 1475 | PmU*fG*mAmCfGmAfCmCmCmUmCmGmCfGmGfCmUmUmUmCmC*mC*mG |
| Duplex NO: 545 | SEQ ID NO: 1476 | mG*mA*mAmAmGmCmCmGfCfGfAmGmGmGmUmCmGmUmC*mC*mA |
| | SEQ ID NO: 1477 | PmU*fG*mGmAfCmGfAmCmCmCmUmCmGfCmGfGmCmUmUmUmC*mC*mC |
| Duplex NO: 546 | SEQ ID NO: 1478 | mA*mA*mAmGmCmCmGmCfGfAfGmGmGmUmCmGmUmCmC*mA*mA |
| | SEQ ID NO: 1479 | PmU*fU*mGmGfAmCfGmAmCmCmCmUmCfGmCfGmGmCmUmUmU*mC*mC |
| Duplex NO: 547 | SEQ ID NO: 1480 | mA*mA*mGmCmCmGmCmGfAfGfGmGmUmCmGmUmCmCmA*mA*mA |
| | SEQ ID NO: 1481 | PmU*fU*mUmGfGmAfCmGmAmCmCmCmUfCmGfCmGmGmCmUmU*mU*mC |
| Duplex NO: 548 | SEQ ID NO: 1482 | mA*mG*mCmCmGmCmGmAfGfGfGmUmCmGmUmCmCmAmA*mU*mA |
| | SEQ ID NO: 1483 | PmU*fA*mUmUfGmGfAmCmGmAmCmCmCfUmCfGmCmGmGmCmU*mU*mU |
| Duplex NO: 549 | SEQ ID NO: 1484 | mG*mC*mCmGmCmGmAmGfGfGfUmCmGmUmCmCmAmAmU*mU*mA |
| | SEQ ID NO: 1485 | PmU*fA*mAmUfUmGfGmAmCmGmAmCmCfCmUfCmGmCmGmGmC*mU*mU |
| Duplex NO: 550 | SEQ ID NO: 1486 | mC*mC*mGmCmGmAmGmGfGfUfCmGmUmCmCmAmAmUmU*mU*mA |
| | SEQ ID NO: 1487 | PmU*fA*mAmAfUmUfGmGmAmCmGmAmCfCmCfUmCmGmCmGmG*mC*mU |
| Duplex NO: 551 | SEQ ID NO: 1488 | mC*mG*mCmGmAmGmGmGfUfCfGmUmCmCmAmAmUmUmU*mG*mA |
| | SEQ ID NO: 1489 | PmU*fC*mAmAfAmUfUmGmGmAmCmGmAfCmCfCmUmCmGmCmG*mG*mC |
| Duplex NO: 552 | SEQ ID NO: 1490 | mC*mC*mCmGmAmGmUmUfUfUfAmGmAmAmGmAmAmGmA*mA*mA |
| | SEQ ID NO: 1491 | PmU*fU*mUmCfUmUfCmUmUmCmUmAmAfAmAfCmUmCmGmGmG*mC*mA |
| Duplex NO: 553 | SEQ ID NO: 1492 | mC*mU*mCmAmGmAmGmGfGfUfCmAmAmGmAmUmGmAmU*mU*mA |
| | SEQ ID NO: 1493 | PmU*fA*mAmUfCmAfUmCmUmUmGmAmCfCmCfUmCmUmGmAmG*mU*mU |
| Duplex NO: 554 | SEQ ID NO: 1494 | mG*mG*mUmCmAmAmGmAfUfGfAmUmUmAmUmGmUmCmU*mC*mA |
| | SEQ ID NO: 1495 | PmU*fG*mAmGfAmCfAmUmAmAmUmCmAfUmCfUmUmGmAmCmC*mC*mU |
| Duplex NO: 555 | SEQ ID NO: 1496 | mG*mU*mCmAmAmGmAmUfGfAfUmUmAmUmGmUmCmUmC*mU*mA |
| | SEQ ID NO: 1497 | PmU*fA*mGmAfGmAfCmAmUmAmAmUmCfAmUfCmUmUmGmAmC*mC*mC |
| Duplex NO: 556 | SEQ ID NO: 1498 | mC*mA*mAmGmAmUmGmAfUfUfAmUmGmUmCmUmCmUmA*mG*mA |
| | SEQ ID NO: 1499 | PmU*fC*mUmAfGmAfGmAmCmAmUmAmAfUmCfAmUmCmUmUmG*mA*mC |
| Duplex NO: 557 | SEQ ID NO: 1500 | mA*mA*mGmAmUmGmAmUfUfAfUmGmUmCmUmCmUmAmG*mG*mA |
| | SEQ ID NO: 1501 | PmU*fC*mCmUfAmGfAmGmAmCmAmUmAfAmUfCmAmUmCmUmU*mG*mA |
| Duplex NO: 558 | SEQ ID NO: 1502 | mA*mG*mAmUmGmAmUmUfAfUfGmUmCmUmCmUmAmGmG*mC*mA |
| | SEQ ID NO: 1503 | PmU*fG*mCmCfUmAfGmAmGmAmCmAmUfAmAfUmCmAmUmCmU*mU*mG |
| Duplex NO: 559 | SEQ ID NO: 1504 | mG*mA*mUmGmAmUmUmAfUfGfUmCmUmCmUmAmGmGmC*mU*mA |
| | SEQ ID NO: 1505 | PmU*fA*mGmCfCmUfAmGmAmGmAmCmAfUmAfAmUmCmAmUmC*mU*mU |
| Duplex NO: 560 | SEQ ID NO: 1506 | mA*mU*mGmAmUmUmAmUfGfUfCmUmCmUmAmGmGmCmU*mU*mA |
| | SEQ ID NO: 1507 | PmU*fA*mAmGfCmCfUmAmGmAmGmAmCfAmUfAmAmUmCmAmU*mC*mU |
| Duplex NO: 561 | SEQ ID NO: 1508 | mU*mG*mAmUmUmAmUmGfUfCfUmCmUmAmGmGmCmUmU*mG*mA |
| | SEQ ID NO: 1509 | PmU*fC*mAmAfGmCfCmUmAmGmAmGmAfCmAfUmAmAmUmCmA*mU*mC |
| Duplex NO: 562 | SEQ ID NO: 1510 | mA*mU*mGmCmUmCmAmCfAfGfUmCmGmCmUmGmGmAmU*mA*mA |
| | SEQ ID NO: 1511 | PmU*fU*mAmUfCmCfAmGmCmGmAmCmUfGmUfGmAmGmCmAmU*mG*mA |
| Duplex NO: 563 | SEQ ID NO: 1512 | mU*mG*mCmUmCmAmCmAfGfUfCmGmCmUmGmGmAmUmA*mG*mA |
| | SEQ ID NO: 1513 | PmU*fC*mUmAfUmCfCmAmGmCmGmAmCfUmGfUmGmAmGmCmA*mU*mG |
| Duplex NO: 564 | SEQ ID NO: 1514 | mG*mC*mUmCmAmCmAmGfUfCfGmCmUmGmGmAmUmAmG*mC*mA |
| | SEQ ID NO: 1515 | PmU*fG*mCmUfAmUfCmCmAmGmCmGmAfCmUfGmUmGmAmGmC*mA*mU |
| Duplex NO: 565 | SEQ ID NO: 1516 | mC*mU*mCmAmCmAmGmUfCfGfCmUmGmGmAmUmAmGmC*mU*mA |
| | SEQ ID NO: 1517 | PmU*fA*mGmCfUmAfUmCmCmAmGmCmGfAmCfUmGmUmGmAmG*mC*mA |
| Duplex NO: 566 | SEQ ID NO: 1518 | mU*mC*mAmCmAmGmUmCfGfCfUmGmGmAmUmAmGmCmU*mG*mA |
| | SEQ ID NO: 1519 | PmU*fC*mAmGfCmUfAmUmCmCmAmGmCfGmAfCmUmGmUmGmA*mG*mC |
| Duplex NO: 567 | SEQ ID NO: 1520 | mC*mA*mCmAmGmUmCmGfCfUfGmGmAmUmAmGmCmUmG*mA*mA |
| | SEQ ID NO: 1521 | PmU*fU*mCmAfGmCfUmAmUmCmCmAmGfCmGfAmCmUmGmUmG*mA*mG |
| Duplex NO: 568 | SEQ ID NO: 1522 | mG*mA*mAmUmUmGmAmAfCfCfAmAmGmUmGmUmUmUmC*mC*mA |
| | SEQ ID NO: 1523 | PmU*fG*mGmAfAmAfCmAmCmUmUmGmGfUmUfCmAmAmUmUmC*mU*mC |
| Duplex NO: 569 | SEQ ID NO: 1524 | mG*mA*mAmCmCmAmAmGfUfGfUmUmUmCmCmCmUmCmU*mG*mA |
| | SEQ ID NO: 1525 | PmU*fC*mAmGfAmGfGmGmAmAmAmCmAfCmUfUmGmGmUmUmC*mA*mA |
| Duplex NO: 570 | SEQ ID NO: 1526 | mA*mA*mAmUmGmAmUmCfAfGfCmAmUmGmGmAmUmAmU*mU*mA |
| | SEQ ID NO: 1527 | PmU*fA*mAmUfAmUfCmCmAmUmGmCmUfGmAfUmCmAmUmUmU*mU*mA |
| Duplex NO: 571 | SEQ ID NO: 1528 | mA*mU*mGmAmUmCmAmGfCfAfUmGmGmAmUmAmUmUmG*mA*mA |
| | SEQ ID NO: 1529 | PmU*fU*mCmAfAmUfAmUmCmCmAmUmGfCmUfGmAmUmCmAmU*mU*mU |
| Duplex NO: 572 | SEQ ID NO: 1530 | mU*mC*mUmCmCmUmUmCfUfGfGmCmUmGmUmCmAmAmG*mU*mA |
| | SEQ ID NO: 1531 | PmU*fA*mCmUfUmGfAmCmAmGmCmCmAfGmAfAmGmGmAmGmA*mG*mC |
| Duplex NO: 573 | SEQ ID NO: 1532 | mC*mU*mCmCmUmUmCmUfGfGfCmUmGmUmCmAmAmGmU*mA*mA |
| | SEQ ID NO: 1533 | PmU*fU*mAmCfUmUfGmAmCmAmGmCmCfAmGfAmAmGmGmAmG*mA*mG |
| Duplex NO: 574 | SEQ ID NO: 1534 | mA*mA*mCmAmGmAmGmAfCfAfGmAmAmUmCmUmAmCmA*mC*mA |
| | SEQ ID NO: 1535 | PmU*fG*mUmGfUmAfGmAmUmUmCmUmGfUmCfUmCmUmGmUmU*mU*mG |
| Duplex NO: 575 | SEQ ID NO: 1536 | mA*mC*mAmGmAmGmAmCfAfGfAmAmUmCmUmAmCmAmC*mU*mA |
| | SEQ ID NO: 1537 | PmU*fA*mGmUfGmUfAmGmAmUmUmCmUfGmUfCmUmCmUmGmU*mU*mU |
| Duplex NO: 576 | SEQ ID NO: 1538 | mA*mU*mUmGmUmUmGmUfAfGfAmCmGmUmGmAmAmCmC*mU*mA |
| | SEQ ID NO: 1539 | PmU*fA*mGmGfUmUfCmAmCmGmUmCmUfAmCfAmAmCmAmAmU*mU*mG |
| Duplex NO: 577 | SEQ ID NO: 1540 | mA*mA*mGmUmUmGmAmGfGfUfUmAmUmAmAmAmAmCmC*mC*mA |
| | SEQ ID NO: 1541 | PmU*fG*mGmGfUmUfUmUmAmUmAmAmCfCmUfCmAmAmCmUmU*mU*mU |
| Duplex NO: 578 | SEQ ID NO: 1542 | mG*mU*mUmGmAmGmGmUfUfAfUmAmAmAmAmCmCmCmU*mU*mA |
| | SEQ ID NO: 1543 | PmU*fA*mAmGfGmGfUmUmUmUmAmUmAfAmCfCmUmCmAmAmC*mU*mU |
| Duplex NO: 579 | SEQ ID NO: 1544 | mU*mU*mGmAmGmGmUmUfAfUfAmAmAmAmCmCmCmUmU*mA*mA |
| | SEQ ID NO: 1545 | PmU*fU*mAmAfGmGfGmUmUmUmUmAmUfAmAfCmCmUmCmAmA*mC*mU |
| Duplex NO: 580 | SEQ ID NO: 1546 | mU*mG*mAmGmGmUmUmAfUfAfAmAmAmCmCmCmUmUmA*mG*mA |
| | SEQ ID NO: 1547 | PmU*fC*mUmAfAmGfGmGmUmUmUmUmAfUmAfAmCmCmUmCmA*mA*mC |
| Duplex NO: 581 | SEQ ID NO: 1548 | mG*mC*mAmGmAmGmAmAfAfGfGmUmGmCmAmAmAmAmU*mU*mA |
| | SEQ ID NO: 1549 | PmU*fA*mAmUfUmUfUmGmCmAmCmCmUfUmUfCmUmCmUmGmC*mA*mU |
| Duplex NO: 582 | SEQ ID NO: 1550 | mC*mA*mGmAmGmAmAmAfGfGfUmGmCmAmAmAmAmUmU*mC*mA |
| | SEQ ID NO: 1551 | PmU*fG*mAmAfUmUfUmUmGmCmAmCmCfUmUfUmCmUmCmUmG*mC*mA |
| Duplex NO: 583 | SEQ ID NO: 1552 | mA*mG*mAmGmAmAmAmGfGfUfGmCmAmAmAmAmUmUmC*mC*mA |
| | SEQ ID NO: 1553 | PmU*fG*mGmAfAmUfUmUmUmGmCmAmCfCmUfUmUmCmUmCmU*mG*mC |
| Duplex NO: 584 | SEQ ID NO: 1554 | mG*mA*mGmAmAmAmGmGfUfGfCmAmAmAmAmUmUmCmC*mU*mA |
| | SEQ ID NO: 1555 | PmU*fA*mGmGfAmAfUmUmUmUmGmCmAfCmCfUmUmUmCmUmC*mU*mG |
| Duplex NO: 585 | SEQ ID NO: 1556 | mA*mG*mAmAmAmGmGmUfGfCfAmAmAmAmUmUmCmCmU*mU*mA |
| | SEQ ID NO: 1557 | PmU*fA*mAmGfGmAfAmUmUmUmUmGmCfAmCfCmUmUmUmCmU*mC*mU |
| Duplex NO: 586 | SEQ ID NO: 1558 | mG*mA*mAmAmGmGmUmGfCfAfAmAmAmUmUmCmCmUmU*mA*mA |
| | SEQ ID NO: 1559 | PmU*fU*mAmAfGmGfAmAmUmUmUmUmGfCmAfCmCmUmUmUmC*mU*mC |
| Duplex NO: 587 | SEQ ID NO: 1560 | mA*mA*mAmGmGmUmGmCfAfAfAmAmUmUmCmCmUmUmA*mG*mA |
| | SEQ ID NO: 1561 | PmU*fC*mUmAfAmGfGmAmAmUmUmUmUfGmCfAmCmCmUmUmU*mC*mU |
| Duplex NO: 588 | SEQ ID NO: 1562 | mA*mA*mGmGmUmGmCmAfAfAfAmUmUmCmCmUmUmAmG*mU*mA |
| | SEQ ID NO: 1563 | PmU*fA*mCmUfAmAfGmGmAmAmUmUmUfUmGfCmAmCmCmUmU*mU*mC |
| Duplex NO: 589 | SEQ ID NO: 1564 | mA*mG*mGmUmGmCmAmAfAfAfUmUmCmCmUmUmAmGmU*mG*mA |
| | SEQ ID NO: 1565 | PmU*fC*mAmCfUmAfAmGmGmAmAmUmUfUmUfGmCmAmCmCmU*mU*mU |
| Duplex NO: 590 | SEQ ID NO: 1566 | mU*mG*mCmAmAmAmAmUfUfCfCmUmUmAmGmUmGmAmU*mG*mA |
| | SEQ ID NO: 1567 | PmU*fC*mAmUfCmAfCmUmAmAmGmGmAfAmUfUmUmUmGmCmA*mC*mC |
| Duplex NO: 591 | SEQ ID NO: 1568 | mG*mC*mAmAmAmAmUmUfCfCfUmUmAmGmUmGmAmUmG*mC*mA |
| | SEQ ID NO: 1569 | PmU*fG*mCmAfUmCfAmCmUmAmAmGmGfAmAfUmUmUmUmGmC*mA*mC |
| Duplex NO: 592 | SEQ ID NO: 1570 | mA*mA*mUmUmCmCmUmUfAfGfUmGmAmUmGmCmUmGmA*mG*mA |
| | SEQ ID NO: 1571 | PmU*fC*mUmCfAmGfCmAmUmCmAmCmUfAmAfGmGmAmAmUmU*mU*mU |
| Duplex NO: 593 | SEQ ID NO: 1572 | mU*mC*mCmUmUmAmGmUfGfAfUmGmCmUmGmAmGmAmU*mC*mA |
| | SEQ ID NO: 1573 | PmU*G*mAmUfCmUfCmAmGmCmAmUmCfAmCfUmAmAmGmGmA*mA*mU |
| Duplex NO: 594 | SEQ ID NO: 1574 | mC*mC*mUmUmAmGmUmGfAfUfGmCmUmGmAmGmAmUmC*mA*mA |
| | SEQ ID NO: 1575 | PmU*fU*mGmAfUmCfUmCmAmGmCmAmUfCmAfCmUmAmAmGmG*mA*mA |
| Duplex NO: 595 | SEQ ID NO: 1576 | mC*mU*mGmAmGmAmUmCfAfUfCmCmAmGmUmUmAmGmU*mC*mA |
| | SEQ ID NO: 1577 | PmU*fG*mAmCfUmAfAmCmUmGmGmAmUfGmAfUmCmUmCmAmG*mC*mA |
| Duplex NO: 596 | SEQ ID NO: 1578 | mU*mG*mAmGmAmUmCmAfUfCfCmAmGmUmUmAmGmUmC*mA*mA |
| | SEQ ID NO: 1579 | PmU*fU*mGmAfCmUfAmAmCmUmGmGmAfUmGfAmUmCmUmCmA*mG*mC |
| Duplex NO: 597 | SEQ ID NO: 1580 | mG*mA*mGmAmUmCmAmUfCfCfAmGmUmUmAmGmUmCmA*mA*mA |
| | SEQ ID NO: 1581 | PmU*fU*mUmGfAmCfUmAmAmCmUmGmGfAmUfGmAmUmCmUmC*mA*mG |
| Duplex NO: 598 | SEQ ID NO: 1582 | mA*mU*mCmAmUmCmCmAfGfUfUmAmGmUmCmAmAmUmG*mC*mA |
| | SEQ ID NO: 1583 | PmU*fG*mCmAfUmUfGmAmCmUmAmAmCfUmGfGmAmUmGmAmU*mC*mU |
| Duplex NO: 599 | SEQ ID NO: 1584 | mG*mU*mCmAmAmUmGmCfUfAfAmGmCmAmUmAmUmCmC*mC*mA |
| | SEQ ID NO: 1585 | PmU*fG*mGmGfAmUfAmUmGmCmUmUmAfGmCfAmUmUmGmAmC*mU*mA |
| Duplex NO: 600 | SEQ ID NO: 1586 | mC*mU*mAmAmGmCmAmUfAfUfCmCmCmAmGmCmCmUmA*mC*mA |
| | SEQ ID NO: 1587 | PmU*fG*mUmAfGmGfCmUmGmGmGmAmUfAmUfGmCmUmUmAmG*mC*mA |
| Duplex NO: 601 | SEQ ID NO: 1588 | mA*mA*mGmCmAmUmAmUfCfCfCmAmGmCmCmUmAmCmA*mA*mA |
| | SEQ ID NO: 1589 | PmU*fU*mUmGfUmAfGmGmCmUmGmGmGfAmUfAmUmGmCmUmU*mA*mG |
| Duplex NO: 602 | SEQ ID NO: 1590 | mU*mG*mGmAmAmAmCmUfCfUfGmAmUmGmGmAmAmAmC*mU*mA |
| | SEQ ID NO: 1591 | PmU*fA*mGmUfUmUfCmCmAmUmCmAmGfAmGfUmUmUmCmCmA*mA*mC |
| Duplex NO: 603 | SEQ ID NO: 1592 | mG*mG*mAmAmAmCmUmCfUfGfAmUmGmGmAmAmAmCmU*mC*mA |
| | SEQ ID NO: 1593 | PmU*fG*mAmGfUmUfUmCmCmAmUmCmAfGmAfGmUmUmUmCmC*mA*mA |
| Duplex NO: 604 | SEQ ID NO: 1594 | mG*mA*mAmAmCmUmCmUfGfAfUmGmGmAmAmAmCmUmC*mA*mA |
| | SEQ ID NO: 1595 | PmU*fU*mGmAfGmUfUmUmCmCmAmUmCfAmGfAmGmUmUmUmC*mC*mA |
| Duplex NO: 605 | SEQ ID NO: 1596 | mA*mA*mAmCmUmCmUmGfAfUfGmGmAmAmAmCmUmCmA*mU*mA |
| | SEQ ID NO: 1597 | PmU*fA*mUmGfAmGfUmUmUmCmCmAmUfCmAfGmAmGmUmUmU*mC*mC |
| Duplex NO: 606 | SEQ ID NO: 1598 | mG*mA*mUmGmGmAmAmAfCfUfCmAmUmGmAmGmCmGmU*mG*mA |
| | SEQ ID NO: 1599 | PmU*fC*mAmCfGmCfUmCmAmUmGmAmGfUmUfUmCmCmAmUmC*mA*mG |
| Duplex NO: 607 | SEQ ID NO: 1600 | mA*mU*mGmGmAmAmAmCfUfCfAmUmGmAmGmCmGmUmG*mG*mA |
| | SEQ ID NO: 1601 | PmU*fC*mCmAfCmGfCmUmCmAmUmGmAfGmUfUmUmCmCmAmU*mC*mA |
| Duplex NO: 608 | SEQ ID NO: 1602 | mU*mG*mGmAmAmAmCmUfCfAfUmGmAmGmCmGmUmGmG*mU*mA |
| | SEQ ID NO: 1603 | PmU*fA*mCmCfAmCfGmCmUmCmAmUmGfAmGfUmUmUmCmCmA*mU*mC |
| Duplex NO: 609 | SEQ ID NO: 1604 | mG*mG*mAmAmAmCmUmCfAfUfGmAmGmCmGmUmGmGmU*mG*mA |
| | SEQ ID NO: 1605 | PmU*fC*mAmCfCmAfCmGmCmUmCmAmUfGmAfGmUmUmUmCmC*mA*mU |
| Duplex NO: 610 | SEQ ID NO: 1606 | mG*mA*mAmAmCmUmCmAfUfGfAmGmCmGmUmGmGmUmG*mU*mA |
| | SEQ ID NO: 1607 | PmU*fA*mCmAfCmCfAmCmGmCmUmCmAfUmGfAmGmUmUmUmC*mC*mA |
| Duplex NO: 611 | SEQ ID NO: 1608 | mA*mG*mCmGmUmGmGmUfGfUfAmUmCmUmAmUmUmCmG*mC*mA |
| | SEQ ID NO: 1609 | PmU*fG*mCmGfAmAfUmAmGmAmUmAmCfAmCfCmAmCmGmCmU*mC*mA |
| Duplex NO: 612 | SEQ ID NO: 1610 | mU*mA*mCmCmUmUmAmCfAfGfGmGmAmUmUmAmUmAmA*mU*mA |
| | SEQ ID NO: 1611 | PmU*fA*mUmUfAmUfAmAmUmCmCmCmUfGmUfAmAmGmGmUmA*mG*mG |
| Duplex NO: 613 | SEQ ID NO: 1612 | mA*mC*mCmUmUmAmCmAfGfGfGmAmUmUmAmUmAmAmU*mU*mA |
| | SEQ ID NO: 1613 | PmU*fA*mAmUfUmAfUmAmAmUmCmCmCfUmGfUmAmAmGmGmU*mA*mG |
| Duplex NO: 614 | SEQ ID NO: 1614 | mG*mG*mGmAmUmUmAmUfAfAfUmUmAmCmUmCmCmUmU*mG*mA |
| | SEQ ID NO: 1615 | PmU*fC*mAmAfGmGfAmGmUmAmAmUmUfAmUfAmAmUmCmCmC*mU*mG |
| Duplex NO: 615 | SEQ ID NO: 1616 | mG*mG*mAmUmUmAmUmAfAfUfUmAmCmUmCmCmUmUmG*mG*mA |
| | SEQ ID NO: 1617 | PmU*fC*mCmAfAmGfGmAmGmUmAmAmUfUmAfUmAmAmUmCmC*mC*mU |
| Duplex NO: 616 | SEQ ID NO: 1618 | mG*mA*mUmUmAmUmAmAfUfUfAmCmUmCmCmUmUmGmG*mU*mA |
| | SEQ ID NO: 1619 | PmU*fA*mCmCfAmAfGmGmAmGmUmAmAfUmUfAmUmAmAmUmC*mC*mC |
| Duplex NO: 617 | SEQ ID NO: 1620 | mA*mU*mUmAmUmAmAmUfUfAfCmUmCmCmUmUmGmGmU*mG*mA |
| | SEQ ID NO: 1621 | PmU*fC*mAmCfCmAfAmGmGmAmGmUmAfAmUfUmAmUmAmAmU*mC*mC |
| Duplex NO: 618 | SEQ ID NO: 1622 | mA*mA*mUmUmAmCmUmCfCfUfUmGmGmUmGmAmUmGmG*mG*mA |
| | SEQ ID NO: 1623 | PmU*fC*mCmCfAmUfCmAmCmCmAmAmGfGmAfGmUmAmAmUmU*mA*mU |
| Duplex NO: 619 | SEQ ID NO: 1624 | mA*mU*mUmAmCmUmCmCfUfUfGmGmUmGmAmUmGmGmG*mA*mA |
| | SEQ ID NO: 1625 | PmU*fU*mCmCfCmAfUmCmAmCmCmAmAfGmGfAmGmUmAmAmU*mU*mA |
| Duplex NO: 620 | SEQ ID NO: 1626 | mC*mC*mUmUmGmGmUmGfAfUfGmGmGmAmGmCmUmUmG*mU*mA |
| | SEQ ID NO: 1627 | PmU*fA*mCmAfAmGfCmUmCmCmCmAmUfCmAfCmCmAmAmGmG*mA*mG |
| Duplex NO: 621 | SEQ ID NO: 1628 | mU*mG*mGmUmGmAmUmGfGfGfAmGmCmUmUmGmUmUmG*mU*mA |
| | SEQ ID NO: 1629 | PmU*fA*mCmAfAmCfAmAmGmCmUmCmCfCmAfUmCmAmCmCmA*mA*mG |
| Duplex NO: 622 | SEQ ID NO: 1630 | mU*mA*mUmUmGmGmAmUfAfUfAmUmGmCmCmCmAmUmC*mC*mA |
| | SEQ ID NO: 1631 | PmU*fG*mGmAfUmGfGmGmCmAmUmAmUfAmUfCmCmAmAmUmA*mA*mC |
| Duplex NO: 623 | SEQ ID NO: 1632 | mA*mU*mUmGmGmAmUmAfUfAfUmGmCmCmCmAmUmCmC*mC*mA |
| | SEQ ID NO: 1633 | PmU*fG*mGmGfAmUfGmGmGmCmAmUmAfUmAfUmCmCmAmAmU*mA*mA |
| Duplex NO: 624 | SEQ ID NO: 1634 | mC*mA*mGmAmAmGmGmUfUfGfUmCmUmUmGmUmGmGmC*mC*mA |
| | SEQ ID NO: 1635 | PmU*fG*mGmCfCmAfCmAmAmGmAmCmAfAmCfCmUmUmCmUmG*mU*mU |
| Duplex NO: 625 | SEQ ID NO: 1636 | mA*mG*mGmUmUmGmUmCfUfUfGmUmGmGmCmCmAmGmC*mA*mA |
| | SEQ ID NO: 1637 | PmU*fU*mGmCfUmGfGmCmCmAmCmAmAfGmAfCmAmAmCmCmU*mU*mC |
| Duplex NO: 626 | SEQ ID NO: 1638 | mU*mU*mGmUmCmUmUmGfUfGfGmCmCmAmGmCmAmCmC*mA*mA |
| | SEQ ID NO: 1639 | PmU*fU*mGmGfUmGfCmUmGmGmCmCmAfCmAfAmGmAmCmAmA*mC*mC |
| Duplex NO: 627 | SEQ ID NO: 1640 | mU*mG*mUmCmUmUmGmUfGfGfCmCmAmGmCmAmCmCmA*mA*mA |
| | SEQ ID NO: 1641 | PmU*fU*mUmGfGmUfGmCmUmGmGmCmCfAmCfAmAmGmAmCmA*mA*mC |
| Duplex NO: 628 | SEQ ID NO: 1642 | mA*mG*mGmUmCmUmUmGfGfUfGmGmAmGmGmUmGmCmC*mA*mA |
| | SEQ ID NO: 1643 | PmU*fU*mGmGfCmAfCmCmUmCmCmAmCfCmAfAmGmAmCmCmU*mA*mU |
| Duplex NO: 629 | SEQ ID NO: 1644 | mG*mG*mUmCmUmUmGmGfUfGfGmAmGmGmUmGmCmCmA*mG*mA |
| | SEQ ID NO: 1645 | PmU*fC*mUmGfGmCfAmCmCmUmCmCmAfCmCfAmAmGmAmCmC*mU*mA |
| Duplex NO: 630 | SEQ ID NO: 1646 | mG*mU*mGmGmAmGmGmUfGfCfCmAmGmCmAmGmCmCmG*mA*mA |
| | SEQ ID NO: 1647 | PmU*fU*mCmGfGmCfUmGmCmUmGmGmCfAmCfCmUmCmCmAmC*mC*mA |
| Duplex NO: 631 | SEQ ID NO: 1648 | mG*mU*mGmCmCmAmGmCfAfGfCmCmGmAmGmUmCmCmU*mU*mA |
| | SEQ ID NO: 1649 | PmU*fA*mAmGfGmAfCmUmCmGmGmCmUfGmCfUmGmGmCmAmC*mC*mU |
| Duplex NO: 632 | SEQ ID NO: 1650 | mC*mA*mGmCmAmGmCmCfGfAfGmUmCmCmUmUmGmCmA*mG*mA |
| | SEQ ID NO: 1651 | PmU*fC*mUmGfCmAfAmGmGmAmCmUmCfGmGfCmUmGmCmUmG*mG*mC |
| Duplex NO: 633 | SEQ ID NO: 1652 | mA*mG*mCmAmGmCmCmGfAfGfUmCmCmUmUmGmCmAmG*mA*mA |
| | SEQ ID NO: 1653 | PmU*fU*mCmUfGmCfAmAmGmGmAmCmUfCmGfGmCmUmGmCmU*mG*mG |
| Duplex NO: 634 | SEQ ID NO: 1654 | mG*mC*mAmGmCmCmGmAfGfUfCmCmUmUmGmCmAmGmA*mU*mA |
| | SEQ ID NO: 1655 | PmU*fA*mUmCfUmGfCmAmAmGmGmAmCfUmCfGmGmCmUmGmC*mU*mG |
| Duplex NO: 635 | SEQ ID NO: 1656 | mC*mA*mGmCmCmGmAmGfUfCfCmUmUmGmCmAmGmAmU*mG*mA |
| | SEQ ID NO: 1657 | PmU*fC*mAmUfCmUfGmCmAmAmGmGmAfCmUfCmGmGmCmUmG*mC*mU |
| Duplex NO: 636 | SEQ ID NO: 1658 | mG*mC*mCmGmAmGmUmCfCfUfUmGmCmAmGmAmUmGmG*mG*mA |
| | SEQ ID NO: 1659 | PmU*fC*mCmCfAmUfCmUmGmCmAmAmGfGmAfCmUmCmGmGmC*mU*mG |
| Duplex NO: 637 | SEQ ID NO: 1660 | mC*mG*mAmGmUmCmCmUfUfGfCmAmGmAmUmGmGmGmA*mU*mA |
| | SEQ ID NO: 1661 | PmU*fA*mUmCfCmCfAmUmCmUmGmCmAfAmGfGmAmCmUmCmG*mG*mC |
| Duplex NO: 638 | SEQ ID NO: 1662 | mU*mU*mGmCmAmGmAmUfGfGfGmAmUmGmAmCmUmCmG*mU*mA |
| | SEQ ID NO: 1663 | PmU*fA*mCmGfAmGfUmCmAmUmCmCmCfAmUfCmUmGmCmAmA*mG*mG |
| Duplex NO: 639 | SEQ ID NO: 1664 | mU*mG*mCmAmGmAmUmGfGfGfAmUmGmAmCmUmCmGmU*mG*mA |
| | SEQ ID NO: 1665 | PmU*fC*mAmCfGmAfGmUmCmAmUmCmCfCmAfUmCmUmGmCmA*mA*mG |
| Duplex NO: 640 | SEQ ID NO: 1666 | mG*mC*mAmGmAmUmGmGfGfAfUmGmAmCmUmCmGmUmG*mG*mA |
| | SEQ ID NO: 1667 | PmU*fC*mCmAfCmGfAmGmUmCmAmUmCfCmCfAmUmCmUmGmC*mA*mA |
| Duplex NO: 641 | SEQ ID NO: 1668 | mC*mA*mGmAmUmGmGmGfAfUfGmAmCmUmCmGmUmGmG*mC*mA |
| | SEQ ID NO: 1669 | PmU*fG*mCmCfAmCfGmAmGmUmCmAmUfCmCfCmAmUmCmUmG*mC*mA |
| Duplex NO: 642 | SEQ ID NO: 1670 | mA*mG*mAmUmGmGmGmAfUfGfAmCmUmCmGmUmGmGmC*mC*mA |
| | SEQ ID NO: 1671 | PmU*fG*mGmCfCmAfCmGmAmGmUmCmAfUmCfCmCmAmUmCmU*mG*mC |
| Duplex NO: 643 | SEQ ID NO: 1672 | mG*mA*mUmGmGmGmAmUfGfAfCmUmCmGmUmGmGmCmC*mC*mA |
| | SEQ ID NO: 1673 | PmU*fG*mGmGfCmCfAmCmGmAmGmUmCfAmUfCmCmCmAmUmC*mU*mG |
| Duplex NO: 644 | SEQ ID NO: 1674 | mA*mU*mGmGmGmAmUmGfAfCfUmCmGmUmGmGmCmCmC*mA*mA |
| | SEQ ID NO: 1675 | PmU*fU*mGmGfGmCfCmAmCmGmAmGmUfCmAfUmCmCmCmAmU*mC*mU |
| Duplex NO: 645 | SEQ ID NO: 1676 | mU*mG*mGmGmAmUmGmAfCfUfCmGmUmGmGmCmCmCmA*mG*mA |
| | SEQ ID NO: 1677 | PmU*fC*mUmGfGmGfCmCmAmCmGmAmGfUmCfAmUmCmCmCmA*mU*mC |
| Duplex NO: 646 | SEQ ID NO: 1678 | mG*mG*mGmAmUmGmAmCfUfCfGmUmGmGmCmCmCmAmG*mU*mA |
| | SEQ ID NO: 1679 | PmU*fA*mCmUfGmGfGmCmCmAmCmGmAfGmUfCmAmUmCmCmC*mA*mU |
| Duplex NO: 647 | SEQ ID NO: 1680 | mG*mU*mGmGmCmCmCmAfGfUfUmGmUmGmCmGmUmCmU*mU*mA |
| | SEQ ID NO: 1681 | PmU*fA*mAmGfAmCfGmCmAmCmAmAmCfUmGfGmGmCmCmAmC*mG*mA |
| Duplex NO: 648 | SEQ ID NO: 1682 | mU*mG*mGmCmCmCmAmGfUfUfGmUmGmCmGmUmCmUmU*mC*mA |
| | SEQ ID NO: 1683 | PmU*fG*mAmAfGmAfCmGmCmAmCmAmAfCmUfGmGmGmCmCmA*mC*mG |
| Duplex NO: 649 | SEQ ID NO: 1684 | mG*mC*mCmCmAmGmUmUfGfUfGmCmGmUmCmUmUmCmC*mA*mA |
| | SEQ ID NO: 1685 | PmU*fU*mGmGfAmAfGmAmCmGmCmAmCfAmAfCmUmGmGmGmC*mC*mA |
| Duplex NO: 650 | SEQ ID NO: 1686 | mC*mC*mCmAmGmUmUmGfUfGfCmGmUmCmUmUmCmCmA*mC*mA |
| | SEQ ID NO: 1687 | PmU*fG*mUmGfGmAfAmGmAmCmGmCmAfCmAfAmCmUmGmGmG*mC*mC |
| Duplex NO: 651 | SEQ ID NO: 1688 | mC*mC*mAmGmUmUmGmUfGfCfGmUmCmUmUmCmCmAmC*mG*mA |
| | SEQ ID NO: 1689 | PmU*fC*mGmUfGmGfAmAmGmAmCmGmCfAmCfAmAmCmUmGmG*mG*mC |
| Duplex NO: 652 | SEQ ID NO: 1690 | mC*mA*mGmUmUmGmUmGfCfGfUmCmUmUmCmCmAmCmG*mU*mA |
| | SEQ ID NO: 1691 | PmU*fA*mCmGfUmGfGmAmAmGmAmCmGfCmAfCmAmAmCmUmG*mG*mG |
| Duplex NO: 653 | SEQ ID NO: 1692 | mA*mG*mUmUmGmUmGmCfGfUfCmUmUmCmCmAmCmGmU*mG*mA |
| | SEQ ID NO: 1693 | PmU*fC*mAmCfGmUfGmGmAmAmGmAmCfGmCfAmCmAmAmCmU*mG*mG |
| Duplex NO: 654 | SEQ ID NO: 1694 | mU*mU*mGmUmGmAmCmUfCfUfGmCmAmGmAmAmGmUmG*mA*mA |
| | SEQ ID NO: 1695 | PmU*fU*mCmAfCmUfUmCmUmGmCmAmGfAmGfUmCmAmCmAmA*mG*mC |
| Duplex NO: 655 | SEQ ID NO: 1696 | mC*mU*mGmAmAmGmGmGfUfUfCmGmCmAmGmUmGmAmU*mA*mA |
| | SEQ ID NO: 1697 | PmU*fU*mAmUfCmAfCmUmGmCmGmAmAfCmCfCmUmUmCmAmG*mA*mU |
| Duplex NO: 656 | SEQ ID NO: 1698 | mU*mG*mAmAmGmGmGmUfUfCfGmCmAmGmUmGmAmUmA*mA*mA |
| | SEQ ID NO: 1699 | PmU*fU*mUmAfUmCfAmCmUmGmCmGmAfAmCfCmCmUmUmCmA*mG*mA |
| Duplex NO: 657 | SEQ ID NO: 1700 | mG*mA*mAmGmGmGmUmUfCfGfCmAmGmUmGmAmUmAmA*mA*mA |
| | SEQ ID NO: 1701 | PmU*fU*mUmUfAmUfCmAmCmUmGmCmGfAmAfCmCmCmUmUmC*mA*mG |
| Duplex NO: 658 | SEQ ID NO: 1702 | mA*mG*mGmGmUmUmCmGfCfAfGmUmGmAmUmAmAmAmG*mG*mA |
| | SEQ ID NO: 1703 | PmU*fC*mCmUfUmUfAmUmCmAmCmUmGfCmGfAmAmCmCmCmU*mU*mC |
| Duplex NO: 659 | SEQ ID NO: 1704 | mG*mG*mGmUmUmCmGmCfAfGfUmGmAmUmAmAmAmGmG*mA*mA |
| | SEQ ID NO: 1705 | PmU*fU*mCmCfUmUfUmAmUmCmAmCmUfGmCfGmAmAmCmCmC*mU*mU |
| Duplex NO: 660 | SEQ ID NO: 1706 | mU*mU*mCmGmCmAmGmUfGfAfUmAmAmAmGmGmAmGmG*mC*mA |
| | SEQ ID NO: 1707 | PmU*fG*mCmCfUmCfCmUmUmUmAmUmCfAmCfUmGmCmGmAmA*mC*mC |
| Duplex NO: 661 | SEQ ID NO: 1708 | mC*mA*mGmUmGmAmUmAfAfAfGmGmAmGmGmCmAmUmU*mU*mA |
| | SEQ ID NO: 1709 | PmU*fA*mAmAfUmGfCmCmUmCmCmUmUfUmAfUmCmAmCmUmG*mC*mG |
| Duplex NO: 662 | SEQ ID NO: 1710 | mC*mU*mAmGmCmAmGmAfUfUfUmGmCmAmCmGmUmCmU*mA*mA |
| | SEQ ID NO: 1711 | PmU*fU*mAmGfAmCfGmUmGmCmAmAmAfUmCfUmGmCmUmAmG*mU*mG |
| Duplex NO: 663 | SEQ ID NO: 1712 | mU*mA*mGmCmAmGmAmUfUfUfGmCmAmCmGmUmCmUmA*mC*mA |
| | SEQ ID NO: 1713 | PmU*fG*mUmAfGmAfCmGmUmGmCmAmAfAmUfCmUmGmCmUmA*mG*mU |
| Duplex NO: 664 | SEQ ID NO: 1714 | mA*mG*mCmAmGmAmUmUfUfGfCmAmCmGmUmCmUmAmC*mA*mA |
| | SEQ ID NO: 1715 | PmU*fU*mGmUfAmGfAmCmGmUmGmCmAfAmAfUmCmUmGmCmU*mA*mG |
| Duplex NO: 665 | SEQ ID NO: 1716 | mG*mC*mAmGmAmUmUmUfGfCfAmCmGmUmCmUmAmCmA*mG*mA |
| | SEQ ID NO: 1717 | PmU*fC*mUmGfUmAfGmAmCmGmUmGmCfAmAfAmUmCmUmGmC*mU*mA |
| Duplex NO: 666 | SEQ ID NO: 1718 | mC*mA*mGmAmUmUmUmGfCfAfCmGmUmCmUmAmCmAmG*mA*mA |
| | SEQ ID NO: 1719 | PmU*fU*mCmUfGmUfAmGmAmCmGmUmGfCmAfAmAmUmCmUmG*mC*mU |
| Duplex NO: 667 | SEQ ID NO: 1720 | mG*mA*mUmUmUmGmCmAfCfGfUmCmUmAmCmAmGmAmA*mA*mA |
| | SEQ ID NO: 1721 | PmU*fU*mUmUfCmUfGmUmAmGmAmCmGfUmGfCmAmAmAmUmC*mU*mG |
| Duplex NO: 668 | SEQ ID NO: 1722 | mA*mU*mUmUmGmCmAmCfGfUfCmUmAmCmAmGmAmAmA*mC*mA |
| | SEQ ID NO: 1723 | PmU*fG*mUmUfUmCfUmGmUmAmGmAmCfGmUfGmCmAmAmAmU*mC*mU |
| Duplex NO: 669 | SEQ ID NO: 1724 | mC*mU*mGmGmAmCmGmCfAfAfCmCmUmUmUmAmUmAmU*mC*mA |
| | SEQ ID NO: 1725 | PmU*fG*mAmUfAmUfAmAmAmGmGmUmUfGmCfGmUmCmCmAmG*mC*mU |
| Duplex NO: 670 | SEQ ID NO: 1726 | mU*mG*mGmAmCmGmCmAfAfCfCmUmUmUmAmUmAmUmC*mC*mA |
| | SEQ ID NO: 1727 | PmU*fG*mGmAfUmAfUmAmAmAmGmGmUfUmGfCmGmUmCmCmA*mG*mC |
| Duplex NO: 671 | SEQ ID NO: 1728 | mG*mG*mAmCmGmCmAmAfCfCfUmUmUmAmUmAmUmCmC*mG*mA |
| | SEQ ID NO: 1729 | PmU*fC*mGmGfAmUfAmUmAmAmAmGmGfUmUfGmCmGmUmCmC*mA*mG |
| Duplex NO: 672 | SEQ ID NO: 1730 | mG*mA*mCmGmCmAmAmCfCfUfUmUmAmUmAmUmCmCmG*mU*mA |
| | SEQ ID NO: 1731 | PmU*fA*mCmGfGmAfUmAmUmAmAmAmGfGmUfUmGmCmGmUmC*mC*mA |
| Duplex NO: 673 | SEQ ID NO: 1732 | mC*mG*mCmAmAmCmCmUfUfUfAmUmAmUmCmCmGmUmU*mU*mA |
| | SEQ ID NO: 1733 | PmU*fA*mAmAfCmGfGmAmUmAmUmAmAfAmGfGmUmUmGmCmG*mU*mC |
| Duplex NO: 674 | SEQ ID NO: 1734 | mG*mC*mAmAmCmCmUmUfUfAfUmAmUmCmCmGmUmUmU*mC*mA |
| | SEQ ID NO: 1735 | PmU*fG*mAmAfAmCfGmGmAmUmAmUmAfAmAfGmGmUmUmGmC*mG*mU |
| Duplex NO: 675 | SEQ ID NO: 1736 | mC*mA*mAmCmCmUmUmUfAfUfAmUmCmCmGmUmUmUmC*mC*mA |
| | SEQ ID NO: 1737 | PmU*fG*mGmAfAmAfCmGmGmAmUmAmUfAmAfAmGmGmUmUmG*mC*mG |
| Duplex NO: 676 | SEQ ID NO: 1738 | mC*mC*mUmUmUmAmUmAfUfCfCmGmUmUmUmCmCmAmG*mU*mA |
| | SEQ ID NO: 1739 | PmU*fA*mCmUfGmGfAmAmAmCmGmGmAfUmAfUmAmAmAmGmG*mU*mU |
| Duplex NO: 677 | SEQ ID NO: 1740 | mC*mU*mUmUmAmUmAmUfCfCfGmUmUmUmCmCmAmGmU*mC*mA |
| | SEQ ID NO: 1741 | PmU*fG*mAmCfUmGfGmAmAmAmCmGmGfAmUfAmUmAmAmAmG*mG*mU |
| Duplex NO: 678 | SEQ ID NO: 1742 | mU*mA*mUmCmCmGmUmUfUfCfCmAmGmUmCmCmAmGmG*mU*mA |
| | SEQ ID NO: 1743 | PmU*fA*mCmCfUmGfGmAmCmUmGmGmAfAmAfCmGmGmAmUmA*mU*mA |
| Duplex NO: 679 | SEQ ID NO: 1744 | mU*mU*mGmUmUmUmGmUfGfAfAmGmCmUmGmUmCmAmU*mU*mA |
| | SEQ ID NO: 1745 | PmU*fA*mAmUfGmAfCmAmGmCmUmUmCfAmCfAmAmAmCmAmA*mC*mA |
| Duplex NO: 680 | SEQ ID NO: 1746 | mU*mU*mUmGmUmGmAmAfGfCfUmGmUmCmAmUmUmCmC*mA*mA |
| | SEQ ID NO: 1747 | PmU*fU*mGmGfAmAfUmGmAmCmAmGmCfUmUfCmAmCmAmAmA*mC*mA |
| Duplex NO: 681 | SEQ ID NO: 1748 | mU*mU*mGmUmGmAmAmGfCfUfGmUmCmAmUmUmCmCmA*mG*mA |
| | SEQ ID NO: 1749 | PmU*fC*mUmGfGmAfAmUmGmAmCmAmGfCmUfUmCmAmCmAmA*mA*mC |
| Duplex NO: 682 | SEQ ID NO: 1750 | mG*mA*mAmGmCmUmGmUfCfAfUmUmCmCmAmGmCmCmA*mA*mA |
| | SEQ ID NO: 1751 | PmU*fU*mUmGfGmCfUmGmGmAmAmUmGfAmCfAmGmCmUmUmC*mA*mC |
| Duplex NO: 683 | SEQ ID NO: 1752 | mG*mC*mUmGmUmCmAmUfUfCfCmAmGmCmCmAmAmGmG*mU*mA |
| | SEQ ID NO: 1753 | PmU*fA*mCmCfUmUfGmGmCmUmGmGmAfAmUfGmAmCmAmGmC*mU*mU |
| Duplex NO: 684 | SEQ ID NO: 1754 | mC*mU*mGmUmCmAmUmUfCfCfAmGmCmCmAmAmGmGmU*mU*mA |
| | SEQ ID NO: 1755 | PmU*fA*mAmCfCmUfUmGmGmCmUmGmGfAmAfUmGmAmCmAmG*mC*mU |
| Duplex NO: 685 | SEQ ID NO: 1756 | mU*mG*mUmCmAmUmUmCfCfAfGmCmCmAmAmGmGmUmU*mG*mA |
| | SEQ ID NO: 1757 | PmU*fC*mAmAfCmCfUmUmGmGmCmUmGfGmAfAmUmGmAmCmA*mG*mC |
| Duplex NO: 686 | SEQ ID NO: 1758 | mG*mU*mCmAmUmUmCmCfAfGfCmCmAmAmGmGmUmUmG*mU*mA |
| | SEQ ID NO: 1759 | PmU*fA*mCmAfAmCfCmUmUmGmGmCmUfGmGfAmAmUmGmAmC*mA*mG |
| Duplex NO: 687 | SEQ ID NO: 1760 | mG*mC*mUmGmGmGmAmGfCfAfUmAmGmGmAmGmGmCmU*mA*mA |
| | SEQ ID NO: 1761 | PmU*fU*mAmGfCmCfUmCmCmUmAmUmGfCmUfCmCmCmAmGmC*mC*mA |
| Duplex NO: 688 | SEQ ID NO: 1762 | mC*mU*mGmGmGmAmGmCfAfUfAmGmGmAmGmGmCmUmA*mC*mA |
| | SEQ ID NO: 1763 | PmU*fG*mUmAfGmCfCmUmCmCmUmAmUfGmCfUmCmCmCmAmG*mC*mC |
| Duplex NO: 689 | SEQ ID NO: 1764 | mG*mG*mGmAmGmCmAmUfAfGfGmAmGmGmCmUmAmCmA*mA*mA |
| | SEQ ID NO: 1765 | PmU*fU*mUmGfUmAfGmCmCmUmCmCmUfAmUfGmCmUmCmCmC*mA*mG |
| Duplex NO: 690 | SEQ ID NO: 1766 | mG*mG*mAmGmCmAmUmAfGfGfAmGmGmCmUmAmCmAmA*mC*mA |
| | SEQ ID NO: 1767 | PmU*fG*mUmUfGmUfAmGmCmCmUmCmCfUmAfUmGmCmUmCmC*mC*mA |
| Duplex NO: 691 | SEQ ID NO: 1768 | mA*mG*mCmAmUmAmGmGfAfGfGmCmUmAmCmAmAmCmG*mC*mA |
| | SEQ ID NO: 1769 | PmU*fG*mCmGfUmUfGmUmAmGmCmCmUfCmCfUmAmUmGmCmU*mC*mC |
| Duplex NO: 692 | SEQ ID NO: 1770 | mG*mC*mAmUmAmGmGmAfGfGfCmUmAmCmAmAmCmGmC*mC*mA |
| | SEQ ID NO: 1771 | PmU*fG*mGmCfGmUfUmGmUmAmGmCmCfUmCfCmUmAmUmGmC*mU*mC |
| Duplex NO: 693 | SEQ ID NO: 1772 | mC*mA*mUmAmGmGmAmGfGfCfUmAmCmAmAmCmGmCmC*mC*mA |
| | SEQ ID NO: 1773 | PmU*fG*mGmGfCmGfUmUmGmUmAmGmCfCmUfCmCmUmAmUmG*mC*mU |
| Duplex NO: 694 | SEQ ID NO: 1774 | mA*mU*mAmGmGmAmGmGfCfUfAmCmAmAmCmGmCmCmC*mA*mA |
| | SEQ ID NO: 1775 | PmU*fU*mGmGfGmCfGmUmUmGmUmAmGfCmCfUmCmCmUmAmU*mG*mC |
| Duplex NO: 695 | SEQ ID NO: 1776 | mG*mA*mGmGmCmUmAmCfAfAfCmGmCmCmCmAmUmGmC*mA*mA |
| | SEQ ID NO: 1777 | PmU*fU*mGmCfAmUfGmGmGmCmGmUmUfGmUfAmGmCmCmUmC*mC*mU |
| Duplex NO: 696 | SEQ ID NO: 1778 | mG*mG*mCmUmAmCmAmAfCfGfCmCmCmAmUmGmCmAmG*mC*mA |
| | SEQ ID NO: 1779 | PmU*fG*mCmUfGmCfAmUmGmGmGmCmGfUmUfGmUmAmGmCmC*mU*mC |
| Duplex NO: 697 | SEQ ID NO: 1780 | mG*mC*mUmAmCmAmAmCfGfCfCmCmAmUmGmCmAmGmC*mA*mA |
| | SEQ ID NO: 1781 | PmU*fU*mGmCfUmGfCmAmUmGmGmGmCfGmUfUmGmUmAmGmC*mC*mU |
| Duplex NO: 698 | SEQ ID NO: 1782 | mC*mU*mAmCmAmAmCmGfCfCfCmAmUmGmCmAmGmCmA*mA*mA |
| | SEQ ID NO: 1783 | PmU*fU*mUmGfCmUfGmCmAmUmGmGmGfCmGfUmUmGmUmAmG*mC*mC |
| Duplex NO: 699 | SEQ ID NO: 1784 | mA*mC*mAmAmCmGmCmCfCfAfUmGmCmAmGmCmAmAmA*mC*mA |
| | SEQ ID NO: 1785 | PmU*fG*mUmUfUmGfCmUmGmCmAmUmGfGmGfCmGmUmUmGmU*mA*mG |
| Duplex NO: 700 | SEQ ID NO: 1786 | mC*mA*mAmCmGmCmCmCfAfUfGmCmAmGmCmAmAmAmC*mA*mA |
| | SEQ ID NO: 1787 | PmU*fU*mGmUfUmUfGmCmUmGmCmAmUfGmGfGmCmGmUmUmG*mU*mA |
| Duplex NO: 701 | SEQ ID NO: 1788 | mA*mA*mCmGmCmCmCmAfUfGfCmAmGmCmAmAmAmCmA*mU*mA |
| | SEQ ID NO: 1789 | PmU*fA*mUmGfUmUfUmGmCmUmGmCmAfUmGfGmGmCmGmUmU*mG*mU |
| Duplex NO: 702 | SEQ ID NO: 1790 | mC*mC*mCmAmUmGmCmAfGfCfAmAmAmCmAmUmUmGmU*mC*mA |
| | SEQ ID NO: 1791 | PmU*fG*mAmCfAmAfUmGmUmUmUmGmCfUmGfCmAmUmGmGmG*mC*mG |
| Duplex NO: 703 | SEQ ID NO: 1792 | mC*mU*mGmUmGmGmAmCfAfGfGmAmUmGmCmAmGmCmA*mC*mA |
| | SEQ ID NO: 1793 | PmU*fG*mUmGfCmUfGmCmAmUmCmCmUfGmUfCmCmAmCmAmG*mG*mC |
| Duplex NO: 704 | SEQ ID NO: 1794 | mG*mG*mAmUmGmCmAmGfCfAfCmAmGmAmAmUmGmUmU*mG*mA |
| | SEQ ID NO: 1795 | PmU*fC*mAmAfCmAfUmUmCmUmGmUmGfCmUfGmCmAmUmCmC*mU*mG |
| Duplex NO: 705 | SEQ ID NO: 1796 | mU*mU*mGmGmUmAmGmUfUfCfAmAmAmCmUmGmUmAmU*mU*mA |
| | SEQ ID NO: 1797 | PmU*fA*mAmUfAmCfAmGmUmUmUmGmAfAmCfUmAmCmCmAmA*mC*mA |
| Duplex NO: 706 | SEQ ID NO: 1798 | mU*mG*mGmUmAmGmUmUfCfAfAmAmCmUmGmUmAmUmU*mA*mA |
| | SEQ ID NO: 1799 | PmU*fU*mAmAfUmAfCmAmGmUmUmUmGfAmAfCmUmAmCmCmA*mA*mC |
| Duplex NO: 707 | SEQ ID NO: 1800 | mG*mG*mUmAmGmUmUmCfAfAfAmCmUmGmUmAmUmUmA*mC*mA |
| | SEQ ID NO: 1801 | PmU*fG*mUmAfAmUfAmCmAmGmUmUmUfGmAfAmCmUmAmCmC*mA*mA |
| Duplex NO: 708 | SEQ ID NO: 1802 | mG*mU*mAmUmUmAmCmUfUfUfAmAmUmGmGmAmAmGmC*mA*mA |
| | SEQ ID NO: 1803 | PmU*fU*mGmCfUmUfCmCmAmUmUmAmAfAmGfUmAmAmUmAmC*mA*mG |
| Duplex NO: 709 | SEQ ID NO: 1804 | mU*mA*mUmUmAmCmUmUfUfAfAmUmGmGmAmAmGmCmA*mA*mA |
| | SEQ ID NO: 1805 | PmU*fU*mUmGfCmUfUmCmCmAmUmUmAfAmAfGmUmAmAmUmA*mC*mA |
| Duplex NO: 710 | SEQ ID NO: 1806 | mU*mU*mAmCmUmUmUmAfAfUfGmGmAmAmGmCmAmAmG*mU*mA |
| | SEQ ID NO: 1807 | PmU*fA*mCmUfUmGfCmUmUmCmCmAmUfUmAfAmAmGmUmAmA*mU*mA |
| Duplex NO: 711 | SEQ ID NO: 1808 | mU*mA*mCmUmUmUmAmAfUfGfGmAmAmGmCmAmAmGmU*mG*mA |
| | SEQ ID NO: 1809 | PmU*fC*mAmCfUmUfGmCmUmUmCmCmAfUmUfAmAmAmGmUmA*mA*mU |
| Duplex NO: 712 | SEQ ID NO: 1810 | mG*mU*mCmCmCmAmCmAfAfAfUmGmAmAmGmAmUmUmU*mA*mA |
| | SEQ ID NO: 1811 | PmU*fU*mAmAfAmUfCmUmUmCmAmUmUfUmGfUmGmGmGmAmC*mC*mA |
| Duplex NO: 713 | SEQ ID NO: 1812 | mA*mA*mGmAmUmUmUmAfUfAfUmAmUmCmAmGmCmUmG*mC*mA |
| | SEQ ID NO: 1813 | PmU*fG*mCmAfGmCfUmGmAmUmAmUmAfUmAfAmAmUmCmUmU*mC*mA |
| Duplex NO: 714 | SEQ ID NO: 1814 | mU*mC*mAmGmCmUmGmCfAfCfCmAmUmGmCmCmAmUmC*mU*mA |
| | SEQ ID NO: 1815 | PmU*fA*mGmAfUmGfGmCmAmUmGmGmUfGmCfAmGmCmUmGmA*mU*mA |
| Duplex NO: 715 | SEQ ID NO: 1816 | mC*mA*mGmCmUmGmCmAfCfCfAmUmGmCmCmAmUmCmU*mA*mA |
| | SEQ ID NO: 1817 | PmU*fU*mAmGfAmUfGmGmCmAmUmGmGfUmGfCmAmGmCmUmG*mA*mU |
| Duplex NO: 716 | SEQ ID NO: 1818 | mA*mG*mCmUmGmCmAmCfCfAfUmGmCmCmAmUmCmUmA*mU*mA |
| | SEQ ID NO: 1819 | PmU*fA*mUmAfGmAfUmGmGmCmAmUmGfGmUfGmCmAmGmCmU*mG*mA |
| Duplex NO: 717 | SEQ ID NO: 1820 | mG*mC*mUmGmCmAmCmCfAfUfGmCmCmAmUmCmUmAmU*mA*mA |
| | SEQ ID NO: 1821 | PmU*fU*mAmUfAmGfAmUmGmGmCmAmUfGmGfUmGmCmAmGmC*mU*mG |
| Duplex NO: 718 | SEQ ID NO: 1822 | mC*mU*mGmCmAmCmCmAfUfGfCmCmAmUmCmUmAmUmA*mG*mA |
| | SEQ ID NO: 1823 | PmU*fC*mUmAfUmAfGmAmUmGmGmCmAfUmGfGmUmGmCmAmG*mC*mU |
| Duplex NO: 719 | SEQ ID NO: 1824 | mU*mG*mCmAmCmCmAmUfGfCfCmAmUmCmUmAmUmAmG*mA*mA |
| | SEQ ID NO: 1825 | PmU*fU*mCmUfAmUfAmGmAmUmGmGmCfAmUfGmGmUmGmCmA*mG*mC |
| Duplex NO: 720 | SEQ ID NO: 1826 | mG*mC*mAmCmCmAmUmGfCfCfAmUmCmUmAmUmAmGmA*mG*mA |
| | SEQ ID NO: 1827 | PmU*fC*mUmCfUmAfUmAmGmAmUmGmGfCmAfUmGmGmUmGmC*mA*mG |
| Duplex NO: 721 | SEQ ID NO: 1828 | mC*mA*mCmCmAmUmGmCfCfAfUmCmUmAmUmAmGmAmG*mA*mA |
| | SEQ ID NO: 1829 | PmU*fU*mCmUfCmUfAmUmAmGmAmUmGfGmCfAmUmGmGmUmG*mC*mA |
| Duplex NO: 722 | SEQ ID NO: 1830 | mA*mU*mGmCmCmAmUmCfUfAfUmAmGmAmGmAmUmAmG*mG*mA |
| | SEQ ID NO: 1831 | PmU*fC*mCmUfAmUfCmUmCmUmAmUmAfGmAfUmGmGmCmAmU*mG*mG |
| Duplex NO: 723 | SEQ ID NO: 1832 | mC*mU*mAmUmAmGmAmGfAfUfAmGmGmAmAmCmGmGmU*mG*mA |
| | SEQ ID NO: 1833 | PmU*fC*mAmCfCmGfUmUmCmCmUmAmUfCmUfCmUmAmUmAmG*mA*mU |
| Duplex NO: 724 | SEQ ID NO: 1834 | mU*mA*mUmAmGmAmGmAfUfAfGmGmAmAmCmGmGmUmG*mG*mA |
| | SEQ ID NO: 1835 | PmU*fC*mCmAfCmCfGmUmUmCmCmUmAfUmCfUmCmUmAmUmA*mG*mA |
| Duplex NO: 725 | SEQ ID NO: 1836 | mA*mU*mAmGmAmGmAmUfAfGfGmAmAmCmGmGmUmGmG*mG*mA |
| | SEQ ID NO: 1837 | PmU*fC*mCmCfAmCfCmGmUmUmCmCmUfAmUfCmUmCmUmAmU*mA*mG |
| Duplex NO: 726 | SEQ ID NO: 1838 | mU*mA*mCmUmAmCmCmUfCfAfGmCmAmAmGmCmCmUmG*mU*mA |
| | SEQ ID NO: 1839 | PmU*fA*mCmAfGmGfCmUmUmGmCmUmGfAmGfGmUmAmGmUmA*mG*mG |
| Duplex NO: 727 | SEQ ID NO: 1840 | mG*mU*mUmUmGmCmAmGfAfUfGmCmUmAmGmGmUmGmU*mU*mA |
| | SEQ ID NO: 1841 | PmU*fA*mAmCfAmCfCmUmAmGmCmAmUfCmUfGmCmAmAmAmC*mA*mG |
| Duplex NO: 728 | SEQ ID NO: 1842 | mU*mU*mCmAmAmGmGmAfGfCfAmUmGmCmAmAmAmGmA*mU*mA |
| | SEQ ID NO: 1843 | PmU*fA*mUmCfUmUfUmGmCmAmUmGmCfUmCfCmUmUmGmAmA*mC*mA |
| Duplex NO: 729 | SEQ ID NO: 1844 | mU*mC*mAmAmGmGmAmGfCfAfUmGmCmAmAmAmGmAmU*mA*mA |
| | SEQ ID NO: 1845 | PmU*fU*mAmUfCmUfUmUmGmCmAmUmGfCmUfCmCmUmUmGmA*mA*mC |
| Duplex NO: 730 | SEQ ID NO: 1846 | mC*mA*mAmGmGmAmGmCfAfUfGmCmAmAmAmGmAmUmA*mA*mA |
| | SEQ ID NO: 1847 | PmU*fU*mUmAfUmCfUmUmUmGmCmAmUfGmCfUmCmCmUmUmG*mA*mA |
| Duplex NO: 731 | SEQ ID NO: 1848 | mA*mA*mGmGmAmGmCmAfUfGfCmAmAmAmGmAmUmAmA*mU*mA |
| | SEQ ID NO: 1849 | PmU*fA*mUmUfAmUfCmUmUmUmGmCmAfUmGfCmUmCmCmUmU*mG*mA |
| Duplex NO: 732 | SEQ ID NO: 1850 | mA*mG*mGmAmGmCmAmUfGfCfAmAmAmGmAmUmAmAmU*mC*mA |
| | SEQ ID NO: 1851 | PmU*fG*mAmUfUmAfUmCmUmUmUmGmCfAmUfGmCmUmCmCmU*mU*mG |
| Duplex NO: 733 | SEQ ID NO: 1852 | mG*mG*mAmGmCmAmUmGfCfAfAmAmGmAmUmAmAmUmC*mC*mA |
| | SEQ ID NO: 1853 | PmU*fG*mGmAfUmUfAmUmCmUmUmUmGfCmAfUmGmCmUmCmC*mU*mU |
| Duplex NO: 734 | SEQ ID NO: 1854 | mU*mG*mCmCmCmGmGmCfAfGfCmUmUmGmCmCmCmGmA*mA*mA |
| | SEQ ID NO: 1855 | PmU*fU*mUmCfGmGfGmCmAmAmGmCmUfGmCfCmGmGmGmCmA*mU*mU |
| Duplex NO: 735 | SEQ ID NO: 1856 | mG*mG*mCmAmGmCmUmUfGfCfCmCmGmAmAmUmUmGmU*mG*mA |
| | SEQ ID NO: 1857 | PmU*fC*mAmCfAmAfUmUmCmGmGmGmCfAmAfGmCmUmGmCmC*mG*mG |
| Duplex NO: 736 | SEQ ID NO: 1858 | mG*mC*mAmGmCmUmUmGfCfCfCmGmAmAmUmUmGmUmG*mU*mA |
| | SEQ ID NO: 1859 | PmU*fA*mCmAfCmAfAmUmUmCmGmGmGfCmAfAmGmCmUmGmC*mC*mG |
| Duplex NO: 737 | SEQ ID NO: 1860 | mC*mA*mGmCmUmUmGmCfCfCfGmAmAmUmUmGmUmGmU*mG*mA |
| | SEQ ID NO: 1861 | PmU*fC*mAmCfAmCfAmAmUmUmCmGmGfGmCfAmAmGmCmUmG*mC*mC |
| Duplex NO: 738 | SEQ ID NO: 1862 | mG*mG*mCmAmGmCmAmUfUfGfGmCmAmGmCmAmGmGmA*mC*mA |
| | SEQ ID NO: 1863 | PmU*fG*mUmCfCmUfGmCmUmGmCmCmAfAmUfGmCmUmGmCmC*mA*mU |
| Duplex NO: 739 | SEQ ID NO: 1864 | mG*mC*mAmGmCmAmUmUfGfGfCmAmGmCmAmGmGmAmC*mA*mA |
| | SEQ ID NO: 1865 | PmU*fU*mGmUfCmCfUmGmCmUmGmCmCfAmAfUmGmCmUmGmC*mC*mA |
| Duplex NO: 740 | SEQ ID NO: 1866 | mC*mA*mGmCmAmUmUmGfGfCfAmGmCmAmGmGmAmCmA*mU*mA |
| | SEQ ID NO: 1867 | PmU*fA*mUmGfUmCfCmUmGmCmUmGmCfCmAfAmUmGmCmUmG*mC*mC |
| Duplex NO: 741 | SEQ ID NO: 1868 | mC*mA*mGmCmAmGmGmAfCfAfUmCmUmUmGmUmCmAmA*mA*mA |
| | SEQ ID NO: 1869 | PmU*fU*mUmUfGmAfCmAmAmGmAmUmGfUmCfCmUmGmCmUmG*mC*mC |
| Duplex NO: 742 | SEQ ID NO: 1870 | mA*mG*mCmAmGmGmAmCfAfUfCmUmUmGmUmCmAmAmA*mA*mA |
| | SEQ ID NO: 1871 | PmU*fU*mUmUfUmGfAmCmAmAmGmAmUfGmUfCmCmUmGmCmU*mG*mC |
| Duplex NO: 743 | SEQ ID NO: 1872 | mC*mA*mGmGmAmCmAmUfCfUfUmGmUmCmAmAmAmAmG*mU*mA |
| | SEQ ID NO: 1873 | PmU*fA*mCmUfUmUfUmGmAmCmAmAmGfAmUfGmUmCmCmUmG*mC*mU |
| Duplex NO: 744 | SEQ ID NO: 1874 | mA*mG*mGmAmCmAmUmCfUfUfGmUmCmAmAmAmAmGmU*mC*mA |
| | SEQ ID NO: 1875 | PmU*fG*mAmCfUmUfUmUmGmAmCmAmAfGmAfUmGmUmCmCmU*mG*mC |
| Duplex NO: 745 | SEQ ID NO: 1876 | mG*mG*mAmCmAmUmCmUfUfGfUmCmAmAmAmAmGmUmC*mA*mA |
| | SEQ ID NO: 1877 | PmU*fU*mGmAfCmUfUmUmUmGmAmCmAfAmGfAmUmGmUmCmC*mU*mG |
| Duplex NO: 746 | SEQ ID NO: 1878 | mU*mA*mAmGmUmCmUmGfGfGfAmUmGmUmAmGmAmAmC*mU*mA |
| | SEQ ID NO: 1879 | PmU*fA*mGmUfUmCfUmAmCmAmUmCmCfCmAfGmAmCmUmUmA*mA*mU |
| Duplex NO: 747 | SEQ ID NO: 1880 | mG*mC*mUmAmUmUmUmGfUfUfCmAmGmAmGmAmGmGmC*mC*mA |
| | SEQ ID NO: 1881 | PmU*fG*mGmCfCmUfCmUmCmUmGmAmAfCmAfAmAmUmAmGmC*mC*mU |
| Duplex NO: 748 | SEQ ID NO: 1882 | mC*mU*mAmUmUmUmGmUfUfCfAmGmAmGmAmGmGmCmC*mU*mA |
| | SEQ ID NO: 1883 | PmU*fA*mGmGfCmCfUmCmUmCmUmGmAfAmCfAmAmAmUmAmG*mC*mC |
| Duplex NO: 749 | SEQ ID NO: 1884 | mU*mU*mGmGmAmAmGmUfGfUfUmCmAmAmGmAmAmAmU*mA*mA |
| | SEQ ID NO: 1885 | PmU*fU*mAmUfUmUfCmUmUmGmAmAmCfAmCfUmUmCmCmAmA*mU*mA |
| Duplex NO: 750 | SEQ ID NO: 1886 | mU*mG*mGmAmAmGmUmGfUfUfCmAmAmGmAmAmAmUmA*mA*mA |
| | SEQ ID NO: 1887 | PmU*fU*mUmAfUmUfUmCmUmUmGmAmAfCmAfCmUmUmCmCmA*mA*mU |
| Duplex NO: 751 | SEQ ID NO: 1888 | mU*mG*mUmAmCmUmGmAfUfAfAmAmAmUmAmCmUmCmU*mA*mA |
| | SEQ ID NO: 1889 | PmU*fU*mAmGfAmGfUmAmUmUmUmUmAfUmCfAmGmUmAmCmA*mC*mA |
| Duplex NO: 752 | SEQ ID NO: 1890 | mA*mG*mGmAmAmAmCmCfCfUfGmGmCmUmUmGmCmUmU*mG*mA |
| | SEQ ID NO: 1891 | PmU*fC*mAmAfGmCfAmAmGmCmCmAmGfGmGfUmUmUmCmCmU*mG*mG |
| Duplex NO: 753 | SEQ ID NO: 1892 | mG*mU*mGmCmCmUmAmGfUfUfCmCmUmGmUmGmGmCmC*mA*mA |
| | SEQ ID NO: 1893 | PmU*fU*mGmGfCmCfAmCmAmGmGmAmAfCmUfAmGmGmCmAmC*mA*mG |
| Duplex NO: 754 | SEQ ID NO: 1894 | mG*mU*mUmCmCmUmGmUfGfGfCmCmAmGmGmAmGmGmU*mU*mA |
| | SEQ ID NO: 1895 | PmU*fA*mAmCfCmUfCmCmUmGmGmCmCfAmCfAmGmGmAmAmC*mU*mA |
| Duplex NO: 755 | SEQ ID NO: 1896 | mC*mU*mGmUmGmGmCmCfAfGfGmAmGmGmUmUmGmGmU*mG*mA |
| | SEQ ID NO: 1897 | PmU*fC*mAmCfCmAfAmCmCmUmCmCmUfGmGfCmCmAmCmAmG*mG*mA |
| Duplex NO: 756 | SEQ ID NO: 1898 | mG*mC*mCmAmGmGmAmGfGfUfUmGmGmUmGmAmCmUmG*mA*mA |
| | SEQ ID NO: 1899 | PmU*fU*mCmAfGmUfCmAmCmCmAmAmCfCmUfCmCmUmGmGmC*mC*mA |
| Duplex NO: 757 | SEQ ID NO: 1900 | mC*mA*mGmAmGmUmUmAfUfUfGmAmAmUmCmUmUmAmA*mU*mA |
| | SEQ ID NO: 1901 | PmU*fA*mUmUfAmAfGmAmUmUmCmAmAfUmAfAmCmUmCmUmG*mC*mU |
| Duplex NO: 758 | SEQ ID NO: 1902 | mA*mG*mAmGmUmUmAmUfUfGfAmAmUmCmUmUmAmAmU*mU*mA |
| | SEQ ID NO: 1903 | PmU*fA*mAmUfUmAfAmGmAmUmUmCmAfAmUfAmAmCmUmCmU*mG*mC |
| Duplex NO: 759 | SEQ ID NO: 1904 | mG*mA*mGmUmUmAmUmUfGfAfAmUmCmUmUmAmAmUmU*mU*mA |
| | SEQ ID NO: 1905 | PmU*fA*mAmAfUmUfAmAmGmAmUmUmCfAmAfUmAmAmCmUmC*mU*mG |
| Duplex NO: 760 | SEQ ID NO: 1906 | mU*mU*mUmUmUmAmAmUfGfUfAmCmAmAmGmUmUmUmU*mG*mA |
| | SEQ ID NO: 1907 | PmU*fC*mAmAfAmAfCmUmUmGmUmAmCfAmUfUmAmAmAmAmA*mA*mA |
| Duplex NO: 761 | SEQ ID NO: 1908 | mU*mU*mUmAmAmUmGmUfAfCfAmAmGmUmUmUmUmGmU*mA*mA |
| | SEQ ID NO: 1909 | PmU*fU*mAmCfAmAfAmAmCmUmUmGmUfAmCfAmUmUmAmAmA*mA*mA |
| Duplex NO: 762 | SEQ ID NO: 1910 | mA*mA*mAmUmAmAmUmAfAfAfGmAmAmCmUmCmCmUmU*mA*mA |
| | SEQ ID NO: 1911 | PmU*fU*mAmAfGmGfAmGmUmUmCmUmUfUmAfUmUmAmUmUmU*mA*mU |
| Duplex NO: 763 | SEQ ID NO: 1912 | mA*mA*mUmAmAmUmAmAfAfGfAmAmCmUmCmCmUmUmA*mU*mA |
| | SEQ ID NO: 1913 | PmU*fA*mUmAfAmGfGmAmGmUmUmCmUfUmUfAmUmUmAmUmU*mU*mA |
| Duplex NO: 764 | SEQ ID NO: 1914 | mA*mU*mAmAmUmAmAmAfGfAfAmCmUmCmCmUmUmAmU*mU*mA |
| | SEQ ID NO: 1915 | PmU*fA*mAmUfAmAfGmGmAmGmUmUmCfUmUfUmAmUmUmAmU*mU*mU |
| Duplex NO: 765 | SEQ ID NO: 1916 | mU*mA*mAmUmAmAmAmGfAfAfCmUmCmCmUmUmAmUmU*mU*mA |
| | SEQ ID NO: 1917 | PmU*fA*mAmAfUmAfAmGmGmAmGmUmUfCmUfUmUmAmUmUmA*mU*mU |
| Duplex NO: 766 | SEQ ID NO: 1918 | mA*mA*mUmAmAmAmGmAfAfCfUmCmCmUmUmAmUmUmU*mU*mA |
| | SEQ ID NO: 1919 | PmU*fA*mAmAfAmUfAmAmGmGmAmGmUfUmCfUmUmUmAmUmU*mA*mU |
| Duplex NO: 767 | SEQ ID NO: 1920 | mA*mC*mAmUmCmUmAmAfUfGfCmUmUmCmAmAmGmUmG*mU*mA |
| | SEQ ID NO: 1921 | PmU*fA*mCmAfCmUfUmGmAmAmGmCmAfUmUfAmGmAmUmGmU*mA*mA |
| Duplex NO: 768 | SEQ ID NO: 1922 | mU*mC*mUmGmAmGmCmUfUfUfUmCmAmUmGmGmAmAmG*mU*mA |
| | SEQ ID NO: 1923 | PmU*fA*mCmUfUmCfCmAmUmGmAmAmAfAmGfCmUmCmAmGmA*mA*mA |
| Duplex NO: 769 | SEQ ID NO: 1924 | mU*mG*mAmGmCmUmUmUfUfCfAmUmGmGmAmAmGmUmG*mG*mA |
| | SEQ ID NO: 1925 | PmU*fC*mCmAfCmUfUmCmCmAmUmGmAfAmAfAmGmCmUmCmA*mG*mA |
| Duplex NO: 770 | SEQ ID NO: 1926 | mA*mG*mCmUmUmUmUmCfAfUfGmGmAmAmGmUmGmGmC*mA*mA |
| | SEQ ID NO: 1927 | PmU*fU*mGmCfCmAfCmUmUmCmCmAmUfGmAfAmAmAmGmCmU*mC*mA |
| Duplex NO: 771 | SEQ ID NO: 1928 | mU*mU*mCmAmUmGmGmAfAfGfUmGmGmCmAmGmUmGmA*mA*mA |
| | SEQ ID NO: 1929 | PmU*fU*mUmCfAmCfUmGmCmCmAmCmUfUmCfCmAmUmGmAmA*mA*mA |
| Duplex NO: 772 | SEQ ID NO: 1930 | mU*mU*mAmAmUmGmUmUfUfUfGmUmAmCmAmAmUmUmA*mC*mA |
| | SEQ ID NO: 1931 | PmU*fG*mUmAfAmUfUmGmUmAmCmAmAfAmAfCmAmUmUmAmA*mU*mC |

P indicates a 5′ phosphate
m indicates 2′O-methyl modified ribose on the listed nucleotide
f indicates 2′F modified ribose on the listed nucleotide
*indicates a phosphorothioate bond (in place of a phosphodiester bond)

TABLE 4B
HMGCR RNAi agents, modified sense and antisense strands

| Duplex | | |
|---|---|---|
| NO: | SEQ ID NO: | Modified Sequence |
| Duplex | SEQ ID NO: 1160 | mG*mC*mUmAmCmAmAmUfGfUfUmGmUmCmAmAmGmAmC*mU*mA |
| NO: 773 | SEQ ID NO: 1932 | mU*fA*mGmUfCmUfUmGmAmCmAmAmCfAmUfUmGmUmAmGmC*mU*mA |
| Duplex | SEQ ID NO: 1162 | mC*mU*mAmCmAmAmUmGfUfUfGmUmCmAmAmGmAmCmU*mU*mA |
| NO: 774 | SEQ ID NO: 1933 | mU*fA*mAmGfUmCfUmUmGmAmCmAmAfCmAfUmUmGmUmAmG*mC*mU |
| Duplex | SEQ ID NO: 1164 | mA*mU*mGmAmUmGmUmCfCfAfUmGmAmAmCmAmUmGmU*mU*mA |
| NO: 775 | SEQ ID NO: 1934 | mU*fA*mAmCfAmUfGmUmUmCmAmUmGfGmAfCmAmUmCmAmU*mG*mC |
| D: 776 | SEQ ID NO: 1166 | mA*mU*mAmUmUmUmAmCfUfUfCmCmAmGmUmUmCmCmA*mG*mA |
| | SEQ ID NO: 1935 | mU*fC*mUmGfGmAfAmCmUmGmGmAmAfGmUfAmAmAmUmAmU*mA*mC |
| D: 777 | SEQ ID NO: 1168 | mA*mU*mUmUmAmCmUmUfCfCfAmGmUmUmCmCmAmGmA*mA*mA |
| | SEQ ID NO: 1936 | mU*fU*mUmCfUmGfGmAmAmCmUmGmGfAmAfGmUmAmAmAmU*mA*mU |
| D: 778 | SEQ ID NO: 1170 | mA*mA*mUmGmAmAmGmCfUfUfUmGmCmCmCmUmUmUmU*mU*mA |
| | SEQ ID NO: 1937 | mU*fA*mAmAfAmAfGmGmGmCmAmAmAfGmCfUmUmCmAmUmU*mC*mA |
| D: 779 | SEQ ID NO: 1172 | mC*mA*mCmAmUmUmAmGfCfAfAmAmGmUmUmUmGmCmC*mC*mA |
| | SEQ ID NO: 1938 | mU*fG*mGmGfCmAfAmAmCmUmUmUmGfCmUfAmAmUmGmUmG*mC*mU |
| D: 780 | SEQ ID NO: 1174 | mA*mC*mAmUmUmAmGmCfAfAfAmGmUmUmUmGmCmCmC*mU*mA |
| | SEQ ID NO: 1939 | mU*fA*mGmGfGmCfAmAmAmCmUmUmUfGmCfUmAmAmUmGmU*mG*mC |
| D: 781 | SEQ ID NO: 1176 | mC*mA*mUmUmAmGmCmAfAfAfGmUmUmUmGmCmCmCmU*mC*mA |
| | SEQ ID NO: 1940 | mU*fG*mAmGfGmGfCmAmAmAmCmUmUfUmGfCmUmAmAmUmG*mU*mG |
| D: 782 | SEQ ID NO: 1178 | mU*mA*mGmCmAmAmAmGfUfUfUmGmCmCmCmUmCmAmG*mU*mA |
| | SEQ ID NO: 1941 | mU*fA*mCmUfGmAfGmGmGmCmAmAmAfCmUfUmUmGmCmUmA*mA*mU |
| D: 783 | SEQ ID NO: 1180 | mG*mC*mAmAmAmGmUmUfUfGfCmCmCmUmCmAmGmUmU*mC*mA |
| | SEQ ID NO: 1942 | mU*fG*mAmAfCmUfGmAmGmGmGmCmAfAmAfCmUmUmUmGmC*mU*mA |
| D: 784 | SEQ ID NO: 1182 | mA*mC*mUmCmAmCmAmGfGfAfUmGmAmAmGmUmAmAmG*mG*mA |
| | SEQ ID NO: 1943 | mU*fC*mCmUfUmAfCmUmUmCmAmUmCfCmUfGmUmGmAmGmU*mU*mG |
| D: 785 | SEQ ID NO: 1184 | mC*mA*mCmAmGmGmAmUfGfAfAmGmUmAmAmGmGmGmA*mA*mA |
| | SEQ ID NO: 1944 | mU*fU*mUmCfCmCfUmUmAmCmUmUmCfAmUfCmCmUmGmUmG*mA*mG |
| D: 786 | SEQ ID NO: 1186 | mC*mA*mGmGmAmUmGmAfAfGfUmAmAmGmGmGmAmAmA*mA*mA |
| | SEQ ID NO: 1945 | mU*fU*mUmUfUmCfCmCmUmUmAmCmUfUmCfAmUmCmCmUmG*mU*mG |
| D: 787 | SEQ ID NO: 1188 | mA*mG*mGmAmUmGmAmAfGfUfAmAmGmGmGmAmAmAmA*mU*mA |
| | SEQ ID NO: 1946 | mU*fA*mUmUfUmUfCmCmCmUmUmAmCfUmUfCmAmUmCmCmU*mG*mU |
| D: 788 | SEQ ID NO: 1190 | mG*mC*mGmAmGmGmGmUfCfGfUmCmCmAmAmUmUmUmG*mG*mA |
| | SEQ ID NO: 1947 | mU*fC*mCmAfAmAfUmUmGmGmAmCmGfAmCfCmCmUmCmGmC*mG*mG |
| D: 789 | SEQ ID NO: 1192 | mC*mG*mAmGmGmGmUmCfGfUfCmCmAmAmUmUmUmGmG*mC*mA |
| | SEQ ID NO: 1948 | mU*fG*mCmCfAmAfAmUmUmGmGmAmCfGmAfCmCmCmUmCmG*mC*mG |
| D: 790 | SEQ ID NO: 1194 | mG*mA*mGmGmGmUmCmGfUfCfCmAmAmUmUmUmGmGmC*mA*mA |
| | SEQ ID NO: 1949 | mU*fU*mGmCfCmAfAmAmUmUmGmGmAfCmGfAmCmCmCmUmC*mG*mC |
| D: 791 | SEQ ID NO: 1196 | mG*mU*mCmGmUmCmCmAfAfUfUmUmGmGmCmAmGmCmU*mC*mA |
| | SEQ ID NO: 1950 | mU*fG*mAmGfCmUfGmCmCmAmAmAmUfUmGfGmAmCmGmAmC*mC*mC |
| D: 792 | SEQ ID NO: 1198 | mU*mC*mGmUmCmCmAmAfUfUfUmGmGmCmAmGmCmUmC*mA*mA |
| | SEQ ID NO: 1951 | mU*fU*mGmAfGmCfUmGmCmCmAmAmAfUmUfGmGmAmCmGmA*mC*mC |
| D: 793 | SEQ ID NO: 1200 | mC*mG*mUmCmCmAmAmUfUfUfGmGmCmAmGmCmUmCmA*mG*mA |
| | SEQ ID NO: 1952 | mU*fC*mUmGfAmGfCmUmGmCmCmAmAfAmUfUmGmGmAmCmG*mA*mC |
| D: 794 | SEQ ID NO: 1202 | mU*mC*mCmAmAmUmUmUfGfGfCmAmGmCmUmCmAmGmC*mC*mA |
| | SEQ ID NO: 1953 | mU*fG*mGmCfUmGfAmGmCmUmGmCmCfAmAfAmUmUmGmGmA*mC*mG |
| D: 795 | SEQ ID NO: 1204 | mA*mA*mUmUmUmGmGmCfAfGfCmUmCmAmGmCmCmAmU*mU*mA |
| | SEQ ID NO: 1954 | mU*fA*mAmUfGmGfCmUmGmAmGmCmUfGmCfCmAmAmAmUmU*mG*mG |
| D: 796 | SEQ ID NO: 1206 | mU*mU*mGmGmCmAmGmCfUfCfAmGmCmCmAmUmUmUmU*mG*mA |
| | SEQ ID NO: 1955 | mU*fC*mAmAfAmAfUmGmGmCmUmGmAfGmCfUmGmCmCmAmA*mA*mU |
| D: 797 | SEQ ID NO: 1208 | mU*mG*mGmCmAmGmCmUfCfAfGmCmCmAmUmUmUmUmG*mC*mA |
| | SEQ ID NO: 1956 | mU*fG*mCmAfAmAfAmUmGmGmCmUmGfAmGfCmUmGmCmCmA*mA*mA |
| D: 798 | SEQ ID NO: 1210 | mC*mC*mGmAmGmUmUmUfUfAfGmAmAmGmAmAmGmAmA*mG*mA |
| | SEQ ID NO: 1957 | mU*fC*mUmUfCmUfUmCmUmUmCmUmAfAmAfAmCmUmCmGmG*mG*mC |
| D: 799 | SEQ ID NO: 1212 | mA*mG*mAmGmGmGmUmCfAfAfGmAmUmGmAmUmUmAmU*mG*mA |
| | SEQ ID NO: 1958 | mU*fC*mAmUfAmAfUmCmAmUmCmUmUfGmAfCmCmCmUmCmU*mG*mA |
| D: 800 | SEQ ID NO: 1214 | mG*mG*mGmUmCmAmAmGfAfUfGmAmUmUmAmUmGmUmC*mU*mA |
| | SEQ ID NO: 1959 | mU*fA*mGmAfCmAfUmAmAmUmCmAmUfCmUfUmGmAmCmCmC*mU*mC |
| D: 801 | SEQ ID NO: 1216 | mA*mG*mUmCmGmCmUmGfGfAfUmAmGmCmUmGmAmUmC*mC*mA |
| | SEQ ID NO: 1960 | mU*fG*mGmAfUmCfAmGmCmUmAmUmCfCmAfGmCmGmAmCmU*mG*mU |
| D: 802 | SEQ ID NO: 1218 | mG*mU*mCmGmCmUmGmGfAfUfAmGmCmUmGmAmUmCmC*mU*mA |
| | SEQ ID NO: 1961 | mU*fA*mGmGfAmUfCmAmGmCmUmAmUfCmCfAmGmCmGmAmC*mU*mG |
| D: 803 | SEQ ID NO: 1220 | mU*mC*mGmCmUmGmGmAfUfAfGmCmUmGmAmUmCmCmU*mU*mA |
| | SEQ ID NO: 1962 | mU*fA*mAmGfGmAfUmCmAmGmCmUmAfUmCfCmAmGmCmGmA*mC*mU |
| D: 804 | SEQ ID NO: 1222 | mA*mA*mAmAmGmUmUmGfAfGfGmUmUmAmUmAmAmAmA*mC*mA |
| | SEQ ID NO: 1963 | mU*fG*mUmUfUmUfAmUmAmAmCmCmUfCmAfAmCmUmUmUmU*mC*mU |
| D: 805 | SEQ ID NO: 1224 | mA*mA*mAmGmUmUmGmAfGfGfUmUmAmUmAmAmAmAmC*mC*mA |
| | SEQ ID NO: 1964 | mU*fG*mGmUfUmUfUmAmUmAmAmCmCfUmCfAmAmCmUmUmU*mU*mC |
| D: 806 | SEQ ID NO: 1226 | mU*mG*mGmAmUmAmUmAfUfGfCmCmCmAmUmCmCmCmU*mG*mA |
| | SEQ ID NO: 1965 | mU*fC*mAmGfGmGfAmUmGmGmGmCmAfUmAfUmAmUmCmCmA*mA*mU |
| D: 807 | SEQ ID NO: 1228 | mG*mG*mAmUmAmUmAmUfGfCfCmCmAmUmCmCmCmUmG*mU*mA |
| | SEQ ID NO: 1966 | mU*fA*mCmAfGmGfGmAmUmGmGmGmCfAmUfAmUmAmUmCmC*mA*mA |
| D: 808 | SEQ ID NO: 1230 | mA*mU*mAmUmAmUmGmCfCfCfAmUmCmCmCmUmGmUmU*mG*mA |
| | SEQ ID NO: 1967 | mU*fC*mAmAfCmAfGmGmGmAmUmGmGfGmCfAmUmAmUmAmU*mC*mC |
| D: 809 | SEQ ID NO: 1232 | mU*mA*mUmAmUmGmCmCfCfAfUmCmCmCmUmGmUmUmG*mG*mA |
| | SEQ ID NO: 1968 | mU*fC*mCmAfAmCfAmGmGmGmAmUmGfGmGfCmAmUmAmUmA*mU*mC |
| D: 810 | SEQ ID NO: 1234 | mA*mU*mAmUmGmCmCmCfAfUfCmCmCmUmGmUmUmGmG*mA*mA |
| | SEQ ID NO: 1969 | mU*fU*mCmCfAmAfCmAmGmGmGmAmUfGmGfGmCmAmUmAmU*mA*mU |
| D: 811 | SEQ ID NO: 1236 | mU*mA*mUmGmCmCmCmAfUfCfCmCmUmGmUmUmGmGmA*mG*mA |
| | SEQ ID NO: 1970 | mU*fC*mUmCfCmAfAmCmAmGmGmGmAfUmGfGmGmCmAmUmA*mU*mA |
| D: 812 | SEQ ID NO: 1238 | mG*mG*mUmGmGmAmGmGfUfGfCmCmAmGmCmAmGmCmC*mG*mA |
| | SEQ ID NO: 1971 | mU*fC*mGmGfCmUfGmCmUmGmGmCmAfCmCfUmCmCmAmCmC*mA*mA |
| D: 813 | SEQ ID NO: 1240 | mG*mU*mCmCmUmUmGmCfAfGfAmUmGmGmGmAmUmGmA*mC*mA |
| | SEQ ID NO: 1972 | mU*fG*mUmCfAmUfCmCmCmAmUmCmUfGmCfAmAmGmGmAmC*mU*mC |
| D: 814 | SEQ ID NO: 1242 | mU*mC*mCmUmUmGmCmAfGfAfUmGmGmGmAmUmGmAmC*mU*mA |
| | SEQ ID NO: 1973 | mU*fA*mGmUfCmAfUmCmCmCmAmUmCfUmGfCmAmAmGmGmA*mC*mU |
| D: 815 | SEQ ID NO: 1244 | mC*mU*mUmGmCmAmGmAfUfGfGmGmAmUmGmAmCmUmC*mG*mA |
| | SEQ ID NO: 1974 | mU*fC*mGmAfGmUfCmAmUmCmCmCmAfUmCfUmGmCmAmAmG*mG*mA |
| D: 816 | SEQ ID NO: 1246 | mU*mG*mCmGmUmCmUmUfCfCfAmCmGmUmGmCmUmUmG*mU*mA |
| | SEQ ID NO: 1975 | mU*fA*mCmAfAmGfCmAmCmGmUmGmGfAmAfGmAmCmGmCmA*mC*mA |
| D: 817 | SEQ ID NO: 1248 | mG*mC*mGmUmCmUmUmCfCfAfCmGmUmGmCmUmUmGmU*mG*mA |
| | SEQ ID NO: 1976 | mU*fC*mAmCfAmAfGmCmAmCmGmUmGfGmAfAmGmAmCmGmC*mA*mC |
| D: 818 | SEQ ID NO: 1250 | mG*mU*mCmUmUmCmCmAfCfGfUmGmCmUmUmGmUmGmA*mC*mA |
| | SEQ ID NO: 1977 | mU*fG*mUmCfAmCfAmAmGmCmAmCmGfUmGfGmAmAmGmAmC*mG*mC |
| D: 819 | SEQ ID NO: 1252 | mC*mA*mCmGmUmGmCmUfUfGfUmGmAmCmUmCmUmGmC*mA*mA |
| | SEQ ID NO: 1978 | mU*fU*mGmCfAmGfAmGmUmCmAmCmAfAmGfCmAmCmGmUmG*mG*mA |
| D: 820 | SEQ ID NO: 1254 | mU*mG*mCmUmUmGmUmGfAfCfUmCmUmGmCmAmGmAmA*mG*mA |
| | SEQ ID NO: 1979 | mU*fC*mUmUfCmUfGmCmAmGmAmGmUfCmAfCmAmAmGmCmA*mC*mG |
| D: 821 | SEQ ID NO: 1256 | mU*mU*mUmAmUmAmUmCfCfGfUmUmUmCmCmAmGmUmC*mC*mA |
| | SEQ ID NO: 1980 | mU*fG*mGmAfCmUfGmGmAmAmAmCmGfGmAfUmAmUmAmAmA*mG*mG |
| D: 822 | SEQ ID NO: 1258 | mU*mU*mAmUmAmUmCmCfGfUfUmUmCmCmAmGmUmCmC*mA*mA |
| | SEQ ID NO: 1981 | mU*fU*mGmGfAmCfUmGmGmAmAmAmCfGmGfAmUmAmUmAmA*mA*mG |
| D: 823 | SEQ ID NO: 1260 | mA*mU*mAmUmCmCmGmUfUfUfCmCmAmGmUmCmCmAmG*mG*mA |
| | SEQ ID NO: 1982 | mU*fC*mCmUfGmGfAmCmUmGmGmAmAfAmCfGmGmAmUmAmU*mA*mA |
| D: 824 | SEQ ID NO: 1262 | mC*mU*mGmCmCmAmUmGfGfCfUmGmGmGmAmGmCmAmU*mA*mA |
| | SEQ ID NO: 1983 | mU*fU*mAmUfGmCfUmCmCmCmAmGmCfCmAfUmGmGmCmAmG*mA*mG |
| D: 825 | SEQ ID NO: 1264 | mU*mG*mCmCmAmUmGmGfCfUfGmGmGmAmGmCmAmUmA*mG*mA |
| | SEQ ID NO: 1984 | mU*fC*mUmAfUmGfCmUmCmCmCmAmGfCmCfAmUmGmGmCmA*mG*mA |
| D: 826 | SEQ ID NO: 1266 | mG*mC*mCmAmUmGmGmCfUfGfGmGmAmGmCmAmUmAmG*mG*mA |
| | SEQ ID NO: 1985 | mU*fC*mCmUfAmUfGmCmUmCmCmCmAfGmCfCmAmUmGmGmC*mA*mG |
| D: 827 | SEQ ID NO: 1268 | mA*mC*mAmGmGmAmUmGfCfAfGmCmAmCmAmGmAmAmU*mG*mA |
| | SEQ ID NO: 1986 | mU*fC*mAmUfUmCfUmGmUmGmCmUmGfCmAfUmCmCmUmGmU*mC*mC |
| D: 828 | SEQ ID NO: 1270 | mG*mU*mAmGmUmUmCmAfAfAfCmUmGmUmAmUmUmAmC*mU*mA |
| | SEQ ID NO: 1987 | mU*fA*mGmUfAmAfUmAmCmAmGmUmUfUmGfAmAmCmUmAmC*mC*mA |
| D: 829 | SEQ ID NO: 1272 | mG*mU*mUmCmAmAmAmCfUfGfUmAmUmUmAmCmUmUmU*mA*mA |
| | SEQ ID NO: 1988 | mU*fU*mAmAfAmGfUmAmAmUmAmCmAfGmUfUmUmGmAmAmC*mU*mA |
| D: 830 | SEQ ID NO: 1274 | mU*mU*mCmAmAmAmCmUfGfUfAmUmUmAmCmUmUmUmA*mA*mA |
| | SEQ ID NO: 1989 | mU*fU*mUmAfAmAfGmUmAmAmUmAmCfAmGfUmUmUmGmAmA*mC*mU |
| D: 831 | SEQ ID NO: 1276 | mU*mC*mAmAmAmCmUmGfUfAfUmUmAmCmUmUmUmAmA*mU*mA |
| | SEQ ID NO: 1990 | mU*fA*mUmUfAmAfAmGmUmAmAmUmAfCmAfGmUmUmUmGmA*mA*mC |
| D: 832 | SEQ ID NO: 1278 | mA*mA*mCmUmGmUmAmUfUfAfCmUmUmUmAmAmUmGmG*mA*mA |
| | SEQ ID NO: 1991 | mU*fU*mCmCfAmUfUmAmAmAmGmUmAfAmUfAmCmAmGmUmU*mU*mG |
| D: 833 | SEQ ID NO: 1280 | mA*mC*mUmGmUmAmUmUfAfCfUmUmUmAmAmUmGmGmA*mA*mA |
| | SEQ ID NO: 1992 | mU*Um*mUmCfCmAfUmUmAmAmAmGmUfAmAfUmAmCmAmGmU*mU*mU |
| D: 834 | SEQ ID NO: 1282 | mC*mU*mGmUmAmUmUmAfCfUfUmUmAmAmUmGmGmAmA*mG*mA |
| | SEQ ID NO: 1993 | mU*fC*mUmUfCmCfAmUmUmAmAmAmGfUmAfAmUmAmCmAmG*mU*mU |
| D: 835 | SEQ ID NO: 1284 | mU*mG*mUmAmUmUmAmCfUfUfUmAmAmUmGmGmAmAmG*mC*mA |
| | SEQ ID NO: 1994 | mU*fG*mCmUfUmCfCmAmUmUmAmAmAfGmUfAmAmUmAmCmA*mG*mU |
| D: 836 | SEQ ID NO: 1286 | mU*mU*mAmUmAmUmAmUfCfAfGmCmUmGmCmAmCmCmA*mU*mA |
| | SEQ ID NO: 1995 | mU*fA*mUmGfGmUfGmCmAmGmCmUmGfAmUfAmUmAmUmAmA*mA*mU |
| D: 837 | SEQ ID NO: 1288 | mU*mA*mUmAmUmAmUmCfAfGfCmUmGmCmAmCmCmAmU*mG*mA |
| | SEQ ID NO: 1996 | mU*fC*mAmUfGmGfUmGmCmAmGmCmUfGmAfUmAmUmAmUmA*mA*mA |
| D: 838 | SEQ ID NO: 1290 | mA*mU*mAmUmAmUmCmAfGfCfUmGmCmAmCmCmAmUmG*mC*mA |
| | SEQ ID NO: 1997 | mU*fG*mCmAfUmGfGmUmGmCmAmGmCfUmGfAmUmAmUmAmU*mA*mA |
| D: 839 | SEQ ID NO: 1292 | mU*mA*mUmAmUmCmAmGfCfUfGmCmAmCmCmAmUmGmC*mC*mA |
| | SEQ ID NO: 1998 | mU*fG*mGmCfAmUfGmGmUmGmCmAmGfCmUfGmAmUmAmUmA*mU*mA |
| D: 840 | SEQ ID NO: 1294 | mA*mU*mAmUmCmAmGmCfUfGfCmAmCmCmAmUmGmCmC*mA*mA |
| | SEQ ID NO: 1999 | mU*fU*mGmGfCmAfUmGmGmUmGmCmAfGmCfUmGmAmUmAmU*mA*mU |
| D: 841 | SEQ ID NO: 1296 | mA*mU*mCmAmGmCmUmGfCfAfCmCmAmUmGmCmCmAmU*mC*mA |
| | SEQ ID NO: 2000 | mU*fG*mAmUfGmGfCmAmUmGmGmUmGfCmAfGmCmUmGmAmU*mA*mU |
| D: 842 | SEQ ID NO: 1298 | mU*mU*mUmGmCmAmGmAfUfGfCmUmAmGmGmUmGmUmU*mC*mA |
| | SEQ ID NO: 2001 | mU*fG*mAmAfCmAfCmCmUmAmGmCmAfUmCfUmGmCmAmAmA*mC*mA |
| D: 843 | SEQ ID NO: 1300 | mU*mG*mCmAmGmAmUmGfCfUfAmGmGmUmGmUmUmCmA*mA*mA |
| | SEQ ID NO: 2002 | mU*fU*mUmGfAmAfCmAmCmCmUmAmGfCmAfUmCmUmGmCmA*mA*mA |
| D: 844 | SEQ ID NO: 1302 | mG*mC*mAmGmAmUmGmCfUfAfGmGmUmGmUmUmCmAmA*mG*mA |
| | SEQ ID NO: 2003 | mU*fC*mUmUfGmAfAmCmAmCmCmUmAfGmCfAmUmCmUmGmC*mA*mA |
| D: 845 | SEQ ID NO: 1304 | mC*mA*mGmAmUmGmCmUfAfGfGmUmGmUmUmCmAmAmG*mG*mA |
| | SEQ ID NO: 2004 | mU*fC*mCmUfUmGfAmAmCmAmCmCmUfAmGfCmAmUmCmUmG*mC*mA |
| D: 846 | SEQ ID NO: 1306 | mA*mG*mAmUmGmCmUmAfGfGfUmGmUmUmCmAmAmGmG*mA*mA |
| | SEQ ID NO: 2005 | mU*fU*mCmCfUmUfGmAmAmCmAmCmCfUmAfGmCmAmUmCmU*mG*mC |
| D: 847 | SEQ ID NO: 1308 | mG*mC*mUmAmGmGmUmGfUfUfCmAmAmGmGmAmGmCmA*mU*mA |
| | SEQ ID NO: 2006 | mU*fA*mUmGfCmUfCmCmUmUmGmAmAfCmAfCmCmUmAmGmC*mA*mU |
| D: 848 | SEQ ID NO: 1310 | mU*mA*mGmGmUmGmUmUfCfAfAmGmGmAmGmCmAmUmG*mC*mA |
| | SEQ ID NO: 2007 | mU*fG*mCmAfUmGfCmUmCmCmUmUmGfAmAfCmAmCmCmUmA*mG*mC |
| D: 849 | SEQ ID NO: 1312 | mA*mG*mGmUmGmUmUmCfAfAfGmGmAmGmCmAmUmGmC*mA*mA |
| | SEQ ID NO: 2008 | mU*fU*mGmCfAmUfGmCmUmCmCmUmUfGmAfAmCmAmCmCmU*mA*mG |
| D: 850 | SEQ ID NO: 1314 | mG*mG*mUmGmUmUmCmAfAfGfGmAmGmCmAmUmGmCmA*mA*mA |
| | SEQ ID NO: 2009 | mU*fU*mUmGfCmAfUmGmCmUmCmCmUfUmGfAmAmCmAmCmC*mU*mA |
| D: 851 | SEQ ID NO: 1316 | mU*mG*mUmUmCmAmAmGfGfAfGmCmAmUmGmCmAmAmA*mG*mA |
| | SEQ ID NO: 2010 | mU*fC*mUmUfUmGfCmAmUmGmCmUmCfCmUfUmGmAmAmCmA*mC*mC |
| D: 852 | SEQ ID NO: 1318 | mG*mU*mUmCmAmAmGmGfAfGfCmAmUmGmCmAmAmAmG*mA*mA |
| | SEQ ID NO: 2011 | mU*fU*mCmUfUmUfGmCmAmUmGmCmUfCmCfUmUmGmAmAmC*mA*mC |
| D: 853 | SEQ ID NO: 1320 | mC*mC*mCmGmGmCmAmGfCfUfUmGmCmCmCmGmAmAmU*mU*mA |
| | SEQ ID NO: 2012 | mU*fA*mAmUfUmCfGmGmGmCmAmAmGfCmUfGmCmCmGmGmG*mC*mA |
| D: 854 | SEQ ID NO: 1322 | mC*mG*mGmCmAmGmCmUfUfGfCmCmCmGmAmAmUmUmG*mU*mA |
| | SEQ ID NO: 2013 | mU*fA*mCmAfAmUfUmCmGmGmGmCmAfAmGfCmUmGmCmCmG*mG*mG |
| D: 855 | SEQ ID NO: 1324 | mC*mA*mUmUmGmGmCmAfGfCfAmGmGmAmCmAmUmCmU*mU*mA |
| | SEQ ID NO: 2014 | mU*fA*mAmGfAmUfGmUmCmCmUmGmCfUmGfCmCmAmAmUmG*mC*mU |
| D: 856 | SEQ ID NO: 1326 | mA*mU*mUmGmGmCmAmGfCfAfGmGmAmCmAmUmCmUmU*mG*mA |
| | SEQ ID NO: 2015 | mU*fC*mAmAfGmAfUmGmUmCmCmUmGfCmUfGmCmCmAmAmU*mG*mC |
| D: 857 | SEQ ID NO: 1328 | mU*mG*mGmCmAmGmCmAfGfGfAmCmAmUmCmUmUmGmU*mC*mA |
| | SEQ ID NO: 2016 | mU*fG*mAmCfAmAfGmAmUmGmUmCmCfUmGfCmUmGmCmCmA*mA*mU |
| D: 858 | SEQ ID NO: 1330 | mU*mA*mUmUmUmGmUmUfCfAfGmAmGmAmGmGmCmCmU*mU*mA |
| | SEQ ID NO: 2017 | mU*fA*mAmGfGmCfCmUmCmUmCmUmGfAmAfCmAmAmAmUmA*mG*mC |
| D: 859 | SEQ ID NO: 1332 | mU*mU*mAmCmUmAmAmAfGfAfUmAmUmCmAmGmAmGmC*mU*mA |
| | SEQ ID NO: 2018 | mU*fA*mGmCfUmCfUmGmAmUmAmUmCfUmUfUmAmGmUmAmA*mA*mG |
| D: 860 | SEQ ID NO: 1334 | mC*mA*mAmAmUmCmUmGfUfAfUmAmUmGmUmUmGmUmA*mA*mA |
| | SEQ ID NO: 2019 | mU*fU*mUmAfCmAfAmCmAmUmAmUmAfCmAfGmAmUmUmUmG*mA*mU |
| D: 861 | SEQ ID NO: 1336 | mA*mA*mAmUmCmUmGmUfAfUfAmUmGmUmUmGmUmAmA*mU*mA |
| | SEQ ID NO: 2020 | mU*fA*mUmUfAmCfAmAmCmAmUmAmUfAmCfAmGmAmUmUmU*mG*mA |
| D: 862 | SEQ ID NO: 1338 | mG*mU*mAmCmUmGmAmUfAfAfAmAmUmAmCmUmCmUmA*mG*mA |
| | SEQ ID NO: 2021 | mU*fC*mUmAfGmAfGmUmAmUmUmUmUfAmUfCmAmGmUmAmC*mA*mC |
| D: 863 | SEQ ID NO: 1340 | mU*mU*mGmUmCmCmAmGfGfAfAmAmCmCmCmUmGmGmC*mU*mA |
| | SEQ ID NO: 2022 | mU*fA*mGmCfCmAfGmGmGmUmUmUmCfCmUfGmGmAmCmAmA*mC*mA |
| D: 864 | SEQ ID NO: 1342 | mU*mC*mCmAmGmGmAmAfAfCfCmCmUmGmGmCmUmUmG*mC*mA |
| | SEQ ID NO: 2023 | mU*fG*mCmAfAmGfCmCmAmGmGmGmUfUmUfCmCmUmGmGmA*mC*mA |
| D: 865 | SEQ ID NO: 1344 | mU*mU*mGmUmAmGmAmAfGfAfUmGmGmAmCmUmCmUmG*mA*mA |
| | SEQ ID NO: 2024 | mU*fU*mCmAfGmAfGmUmCmCmAmUmCfUmUfCmUmAmCmAmA*mG*mA |
| D: 866 | SEQ ID NO: 1346 | mU*mG*mUmAmGmAmAmGfAfUfGmGmAmCmUmCmUmGmA*mA*mA |
| | SEQ ID NO: 2025 | mU*fU*mUmCfAmGfAmGmUmCmCmAmUfCmUfUmCmUmAmCmA*mA*mG |
| D: 867 | SEQ ID NO: 1348 | mU*mA*mAmUmGmUmUmUfUfGfUmAmCmAmAmUmUmAmC*mU*mA |
| | SEQ ID NO: 2026 | mU*fA*mGmUfAmAfUmUmGmUmAmCmAfAmAfAmCmAmUmUmA*mA*mU |
| D: 868 | SEQ ID NO: 1350 | mU*mA*mUmAmCmAmUmUfUfUfGmUmUmAmUmAmGmAmA*mU*mA |
| | SEQ ID NO: 2027 | mU*fA*mUmUfCmUfAmUmAmAmCmAmAfAmAfUmGmUmAmUmA*mC*mA |
| D: 869 | SEQ ID NO: 1352 | mU*mA*mCmAmUmUmUmUfGfUfUmAmUmAmGmAmAmUmA*mC*mA |
| | SEQ ID NO: 2028 | mU*fG*mUmAfUmUfCmUmAmUmAmAmCfAmAfAmAmUmGmUmA*mU*mA |
| D: 870 | SEQ ID NO: 1354 | mA*mC*mAmUmUmUmUmGfUfUfAmUmAmGmAmAmUmAmC*mU*mA |
| | SEQ ID NO: 2029 | mU*fA*mGmUfAmUfUmCmUmAmUmAmAfCmAfAmAmAmUmGmU*mA*mU |
| D: 871 | SEQ ID NO: 1356 | mC*mA*mAmUmGmUmUmGfUfCfAmAmGmAmCmUmUmUmU*mU*mA |
| | SEQ ID NO: 2030 | mU*fA*mAmAfAmAfGmUmCmUmUmGmAfCmAfAmCmAmUmUmG*mU*mA |
| D: 872 | SEQ ID NO: 1358 | mA*mA*mUmGmUmUmGmUfCfAfAmGmAmCmUmUmUmUmU*mC*mA |
| | SEQ ID NO: 2031 | mU*fG*mAmAfAmAfAmGmUmCmUmUmGfAmCfAmAmCmAmUmU*mG*mU |
| D: 873 | SEQ ID NO: 1360 | mA*mU*mGmUmUmGmUmCfAfAfGmAmCmUmUmUmUmUmC*mG*mA |
| | SEQ ID NO: 2032 | mU*fC*mGmAfAmAfAmAmGmUmCmUmUfGmAfCmAmAmCmAmU*mU*mG |
| D: 874 | SEQ ID NO: 1362 | mU*mC*mUmGmCmAmUmGfAfUfGmUmCmCmAmUmGmAmA*mC*mA |
| | SEQ ID NO: 2033 | mU*fG*mUmUfCmAfUmGmGmAmCmAmUfCmAfUmGmCmAmGmA*mU*mG |
| D: 875 | SEQ ID NO: 1364 | mC*mU*mGmCmAmUmGmAfUfGfUmCmCmAmUmGmAmAmC*mA*mA |
| | SEQ ID NO: 2034 | mU*fU*mGmUfUmCfAmUmGmGmAmCmAfUmCfAmUmGmCmAmG*mA*mU |
| D: 876 | SEQ ID NO: 1366 | mU*mA*mAmGmAmUmCmUfGfUfGmGmUmUmGmGmAmAmU*mU*mA |
| | SEQ ID NO: 2035 | mU*fA*mAmUfUmCfCmAmAmCmCmAmCfAmGfAmUmCmUmUmA*mU*mU |
| D: 877 | SEQ ID NO: 1368 | mA*mA*mGmAmUmCmUmGfUfGfGmUmUmGmGmAmAmUmU*mA*mA |
| | SEQ ID NO: 2036 | mU*fU*mAmAfUmUfCmCmAmAmCmCmAfCmAfGmAmUmCmUmU*mA*mU |
| D: 878 | SEQ ID NO: 1370 | mA*mG*mAmUmCmUmGmUfGfGfUmUmGmGmAmAmUmUmA*mU*mA |
| | SEQ ID NO: 2037 | mU*fA*mUmAfAmUfUmCmCmAmAmCmCfAmCfAmGmAmUmCmU*mU*mA |
| D: 879 | SEQ ID NO: 1372 | mU*mG*mUmAmUmAmUmUfUfAfCmUmUmCmCmAmGmUmU*mC*mA |
| | SEQ ID NO: 2038 | mU*fG*mAmAfCmUfGmGmAmAmGmUmAfAmAfUmAmUmAmCmA*mG*mG |
| D: 880 | SEQ ID NO: 1374 | mG*mU*mAmUmAmUmUmUfAfCfUmUmCmCmAmGmUmUmC*mC*mA |
| | SEQ ID NO: 2039 | mU*fG*mGmAfAmCfUmGmGmAmAmGmUfAmAfAmUmAmUmAmC*mA*mG |
| D: 881 | SEQ ID NO: 1376 | mU*mA*mUmAmUmUmUmAfCfUfUmCmCmAmGmUmUmCmC*mA*mA |
| | SEQ ID NO: 2040 | mU*fU*mGmGfAmAfCmUmGmGmAmAmGfUmAfAmAmUmAmUmA*mC*mA |
| D: 882 | SEQ ID NO: 1378 | mU*mU*mUmAmCmUmUmCfCfAfGmUmUmCmCmAmGmAmA*mU*mA |
| | SEQ ID NO: 2041 | mU*fA*mUmUfCmUfGmGmAmAmCmUmGfGmAfAmGmUmAmAmA*mU*mA |
| D: 883 | SEQ ID NO: 1380 | mU*mA*mUmUmUmUmGmGfGfUfAmUmUmGmCmUmGmGmC*mC*mA |
| | SEQ ID NO: 2042 | mU*fG*mGmCfCmAfGmCmAmAmUmAmCfCmCfAmAmAmAmUmA*mU*mA |
| D: 884 | SEQ ID NO: 1382 | mU*mA*mUmUmCmAmGmUfAfCfAmGmUmUmGmUmCmAmU*mU*mA |
| | SEQ ID NO: 2043 | mU*fA*mAmUfGmAfCmAmAmCmUmGmUfAmCfUmGmAmAmUmA*mC*mA |
| D: 885 | SEQ ID NO: 1384 | mA*mU*mUmCmAmGmUmAfCfAfGmUmUmGmUmCmAmUmU*mC*mA |
| | SEQ ID NO: 2044 | mU*fG*mAmAfUmGfAmCmAmAmCmUmGfUmAfCmUmGmAmAmU*mA*mC |
| D: 886 | SEQ ID NO: 1386 | mA*mG*mAmCmAmAmAmGfAfAfUmUmGmAmCmAmGmGmC*mU*mA |
| | SEQ ID NO: 2045 | mU*fA*mGmCfCmUfGmUmCmAmAmUmUfCmUfUmUmGmUmCmU*mA*mA |
| D: 887 | SEQ ID NO: 1388 | mG*mA*mCmAmAmAmGmAfAfUfUmGmAmCmAmGmGmCmU*mU*mA |
| | SEQ ID NO: 2046 | mU*fA*mAmGfCmCfUmGmUmCmAmAmUfUmCfUmUmUmGmUmC*mU*mA |
| D: 888 | SEQ ID NO: 1390 | mG*mC*mUmUmGmAmAmUfGfAfAmGmCmUmUmUmGmCmC*mC*mA |
| | SEQ ID NO: 2047 | mU*fG*mGmGfCmAfAmAmGmCmUmUmCfAmUfUmCmAmAmGmC*mC*mU |
| D: 889 | SEQ ID NO: 1392 | mC*mU*mUmGmAmAmUmGfAfAfGmCmUmUmUmGmCmCmC*mU*mA |
| | SEQ ID NO: 2048 | mU*fA*mGmGfGmCfAmAmAmGmCmUmUfCmAfUmUmCmAmAmG*mC*mC |
| D: 890 | SEQ ID NO: 1394 | mU*mU*mGmAmAmUmGmAfAfGfCmUmUmUmGmCmCmCmU*mU*mA |
| | SEQ ID NO: 2049 | mU*fA*mAmGfGmGfCmAmAmAmGmCmUfUmCfAmUmUmCmAmA*mG*mC |
| D: 891 | SEQ ID NO: 1396 | mA*mU*mGmAmAmGmCmUfUfUfGmCmCmCmUmUmUmUmU*mC*mA |
| | SEQ ID NO: 2050 | mU*fG*mAmAfAmAfAmGmGmGmCmAmAfAmGfCmUmUmCmAmU*mU*mC |
| D: 892 | SEQ ID NO: 1398 | mC*mA*mAmAmGmUmUmUfGfCfCmCmUmCmAmGmUmUmC*mC*mA |
| | SEQ ID NO: 2051 | mU*fG*mGmAfAmCfUmGmAmGmGmGmCfAmAfAmCmUmUmUmG*mC*mU |
| D: 893 | SEQ ID NO: 1400 | mA*mA*mGmUmUmUmGmCfCfCfUmCmAmGmUmUmCmCmA*mA*mA |
| | SEQ ID NO: 2052 | mU*fU*mUmGfGmAfAmCmUmGmAmGmGfGmCfAmAmAmCmUmU*mU*mG |
| D: 894 | SEQ ID NO: 1402 | mC*mA*mAmCmUmCmAmCfAfGfGmAmUmGmAmAmGmUmA*mA*mA |
| | SEQ ID NO: 2053 | mU*fU*mUmAfCmUfUmCmAmUmCmCmUfGmUfGmAmGmUmUmG*mG*mA |
| D: 895 | SEQ ID NO: 1404 | mA*mA*mCmUmCmAmCmAfGfGfAmUmGmAmAmGmUmAmA*mG*mA |
| | SEQ ID NO: 2054 | mU*fC*mUmUfAmCfUmUmCmAmUmCmCfUmGfUmGmAmGmUmU*mG*mG |
| D: 896 | SEQ ID NO: 1406 | mG*mA*mAmGmUmAmAmGfGfGfAmAmAmAmUmAmUmUmG*mC*mA |
| | SEQ ID NO: 2055 | mU*fG*mCmAfAmUfAmUmUmUmUmCmCfCmUfUmAmCmUmUmC*mA*mU |
| D: 897 | SEQ ID NO: 1408 | mA*mA*mGmUmAmAmGmGfGfAfAmAmAmUmAmUmUmGmC*mU*mA |
| | SEQ ID NO: 2056 | mU*fA*mGmCfAmAfUmAmUmUmUmUmCfCmCfUmUmAmCmUmU*mC*mA |
| D: 898 | SEQ ID NO: 1410 | mG*mU*mAmAmGmGmGmAfAfAfAmUmAmUmUmGmCmUmC*mG*mA |
| | SEQ ID NO: 2057 | mU*fC*mGmAfGmCfAmAmUmAmUmUmUfUmCfCmCmUmUmAmC*mU*mU |
| D: 899 | SEQ ID NO: 1412 | mA*mA*mGmGmGmAmAmAfAfUfAmUmUmGmCmUmCmGmU*mG*mA |
| | SEQ ID NO: 2058 | mU*fC*mAmCfGmAfGmCmAmAmUmAmUfUmUfUmCmCmCmUmU*mA*mC |
| D: 900 | SEQ ID NO: 1414 | mA*mG*mGmGmAmAmAmAfUfAfUmUmGmCmUmCmGmUmG*mG*mA |
| | SEQ ID NO: 2059 | mU*fC*mCmAfCmGfAmGmCmAmAmUmAfUmUfUmUmCmCmCmU*mU*mA |
| D: 901 | SEQ ID NO: 1416 | mG*mG*mGmAmAmAmAmUfAfUfUmGmCmUmCmGmUmGmG*mA*mA |
| | SEQ ID NO: 2060 | mU*fU*mCmCfAmCfGmAmGmCmAmAmUfAmUfUmUmUmCmCmC*mU*mU |
| D: 902 | SEQ ID NO: 1418 | mG*mG*mAmAmAmAmUmAfUfUfGmCmUmCmGmUmGmGmA*mA*mA |
| | SEQ ID NO: 2061 | mU*fU*mUmCfCmAfCmGmAmGmCmAmAfUmAfUmUmUmUmCmC*mC*mU |
| D: 903 | SEQ ID NO: 1420 | mA*mA*mAmUmAmUmUmGfCfUfCmGmUmGmGmAmAmUmG*mG*mA |
| | SEQ ID NO: 2062 | mU*fC*mCmAfUmUfCmCmAmCmGmAmGfCmAfAmUmAmUmUmU*mU*mC |
| D: 904 | SEQ ID NO: 1422 | mA*mA*mUmAmUmUmGmCfUfCfGmUmGmGmAmAmUmGmG*mC*mA |
| | SEQ ID NO: 2063 | mU*fG*mCmCfAmUfUmCmCmAmCmGmAfGmCfAmAmUmAmUmU*mU*mU |
| D: 905 | SEQ ID NO: 1424 | mA*mU*mAmUmUmGmCmUfCfGfUmGmGmAmAmUmGmGmC*mA*mA |
| | SEQ ID NO: 2064 | mU*fU*mGmCfCmAfUmUmCmCmAmCmGfAmGfCmAmAmUmAmU*mU*mU |
| D: 906 | SEQ ID NO: 1426 | mU*mA*mUmUmGmCmUmCfGfUfGmGmAmAmUmGmGmCmA*mA*mA |
| | SEQ ID NO: 2065 | mU*fU*mUmGfCmCfAmUmUmCmCmAmCfGmAfGmCmAmAmUmA*mU*mU |
| D: 907 | SEQ ID NO: 1428 | mA*mU*mUmGmCmUmCmGfUfGfGmAmAmUmGmGmCmAmA*mU*mA |
| | SEQ ID NO: 2066 | mU*fA*mUmUfGmCfCmAmUmUmCmCmAfCmGfAmGmCmAmAmU*mA*mU |
| D: 908 | SEQ ID NO: 1430 | mU*mU*mGmCmUmCmGmUfGfGfAmAmUmGmGmCmAmAmU*mU*mA |
| | SEQ ID NO: 2067 | mU*fA*mAmUfUmGfCmCmAmUmUmCmCfAmCfGmAmGmCmAmA*mU*mA |
| D: 909 | SEQ ID NO: 1432 | mU*mU*mGmAmAmUmGmUfCfUfUmGmUmGmAmUmUmGmG*mA*mA |
| | SEQ ID NO: 2068 | mU*fU*mCmCfAmAfUmCmAmCmAmAmGfAmCfAmUmUmCmAmA*mC*mA |
| D: 910 | SEQ ID NO: 1434 | mU*mG*mAmAmUmGmUmCfUfUfGmUmGmAmUmUmGmGmA*mG*mA |
| | SEQ ID NO: 2069 | mU*fC*mUmCfCmAfAmUmCmAmCmAmAfGmAfCmAmUmUmCmA*mA*mC |
| D: 911 | SEQ ID NO: 1436 | mU*mC*mUmUmGmUmGmAfUfUfGmGmAmGmUmUmGmGmU*mA*mA |
| | SEQ ID NO: 2070 | mU*fU*mAmCfCmAfAmCmUmCmCmAmAfUmCfAmCmAmAmGmA*mC*mA |
| D: 912 | SEQ ID NO: 1438 | mC*mU*mUmGmUmGmAmUfUfGfGmAmGmUmUmGmGmUmA*mC*mA |
| | SEQ ID NO: 2071 | mU*fG*mUmAfCmCfAmAmCmUmCmCmAfAmUfCmAmCmAmAmG*mA*mC |
| D: 913 | SEQ ID NO: 1440 | mU*mU*mGmUmGmAmUmUfGfGfAmGmUmUmGmGmUmAmC*mC*mA |
| | SEQ ID NO: 2072 | mU*fG*mGmUfAmCfCmAmAmCmUmCmCfAmAfUmCmAmCmAmA*mG*mA |
| D: 914 | SEQ ID NO: 1442 | mG*mU*mGmAmUmUmGmGfAfGfUmUmGmGmUmAmCmCmA*mU*mA |
| | SEQ ID NO: 2073 | mU*fA*mUmGfGmUfAmCmCmAmAmCmUfCmCfAmAmUmCmAmC*mA*mA |
| D: 915 | SEQ ID NO: 1444 | mG*mA*mUmUmGmGmAmGfUfUfGmGmUmAmCmCmAmUmG*mU*mA |
| | SEQ ID NO: 2074 | mU*fA*mCmAfUmGfGmUmAmCmCmAmAfCmUfCmCmAmAmUmC*mA*mC |
| D: 916 | SEQ ID NO: 1446 | mA*mU*mUmGmGmAmGmUfUfGfGmUmAmCmCmAmUmGmU*mC*mA |
| | SEQ ID NO: 2075 | mU*fG*mAmCfAmUfGmGmUmAmCmCmAfAmCfUmCmCmAmAmU*mC*mA |
| D: 917 | SEQ ID NO: 1448 | mU*mG*mGmAmGmUmUmGfGfUfAmCmCmAmUmGmUmCmA*mG*mA |
| | SEQ ID NO: 2076 | mU*fC*mUmGfAmCfAmUmGmGmUmAmCfCmAfAmCmUmCmCmA*mA*mU |
| D: 918 | SEQ ID NO: 1450 | mG*mG*mAmGmUmUmGmGfUfAfCmCmAmUmGmUmCmAmG*mG*mA |
| | SEQ ID NO: 2077 | mU*fC*mCmUfGmAfCmAmUmGmGmUmAfCmCfAmAmCmUmCmC*mA*mA |
| D: 919 | SEQ ID NO: 1452 | mU*mA*mUmGmUmGmCmUfGfCfUmUmUmGmGmCmUmGmC*mA*mA |
| | SEQ ID NO: 2078 | mU*fU*mGmCfAmGfCmCmAmAmAmGmCfAmGfCmAmCmAmUmA*mA*mU |
| D: 920 | SEQ ID NO: 1454 | mU*mU*mGmCmCmAmAmCfUfAfCmUmUmCmGmUmGmUmU*mC*mA |
| | SEQ ID NO: 2079 | mU*fG*mAmAfCmAfCmGmAmAmGmUmAfGmUfUmGmGmCmAmA*mG*mA |
| D: 921 | SEQ ID NO: 1456 | mU*mG*mCmCmAmAmCmUfAfCfUmUmCmGmUmGmUmUmC*mA*mA |
| | SEQ ID NO: 2080 | mU*fU*mGmAfAmCfAmCmGmAmAmGmUfAmGfUmUmGmGmCmA*mA*mG |
| D: 922 | SEQ ID NO: 1458 | mG*mC*mCmAmAmCmUmAfCfUfUmCmGmUmGmUmUmCmA*mU*mA |
| | SEQ ID NO: 2081 | mU*fA*mUmGfAmAfCmAmCmGmAmAmGfUmAfGmUmUmGmGmC*mA*mA |
| D: 923 | SEQ ID NO: 1460 | mC*mC*mAmAmCmUmAmCfUfUfCmGmUmGmUmUmCmAmU*mG*mA |
| | SEQ ID NO: 2082 | mU*fC*mAmUfGmAfAmCmAmCmGmAmAfGmUfAmGmUmUmGmG*mC*mA |
| D: 924 | SEQ ID NO: 1462 | mA*mA*mCmUmAmCmUmUfCfGfUmGmUmUmCmAmUmGmA*mC*mA |
| | SEQ ID NO: 2083 | mU*fG*mUmCfAmUfGmAmAmCmAmCmGfAmAfGmUmAmGmUmU*mG*mG |
| D: 925 | SEQ ID NO: 1464 | mA*mU*mUmAmGmAmGmCfUfUfUmCmUmCmGmGmGmAmA*mA*mA |
| | SEQ ID NO: 2084 | mU*fU*mUmUfCmCfCmGmAmGmAmAmAfGmCfUmCmUmAmAmU*mA*mC |
| D: 926 | SEQ ID NO: 1466 | mU*mU*mAmGmAmGmCmUfUfUfCmUmCmGmGmGmAmAmA*mG*mA |
| | SEQ ID NO: 2085 | mU*fC*mUmUfUmCfCmCmGmAmGmAmAfAmGfCmUmCmUmAmA*mU*mA |
| D: 927 | SEQ ID NO: 1468 | mU*mA*mGmAmGmCmUmUfUfCfUmCmGmGmGmAmAmAmG*mC*mA |
| | SEQ ID NO: 2086 | mU*fG*mCmUfUmUfCmCmCmGmAmGmAfAmAfGmCmUmCmUmA*mA*mU |
| D: 928 | SEQ ID NO: 1470 | mG*mA*mGmCmUmUmUmCfUfCfGmGmGmAmAmAmGmCmC*mG*mA |
| | SEQ ID NO: 2087 | mU*fC*mGmGfCmUfUmUmCmCmCmGmAfGmAfAmAmGmCmUmC*mU*mA |
| D: 929 | SEQ ID NO: 1472 | mG*mG*mGmAmAmAmGmCfCfGfCmGmAmGmGmGmUmCmG*mU*mA |
| | SEQ ID NO: 2088 | mU*fA*mCmGfAmCfCmCmUmCmGmCmGfGmCfUmUmUmCmCmC*mG*mA |
| D: 930 | SEQ ID NO: 1474 | mG*mG*mAmAmAmGmCmCfGfCfGmAmGmGmGmUmCmGmU*mC*mA |
| | SEQ ID NO: 2089 | mU*fG*mAmCfGmAfCmCmCmUmCmGmCfGmGfCmUmUmUmCmC*mC*mG |
| D: 931 | SEQ ID NO: 1476 | mG*mA*mAmAmGmCmCmGfCfGfAmGmGmGmUmCmGmUmC*mC*mA |
| | SEQ ID NO: 2090 | mU*fG*mGmAfCmGfAmCmCmCmUmCmGfCmGfGmCmUmUmUmC*mC*mC |
| D: 932 | SEQ ID NO: 1478 | mA*mA*mAmGmCmCmGmCfGfAfGmGmGmUmCmGmUmCmC*mA*mA |
| | SEQ ID NO: 2091 | mU*fU*mGmGfAmCfGmAmCmCmCmUmCfGmCfGmGmCmUmUmU*mC*mC |
| D: 933 | SEQ ID NO: 1480 | mA*mA*mGmCmCmGmCmGfAfGfGmGmUmCmGmUmCmCmA*mA*mA |
| | SEQ ID NO: 2092 | mU*fU*mUmGfGmAfCmGmAmCmCmCmUfCmGfCmGmGmCmUmU*mU*mC |
| D: 934 | SEQ ID NO: 1482 | mA*mG*mCmCmGmCmGmAfGfGfGmUmCmGmUmCmCmAmA*mU*mA |
| | SEQ ID NO: 2093 | mU*fA*mUmUfGmGfAmCmGmAmCmCmCfUmCfGmCmGmGmCmU*mU*mU |
| D: 935 | SEQ ID NO: 1484 | mG*mC*mCmGmCmGmAmGfGfGfUmCmGmUmCmCmAmAmU*mU*mA |
| | SEQ ID NO: 2094 | mU*fA*mAmUfUmGfGmAmCmGmAmCmCfCmUfCmGmCmGmGmC*mU*mU |
| D: 936 | SEQ ID NO: 1486 | mC*mC*mGmCmGmAmGmGfGfUfCmGmUmCmCmAmAmUmU*mU*mA |
| | SEQ ID NO: 2095 | mU*fA*mAmAfUmUfGmGmAmCmGmAmCfCmCfUmCmGmCmGmG*mC*mU |
| D: 937 | SEQ ID NO: 1488 | mC*mG*mCmGmAmGmGmGfUfCfGmUmCmCmAmAmUmUmU*mG*mA |
| | SEQ ID NO: 2096 | mU*fC*mAmAfAmUfUmGmGmAmCmGmAfCmCfCmUmCmGmCmG*mG*mC |
| D: 938 | SEQ ID NO: 1490 | mC*mC*mCmGmAmGmUmUfUfUfAmGmAmAmGmAmAmGmA*mA*mA |
| | SEQ ID NO: 2097 | mU*fU*mUmCfUmUfCmUmUmCmUmAmAfAmAfCmUmCmGmGmG*mC*mA |
| D: 939 | SEQ ID NO: 1492 | mC*mU*mCmAmGmAmGmGfGfUfCmAmAmGmAmUmGmAmU*mU*mA |
| | SEQ ID NO: 2098 | mU*fA*mAmUfCmAfUmCmUmUmGmAmCfCmCfUmCmUmGmAmG*mU*mU |
| D: 940 | SEQ ID NO: 1494 | mG*mG*mUmCmAmAmGmAfUfGfAmUmUmAmUmGmUmCmU*mC*mA |
| | SEQ ID NO: 2099 | mU*fG*mAmGfAmCfAmUmAmAmUmCmAfUmCfUmUmGmAmCmC*mC*mU |
| D: 941 | SEQ ID NO: 1496 | mG*mU*mCmAmAmGmAmUfGfAfUmUmAmUmGmUmCmUmC*mU*mA |
| | SEQ ID NO: 2100 | mU*fA*mGmAfGmAfCmAmUmAmAmUmCfAmUfCmUmUmGmAmC*mC*mC |
| D: 942 | SEQ ID NO: 1498 | mC*mA*mAmGmAmUmGmAfUfUfAmUmGmUmCmUmCmUmA*mG*mA |
| | SEQ ID NO: 2101 | mU*fC*mUmAfGmAfGmAmCmAmUmAmAfUmCfAmUmCmUmUmG*mA*mC |
| D: 943 | SEQ ID NO: 1500 | mA*mA*mGmAmUmGmAmUfUfAfUmGmUmCmUmCmUmAmG*mG*mA |
| | SEQ ID NO: 2102 | mU*fC*mCmUfAmGfAmGmAmCmAmUmAfAmUfCmAmUmCmUmU*mG*mA |
| D: 944 | SEQ ID NO: 1502 | mA*mG*mAmUmGmAmUmUfAfUfGmUmCmUmCmUmAmGmG*mC*mA |
| | SEQ ID NO: 2103 | mU*fG*mCmCfUmAfGmAmGmAmCmAmUfAmAfUmCmAmUmCmU*mU*mG |
| D: 945 | SEQ ID NO: 1504 | mG*mA*mUmGmAmUmUmAfUfGfUmCmUmCmUmAmGmGmC*mU*mA |
| | SEQ ID NO: 2104 | mU*fA*mGmCfCmUfAmGmAmGmAmCmAfUmAfAmUmCmAmUmC*mU*mU |
| D: 946 | SEQ ID NO: 1506 | mA*mU*mGmAmUmUmAmUfGfUfCmUmCmUmAmGmGmCmU*mU*mA |
| | SEQ ID NO: 2105 | mU*fA*mAmGfCmCfUmAmGmAmGmAmCfAmUfAmAmUmCmAmU*mC*mU |
| D: 947 | SEQ ID NO: 1508 | mU*mG*mAmUmUmAmUmGfUfCfUmCmUmAmGmGmCmUmU*mG*mA |
| | SEQ ID NO: 2106 | mU*fC*mAmAfGmCfCmUmAmGmAmGmAfCmAfUmAmAmUmCmA*mU*mC |
| D: 948 | SEQ ID NO: 1510 | mA*mU*mGmCmUmCmAmCfAfGfUmCmGmCmUmGmGmAmU*mA*mA |
| | SEQ ID NO: 2107 | mU*fU*mAmUfCmCfAmGmCmGmAmCmUfGmUfGmAmGmCmAmU*mG*mA |
| D: 949 | SEQ ID NO: 1512 | mU*mG*mCmUmCmAmCmAfGfUfCmGmCmUmGmGmAmUmA*mG*mA |
| | SEQ ID NO: 2108 | mU*fC*mUmAfUmCfCmAmGmCmGmAmCfUmGfUmGmAmGmCmA*mU*mG |
| D: 950 | SEQ ID NO: 1514 | mG*mC*mUmCmAmCmAmGfUfCfGmCmUmGmGmAmUmAmG*mC*mA |
| | SEQ ID NO: 2109 | mU*fG*mCmUfAmUfCmCmAmGmCmGmAfCmUfGmUmGmAmGmC*mA*mU |
| D: 951 | SEQ ID NO: 1516 | mC*mU*mCmAmCmAmGmUfCfGfCmUmGmGmAmUmAmGmC*mU*mA |
| | SEQ ID NO: 2110 | mU*fA*mGmCfUmAfUmCmCmAmGmCmGfAmCfUmGmUmGmAmG*mC*mA |
| D: 952 | SEQ ID NO: 1518 | mU*mC*mAmCmAmGmUmCfGfCfUmGmGmAmUmAmGmCmU*mG*mA |
| | SEQ ID NO: 2111 | mU*fC*mAmGfCmUfAmUmCmCmAmGmCfGmAfCmUmGmUmGmA*mG*mC |
| D: 953 | SEQ ID NO: 1520 | mC*mA*mCmAmGmUmCmGfCfUfGmGmAmUmAmGmCmUmG*mA*mA |
| | SEQ ID NO: 2112 | mU*fU*mCmAfGmCfUmAmUmCmCmAmGfCmGfAmCmUmGmUmG*mA*mG |
| D: 954 | SEQ ID NO: 1522 | mG*mA*mAmUmUmGmAmAfCfCfAmAmGmUmGmUmUmUmC*mC*mA |
| | SEQ ID NO: 2113 | mU*fG*mGmAfAmAfCmAmCmUmUmGmGfUmUfCmAmAmUmUmC*mU*mC |
| D: 955 | SEQ ID NO: 1524 | mG*mA*mAmCmCmAmAmGfUfGfUmUmUmCmCmCmUmCmU*mG*mA |
| | SEQ ID NO: 2114 | mU*fC*mAmGfAmGfGmGmAmAmAmCmAfCmUfUmGmGmUmUmC*mA*mA |
| D: 956 | SEQ ID NO: 1526 | mA*mA*mAmUmGmAmUmCfAfGfCmAmUmGmGmAmUmAmU*mU*mA |
| | SEQ ID NO: 2115 | mU*fA*mAmUfAmUfCmCmAmUmGmCmUfGmAfUmCmAmUmUmU*mU*mA |
| D: 957 | SEQ ID NO: 1528 | mA*mU*mGmAmUmCmAmGfCfAfUmGmGmAmUmAmUmUmG*mA*mA |
| | SEQ ID NO: 2116 | mU*fU*mCmAfAmUfAmUmCmCmAmUmGfCmUfGmAmUmCmAmU*mU*mU |
| D: 958 | SEQ ID NO: 1530 | mU*mC*mUmCmCmUmUmCfUfGfGmCmUmGmUmCmAmAmG*mU*mA |
| | SEQ ID NO: 2117 | mU*fA*mCmUfUmGfAmCmAmGmCmCmAfGmAfAmGmGmAmGmA*mG*mC |
| D: 959 | SEQ ID NO: 1532 | mC*mU*mCmCmUmUmCmUfGfGfCmUmGmUmCmAmAmGmU*mA*mA |
| | SEQ ID NO: 2118 | mU*fU*mAmCfUmUfGmAmCmAmGmCmCfAmGfAmAmGmGmAmG*mA*mG |
| D: 960 | SEQ ID NO: 1534 | mA*mA*mCmAmGmAmGmAfCfAfGmAmAmUmCmUmAmCmA*mC*mA |
| | SEQ ID NO: 2119 | mU*fG*mUmGfUmAfGmAmUmUmCmUmGfUmCfUmCmUmGmUmU*mU*mG |
| D: 961 | SEQ ID NO: 1536 | mA*mC*mAmGmAmGmAmCfAfGfAmAmUmCmUmAmCmAmC*mU*mA |
| | SEQ ID NO: 2120 | mU*fA*mGmUfGmUfAmGmAmUmUmCmUfGmUfCmUmCmUmGmU*mU*mU |
| D: 962 | SEQ ID NO: 1538 | mA*mU*mUmGmUmUmGmUfAfGfAmCmGmUmGmAmAmCmC*mU*mA |
| | SEQ ID NO: 2121 | mU*fA*mGmGfUmUfCmAmCmGmUmCmUfAmCfAmAmCmAmAmU*mU*mG |
| D: 963 | SEQ ID NO: 1540 | mA*mA*mGmUmUmGmAmGfGfUfUmAmUmAmAmAmAmCmC*mC*mA |
| | SEQ ID NO: 2122 | mU*fG*mGmGfUmUfUmUmAmUmAmAmCfCmUfCmAmAmCmUmU*mU*mU |
| D: 964 | SEQ ID NO: 1542 | mG*mU*mUmGmAmGmGmUfUfAfUmAmAmAmAmCmCmCmU*mU*mA |
| | SEQ ID NO: 2123 | mU*fA*mAmGfGmGfUmUmUmUmAmUmAfAmCfCmUmCmAmAmC*mU*mU |
| D: 965 | SEQ ID NO: 1544 | mU*mU*mGmAmGmGmUmUfAfUfAmAmAmAmCmCmCmUmU*mA*mA |
| | SEQ ID NO: 2124 | mU*fU*mAmAfGmGfGmUmUmUmUmAmUfAmAfCmCmUmCmAmA*mC*mU |
| D: 966 | SEQ ID NO: 1546 | mU*mG*mAmGmGmUmUmAfUfAfAmAmAmCmCmCmUmUmA*mG*mA |
| | SEQ ID NO: 2125 | mU*fC*mUmAfAmGfGmGmUmUmUmUmAfUmAfAmCmCmUmCmA*mA*mC |
| D: 967 | SEQ ID NO: 1548 | mG*mC*mAmGmAmGmAmAfAfGfGmUmGmCmAmAmAmAmU*mU*mA |
| | SEQ ID NO: 2126 | mU*fA*mAmUfUmUfUmGmCmAmCmCmUfUmUfCmUmCmUmGmC*mA*mU |
| D: 968 | SEQ ID NO: 1550 | mC*mA*mGmAmGmAmAmAfGfGfUmGmCmAmAmAmAmUmU*mC*mA |
| | SEQ ID NO: 2127 | mU*fG*mAmAfUmUfUmUmGmCmAmCmCfUmUfUmCmUmCmUmG*mC*mA |
| D: 969 | SEQ ID NO: 1552 | mA*mG*mAmGmAmAmAmGfGfUfGmCmAmAmAmAmUmUmC*mC*mA |
| | SEQ ID NO: 2128 | mU*fG*mGmAfAmUfUmUmUmGmCmAmCfCmUfUmUmCmUmCmU*mG*mC |
| D: 970 | SEQ ID NO: 1554 | mG*mA*mGmAmAmAmGmGfUfGfCmAmAmAmAmUmUmCmC*mU*mA |
| | SEQ ID NO: 2129 | mU*fA*mGmGfAmAfUmUmUmUmGmCmAfCmCfUmUmUmCmUmC*mU*mG |
| D: 971 | SEQ ID NO: 1556 | mA*mG*mAmAmAmGmGmUfGfCfAmAmAmAmUmUmCmCmU*mU*mA |
| | SEQ ID NO: 2130 | mU*fA*mAmGfGmAfAmUmUmUmUmGmCfAmCfCmUmUmUmCmU*mC*mU |
| D: 972 | SEQ ID NO: 1558 | mG*mA*mAmAmGmGmUmGfCfAfAmAmAmUmUmCmCmUmU*mA*mA |
| | SEQ ID NO: 2131 | mU*fU*mAmAfGmGfAmAmUmUmUmUmGfCmAfCmCmUmUmUmC*mU*mC |
| D: 973 | SEQ ID NO: 1560 | mA*mA*mAmGmGmUmGmCfAfAfAmAmUmUmCmCmUmUmA*mG*mA |
| | SEQ ID NO: 2132 | mU*fC*mUmAfAmGfGmAmAmUmUmUmUfGmCfAmCmCmUmUmU*mC*mU |
| D: 974 | SEQ ID NO: 1562 | mA*mA*mGmGmUmGmCmAfAfAfAmUmUmCmCmUmUmAmG*mU*mA |
| | SEQ ID NO: 2133 | mU*fA*mCmUfAmAfGmGmAmAmUmUmUfUmGfCmAmCmCmUmU*mU*mC |
| D: 975 | SEQ ID NO: 1564 | mA*mG*mGmUmGmCmAmAfAfAfUmUmCmCmUmUmAmGmU*mG*mA |
| | SEQ ID NO: 2134 | mU*fC*mAmCfUmAfAmGmGmAmAmUmUfUmUfGmCmAmCmCmU*mU*mU |
| D: 976 | SEQ ID NO: 1566 | mU*mG*mCmAmAmAmAmUfUfCfCmUmUmAmGmUmGmAmU*mG*mA |
| | SEQ ID NO: 2135 | mU*fC*mAmUfCmAfCmUmAmAmGmGmAfAmUfUmUmUmGmCmA*mC*mC |
| D: 977 | SEQ ID NO: 1568 | mG*mC*mAmAmAmAmUmUfCfCfUmUmAmGmUmGmAmUmG*mC*mA |
| | SEQ ID NO: 2136 | mU*fG*mCmAfUmCfAmCmUmAmAmGmGfAmAfUmUmUmUmGmC*mA*mC |
| D: 978 | SEQ ID NO: 1570 | mA*mA*mUmUmCmCmUmUfAfGfUmGmAmUmGmCmUmGmA*mG*mA |
| | SEQ ID NO: 2137 | mU*fC*mUmCfAmGfCmAmUmCmAmCmUfAmAfGmGmAmAmUmU*mU*mU |
| D: 979 | SEQ ID NO: 1572 | mU*mC*mCmUmUmAmGmUfGfAfUmGmCmUmGmAmGmAmU*mC*mA |
| | SEQ ID NO: 2138 | mU*fG*mAmUfCmUfCmAmGmCmAmUmCfAmCfUmAmAmGmGmA*mA*mU |
| D: 980 | SEQ ID NO: 1574 | mC*mC*mUmUmAmGmUmGfAfUfGmCmUmGmAmGmAmUmC*mA*mA |
| | SEQ ID NO: 2139 | mU*fU*mGmAfUmCfUmCmAmGmCmAmUfCmAfCmUmAmAmGmG*mA*mA |
| D: 981 | SEQ ID NO: 1576 | mC*mU*mGmAmGmAmUmCfAfUfCmCmAmGmUmUmAmGmU*mC*mA |
| | SEQ ID NO: 2140 | mU*fG*mAmCfUmAfAmCmUmGmGmAmUfGmAfUmCmUmCmAmG*mC*mA |
| D: 982 | SEQ ID NO: 1578 | mU*mG*mAmGmAmUmCmAfUfCfCmAmGmUmUmAmGmUmC*mA*mA |
| | SEQ ID NO: 2141 | mU*fU*mGmAfCmUfAmAmCmUmGmGmAfUmGfAmUmCmUmCmA*mG*mC |
| D: 983 | SEQ ID NO: 1580 | mG*mA*mGmAmUmCmAmUfCfCfAmGmUmUmAmGmUmCmA*mA*mA |
| | SEQ ID NO: 2142 | mU*fU*mUmGfAmCfUmAmAmCmUmGmGfAmUfGmAmUmCmUmC*mA*mG |
| D: 984 | SEQ ID NO: 1582 | mA*mU*mCmAmUmCmCmAfGfUfUmAmGmUmCmAmAmUmG*mC*mA |
| | SEQ ID NO: 2143 | mU*fG*mCmAfUmUfGmAmCmUmAmAmCfUmGfGmAmUmGmAmU*mC*mU |
| D: 985 | SEQ ID NO: 1584 | mG*mU*mCmAmAmUmGmCfUfAfAmGmCmAmUmAmUmCmC*mC*mA |
| | SEQ ID NO: 2144 | mU*fG*mGmGfAmUfAmUmGmCmUmUmAfGmCfAmUmUmGmAmC*mU*mA |
| D: 986 | SEQ ID NO: 1586 | mC*mU*mAmAmGmCmAmUfAfUfCmCmCmAmGmCmCmUmA*mC*mA |
| | SEQ ID NO: 2145 | mU*fG*mUmAfGmGfCmUmGmGmGmAmUfAmUfGmCmUmUmAmG*mC*mA |
| D: 987 | SEQ ID NO: 1588 | mA*mA*mGmCmAmUmAmUfCfCfCmAmGmCmCmUmAmCmA*mA*mA |
| | SEQ ID NO: 2146 | mU*fU*mUmGfUmAfGmGmCmUmGmGmGfAmUfAmUmGmCmUmU*mA*mG |
| D: 988 | SEQ ID NO: 1590 | mU*mG*mGmAmAmAmCmUfCfUfGmAmUmGmGmAmAmAmC*mU*mA |
| | SEQ ID NO: 2147 | mU*fA*mGmUfUmUfCmCmAmUmCmAmGfAmGfUmUmUmCmCmA*mA*mC |
| D: 989 | SEQ ID NO: 1592 | mG*mG*mAmAmAmCmUmCfUfGfAmUmGmGmAmAmAmCmU*mC*mA |
| | SEQ ID NO: 2148 | mU*fG*mAmGfUmUfUmCmCmAmUmCmAfGmAfGmUmUmUmCmC*mA*mA |
| D: 990 | SEQ ID NO: 1594 | mG*mA*mAmAmCmUmCmUfGfAfUmGmGmAmAmAmCmUmC*mA*mA |
| | SEQ ID NO: 2149 | mU*fU*mGmAfGmUfUmUmCmCmAmUmCfAmGfAmGmUmUmUmC*mC*mA |
| D: 991 | SEQ ID NO: 1596 | mA*mA*mAmCmUmCmUmGfAfUfGmGmAmAmAmCmUmCmA*mU*mA |
| | SEQ ID NO: 2150 | mU*fA*mUmGfAmGfUmUmUmCmCmAmUfCmAfGmAmGmUmUmU*mC*mC |
| D: 992 | SEQ ID NO: 1598 | mG*mA*mUmGmGmAmAmAfCfUfCmAmUmGmAmGmCmGmU*mG*mA |
| | SEQ ID NO: 2151 | mU*fC*mAmCfGmCfUmCmAmUmGmAmGfUmUfUmCmCmAmUmC*mA*mG |
| D: 993 | SEQ ID NO: 1600 | mA*mU*mGmGmAmAmAmCfUfCfAmUmGmAmGmCmGmUmG*mG*mA |
| | SEQ ID NO: 2152 | mU*fC*mCmAfCmGfCmUmCmAmUmGmAfGmUfUmUmCmCmAmU*mC*mA |
| D: 994 | SEQ ID NO: 1602 | mU*mG*mGmAmAmAmCmUfCfAfUmGmAmGmCmGmUmGmG*mU*mA |
| | SEQ ID NO: 2153 | mU*fA*mCmCfAmCfGmCmUmCmAmUmGfAmGfUmUmUmCmCmA*mU*mC |
| D: 995 | SEQ ID NO: 1604 | mG*mG*mAmAmAmCmUmCfAfUfGmAmGmCmGmUmGmGmU*mG*mA |
| | SEQ ID NO: 2154 | mU*fC*mAmCfCmAfCmGmCmUmCmAmUfGmAfGmUmUmUmCmC*mA*mU |
| D: 996 | SEQ ID NO: 1606 | mG*mA*mAmAmCmUmCmAfUfGfAmGmCmGmUmGmGmUmG*mU*mA |
| | SEQ ID NO: 2155 | mU*fA*mCmAfCmCfAmCmGmCmUmCmAfUmGfAmGmUmUmUmC*mC*mA |
| D: 997 | SEQ ID NO: 1608 | mA*mG*mCmGmUmGmGmUfGfUfAmUmCmUmAmUmUmCmG*mC*mA |
| | SEQ ID NO: 2156 | mU*fG*mCmGfAmAfUmAmGmAmUmAmCfAmCfCmAmCmGmCmU*mC*mA |
| D: 998 | SEQ ID NO: 1610 | mU*mA*mCmCmUmUmAmCfAfGfGmGmAmUmUmAmUmAmA*mU*mA |
| | SEQ ID NO: 2157 | mU*fA*mUmUfAmUfAmAmUmCmCmCmUfGmUfAmAmGmGmUmA*mG*mG |
| D: 999 | SEQ ID NO: 1612 | mA*mC*mCmUmUmAmCmAfGfGfGmAmUmUmAmUmAmAmU*mU*mA |
| | SEQ ID NO: 2158 | mU*fA*mAmUfUmAfUmAmAmUmCmCmCfUmGfUmAmAmGmGmU*mA*mG |
| D: 1000 | SEQ ID NO: 1614 | mG*mG*mGmAmUmUmAmUfAfAfUmUmAmCmUmCmCmUmU*mG*mA |
| | SEQ ID NO: 2159 | mU*fC*mAmAfGmGfAmGmUmAmAmUmUfAmUfAmAmUmCmCmC*mU*mG |
| D: 1001 | SEQ ID NO: 1616 | mG*mG*mAmUmUmAmUmAfAfUfUmAmCmUmCmCmUmUmG*mG*mA |
| | SEQ ID NO: 2160 | mU*fC*mCmAfAmGfGmAmGmUmAmAmUfUmAfUmAmAmUmCmC*mC*mU |
| D: 1002 | SEQ ID NO: 1618 | mG*mA*mUmUmAmUmAmAfUfUfAmCmUmCmCmUmUmGmG*mU*mA |
| | SEQ ID NO: 2161 | mU*fA*mCmCfAmAfGmGmAmGmUmAmAfUmUfAmUmAmAmUmC*mC*mC |
| D: 1003 | SEQ ID NO: 1620 | mA*mU*mUmAmUmAmAmUfUfAfCmUmCmCmUmUmGmGmU*mG*mA |
| | SEQ ID NO: 2162 | mU*fC*mAmCfCmAfAmGmGmAmGmUmAfAmUfUmAmUmAmAmU*mC*mC |
| D: 1004 | SEQ ID NO: 1622 | mA*mA*mUmUmAmCmUmCfCfUfUmGmGmUmGmAmUmGmG*mG*mA |
| | SEQ ID NO: 2163 | mU*fC*mCmCfAmUfCmAmCmCmAmAmGfGmAfGmUmAmAmUmU*mA*mU |
| D: 1005 | SEQ ID NO: 1624 | mA*mU*mUmAmCmUmCmCfUfUfGmGmUmGmAmUmGmGmG*mA*mA |
| | SEQ ID NO: 2164 | mU*fU*mCmCfCmAfUmCmAmCmCmAmAfGmGfAmGmUmAmAmU*mU*mA |
| D: 1006 | SEQ ID NO: 1626 | mC*mC*mUmUmGmGmUmGfAfUfGmGmGmAmGmCmUmUmG*mU*mA |
| | SEQ ID NO: 2165 | mU*fA*mCmAfAmGfCmUmCmCmCmAmUfCmAfCmCmAmAmGmG*mA*mG |
| D: 1007 | SEQ ID NO: 1628 | mU*mG*mGmUmGmAmUmGfGfGfAmGmCmUmUmGmUmUmG*mU*mA |
| | SEQ ID NO: 2166 | mU*fA*mCmAfAmCfAmAmGmCmUmCmCfCmAfUmCmAmCmCmA*mA*mG |
| D: 1008 | SEQ ID NO: 1630 | mU*mA*mUmUmGmGmAmUfAfUfAmUmGmCmCmCmAmUmC*mC*mA |
| | SEQ ID NO: 2167 | mU*fG*mGmAfUmGfGmGmCmAmUmAmUfAmUfCmCmAmAmUmA*mA*mC |
| D: 1009 | SEQ ID NO: 1632 | mA*mU*mUmGmGmAmUmAfUfAfUmGmCmCmCmAmUmCmC*mC*mA |
| | SEQ ID NO: 2168 | mU*fG*mGmGfAmUfGmGmGmCmAmUmAfUmAfUmCmCmAmAmU*mA*mA |
| D: 1010 | SEQ ID NO: 1634 | mC*mA*mGmAmAmGmGmUfUfGfUmCmUmUmGmUmGmGmC*mC*mA |
| | SEQ ID NO: 2169 | mU*fG*mGmCfCmAfCmAmAmGmAmCmAfAmCfCmUmUmCmUmG*mU*mU |
| D: 1011 | SEQ ID NO: 1636 | mA*mG*mGmUmUmGmUmCfUfUfGmUmGmGmCmCmAmGmC*mA*mA |
| | SEQ ID NO: 2170 | mU*fU*mGmCfUmGfGmCmCmAmCmAmAfGmAfCmAmAmCmCmU*mU*mC |
| D: 1012 | SEQ ID NO: 1638 | mU*mU*mGmUmCmUmUmGfUfGfGmCmCmAmGmCmAmCmC*mA*mA |
| | SEQ ID NO: 2171 | mU*fU*mGmGfUmGfCmUmGmGmCmCmAfCmAfAmGmAmCmAmA*mC*mC |
| D: 1013 | SEQ ID NO: 1640 | mU*mG*mUmCmUmUmGmUfGfGfCmCmAmGmCmAmCmCmA*mA*mA |
| | SEQ ID NO: 2172 | mU*fU*mUmGfGmUfGmCmUmGmGmCmCfAmCfAmAmGmAmCmA*mA*mC |
| D: 1014 | SEQ ID NO: 1642 | mA*mG*mGmUmCmUmUmGfGfUfGmGmAmGmGmUmGmCmC*mA*mA |
| | SEQ ID NO: 2173 | mU*fU*mGmGfCmAfCmCmUmCmCmAmCfCmAfAmGmAmCmCmU*mA*mU |
| D: 1015 | SEQ ID NO: 1644 | mG*mG*mUmCmUmUmGmGfUfGfGmAmGmGmUmGmCmCmA*mG*mA |
| | SEQ ID NO: 2174 | mU*fC*mUmGfGmCfAmCmCmUmCmCmAfCmCfAmAmGmAmCmC*mU*mA |
| D: 1016 | SEQ ID NO: 1646 | mG*mU*mGmGmAmGmGmUfGfCfCmAmGmCmAmGmCmCmG*mA*mA |
| | SEQ ID NO: 2175 | mU*fU*mCmGfGmCfUmGmCmUmGmGmCfAmCfCmUmCmCmAmC*mC*mA |
| D: 1017 | SEQ ID NO: 1648 | mG*mU*mGmCmCmAmGmCfAfGfCmCmGmAmGmUmCmCmU*mU*mA |
| | SEQ ID NO: 2176 | mU*fA*mAmGfGmAfCmUmCmGmGmCmUfGmCfUmGmGmCmAmC*mC*mU |
| D: 1018 | SEQ ID NO: 1650 | mC*mA*mGmCmAmGmCmCfGfAfGmUmCmCmUmUmGmCmA*mG*mA |
| | SEQ ID NO: 2177 | mU*fC*mUmGfCmAfAmGmGmAmCmUmCfGmGfCmUmGmCmUmG*mG*mC |
| D: 1019 | SEQ ID NO: 1652 | mA*mG*mCmAmGmCmCmGfAfGfUmCmCmUmUmGmCmAmG*mA*mA |
| | SEQ ID NO: 2178 | mU*fU*mCmUfGmCfAmAmGmGmAmCmUfCmGfGmCmUmGmCmU*mG*mG |
| D: 1020 | SEQ ID NO: 1654 | mG*mC*mAmGmCmCmGmAfGfUfCmCmUmUmGmCmAmGmA*mU*mA |
| | SEQ ID NO: 2179 | mU*fA*mUmCfUmGfCmAmAmGmGmAmCfUmCfGmGmCmUmGmC*mU*mG |
| D: 1021 | SEQ ID NO: 1656 | mC*mA*mGmCmCmGmAmGfUfCfCmUmUmGmCmAmGmAmU*mG*mA |
| | SEQ ID NO: 2180 | mU*fC*mAmUfCmUfGmCmAmAmGmGmAfCmUfCmGmGmCmUmG*mC*mU |
| D: 1022 | SEQ ID NO: 1658 | mG*mC*mCmGmAmGmUmCfCfUfUmGmCmAmGmAmUmGmG*mG*mA |
| | SEQ ID NO: 2181 | mU*fC*mCmCfAmUfCmUmGmCmAmAmGfGmAfCmUmCmGmGmC*mU*mG |
| D: 1023 | SEQ ID NO: 1660 | mC*mG*mAmGmUmCmCmUfUfGfCmAmGmAmUmGmGmGmA*mU*mA |
| | SEQ ID NO: 2182 | mU*fA*mUmCfCmCfAmUmCmUmGmCmAfAmGfGmAmCmUmCmG*mG*mC |
| D: 1024 | SEQ ID NO: 1662 | mU*mU*mGmCmAmGmAmUfGfGfGmAmUmGmAmCmUmCmG*mU*mA |
| | SEQ ID NO: 2183 | mU*fA*mCmGfAmGfUmCmAmUmCmCmCfAmUfCmUmGmCmAmA*mG*mG |
| D: 1025 | SEQ ID NO: 1664 | mU*mG*mCmAmGmAmUmGfGfGfAmUmGmAmCmUmCmGmU*mG*mA |
| | SEQ ID NO: 2184 | mU*fC*mAmCfGmAfGmUmCmAmUmCmCfCmAfUmCmUmGmCmA*mA*mG |
| D: 1026 | SEQ ID NO: 1666 | mG*mC*mAmGmAmUmGmGfGfAfUmGmAmCmUmCmGmUmG*mG*mA |
| | SEQ ID NO: 2185 | mU*fC*mCmAfCmGfAmGmUmCmAmUmCfCmCfAmUmCmUmGmC*mA*mA |
| D: 1027 | SEQ ID NO: 1668 | mC*mA*mGmAmUmGmGmGfAfUfGmAmCmUmCmGmUmGmG*mC*mA |
| | SEQ ID NO: 2186 | mU*fG*mCmCfAmCfGmAmGmUmCmAmUfCmCfCmAmUmCmUmG*mC*mA |
| D: 1028 | SEQ ID NO: 1670 | mA*mG*mAmUmGmGmGmAfUfGfAmCmUmCmGmUmGmGmC*mC*mA |
| | SEQ ID NO: 2187 | mU*fG*mGmCfCmAfCmGmAmGmUmCmAfUmCfCmCmAmUmCmU*mG*mC |
| D: 1029 | SEQ ID NO: 1672 | mG*mA*mUmGmGmGmAmUfGfAfCmUmCmGmUmGmGmCmC*mC*mA |
| | SEQ ID NO: 2188 | mU*fG*mGmGfCmCfAmCmGmAmGmUmCfAmUfCmCmCmAmUmC*mU*mG |
| D: 1030 | SEQ ID NO: 1674 | mA*mU*mGmGmGmAmUmGfAfCfUmCmGmUmGmGmCmCmC*mA*mA |
| | SEQ ID NO: 2189 | mU*fU*mGmGfGmCfCmAmCmGmAmGmUfCmAfUmCmCmCmAmU*mC*mU |
| D: 1031 | SEQ ID NO: 1676 | mU*mG*mGmGmAmUmGmAfCfUfCmGmUmGmGmCmCmCmA*mG*mA |
| | SEQ ID NO: 2190 | mU*fC*mUmGfGmGfCmCmAmCmGmAmGfUmCfAmUmCmCmCmA*mU*mC |
| D: 1032 | SEQ ID NO: 1678 | mG*mG*mGmAmUmGmAmCfUfCfGmUmGmGmCmCmCmAmG*mU*mA |
| | SEQ ID NO: 2191 | mU*fA*mCmUfGmGfGmCmCmAmCmGmAfGmUfCmAmUmCmCmC*mA*mU |
| D: 1033 | SEQ ID NO: 1680 | mG*mU*mGmGmCmCmCmAfGfUfUmGmUmGmCmGmUmCmU*mU*mA |
| | SEQ ID NO: 2192 | mU*fA*mAmGfAmCfGmCmAmCmAmAmCfUmGfGmGmCmCmAmC*mG*mA |
| D: 1034 | SEQ ID NO: 1682 | mU*mG*mGmCmCmCmAmGfUfUfGmUmGmCmGmUmCmUmU*mC*mA |
| | SEQ ID NO: 2193 | mU*fG*mAmAfGmAfCmGmCmAmCmAmAfCmUfGmGmGmCmCmA*mC*mG |
| D: 1035 | SEQ ID NO: 1684 | mG*mC*mCmCmAmGmUmUfGfUfGmCmGmUmCmUmUmCmC*mA*mA |
| | SEQ ID NO: 2194 | mU*fU*mGmGfAmAfGmAmCmGmCmAmCfAmAfCmUmGmGmGmC*mC*mA |
| D: 1036 | SEQ ID NO: 1686 | mC*mC*mCmAmGmUmUmGfUfGfCmGmUmCmUmUmCmCmA*mC*mA |
| | SEQ ID NO: 2195 | mU*fG*mUmGfGmAfAmGmAmCmGmCmAfCmAfAmCmUmGmGmG*mC*mC |
| D: 1037 | SEQ ID NO: 1688 | mC*mC*mAmGmUmUmGmUfGfCfGmUmCmUmUmCmCmAmC*mG*mA |
| | SEQ ID NO: 2196 | mU*fC*mGmUfGmGfAmAmGmAmCmGmCfAmCfAmAmCmUmGmG*mG*mC |
| D: 1038 | SEQ ID NO: 1690 | mC*mA*mGmUmUmGmUmGfCfGfUmCmUmUmCmCmAmCmG*mU*mA |
| | SEQ ID NO: 2197 | mU*fA*mCmGfUmGfGmAmAmGmAmCmGfCmAfCmAmAmCmUmG*mG*mG |
| D: 1039 | SEQ ID NO: 1692 | mA*mG*mUmUmGmUmGmCfGfUfCmUmUmCmCmAmCmGmU*mG*mA |
| | SEQ ID NO: 2198 | mU*fC*mAmCfGmUfGmGmAmAmGmAmCfGmCfAmCmAmAmCmU*mG*mG |
| D: 1040 | SEQ ID NO: 1694 | mU*mU*mGmUmGmAmCmUfCfUfGmCmAmGmAmAmGmUmG*mA*mA |
| | SEQ ID NO: 2199 | mU*fU*mCmAfCmUfUmCmUmGmCmAmGfAmGfUmCmAmCmAmA*mG*mC |
| D: 1041 | SEQ ID NO: 1696 | mC*mU*mGmAmAmGmGmGfUfUfCmGmCmAmGmUmGmAmU*mA*mA |
| | SEQ ID NO: 2200 | mU*fU*mAmUfCmAfCmUmGmCmGmAmAfCmCfCmUmUmCmAmG*mA*mU |
| D: 1042 | SEQ ID NO: 1698 | mU*mG*mAmAmGmGmGmUfUfCfGmCmAmGmUmGmAmUmA*mA*mA |
| | SEQ ID NO: 2201 | mU*fU*mUmAfUmCfAmCmUmGmCmGmAfAmCfCmCmUmUmCmA*mG*mA |
| D: 1043 | SEQ ID NO: 1700 | mG*mA*mAmGmGmGmUmUfCfGfCmAmGmUmGmAmUmAmA*mA*mA |
| | SEQ ID NO: 2202 | mU*fU*mUmUfAmUfCmAmCmUmGmCmGfAmAfCmCmCmUmUmC*mA*mG |
| D: 1044 | SEQ ID NO: 1702 | mA*mG*mGmGmUmUmCmGfCfAfGmUmGmAmUmAmAmAmG*mG*mA |
| | SEQ ID NO: 2203 | mU*fC*mCmUfUmUfAmUmCmAmCmUmGfCmGfAmAmCmCmCmU*mU*mC |
| D: 1045 | SEQ ID NO: 1704 | mG*mG*mGmUmUmCmGmCfAfGfUmGmAmUmAmAmAmGmG*mA*mA |
| | SEQ ID NO: 2204 | mU*fU*mCmCfUmUfUmAmUmCmAmCmUfGmCfGmAmAmCmCmC*mU*mU |
| D: 1046 | SEQ ID NO: 1706 | mU*mU*mCmGmCmAmGmUfGfAfUmAmAmAmGmGmAmGmG*mC*mA |
| | SEQ ID NO: 2205 | mU*fG*mCmCfUmCfCmUmUmUmAmUmCfAmCfUmGmCmGmAmA*mC*mC |
| D: 1047 | SEQ ID NO: 1708 | mC*mA*mGmUmGmAmUmAfAfAfGmGmAmGmGmCmAmUmU*mU*mA |
| | SEQ ID NO: 2206 | mU*fA*mAmAfUmGfCmCmUmCmCmUmUfUmAfUmCmAmCmUmG*mC*mG |
| D: 1048 | SEQ ID NO: 1710 | mC*mU*mAmGmCmAmGmAfUfUfUmGmCmAmCmGmUmCmU*mA*mA |
| | SEQ ID NO: 2207 | mU*fU*mAmGfAmCfGmUmGmCmAmAmAfUmCfUmGmCmUmAmG*mU*mG |
| D: 1049 | SEQ ID NO: 1712 | mU*mA*mGmCmAmGmAmUfUfUfGmCmAmCmGmUmCmUmA*mC*mA |
| | SEQ ID NO: 2208 | mU*fG*mUmAfGmAfCmGmUmGmCmAmAfAmUfCmUmGmCmUmA*mG*mU |
| D: 1050 | SEQ ID NO: 1714 | mA*mG*mCmAmGmAmUmUfUfGfCmAmCmGmUmCmUmAmC*mA*mA |
| | SEQ ID NO: 2209 | mU*fU*mGmUfAmGfAmCmGmUmGmCmAfAmAfUmCmUmGmCmU*mA*mG |
| D: 1051 | SEQ ID NO: 1716 | mG*mC*mAmGmAmUmUmUfGfCfAmCmGmUmCmUmAmCmA*mG*mA |
| | SEQ ID NO: 2210 | mU*fC*mUmGfUmAfGmAmCmGmUmGmCfAmAfAmUmCmUmGmC*mU*mA |
| D: 1052 | SEQ ID NO: 1718 | mC*mA*mGmAmUmUmUmGfCfAfCmGmUmCmUmAmCmAmG*mA*mA |
| | SEQ ID NO: 2211 | mU*fU*mCmUfGmUfAmGmAmCmGmUmGfCmAfAmAmUmCmUmG*mC*mU |
| D: 1053 | SEQ ID NO: 1720 | mG*mA*mUmUmUmGmCmAfCfGfUmCmUmAmCmAmGmAmA*mA*mA |
| | SEQ ID NO: 2212 | mU*fU*mUmUfCmUfGmUmAmGmAmCmGfUmGfCmAmAmAmUmC*mU*mG |
| D: 1054 | SEQ ID NO: 1722 | mA*mU*mUmUmGmCmAmCfGfUfCmUmAmCmAmGmAmAmA*mC*mA |
| | SEQ ID NO: 2213 | mU*fG*mUmUfUmCfUmGmUmAmGmAmCfGmUfGmCmAmAmAmU*mC*mU |
| D: 1055 | SEQ ID NO: 1724 | mC*mU*mGmGmAmCmGmCfAfAfCmCmUmUmUmAmUmAmU*mC*mA |
| | SEQ ID NO: 2214 | mU*fG*mAmUfAmUfAmAmAmGmGmUmUfGmCfGmUmCmCmAmG*mC*mU |
| D: 1056 | SEQ ID NO: 1726 | mU*mG*mGmAmCmGmCmAfAfCfCmUmUmUmAmUmAmUmC*mC*mA |
| | SEQ ID NO: 2215 | mU*fG*mGmAfUmAfUmAmAmAmGmGmUfUmGfCmGmUmCmCmA*mG*mC |
| D: 1057 | SEQ ID NO: 1728 | mG*mG*mAmCmGmCmAmAfCfCfUmUmUmAmUmAmUmCmC*mG*mA |
| | SEQ ID NO: 2216 | mU*fC*mGmGfAmUfAmUmAmAmAmGmGfUmUfGmCmGmUmCmC*mA*mG |
| D: 1058 | SEQ ID NO: 1730 | mG*mA*mCmGmCmAmAmCfCfUfUmUmAmUmAmUmCmCmG*mU*mA |
| | SEQ ID NO: 2217 | mU*fA*mCmGfGmAfUmAmUmAmAmAmGfGmUfUmGmCmGmUmC*mC*mA |
| D: 1059 | SEQ ID NO: 1732 | mC*mG*mCmAmAmCmCmUfUfUfAmUmAmUmCmCmGmUmU*mU*mA |
| | SEQ ID NO: 2218 | mU*fA*mAmAfCmGfGmAmUmAmUmAmAfAmGfGmUmUmGmCmG*mU*mC |
| D: 1060 | SEQ ID NO: 1734 | mG*mC*mAmAmCmCmUmUfUfAfUmAmUmCmCmGmUmUmU*mC*mA |
| | SEQ ID NO: 2219 | mU*fG*mAmAfAmCfGmGmAmUmAmUmAfAmAfGmGmUmUmGmC*mG*mU |
| D: 1061 | SEQ ID NO: 1736 | mC*mA*mAmCmCmUmUmUfAfUfAmUmCmCmGmUmUmUmC*mC*mA |
| | SEQ ID NO: 2220 | mU*fG*mGmAfAmAfCmGmGmAmUmAmUfAmAfAmGmGmUmUmG*mC*mG |
| D: 1062 | SEQ ID NO: 1738 | mC*mC*mUmUmUmAmUmAfUfCfCmGmUmUmUmCmCmAmG*mU*mA |
| | SEQ ID NO: 2221 | mU*fA*mCmUfGmGfAmAmAmCmGmGmAfUmAfUmAmAmAmGmG*mU*mU |
| D: 1063 | SEQ ID NO: 1740 | mC*mU*mUmUmAmUmAmUfCfCfGmUmUmUmCmCmAmGmU*mC*mA |
| | SEQ ID NO: 2222 | mU*fG*mAmCfUmGfGmAmAmAmCmGmGfAmUfAmUmAmAmAmG*mG*mU |
| D: 1064 | SEQ ID NO: 1742 | mU*mA*mUmCmCmGmUmUfUfCfCmAmGmUmCmCmAmGmG*mU*mA |
| | SEQ ID NO: 2223 | mU*fA*mCmCfUmGfGmAmCmUmGmGmAfAmAfCmGmGmAmUmA*mU*mA |
| D: 1065 | SEQ ID NO: 1744 | mU*mU*mGmUmUmUmGmUfGfAfAmGmCmUmGmUmCmAmU*mU*mA |
| | SEQ ID NO: 2224 | mU*fA*mAmUfGmAfCmAmGmCmUmUmCfAmCfAmAmAmCmAmA*mC*mA |
| D: 1066 | SEQ ID NO: 1746 | mU*mU*mUmGmUmGmAmAfGfCfUmGmUmCmAmUmUmCmC*mA*mA |
| | SEQ ID NO: 2225 | mU*fU*mGmGfAmAfUmGmAmCmAmGmCfUmUfCmAmCmAmAmA*mC*mA |
| D: 1067 | SEQ ID NO: 1748 | mU*mU*mGmUmGmAmAmGfCfUfGmUmCmAmUmUmCmCmA*mG*mA |
| | SEQ ID NO: 2226 | mU*fC*mUmGfGmAfAmUmGmAmCmAmGfCmUfUmCmAmCmAmA*mA*mC |
| D: 1068 | SEQ ID NO: 1750 | mG*mA*mAmGmCmUmGmUfCfAfUmUmCmCmAmGmCmCmA*mA*mA |
| | SEQ ID NO: 2227 | mU*fU*mUmGfGmCfUmGmGmAmAmUmGfAmCfAmGmCmUmUmC*mA*mC |
| D: 1069 | SEQ ID NO: 1752 | mG*mC*mUmGmUmCmAmUfUfCfCmAmGmCmCmAmAmGmG*mU*mA |
| | SEQ ID NO: 2228 | mU*fA*mCmCfUmUfGmGmCmUmGmGmAfAmUfGmAmCmAmGmC*mU*mU |
| D: 1070 | SEQ ID NO: 1754 | mC*mU*mGmUmCmAmUmUfCfCfAmGmCmCmAmAmGmGmU*mU*mA |
| | SEQ ID NO: 2229 | mU*fA*mAmCfCmUfUmGmGmCmUmGmGfAmAfUmGmAmCmAmG*mC*mU |
| D: 1071 | SEQ ID NO: 1756 | mU*mG*mUmCmAmUmUmCfCfAfGmCmCmAmAmGmGmUmU*mG*mA |
| | SEQ ID NO: 2230 | mU*fC*mAmAfCmCfUmUmGmGmCmUmGfGmAfAmUmGmAmCmA*mG*mC |
| D: 1072 | SEQ ID NO: 1758 | mG*mU*mCmAmUmUmCmCfAfGfCmCmAmAmGmGmUmUmG*mU*mA |
| | SEQ ID NO: 2231 | mU*fA*mCmAfAmCfCmUmUmGmGmCmUfGmGfAmAmUmGmAmC*mA*mG |
| D: 1073 | SEQ ID NO: 1760 | mG*mC*mUmGmGmGmAmGfCfAfUmAmGmGmAmGmGmCmU*mA*mA |
| | SEQ ID NO: 2232 | mU*fU*mAmGfCmCfUmCmCmUmAmUmGfCmUfCmCmCmAmGmC*mC*mA |
| D: 1074 | SEQ ID NO: 1762 | mC*mU*mGmGmGmAmGmCfAfUfAmGmGmAmGmGmCmUmA*mC*mA |
| | SEQ ID NO: 2233 | mU*fG*mUmAfGmCfCmUmCmCmUmAmUfGmCfUmCmCmCmAmG*mC*mC |
| D: 1075 | SEQ ID NO: 1764 | mG*mG*mGmAmGmCmAmUfAfGfGmAmGmGmCmUmAmCmA*mA*mA |
| | SEQ ID NO: 2234 | mU*fU*mUmGfUmAfGmCmCmUmCmCmUfAmUfGmCmUmCmCmC*mA*mG |
| D: 1076 | SEQ ID NO: 1766 | mG*mG*mAmGmCmAmUmAfGfGfAmGmGmCmUmAmCmAmA*mC*mA |
| | SEQ ID NO: 2235 | mU*fG*mUmUfGmUfAmGmCmCmUmCmCfUmAfUmGmCmUmCmC*mC*mA |
| D: 1077 | SEQ ID NO: 1768 | mA*mG*mCmAmUmAmGmGfAfGfGmCmUmAmCmAmAmCmG*mC*mA |
| | SEQ ID NO: 2236 | mU*fG*mCmGfUmUfGmUmAmGmCmCmUfCmCfUmAmUmGmCmU*mC*mC |
| D: 1078 | SEQ ID NO: 1770 | mG*mC*mAmUmAmGmGmAfGfGfCmUmAmCmAmAmCmGmC*mC*mA |
| | SEQ ID NO: 2237 | mU*fG*mGmCfGmUfUmGmUmAmGmCmCfUmCfCmUmAmUmGmC*mU*mC |
| D: 1079 | SEQ ID NO: 1772 | mC*mA*mUmAmGmGmAmGfGfCfUmAmCmAmAmCmGmCmC*mC*mA |
| | SEQ ID NO: 2238 | mU*fG*mGmGfCmGfUmUmGmUmAmGmCfCmUfCmCmUmAmUmG*mC*mU |
| D: 1080 | SEQ ID NO: 1774 | mA*mU*mAmGmGmAmGmGfCfUfAmCmAmAmCmGmCmCmC*mA*mA |
| | SEQ ID NO: 2239 | mU*fU*mGmGfGmCfGmUmUmGmUmAmGfCmCfUmCmCmUmAmU*mG*mC |
| D: 1081 | SEQ ID NO: 1776 | mG*mA*mGmGmCmUmAmCfAfAfCmGmCmCmCmAmUmGmC*mA*mA |
| | SEQ ID NO: 2240 | mU*fU*mGmCfAmUfGmGmGmCmGmUmUfGmUfAmGmCmCmUmC*mC*mU |
| D: 1082 | SEQ ID NO: 1778 | mG*mG*mCmUmAmCmAmAfCfGfCmCmCmAmUmGmCmAmG*mC*mA |
| | SEQ ID NO: 2241 | mU*fG*mCmUfGmCfAmUmGmGmGmCmGfUmUfGmUmAmGmCmC*mU*mC |
| D: 1083 | SEQ ID NO: 1780 | mG*mC*mUmAmCmAmAmCfGfCfCmCmAmUmGmCmAmGmC*mA*mA |
| | SEQ ID NO: 2242 | mU*fU*mGmCfUmGfCmAmUmGmGmGmCfGmUfUmGmUmAmGmC*mC*mU |
| D: 1084 | SEQ ID NO: 1782 | mC*mU*mAmCmAmAmCmGfCfCfCmAmUmGmCmAmGmCmA*mA*mA |
| | SEQ ID NO: 2243 | mU*fU*mUmGfCmUfGmCmAmUmGmGmGfCmGfUmUmGmUmAmG*mC*mC |
| D: 1085 | SEQ ID NO: 1784 | mA*mC*mAmAmCmGmCmCfCfAfUmGmCmAmGmCmAmAmA*mC*mA |
| | SEQ ID NO: 2244 | mU*fG*mUmUfUmGfCmUmGmCmAmUmGfGmGfCmGmUmUmGmU*mA*mG |
| D: 1086 | SEQ ID NO: 1786 | mC*mA*mAmCmGmCmCmCfAfUfGmCmAmGmCmAmAmAmC*mA*mA |
| | SEQ ID NO: 2245 | mU*fU*mGmUfUmUfGmCmUmGmCmAmUfGmGfGmCmGmUmUmG*mU*mA |
| D: 1087 | SEQ ID NO: 1788 | mA*mA*mCmGmCmCmCmAfUfGfCmAmGmCmAmAmAmCmA*mU*mA |
| | SEQ ID NO: 2246 | mU*fA*mUmGfUmUfUmGmCmUmGmCmAfUmGfGmGmCmGmUmU*mG*mU |
| D: 1088 | SEQ ID NO: 1790 | mC*mC*mCmAmUmGmCmAfGfCfAmAmAmCmAmUmUmGmU*mC*mA |
| | SEQ ID NO: 2247 | mU*fG*mAmCfAmAfUmGmUmUmUmGmCfUmGfCmAmUmGmGmG*mC*mG |
| D: 1089 | SEQ ID NO: 1792 | mC*mU*mGmUmGmGmAmCfAfGfGmAmUmGmCmAmGmCmA*mC*mA |
| | SEQ ID NO: 2248 | mU*fG*mUmGfCmUfGmCmAmUmCmCmUfGmUfCmCmAmCmAmG*mG*mC |
| D: 1090 | SEQ ID NO: 1794 | mG*mG*mAmUmGmCmAmGfCfAfCmAmGmAmAmUmGmUmU*mG*mA |
| | SEQ ID NO: 2249 | mU*fC*mAmAfCmAfUmUmCmUmGmUmGfCmUfGmCmAmUmCmC*mU*mG |
| D: 1091 | SEQ ID NO: 1796 | mU*mU*mGmGmUmAmGmUfUfCfAmAmAmCmUmGmUmAmU*mU*mA |
| | SEQ ID NO: 2250 | mU*fA*mAmUfAmCfAmGmUmUmUmGmAfAmCfUmAmCmCmAmA*mC*mA |
| D: 1092 | SEQ ID NO: 1798 | mU*mG*mGmUmAmGmUmUfCfAfAmAmCmUmGmUmAmUmU*mA*mA |
| | SEQ ID NO: 2251 | mU*fU*mAmAfUmAfCmAmGmUmUmUmGfAmAfCmUmAmCmCmA*mA*mC |
| D: 1093 | SEQ ID NO: 1800 | mG*mG*mUmAmGmUmUmCfAfAfAmCmUmGmUmAmUmUmA*mC*mA |
| | SEQ ID NO: 2252 | mU*fG*mUmAfAmUfAmCmAmGmUmUmUfGmAfAmCmUmAmCmC*mA*mA |
| D: 1094 | SEQ ID NO: 1802 | mG*mU*mAmUmUmAmCmUfUfUfAmAmUmGmGmAmAmGmC*mA*mA |
| | SEQ ID NO: 2253 | mU*fU*mGmCfUmUfCmCmAmUmUmAmAfAmGfUmAmAmUmAmC*mA*mG |
| D: 1095 | SEQ ID NO: 1804 | mU*mA*mUmUmAmCmUmUfUfAfAmUmGmGmAmAmGmCmA*mA*mA |
| | SEQ ID NO: 2254 | mU*fU*mUmGfCmUfUmCmCmAmUmUmAfAmAfGmUmAmAmUmA*mC*mA |
| D: 1096 | SEQ ID NO: 1806 | mU*mU*mAmCmUmUmUmAfAfUfGmGmAmAmGmCmAmAmG*mU*mA |
| | SEQ ID NO: 2255 | mU*fA*mCmUfUmGfCmUmUmCmCmAmUfUmAfAmAmGmUmAmA*mU*mA |
| D: 1097 | SEQ ID NO: 1808 | mU*mA*mCmUmUmUmAmAfUfGfGmAmAmGmCmAmAmGmU*mG*mA |
| | SEQ ID NO: 2256 | mU*fC*mAmCfUmUfGmCmUmUmCmCmAfUmUfAmAmAmGmUmA*mA*mU |
| D: 1098 | SEQ ID NO: 1810 | mG*mU*mCmCmCmAmCmAfAfAfUmGmAmAmGmAmUmUmU*mA*mA |
| | SEQ ID NO: 2257 | mU*fU*mAmAfAmUfCmUmUmCmAmUmUfUmGfUmGmGmGmAmC*mC*mA |
| D: 1099 | SEQ ID NO: 1812 | mA*mA*mGmAmUmUmUmAfUfAfUmAmUmCmAmGmCmUmG*mC*mA |
| | SEQ ID NO: 2258 | mU*fG*mCmAfGmCfUmGmAmUmAmUmAfUmAfAmAmUmCmUmU*mC*mA |
| D: 1100 | SEQ ID NO: 1814 | mU*mC*mAmGmCmUmGmCfAfCfCmAmUmGmCmCmAmUmC*mU*mA |
| | SEQ ID NO: 2259 | mU*fA*mGmAfUmGfGmCmAmUmGmGmUfGmCfAmGmCmUmGmA*mU*mA |
| D: 1101 | SEQ ID NO: 1816 | mC*mA*mGmCmUmGmCmAfCfCfAmUmGmCmCmAmUmCmU*mA*mA |
| | SEQ ID NO: 2260 | mU*fU*mAmGfAmUfGmGmCmAmUmGmGfUmGfCmAmGmCmUmG*mA*mU |
| D: 1102 | SEQ ID NO: 1818 | mA*mG*mCmUmGmCmAmCfCfAfUmGmCmCmAmUmCmUmA*mU*mA |
| | SEQ ID NO: 2261 | mU*fA*mUmAfGmAfUmGmGmCmAmUmGfGmUfGmCmAmGmCmU*mG*mA |
| D: 1103 | SEQ ID NO: 1820 | mG*mC*mUmGmCmAmCmCfAfUfGmCmCmAmUmCmUmAmU*mA*mA |
| | SEQ ID NO: 2262 | mU*fU*mAmUfAmGfAmUmGmGmCmAmUfGmGfUmGmCmAmGmC*mU*mG |
| D: 1104 | SEQ ID NO: 1822 | mC*mU*mGmCmAmCmCmAfUfGfCmCmAmUmCmUmAmUmA*mG*mA |
| | SEQ ID NO: 2263 | mU*fC*mUmAfUmAfGmAmUmGmGmCmAfUmGfGmUmGmCmAmG*mC*mU |
| D: 1105 | SEQ ID NO: 1824 | mU*mG*mCmAmCmCmAmUfGfCfCmAmUmCmUmAmUmAmG*mA*mA |
| | SEQ ID NO: 2264 | mU*fU*mCmUfAmUfAmGmAmUmGmGmCfAmUfGmGmUmGmCmA*mG*mC |
| D: 1106 | SEQ ID NO: 1826 | mG*mC*mAmCmCmAmUmGfCfCfAmUmCmUmAmUmAmGmA*mG*mA |
| | SEQ ID NO: 2265 | mU*fC*mUmCfUmAfUmAmGmAmUmGmGfCmAfUmGmGmUmGmC*mA*mG |
| D: 1107 | SEQ ID NO: 1828 | mC*mA*mCmCmAmUmGmCfCfAfUmCmUmAmUmAmGmAmG*mA*mA |
| | SEQ ID NO: 2266 | mU*fU*mCmUfCmUfAmUmAmGmAmUmGfGmCfAmUmGmGmUmG*mC*mA |
| D: 1108 | SEQ ID NO: 1830 | mA*mU*mGmCmCmAmUmCfUfAfUmAmGmAmGmAmUmAmG*mG*mA |
| | SEQ ID NO: 2267 | mU*fC*mCmUfAmUfCmUmCmUmAmUmAfGmAfUmGmGmCmAmU*mG*mG |
| D: 1109 | SEQ ID NO: 1832 | mC*mU*mAmUmAmGmAmGfAfUfAmGmGmAmAmCmGmGmU*mG*mA |
| | SEQ ID NO: 2268 | mU*fC*mAmCfCmGfUmUmCmCmUmAmUfCmUfCmUmAmUmAmG*mA*mU |
| D: 1110 | SEQ ID NO: 1834 | mU*mA*mUmAmGmAmGmAfUfAfGmGmAmAmCmGmGmUmG*mG*mA |
| | SEQ ID NO: 2269 | mU*fC*mCmAfCmCfGmUmUmCmCmUmAfUmCfUmCmUmAmUmA*mG*mA |
| D: 1111 | SEQ ID NO: 1836 | mA*mU*mAmGmAmGmAmUfAfGfGmAmAmCmGmGmUmGmG*mG*mA |
| | SEQ ID NO: 2270 | mU*fC*mCmCfAmCfCmGmUmUmCmCmUfAmUfCmUmCmUmAmU*mA*mG |
| D: 1112 | SEQ ID NO: 1838 | mU*mA*mCmUmAmCmCmUfCfAfGmCmAmAmGmCmCmUmG*mU*mA |
| | SEQ ID NO: 2271 | mU*fA*mCmAfGmGfCmUmUmGmCmUmGfAmGfGmUmAmGmUmA*mG*mG |
| D: 1113 | SEQ ID NO: 1840 | mG*mU*mUmUmGmCmAmGfAfUfGmCmUmAmGmGmUmGmU*mU*mA |
| | SEQ ID NO: 2272 | mU*fA*mAmCfAmCfCmUmAmGmCmAmUfCmUfGmCmAmAmAmC*mA*mG |
| D: 1114 | SEQ ID NO: 1842 | mU*mU*mCmAmAmGmGmAfGfCfAmUmGmCmAmAmAmGmA*mU*mA |
| | SEQ ID NO: 2273 | mU*fA*mUmCfUmUfUmGmCmAmUmGmCfUmCfCmUmUmGmAmA*mC*mA |
| D: 1115 | SEQ ID NO: 1844 | mU*mC*mAmAmGmGmAmGfCfAfUmGmCmAmAmAmGmAmU*mA*mA |
| | SEQ ID NO: 2274 | mU*fU*mAmUfCmUfUmUmGmCmAmUmGfCmUfCmCmUmUmGmA*mA*mC |
| D: 1116 | SEQ ID NO: 1846 | mC*mA*mAmGmGmAmGmCfAfUfGmCmAmAmAmGmAmUmA*mA*mA |
| | SEQ ID NO: 2275 | mU*fU*mUmAfUmCfUmUmUmGmCmAmUfGmCfUmCmCmUmUmG*mA*mA |
| D: 1117 | SEQ ID NO: 1848 | mA*mA*mGmGmAmGmCmAfUfGfCmAmAmAmGmAmUmAmA*mU*mA |
| | SEQ ID NO: 2276 | mU*fA*mUmUfAmUfCmUmUmUmGmCmAfUmGfCmUmCmCmUmU*mG*mA |
| D: 1118 | SEQ ID NO: 1850 | mA*mG*mGmAmGmCmAmUfGfCfAmAmAmGmAmUmAmAmU*mC*mA |
| | SEQ ID NO: 2277 | mU*fG*mAmUfUmAfUmCmUmUmUmGmCfAmUfGmCmUmCmCmU*mU*mG |
| D: 1119 | SEQ ID NO: 1852 | mG*mG*mAmGmCmAmUmGfCfAfAmAmGmAmUmAmAmUmC*mC*mA |
| | SEQ ID NO: 2278 | mU*fG*mGmAfUmUfAmUmCmUmUmUmGfCmAfUmGmCmUmCmC*mU*mU |
| D: 1120 | SEQ ID NO: 1854 | mU*mG*mCmCmCmGmGmCfAfGfCmUmUmGmCmCmCmGmA*mA*mA |
| | SEQ ID NO: 2279 | mU*fU*mUmCfGmGfGmCmAmAmGmCmUfGmCfCmGmGmGmCmA*mU*mU |
| D: 1121 | SEQ ID NO: 1856 | mG*mG*mCmAmGmCmUmUfGfCfCmCmGmAmAmUmUmGmU*mG*mA |
| | SEQ ID NO: 2280 | mU*fC*mAmCfAmAfUmUmCmGmGmGmCfAmAfGmCmUmGmCmC*mG*mG |
| D: 1122 | SEQ ID NO: 1858 | mG*mC*mAmGmCmUmUmGfCfCfCmGmAmAmUmUmGmUmG*mU*mA |
| | SEQ ID NO: 2281 | mU*fA*mCmAfCmAfAmUmUmCmGmGmGfCmAfAmGmCmUmGmC*mC*mG |
| D: 1123 | SEQ ID NO: 1860 | mC*mA*mGmCmUmUmGmCfCfCfGmAmAmUmUmGmUmGmU*mG*mA |
| | SEQ ID NO: 2282 | mU*fC*mAmCfAmCfAmAmUmUmCmGmGfGmCfAmAmGmCmUmG*mC*mC |
| D: 1124 | SEQ ID NO: 1862 | mG*mG*mCmAmGmCmAmUfUfGfGmCmAmGmCmAmGmGmA*mC*mA |
| | SEQ ID NO: 2283 | mU*fG*mUmCfCmUfGmCmUmGmCmCmAfAmUfGmCmUmGmCmC*mA*mU |
| D: 1125 | SEQ ID NO: 1864 | mG*mC*mAmGmCmAmUmUfGfGfCmAmGmCmAmGmGmAmC*mA*mA |
| | SEQ ID NO: 2284 | mU*fU*mGmUfCmCfUmGmCmUmGmCmCfAmAfUmGmCmUmGmC*mC*mA |
| D: 1126 | SEQ ID NO: 1866 | mC*mA*mGmCmAmUmUmGfGfCfAmGmCmAmGmGmAmCmA*mU*mA |
| | SEQ ID NO: 2285 | mU*fA*mUmGfUmCfCmUmGmCmUmGmCfCmAfAmUmGmCmUmG*mC*mC |
| D: 1127 | SEQ ID NO: 1868 | mC*mA*mGmCmAmGmGmAfCfAfUmCmUmUmGmUmCmAmA*mA*mA |
| | SEQ ID NO: 2286 | mU*fU*mUmUfGmAfCmAmAmGmAmUmGfUmCfCmUmGmCmUmG*mC*mC |
| D: 1128 | SEQ ID NO: 1870 | mA*mG*mCmAmGmGmAmCfAfUfCmUmUmGmUmCmAmAmA*mA*mA |
| | SEQ ID NO: 2287 | mU*fU*mUmUfUmGfAmCmAmAmGmAmUfGmUfCmCmUmGmCmU*mG*mC |
| D: 1129 | SEQ ID NO: 1872 | mC*mA*mGmGmAmCmAmUfCfUfUmGmUmCmAmAmAmAmG*mU*mA |
| | SEQ ID NO: 2288 | mU*fA*mCmUfUmUfUmGmAmCmAmAmGfAmUfGmUmCmCmUmG*mC*mU |
| D: 1130 | SEQ ID NO: 1874 | mA*mG*mGmAmCmAmUmCfUfUfGmUmCmAmAmAmAmGmU*mC*mA |
| | SEQ ID NO: 2289 | mU*fG*mAmCfUmUfUmUmGmAmCmAmAfGmAfUmGmUmCmCmU*mG*mC |
| D: 1131 | SEQ ID NO: 1876 | mG*mG*mAmCmAmUmCmUfUfGfUmCmAmAmAmAmGmUmC*mA*mA |
| | SEQ ID NO: 2290 | mU*fU*mGmAfCmUfUmUmUmGmAmCmAfAmGfAmUmGmUmCmC*mU*mG |
| D: 1132 | SEQ ID NO: 1878 | mU*mA*mAmGmUmCmUmGfGfGfAmUmGmUmAmGmAmAmC*mU*mA |
| | SEQ ID NO: 2291 | mU*fA*mGmUfUmCfUmAmCmAmUmCmCfCmAfGmAmCmUmUmA*mA*mU |
| D: 1133 | SEQ ID NO: 1880 | mG*mC*mUmAmUmUmUmGfUfUfCmAmGmAmGmAmGmGmC*mC*mA |
| | SEQ ID NO: 2292 | mU*fG*mGmCfCmUfCmUmCmUmGmAmAfCmAfAmAmUmAmGmC*mC*mU |
| D: 1134 | SEQ ID NO: 1882 | mC*mU*mAmUmUmUmGmUfUfCfAmGmAmGmAmGmGmCmC*mU*mA |
| | SEQ ID NO: 2293 | mU*fA*mGmGfCmCfUmCmUmCmUmGmAfAmCfAmAmAmUmAmG*mC*mC |
| D: 1135 | SEQ ID NO: 1884 | mU*mU*mGmGmAmAmGmUfGfUfUmCmAmAmGmAmAmAmU*mA*mA |
| | SEQ ID NO: 2294 | mU*fU*mAmUfUmUfCmUmUmGmAmAmCfAmCfUmUmCmCmAmA*mU*mA |
| D: 1136 | SEQ ID NO: 1886 | mU*mG*mGmAmAmGmUmGfUfUfCmAmAmGmAmAmAmUmA*mA*mA |
| | SEQ ID NO: 2295 | mU*fU*mUmAfUmUfUmCmUmUmGmAmAfCmAfCmUmUmCmCmA*mA*mU |
| D: 1137 | SEQ ID NO: 1888 | mU*mG*mUmAmCmUmGmAfUfAfAmAmAmUmAmCmUmCmU*mA*mA |
| | SEQ ID NO: 2296 | mU*fU*mAmGfAmGfUmAmUmUmUmUmAfUmCfAmGmUmAmCmA*mC*mA |
| D: 1138 | SEQ ID NO: 1890 | mA*mG*mGmAmAmAmCmCfCfUfGmGmCmUmUmGmCmUmU*mG*mA |
| | SEQ ID NO: 2297 | mU*fC*mAmAfGmCfAmAmGmCmCmAmGfGmGfUmUmUmCmCmU*mG*mG |
| D: 1139 | SEQ ID NO: 1892 | mG*mU*mGmCmCmUmAmGfUfUfCmCmUmGmUmGmGmCmC*mA*mA |
| | SEQ ID NO: 2298 | mU*fU*mGmGfCmCfAmCmAmGmGmAmAfCmUfAmGmGmCmAmC*mA*mG |
| D: 1140 | SEQ ID NO: 1894 | mG*mU*mUmCmCmUmGmUfGfGfCmCmAmGmGmAmGmGmU*mU*mA |
| | SEQ ID NO: 2299 | mU*fA*mAmCfCmUfCmCmUmGmGmCmCfAmCfAmGmGmAmAmC*mU*mA |
| D: 1141 | SEQ ID NO: 1896 | mC*mU*mGmUmGmGmCmCfAfGfGmAmGmGmUmUmGmGmU*mG*mA |
| | SEQ ID NO: 2300 | mU*fC*mAmCfCmAfAmCmCmUmCmCmUfGmGfCmCmAmCmAmG*mG*mA |
| D: 1142 | SEQ ID NO: 1898 | mG*mC*mCmAmGmGmAmGfGfUfUmGmGmUmGmAmCmUmG*mA*mA |
| | SEQ ID NO: 2301 | mU*fU*mCmAfGmUfCmAmCmCmAmAmCfCmUfCmCmUmGmGmC*mC*mA |
| D: 1143 | SEQ ID NO: 1900 | mC*mA*mGmAmGmUmUmAfUfUfGmAmAmUmCmUmUmAmA*mU*mA |
| | SEQ ID NO: 2302 | mU*fA*mUmUfAmAfGmAmUmUmCmAmAfUmAfAmCmUmCmUmG*mC*mU |
| D: 1144 | SEQ ID NO: 1902 | mA*mG*mAmGmUmUmAmUfUfGfAmAmUmCmUmUmAmAmU*mU*mA |
| | SEQ ID NO: 2303 | mU*fA*mAmUfUmAfAmGmAmUmUmCmAfAmUfAmAmCmUmCmU*mG*mC |
| D: 1145 | SEQ ID NO: 1904 | mG*mA*mGmUmUmAmUmUfGfAfAmUmCmUmUmAmAmUmU*mU*mA |
| | SEQ ID NO: 2304 | mU*fA*mAmAfUmUfAmAmGmAmUmUmCfAmAfUmAmAmCmUmC*mU*mG |
| D: 1146 | SEQ ID NO: 1906 | mU*mU*mUmUmUmAmAmUfGfUfAmCmAmAmGmUmUmUmU*mG*mA |
| | SEQ ID NO: 2305 | mU*fC*mAmAfAmAfCmUmUmGmUmAmCfAmUfUmAmAmAmAmA*mA*mA |
| D: 1147 | SEQ ID NO: 1908 | mU*mU*mUmAmAmUmGmUfAfCfAmAmGmUmUmUmUmGmU*mA*mA |
| | SEQ ID NO: 2306 | mU*fU*mAmCfAmAfAmAmCmUmUmGmUfAmCfAmUmUmAmAmA*mA*mA |
| D: 1148 | SEQ ID NO: 1910 | mA*mA*mAmUmAmAmUmAfAfAfGmAmAmCmUmCmCmUmU*mA*mA |
| | SEQ ID NO: 2307 | mU*fU*mAmAfGmGfAmGmUmUmCmUmUfUmAfUmUmAmUmUmU*mA*mU |
| D: 1149 | SEQ ID NO: 1912 | mA*mA*mUmAmAmUmAmAfAfGfAmAmCmUmCmCmUmUmA*mU*mA |
| | SEQ ID NO: 2308 | mU*fA*mUmAfAmGfGmAmGmUmUmCmUfUmUfAmUmUmAmUmU*mU*mA |
| D: 1150 | SEQ ID NO: 1914 | mA*mU*mAmAmUmAmAmAfGfAfAmCmUmCmCmUmUmAmU*mU*mA |
| | SEQ ID NO: 2309 | mU*fA*mAmUfAmAfGmGmAmGmUmUmCfUmUfUmAmUmUmAmU*mU*mU |
| D: 1151 | SEQ ID NO: 1916 | mU*mA*mAmUmAmAmAmGfAfAfCmUmCmCmUmUmAmUmU*mU*mA |
| | SEQ ID NO: 2310 | mU*fA*mAmAfUmAfAmGmGmAmGmUmUfCmUfUmUmAmUmUmA*mU*mU |
| D: 1152 | SEQ ID NO: 1918 | mA*mA*mUmAmAmAmGmAfAfCfUmCmCmUmUmAmUmUmU*mU*mA |
| | SEQ ID NO: 2311 | mU*fA*mAmAfAmUfAmAmGmGmAmGmUfUmCfUmUmUmAmUmU*mA*mU |
| D: 1153 | SEQ ID NO: 1920 | mA*mC*mAmUmCmUmAmAfUfGfCmUmUmCmAmAmGmUmG*mU*mA |
| | SEQ ID NO: 2312 | mU*fA*mCmAfCmUfUmGmAmAmGmCmAfUmUfAmGmAmUmGmU*mA*mA |
| D: 1154 | SEQ ID NO: 1922 | mU*mC*mUmGmAmGmCmUfUfUfUmCmAmUmGmGmAmAmG*mU*mA |
| | SEQ ID NO: 2313 | mU*fA*mCmUfUmCfCmAmUmGmAmAmAfAmGfCmUmCmAmGmA*mA*mA |
| D: 1155 | SEQ ID NO: 1924 | mU*mG*mAmGmCmUmUmUfUfCfAmUmGmGmAmAmGmUmG*mG*mA |
| | SEQ ID NO: 2314 | mU*fC*mCmAfCmUfUmCmCmAmUmGmAfAmAfAmGmCmUmCmA*mG*mA |
| D: 1156 | SEQ ID NO: 1926 | mA*mG*mCmUmUmUmUmCfAfUfGmGmAmAmGmUmGmGmC*mA*mA |
| | SEQ ID NO: 2315 | mU*fU*mGmCfCmAfCmUmUmCmCmAmUfGmAfAmAmAmGmCmU*mC*mA |
| D: 1157 | SEQ ID NO: 1928 | mU*mU*mCmAmUmGmGmAfAfGfUmGmGmCmAmGmUmGmA*mA*mA |
| | SEQ ID NO: 2316 | mU*fU*mUmCfAmCfUmGmCmCmAmCmUfUmCfCmAmUmGmAmA*mA*mA |
| D: 1158 | SEQ ID NO: 1930 | mU*mU*mAmAmUmGmUmUfUfUfGmUmAmCmAmAmUmUmA*mC*mA |
| | SEQ ID NO: 2317 | mU*fG*mUmAfAmUfUmGmUmAmCmAmAfAmAfCmAmUmUmAmA*mU*mC |

m indicates 2′O-methyl modified ribose on the listed nucleotide
f indicates 2′F modified ribose on the listed nucleotide
*indicates a phosphorothioate bond (in place of a phosphodiester bond)

### Example 4

In vitro knockdown of human HMGCR in Hep3B cells with cholesterol-conjugated HMGCR siRNA

Knockdown of human HMGCR expression by the cholesterol-conjugated HMGCR siRNA was assayed using the following procedure: On day 1, Hep3B cells (ATCC) were added to Corning 96-well plates at 5,000 cells per well in growth media, on day 2, the culture media was replaced with ACCELL Media (Dharmacon) and siRNA were added directly to the well. For single point (SP) screening, 1 μM (1,000 nM) of cholesterol-conjugated siRNA was used. To generate concentration/dose response curves final concentrations of 1000, 200, 40, 8, 1.6, 0.32, and 0.064 nM of cholesterol-conjugated siRNA concentration was used. Treated cells were lysed and followed with gene expression by using the TaqMan Fast Advanced Cells-to-Ct Kit (Invitrogen). The cell lysates were used immediately for cDNA synthesis using Fast Advanced RT Master Mix (Invitrogen) and using the following steps in a thermocycler: 37° C. for 30 minutes, 95° C. for 5 minutes, and 4° C. hold. Quantitative Polymerase Chain Reaction (qPCR) was performed via TaqMan Gene Expression Assay (Invitrogen) using the following cycles temperatures and times: 50° C. for 2 minutes, 95° C. for 20 seconds, 40 cycles of 95° C. for 1 seconds and 60° C. for 20 seconds. The human HMGCR levels were normalized to human Rplp0 (Life Technologies) and represent the relative knockdown of human HMGCR mRNA expression as compared to vehicle-treated control cells. IC50 values were calculated using a 4-parameter fit model using XLFit.

TABLE 5
Percent Inhibition of human HMGCR expression in Hep3B cells

| | |
|---|---|
| | In vitro knockdown |
| In vitro knockdown | of hHMGCR |
| of hHMGCR | in Hep3B cells |

| | in Hep3B cells | | % Max |
|---|---|---|---|
| Duplex No: | % KD | IC50 (nM) | KD |
| Duplex No: 387 | 13.23 | | |
| Duplex No: 388 | 46.42 | 762.00 | 51.78 |
| Duplex No: 389 | 34.08 | | |
| Duplex No: 390 | 41.88 | | |
| Duplex No: 391 | 14.33 | | |
| Duplex No: 392 | 23.32 | | |
| Duplex No: 393 | 25.68 | | |
| Duplex No: 394 | 12.53 | | |
| Duplex No: 395 | 15.44 | | |
| Duplex No: 396 | 8.80 | | |
| Duplex No: 397 | 25.25 | | |
| Duplex No: 398 | 7.52 | | |
| Duplex No: 399 | 13.78 | | |
| Duplex No: 400 | 15.06 | | |
| Duplex No: 401 | 30.21 | | |
| Duplex No: 402 | 22.46 | | |
| Duplex No: 403 | 0.41 | | |
| Duplex No: 404 | 10.90 | | |
| Duplex No: 405 | 26.99 | | |
| Duplex No: 406 | 3.36 | | |
| Duplex No: 407 | 5.73 | | |
| Duplex No: 408 | 10.67 | | |
| Duplex No: 409 | 13.04 | | |
| Duplex No: 410 | 8.67 | | |
| Duplex No: 411 | 1.70 | | |
| Duplex No: 412 | 23.08 | | |
| Duplex No: 413 | 10.62 | | |
| Duplex No: 414 | 13.93 | | |
| Duplex No: 415 | 9.14 | | |
| Duplex No: 416 | 13.65 | | |
| Duplex No: 417 | 21.52 | | |
| Duplex No: 418 | 24.88 | | |
| Duplex No: 419 | 14.20 | | |
| Duplex No: 420 | 16.23 | | |
| Duplex No: 421 | 12.44 | | |
| Duplex No: 422 | 7.75 | | |
| Duplex No: 423 | 7.70 | | |
| Duplex No: 424 | 14.80 | | |
| Duplex No: 425 | 23.15 | | |
| Duplex No: 426 | 18.49 | | |
| Duplex No: 427 | −2.29 | | |
| Duplex No: 428 | 10.64 | | |
| Duplex No: 429 | −4.12 | | |
| Duplex No: 430 | 14.25 | | |
| Duplex No: 431 | 1.60 | | |
| Duplex No: 432 | 8.05 | | |
| Duplex No: 433 | 4.82 | | |
| Duplex No: 434 | 4.53 | | |
| Duplex No: 435 | 4.47 | | |
| Duplex No: 436 | 0.47 | | |
| Duplex No: 437 | −10.58 | | |
| Duplex No: 438 | −4.59 | | |
| Duplex No: 439 | −3.83 | | |
| Duplex No: 440 | 2.19 | | |
| Duplex No: 441 | 12.76 | | |
| Duplex No: 442 | 62.11 | 277.90 | 69.48 |
| Duplex No: 443 | 11.88 | N/A | 27.15 |
| Duplex No: 444 | 47.64 | 1348.00 | 52.74 |
| Duplex No: 445 | 57.54 | 525.80 | 63.76 |
| Duplex No: 446 | 54.19 | 715.30 | 64.75 |
| Duplex No: 447 | 43.92 | 578.80 | 52.45 |
| Duplex No: 448 | 52.15 | 939.10 | 61.38 |
| Duplex No: 449 | 4.08 | | |
| Duplex No: 450 | 6.85 | | |
| Duplex No: 451 | −7.04 | | |
| Duplex No: 452 | 1.88 | | |
| Duplex No: 453 | 0.79 | | |
| Duplex No: 454 | 1.05 | | |
| Duplex No: 455 | −3.28 | | |
| Duplex No: 456 | 37.60 | | |
| Duplex No: 457 | 11.68 | | |
| Duplex No: 458 | 13.31 | | |
| Duplex No: 459 | 33.54 | | |
| Duplex No: 460 | 18.92 | | |
| Duplex No: 461 | 17.06 | | |
| Duplex No: 462 | 30.31 | | |
| Duplex No: 463 | 9.99 | | |
| Duplex No: 464 | 43.96 | | |
| Duplex No: 465 | 6.07 | | |
| Duplex No: 466 | 16.21 | | |
| Duplex No: 467 | 0.51 | | |
| Duplex No: 468 | 23.02 | | |
| Duplex No: 469 | 14.12 | | |
| Duplex No: 470 | −2.99 | | |
| Duplex No: 471 | 44.32 | | |
| Duplex No: 472 | 27.25 | | |
| Duplex No: 473 | 6.83 | | |
| Duplex No: 474 | 7.57 | | |
| Duplex No: 475 | 16.62 | | |
| Duplex No: 476 | 19.67 | | |
| Duplex No: 477 | −7.45 | | |
| Duplex No: 478 | −15.39 | | |
| Duplex No: 479 | −2.76 | | |
| Duplex No: 480 | −8.95 | | |
| Duplex No: 481 | 5.65 | | |
| Duplex No: 482 | 0.43 | | |
| Duplex No: 483 | −2.74 | | |
| Duplex No: 484 | 2.76 | | |
| Duplex No: 485 | 33.49 | | |
| Duplex No: 486 | 51.41 | 494.40 | 66.02 |
| Duplex No: 487 | 23.83 | | |
| Duplex No: 488 | 17.62 | | |
| Duplex No: 489 | 40.85 | | |
| Duplex No: 490 | −6.31 | | |
| Duplex No: 491 | 7.85 | | |
| Duplex No: 492 | 31.11 | | |
| Duplex No: 493 | 36.88 | | |
| Duplex No: 494 | 22.56 | | |
| Duplex No: 495 | 10.89 | | |
| Duplex No: 496 | 11.43 | | |
| Duplex No: 497 | −1.59 | | |
| Duplex No: 498 | 19.73 | | |
| Duplex No: 499 | 7.55 | | |
| Duplex No: 500 | 12.45 | | |
| Duplex No: 501 | −1.95 | | |
| Duplex No: 502 | −6.74 | | |
| Duplex No: 503 | 29.43 | | |
| Duplex No: 504 | 25.83 | | |
| Duplex No: 505 | 44.02 | | |
| Duplex No: 506 | 36.31 | | |
| Duplex No: 507 | −9.44 | | |
| Duplex No: 508 | 2.36 | | |
| Duplex No: 509 | 18.13 | | |
| Duplex No: 510 | −1.01 | | |
| Duplex No: 511 | 6.94 | | |
| Duplex No: 512 | 34.04 | | |
| Duplex No: 513 | 14.93 | | |
| Duplex No: 514 | 21.16 | | |
| Duplex No: 515 | 1.21 | | |
| Duplex No: 516 | 9.40 | | |
| Duplex No: 517 | −7.15 | | |
| Duplex No: 518 | 6.16 | | |
| Duplex No: 519 | −2.27 | | |
| Duplex No: 520 | −5.11 | | |
| Duplex No: 521 | 0.28 | | |
| Duplex No: 522 | −2.56 | | |
| Duplex No: 523 | 13.59 | | |
| Duplex No: 524 | 19.50 | | |
| Duplex No: 525 | 2.06 | | |
| Duplex No: 526 | 8.65 | | |
| Duplex No: 527 | −7.83 | | |
| Duplex No: 528 | 19.94 | | |
| Duplex No: 529 | 2.87 | | |
| Duplex No: 530 | 14.31 | | |
| Duplex No: 531 | −0.30 | | |
| Duplex No: 532 | 5.79 | | |
| Duplex No: 533 | 8.21 | | |
| Duplex No: 534 | 25.52 | | |
| Duplex No: 535 | 31.12 | | |
| Duplex No: 536 | 15.22 | | |
| Duplex No: 537 | 10.26 | | |
| Duplex No: 538 | 13.48 | | |
| Duplex No: 539 | −21.78 | | |
| Duplex No: 540 | 10.34 | | |
| Duplex No: 541 | 3.92 | | |
| Duplex No: 542 | 3.15 | | |
| Duplex No: 543 | 5.57 | | |
| Duplex No: 544 | 14.47 | | |
| Duplex No: 545 | 16.30 | | |
| Duplex No: 546 | −38.40 | | |
| Duplex No: 547 | −5.49 | | |
| Duplex No: 548 | 8.41 | | |
| Duplex No: 549 | −2.80 | | |
| Duplex No: 550 | −2.32 | | |
| Duplex No: 551 | −0.39 | | |
| Duplex No: 552 | 6.96 | | |
| Duplex No: 553 | 0.72 | | |
| Duplex No: 554 | 31.04 | | |
| Duplex No: 555 | 33.71 | | |
| Duplex No: 556 | 8.14 | | |
| Duplex No: 557 | −2.11 | | |
| Duplex No: 558 | 10.27 | | |
| Duplex No: 559 | 0.11 | | |
| Duplex No: 560 | 20.40 | | |
| Duplex No: 561 | 6.11 | | |
| Duplex No: 562 | 3.45 | | |
| Duplex No: 563 | −8.65 | | |
| Duplex No: 564 | 2.85 | | |
| Duplex No: 565 | 9.37 | | |
| Duplex No: 566 | −6.71 | | |
| Duplex No: 567 | −9.44 | | |
| Duplex No: 568 | 8.50 | | |
| Duplex No: 569 | 1.90 | | |
| Duplex No: 570 | 5.87 | | |
| Duplex No: 571 | 2.72 | | |
| Duplex No: 572 | 4.93 | | |
| Duplex No: 573 | 12.61 | | |
| Duplex No: 574 | 17.06 | | |
| Duplex No: 575 | −2.71 | | |
| Duplex No: 576 | 6.32 | | |
| Duplex No: 577 | −7.17 | | |
| Duplex No: 578 | 3.12 | | |
| Duplex No: 579 | −8.76 | | |
| Duplex No: 580 | 9.70 | | |
| Duplex No: 581 | 13.88 | | |
| Duplex No: 582 | 2.43 | | |
| Duplex No: 583 | −3.48 | | |
| Duplex No: 584 | 15.98 | | |
| Duplex No: 585 | 16.00 | | |
| Duplex No: 586 | 2.13 | | |
| Duplex No: 587 | 15.02 | | |
| Duplex No: 588 | 41.82 | | |
| Duplex No: 589 | 27.27 | | |
| Duplex No: 590 | 27.78 | | |
| Duplex No: 591 | 10.52 | | |
| Duplex No: 592 | 8.12 | | |
| Duplex No: 593 | 19.13 | | |
| Duplex No: 594 | 12.68 | | |
| Duplex No: 595 | 24.75 | | |
| Duplex No: 596 | 24.42 | | |
| Duplex No: 597 | 18.54 | | |
| Duplex No: 598 | 21.52 | | |
| Duplex No: 599 | −1.10 | | |
| Duplex No: 600 | 9.97 | | |
| Duplex No: 601 | 7.19 | | |
| Duplex No: 602 | 19.86 | | |
| Duplex No: 603 | 31.28 | | |
| Duplex No: 604 | 31.13 | | |
| Duplex No: 605 | 9.23 | | |
| Duplex No: 606 | 9.18 | | |
| Duplex No: 607 | 23.40 | | |
| Duplex No: 608 | 25.97 | | |
| Duplex No: 609 | 8.04 | | |
| Duplex No: 610 | 16.58 | | |
| Duplex No: 611 | 36.53 | | |
| Duplex No: 612 | 11.41 | | |
| Duplex No: 613 | 20.99 | | |
| Duplex No: 614 | 19.61 | | |
| Duplex No: 615 | −1.31 | | |
| Duplex No: 616 | 3.38 | | |
| Duplex No: 617 | 19.43 | | |
| Duplex No: 618 | 5.25 | | |
| Duplex No: 619 | 8.16 | | |
| Duplex No: 620 | 14.90 | | |
| Duplex No: 621 | 11.39 | | |
| Duplex No: 622 | 5.46 | | |
| Duplex No: 623 | −7.56 | | |
| Duplex No: 624 | 14.04 | | |
| Duplex No: 625 | 4.18 | | |
| Duplex No: 626 | 10.89 | | |
| Duplex No: 627 | −3.90 | | |
| Duplex No: 628 | 6.14 | | |
| Duplex No: 629 | 3.66 | | |
| Duplex No: 630 | 1.99 | | |
| Duplex No: 631 | 2.73 | | |
| Duplex No: 632 | −0.28 | | |
| Duplex No: 633 | 4.71 | | |
| Duplex No: 634 | 11.66 | | |
| Duplex No: 635 | 0.74 | | |
| Duplex No: 636 | −11.88 | | |
| Duplex No: 637 | 4.14 | | |
| Duplex No: 638 | 3.13 | | |
| Duplex No: 639 | 3.86 | | |
| Duplex No: 640 | 0.60 | | |
| Duplex No: 641 | 11.44 | | |
| Duplex No: 642 | 13.46 | | |
| Duplex No: 643 | −2.43 | | |
| Duplex No: 644 | −4.35 | | |
| Duplex No: 645 | −4.81 | | |
| Duplex No: 646 | 2.61 | | |
| Duplex No: 647 | 6.41 | | |
| Duplex No: 648 | 5.11 | | |
| Duplex No: 649 | 7.21 | | |
| Duplex No: 650 | 6.31 | | |
| Duplex No: 651 | 3.24 | | |
| Duplex No: 652 | 5.36 | | |
| Duplex No: 653 | −1.82 | | |
| Duplex No: 654 | 12.07 | | |
| Duplex No: 655 | 8.11 | | |
| Duplex No: 656 | 22.87 | | |
| Duplex No: 657 | 10.42 | | |
| Duplex No: 658 | 26.26 | | |
| Duplex No: 659 | −5.00 | | |
| Duplex No: 660 | 0.38 | | |
| Duplex No: 661 | −3.12 | | |
| Duplex No: 662 | 13.23 | | |
| Duplex No: 663 | 38.31 | | |
| Duplex No: 664 | 25.80 | | |
| Duplex No: 665 | 47.53 | 451.40 | 64.94 |
| Duplex No: 666 | 21.80 | | |
| Duplex No: 667 | 26.57 | | |
| Duplex No: 668 | 17.67 | | |
| Duplex No: 669 | 31.81 | | |
| Duplex No: 670 | 34.65 | | |
| Duplex No: 671 | 26.48 | | |
| Duplex No: 672 | 8.84 | | |
| Duplex No: 673 | 49.49 | 1200.00 | 59.73 |
| Duplex No: 674 | 40.69 | | |
| Duplex No: 675 | 46.17 | 2908.00 | 49.25 |
| Duplex No: 676 | 6.92 | | |
| Duplex No: 677 | 14.98 | | |
| Duplex No: 678 | 15.26 | | |
| Duplex No: 679 | 42.03 | | |
| Duplex No: 680 | 16.55 | | |
| Duplex No: 681 | 27.23 | | |
| Duplex No: 682 | 13.66 | | |
| Duplex No: 683 | 18.85 | | |
| Duplex No: 684 | 15.46 | | |
| Duplex No: 685 | 14.74 | | |
| Duplex No: 686 | 24.53 | | |
| Duplex No: 687 | 15.87 | | |
| Duplex No: 688 | 5.10 | | |
| Duplex No: 689 | 17.03 | | |
| Duplex No: 690 | 20.51 | | |
| Duplex No: 691 | 14.14 | | |
| Duplex No: 692 | 17.03 | | |
| Duplex No: 693 | 3.63 | | |
| Duplex No: 694 | 12.25 | | |
| Duplex No: 695 | 13.06 | | |
| Duplex No: 696 | 16.09 | | |
| Duplex No: 697 | 19.25 | | |
| Duplex No: 698 | 18.97 | | |
| Duplex No: 699 | 3.34 | | |
| Duplex No: 700 | 3.19 | | |
| Duplex No: 701 | 16.14 | | |
| Duplex No: 702 | 58.66 | 1187.00 | 62.35 |
| Duplex No: 703 | 20.41 | N/A | 27.83 |
| Duplex No: 704 | 37.09 | 783.70 | 42.38 |
| Duplex No: 705 | 70.31 | 248.00 | 74.74 |
| Duplex No: 706 | 60.00 | 611.60 | 63.64 |
| Duplex No: 707 | 68.64 | 303.80 | 72.68 |
| Duplex No: 708 | 29.38 | N/A | 35.98 |
| Duplex No: 709 | 45.92 | 686.90 | 57.23 |
| Duplex No: 710 | 3.55 | | |
| Duplex No: 711 | 7.26 | | |
| Duplex No: 712 | 29.77 | | |
| Duplex No: 713 | 15.73 | | |
| Duplex No: 714 | 17.49 | | |
| Duplex No: 715 | 43.64 | | |
| Duplex No: 716 | 12.56 | | |
| Duplex No: 717 | 2.20 | | |
| Duplex No: 718 | 34.02 | | |
| Duplex No: 719 | 14.30 | | |
| Duplex No: 720 | 15.77 | | |
| Duplex No: 721 | 26.74 | | |
| Duplex No: 722 | 20.58 | | |
| Duplex No: 723 | 20.83 | | |
| Duplex No: 724 | 11.27 | | |
| Duplex No: 725 | 4.56 | | |
| Duplex No: 726 | 16.20 | | |
| Duplex No: 727 | 22.49 | | |
| Duplex No: 728 | 36.14 | | |
| Duplex No: 729 | 22.28 | | |
| Duplex No: 730 | 18.83 | | |
| Duplex No: 731 | 61.63 | 320.90 | 70.18 |
| Duplex No: 732 | 30.26 | N/A | 35.54 |
| Duplex No: 733 | 47.31 | 1293.00 | 58.60 |
| Duplex No: 734 | 3.94 | N/A | 15.41 |
| Duplex No: 735 | 46.17 | 2154.00 | 51.25 |
| Duplex No: 736 | 24.25 | | |
| Duplex No: 737 | 18.86 | | |
| Duplex No: 738 | 26.23 | | |
| Duplex No: 739 | 26.24 | | |
| Duplex No: 740 | 13.30 | | |
| Duplex No: 741 | 25.65 | | |
| Duplex No: 742 | 14.77 | | |
| Duplex No: 743 | 28.64 | | |
| Duplex No: 744 | 23.39 | | |
| Duplex No: 745 | 57.11 | 380.40 | 64.50 |
| Duplex No: 746 | 34.00 | | |
| Duplex No: 747 | 28.79 | | |
| Duplex No: 748 | 38.96 | | |
| Duplex No: 749 | 37.46 | | |
| Duplex No: 750 | 39.39 | | |
| Duplex No: 751 | 33.99 | | |
| Duplex No: 752 | 0.20 | | |
| Duplex No: 753 | 10.32 | N/A | 30.26 |
| Duplex No: 754 | 9.14 | | |
| Duplex No: 755 | 15.83 | | |
| Duplex No: 756 | 15.98 | | |
| Duplex No: 757 | 35.90 | | |
| Duplex No: 758 | 29.49 | | |
| Duplex No: 759 | 22.98 | | |
| Duplex No: 760 | −2.21 | | |
| Duplex No: 761 | 29.14 | | |
| Duplex No: 762 | 11.12 | | |
| Duplex No: 763 | 32.27 | | |
| Duplex No: 764 | 32.72 | | |
| Duplex No: 765 | 12.28 | | |
| Duplex No: 766 | 16.57 | | |
| Duplex No: 767 | −3.77 | | |
| Duplex No: 768 | 6.49 | | |
| Duplex No: 769 | 15.02 | | |
| Duplex No: 770 | 4.25 | | |
| Duplex No: 771 | 11.50 | | |
| Duplex No: 772 | −3.65 | | |

Table 5: shows the result of a single dose screen in Hep3B cells by free uptake with the indicated cholesterol conjugated HMGCR siRNA. Data are expressed as percent of message knockdown relative to untreated cells. The IC50 and percent maximum knockdown of top hits from single point screening followed by concentration/dose response curves are included as well.

### Example 5

In vitro knockdown of HMGCR in wildtype mouse primary hepatocytes (MPH) and Hep3B cells with GalNAc-conjugated HMGCR siRNA

Knockdown of mouse HMGCR expression by the LYGal1-conjugated HMGCR siRNA was assayed using the following procedure: mouse primary hepatocytes (MPH) were freshly isolated from a wildtype mouse, added to Corning plates at 15,000 per well, and siRNA were added directly to the well. For Hep3B (ATCC) cells, transfection reagent RNAiMAX (Life Technologies) at 0.3 μL/well was mixed with siRNA in Corning plates before adding cells at 20,000 per well. To generate concentration/dose response curves final concentrations of 1000, 333, 111, 37, 12, 4, 1.37, 0.46, 0.15, 0.05, and 0.017 nM of GalNAc-conjugated siRNA concentration was used for MPH. For Hep3B, concentration/dose response curves final concentrations of 10, 3.33, 1.11, 0.37, 0.12, 0.04, 0.014, 0.005, 0.002, 0.0005, and 0.0002 nM of GalNAc-conjugated siRNA concentration was used.

Treated cells were lysed and RNA was isolated using the Quick-RNA 96 Kit (Zymo Research) directly into the 96 well plate. The eluted RNA was used immediately or stored frozen. cDNA was synthesized using Fast Advanced RT Master Mix (Invitrogen) and using the following steps in a thermocycler: 37° C. for 30 minutes, 95° C. for 5 minutes, and 4° C. hold. Polymerase Chain Reaction (PCR) was performed via TaqMan RT PCR (Life Technologies) using the following cycles temperatures and times: 50° C. for 2 minutes, 95° C. for 10 minutes, 40 cycles of 95° C. for 15 seconds and 60° C. for 1 minute.

The mouse or human HMGCR levels were normalized to mouse (for MPH) or human (for Hep3B) Rplp0 (Life Technologies) and represent the relative knockdown of mouse or human HMGCR mRNA expression as compared to vehicle-treated control cells. IC50 values are calculated using a 4-parameter fit model using XLFit.

TABLE 6
In vitro knockdown of HMGCR in wildtype mouse
primary hepatocytes (MPH) and Hep3B cells
with GalNAc-conjugated HMGCR siRNA

| | In vitro knockdown | | In vitro knockdown | |
|---|---|---|---|---|
| | of mHmgcr in MPH | | of hHMGCR in Hep3B/RNAiMAX | |
| Duplex | IC50 | % Max | IC50 | % Max |
| NO: | (nM) | KD | (nM) | KD |
| D: 388 | 10.26 | 64.46 | 0.011 | 89.27 |
| D: 442 | 4.65 | 85.36 | 0.014 | 85.14 |
| D: 443 | 9.22 | 71.97 | | |
| D: 444 | 6.05 | 78.73 | 0.029 | 91.38 |
| D: 445 | 6.33 | 71.57 | | |
| D: 446 | 15.90 | 67.94 | | |
| D: 447 | 4.06 | 86.52 | | |
| D: 448 | 5.18 | 76.92 | 0.050 | 92.69 |
| D: 486 | 6.13 | 70.19 | | |
| D: 665 | 0.05 | 19.00 | | |
| D: 673 | 994.50 | 41.00 | | |
| D: 675 | 0.40 | 25.66 | | |
| D: 702 | 1.82 | 81.81 | | |
| D: 703 | 328.10 | 8.40 | | |
| D: 704 | 0.02 | 3.66 | | |
| D: 705 | 22.89 | 65.70 | | |
| D: 706 | 8.80 | 82.97 | 0.018 | 86.72 |
| D: 707 | 11.33 | 79.84 | 0.012 | 86.05 |
| D: 708 | 89.29 | 23.37 | | |
| D: 709 | 13.93 | 63.62 | | |
| D: 731 | 19.33 | 71.49 | | |
| D: 732 | 2.70 | 66.14 | | |
| D: 733 | 16.80 | 51.62 | | |
| D: 734 | 144.70 | 46.15 | | |
| D: 735 | 246.60 | 56.51 | 0.006 | 86.22 |
| D: 745 | 3.36 | 68.72 | | |
| D: 753 | 1000.00 | 37.00 | | |
| D: 774 | 2.347 | 76.53 | | |
| D1121 | 30.5 | 62.42 | | |
| D: 830 | 1.521 | 87.12 | 0.09439 | 91.737 |
| D: 834 | 4.726 | 88.79 | 0.2684 | 90.154 |
| D: 828 | 4.749 | 79.04 | 0.07811 | 87.94 |
| D: 1092 | 8.909 | 71.37 | 0.09661 | 90.06 |
| D: 1093 | 14.73 | 67.58 | 0.2028 | 93.36 |

Table 6: shows the result of IC50 with percent maximum knockdown of top hits from single point screen in wildtype mouse primary hepatocytes by free uptake and Hep3B cells by transfection reagent, RNAiMAX, with the indicated HMGCR siRNA. Data are expressed as percent of HMGCR message knockdown relative to untreated cells.

### Example 6: In Vivo Single Dose Screen Mouse HMGCR KD

GalNAc-siRNA were tested in male C57bl/6 mice (n=7) (Taconic farms). Mice were assigned to groups with similar body weight. Either PBS or GalNac-siRNA test article, at a dose of 5 mg/kg, was administered subcutaneously to mice. Seven days post subcutaneous injection, blood was collected from all mice. Fourteen days post subcutaneous injection mice were euthanized under isoflurane anesthesia. Blood was collected by cardiac puncture. Liver was collected from the mice and frozen in liquid nitrogen. Livers were homogenized in TriZol (Invitrogen) using Lysing Matrix D bead tubes on a FastPrep-24 (MP Bio). Chloroform was added and the aqueous phase was mixed with ethanol to precipitate the RNA. RNA was isolated on columns using PureLink Pro96 Total RNA purification kit (Invitrogen) according to manufacturer's protocol and quantified on a NanoDrop (Thermo Fisher). Equal amounts (1 ug) of RNA were reverse transcribed to cDNA using High-Capacity cDNA Reverse Transcription kit (Life Technologies) on Mastercycler Nexus (Eppendorf). Thermocycler settings were 25° C. for 10 min, 37° C. for 2 hrs, then 85° C. for 5 min. Template cDNA was combined with Taqman Universal Master Mix and Assays on Demand primer/probe sets and RT-PCR was performed on the QuantStudio Pro7 (Thermo Fisher) with the following parameters: 50° C. for 2 min, 95° C. for 10 min then 40 cycles of 95° C. for 15 sec and 60° C. for 1 min. Fold changes (FC) were calculated as follows: the CT value of mouse Rplp0 was subtracted from CT value of mouse HMGCR to obtain the delta CT value. Then the delta delta CT value was calculated by subtracting the delta CT value of the untreated sample (average of PBS control) from the delta CT value of each test sample. Fold change was calculated by taking the log base 2 of the negative delta delta CT value. mRNA percent Knockdown (KD) was calculated by subtracting the fold change from the fold change of the PBS group and then multiplying by −100. Data was shown in Table 7.

TABLE 7
In vivo single dose Screen mouse HMGCR KD

| Duplex | Dose | 2-week % |
|---|---|---|
| NO: | mg/kg | KD |
| D: 447 | 5 | −56.8 |
| D: 442 | 5 | −63.3 |
| D: 706 | 5 | −53.2 |
| D: 702 | 5 | −26.2 |
| D: 707 | 5 | −57.7 |
| D: 444 | 5 | −58.7 |
| D: 448 | 5 | −64.1 |
| D: 443 | 5 | 24.3 |
| D: 445 | 5 | −0.1 |
| D: 731 | 5 | −15.3 |
| D: 486 | 5 | −7.0 |
| D: 745 | 5 | −21.2 |
| D: 446 | 5 | −37.3 |
| D: 732 | 5 | −23.4 |
| D: 705 | 5 | −52.6 |
| D: 388 | 5 | −65.3 |
| D: 709 | 5 | −17.5 |
| D: 735 | 5 | −54.8 |
| D: 733 | 5 | −46.1 |
| D: 734 | 5 | −45.0 |
| D: 673 | 5 | −18.6 |
| D: 753 | 5 | −38.4 |
| D: 707 | 5 | −34.5 |
| D: 706 | 5 | −31.1 |
| D: 442 | 5 | −58.6 |
| D: 448 | 5 | −65.7 |
| D: 444 | 5 | −47.7 |
| D: 388 | 5 | −45.5 |
| D: 735 | 5 | −42.5 |

### Example 7: In Vivo Durability 8-Week Mouse HMGCR KD

GalNAc-siRNA were tested in male mice C57/BL6 mice (n=9) (Taconic Farms). Body weight of mice was measured and mice were assigned to groups with similar body weight. PBS or test article GalNac-siRNA, at doses of 0.3, 1.75 and 10 mg/kg were administered subcutaneously to mice. At 2 weeks post siRNA administration, 3 mice from each group were euthanized under isoflurane anesthesia, blood was collected. Liver was collected from the mice and frozen in liquid nitrogen. Blood was collected from the remaining mice (n=6/group) at 2, 4, and 6-weeks post siRNA administration under isoflurane anesthesia. At 8 weeks post siRNA administration, the remaining mice (n=6) were euthanized under isoflurane anesthesia. Blood and liver were collected from mice. Livers were processed and mRNA percent KD was calculated as described in the in vivo single dose screen.

TABLE 8
In vivo durability 8-week mouse HMGCR KD

| Duplex | Dose | 2-week % | 8-week % |
|---|---|---|---|
| No: | mg/kg | KD | KD |
| D: 388 | 0.3 | 37 | −1 |
| D: 388 | 1.75 | 39 | −9 |
| D: 388 | 10 | 47 | −41 |
| D: 735 | 0.3 | 7 | 6 |
| D: 735 | 1.75 | 20 | 4 |
| D: 735 | 10 | 28 | −44 |
| D: 734 | 0.3 | −18 | 0 |
| D: 734 | 1.75 | −1 | 38 |
| D: 734 | 10 | 18 | 45 |
| D: 444 | 0.3 | −38 | 2 |
| D: 444 | 1.75 | −36 | −21 |
| D: 444 | 10 | −64 | −30 |
| D: 448 | 0.3 | −30 | 2 |
| D: 448 | 1.75 | −4 | 5 |
| D: 448 | 10 | −64 | −45 |
| D: 442 | 0.3 | −29 | −14 |
| D: 442 | 1.75 | −35 | −34 |
| D: 442 | 10 | −44 | −33 |
| D: 706 | 0.3 | 26 | −12 |
| D: 706 | 1.75 | −47 | 11 |
| D: 706 | 10 | −57 | −43 |
| D: 707 | 0.3 | −36 | 6 |
| D: 707 | 1.75 | −55 | −5 |
| D: 707 | 10 | −76 | −38 |

### Example 8: In vivo with AAV8 Single dose Screen human HMGCR KD

GalNAc-siRNA were tested in male C57bl/6 mice (n=7) (Taconic farms). The siRNAs were tested in a single study. Mice were dosed by retro-orbital injection with an adeno-associated virus (AAV) vector containing a plasmid with a TBGS1 promoter and the coding sequence and 3′UTR for human HMGCR (NM_000859.3) (Vector BioLabs). The body weight of mice was measured about 4 weeks post AAV administration. Mice were assigned to groups with similar body weight. Either PBS or GalNAc-siRNA test article, at a dose of 5 mg/kg, was administered subcutaneously to mice. Seven days post subcutaneous injection, blood was collected from the retro-orbital sinus from all mice. Fourteen days post subcutaneous injection mice were euthanized under isoflurane anesthesia. Blood was collected by cardiac puncture. Liver was collected from the mice and frozen in liquid nitrogen. Human HMGCR mRNA was quantified as described here. All reagents mentioned in the following sections come from the QuantiGene Singleplex assay kit made by Invitrogen. Approximately 10 mg of liver was weighed into a 96 well cluster tube plate. 300 μL of homogenizing buffer with proteinase k was added to each liver sample and homogenized on the Qiagen homogenizer for 12 minutes. The plate was centrifuged at 3500 rpm for 10 minutes and then heated at 60° C. for 30 minutes, with a vortexing step every 10 minutes. Samples were centrifuged again at 3500 rpm for 10 minutes and then diluted or used neat in the following steps. The working probe set for each gene of interest was prepared in separate tubes by combining the following reagents, in the order listed and scaled according to the number of wells to be run with required overage: nuclease-free water (25.4 μL), Lysis mixture (33.3 μL), Blocking Reagent (1 μL), QuantiGene Singleplex Probe Set (0.3 μL) per 1 well. The capture plate was prepared by dispensing 60 μL of the working probe sets into each well of the plate. Probes sets for mGAPDH (SB-10001) and hHMGCR (SA-11011) were aliquoted into the plate separately and then 60 μL of neat liver homogenate was added to the hHMGCR working set in the plate while 60 μL of the 20-fold dilution of RNA isolate was added to the mGAPDH probe set which was previously aliquoted into the capture plate. Introduction of bubbles was avoided, and the plate was not mixed. An adhesive seal was placed tightly on the plate and then it was incubated at 55±1° C. for 20.5 hours in order to hybridize the probes to the RNA targets. After 20.5 hours, 200 μL of 1× wash buffer was added to the capture plate and then inverted to remove the wash. The plate was then washed two more times with 300 μL of wash buffer for each wash. Next, 100 μL of pre-amplifier solution was added to the plate. It was sealed and incubated at 55±1° C. for 60 minutes. After 1 hour, the wash procedure above was repeated and 100 μL of amplifier solution was added to the plate. It was sealed and incubated at 55±1° C. for 60 minutes. After the 1-hour incubation, the wash steps were performed again and then 100 μL of label probe was added. It was sealed and incubated at 50±1° C. for 60 minutes. The wash steps were performed one additional time and then 100 μL of substrate was added at room temperature and incubated for 5 minutes while being protected from light. The plate was then read on a luminometer with the integration time set to 0.2 seconds. Gene knockdown was calculated by first dividing hHMGCR chemiluminescent signal by mGAPDH signal. The fold change from the PBS (control) group was calculated by dividing all groups by the average signal of the control group. Then % hHMGCR gene knockdown was calculated by subtracting the average control group fold change from all groups followed by dividing all of those group by the average signal from the control group.

TABLE 9
In vivo with AAV8 single dose screen human HMGCR KD

| Duplex | Dose | 2 Week |
|---|---|---|
| NO: | mg/kg | % KD |
| D: 448 | 5 | −62.5 |
| D: 442 | 5 | −66.4 |
| D: 707 | 5 | −59.6 |
| D: 735 | 5 | −33.6 |
| D: 705 | 5 | −58.0 |
| D: 731 | 5 | −64.1 |
| D: 745 | 5 | −55.2 |
| D: 486 | 5 | −54.8 |
| D: 665 | 5 | −63.9 |

1. An RNAi agent for reducing HMGCR gene expression, wherein the RNAi agent comprises a delivery moiety of Formula I conjugated to R, wherein R is a double stranded RNA (dsRNA) comprising an antisense strand and a sense strand: 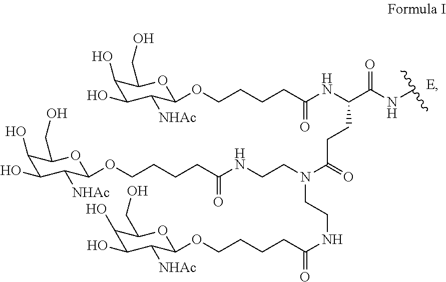
wherein R is conjugated to connection point E of Formula I, optionally via a linker, wherein the sense strand and the antisense strand form a duplex region, and wherein the antisense strand comprises a region of complementarity to a HMGCR mRNA target sequence of SEQ ID NO: 1, and wherein the sense and antisense strand each optionally comprise one or more modified nucleotides and one or more modified internucleotide linkages.

2. The RNAi agent of claim 1, wherein Formula I is conjugated to the 3′ terminal nucleotide of the sense strand, optionally via a linker.

3. (canceled)

4. The RNAi agent of claim 1, wherein the antisense strand is 15 to 50 nucleotides in length.

5. The RNAi agent of claim 1, wherein the sense strand is 15 to 50 nucleotides in length.

6. The RNAi agent of claim 1, wherein the antisense strand is between 18 and 23 nucleotides in length.

7. The RNAi agent of claim 1, wherein the sense strand is between 18 and 21 nucleotides in length.

8. The RNAi agent of claim 1, wherein the antisense strand is 23 nucleotides in length and the sense strand is 21 nucleotides in length.

9. The RNAi agent of claim 1, wherein the region of complementarity is at least 18 nucleotides in length.

10. The RNAi agent of claim 1, wherein the antisense strand comprises a sequence selected from the group consisting of SEQ ID NOs: 2 to 387.

11. The RNAi agent of claim 1, wherein the antisense strand has a nucleotide sequence selected from the group consisting of SEQ ID NOs: 774 to 1159, or a sequence having at least 90% sequence identity thereto.

12. The RNAi agent of claim 1, wherein the sense strand is selected from the group consisting of SEQ ID NOs: 388 to 773, or a sequence having at least 90% sequence identity thereto.

13. The RNAi agent of claim 1, wherein the sense strand or the antisense strand each independently comprise one or more modified nucleotides, and the modified nucleotides are independently 2′ fluoro modified nucleotide residues or 2′-O-methyl modified nucleotides.

14. (canceled)

15. The RNAi agent of claim 1, wherein each nucleotide of the sense strand and each nucleotide of the antisense strand is a modified nucleotide.

16. The RNAi agent of claim 1, wherein the antisense strand is 23 nucleotides in length and wherein each nucleotide of the antisense strand is a modified nucleotide, and 2′ fluoro modified nucleotides are present at
a. positions 2, 3, 7, 14, and 16 from the 5′ end of the antisense strand; or
b. positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.

17. The RNAi agent of claim 1, wherein the sense strand and antisense strand each independently comprise one or more modified internucleotide linkages, and wherein each modified internucleotide linkage is a phosphorothioate linkage.

18.-19. (canceled)

20. The RNAi agent of claim 1, wherein the antisense strand comprises a sequence selected from the group consisting of SEQ ID NOs: 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, or a sequence having at least 90% sequence identity thereto, wherein the 5′ terminal nucleotide of the antisense strand comprises a vinyl phosphonate, a phosphate, or a hydroxyl group.

21. (canceled)

22. The RNAi agent of claim 1, wherein the sense strand comprises a sequence selected from the group consisting of SEQ ID NOs: 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, or a sequence having at least 90% sequence identity thereto.

23. (canceled)

24. The RNAi agent of claim 1, wherein the sense strand comprises a sequence selected from the group consisting of SEQ ID NOs: 1932-2317, or a sequence having at least 90% sequence identity thereto.

25. The RNAi agent of claim 1, wherein the sense strand comprises a sequence selected from the group consisting of SEQ ID NOs: 1932-2317, or a sequence having at least 95% sequence identity thereto.

26. The RNAi agent of claim 1, wherein the RNAi agent antisense strand comprises a first nucleic acid sequence that has at least 90% sequence identity to an antisense sequence of any one of Duplex NOs: 387-772 in Table 4A, and the sense strand comprises a second nucleic acid sequence that has at least 90% sequence identity to a sense sequence of the same Duplex in Table 4A.

27. The RNAi agent of claim 1, wherein the RNAi agent antisense strand comprises a first nucleic acid sequence that has at least 90% sequence identity to an antisense sequence of any one of Duplex NOs: 773-1158 in Table 4B, and the sense strand comprises a second nucleic acid sequence that has at least 90% sequence identity to a sense sequence of the same Duplex in Table 4B.

28.-29. (canceled)

30. The RNAi agent of claim 1, wherein R is conjugated to Formula I via a linker, and wherein linker comprises Formula II having connection points A and B or the linker comprises Formula III having connection points C and D, and wherein: 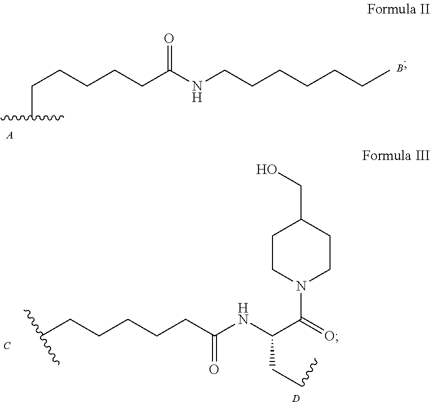
a. Formula I at connection point E is conjugated to Formula II at connection point A and Formula II is conjugated to a phosphate group or a phosphorothioate group at connection point B, and the phosphate group or phosphorothioate group is conjugated to R; or
b. Formula I at connection point E is conjugated to Formula III at connection point C and Formula III is conjugated to a phosphate group or phosphorothioate group at connection point D, and the phosphate group or phosphorothioate group is further conjugated to R.

31. The RNAi agent of claim 1, wherein R is conjugated to Formula I via a linker, and wherein the linker comprises Formula III having connection points C and D: 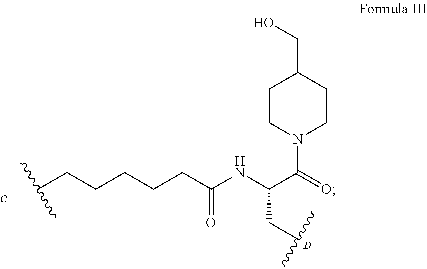
wherein Formula I at connection point E is conjugated to Formula III at connection point C and Formula III is conjugated to a phosphate group or a phosphorothioate group at connection point D, and the phosphate group or the phosphorothioate group is further conjugated to R.

32. A pharmaceutical composition comprising the RNAi agent of claim 1, and one or more pharmaceutically acceptable excipients.

33.-41. (canceled)

42. A method of treating a disease or disorder associated with ASCVD in a patient in need thereof, comprising administering to the patient the RNAi agent of claim 1, or a pharmaceutical composition thereof.

43. The method of claim 42, wherein the disease or disorder is dyslipidemia, primary dysbetalipoproteinemia, hypertriglyceridemia, or atherosclerosis.

44. The method of claim 42, wherein the disease or disorder is dyslipidemia.

45. The method of claim 44, wherein the dyslipidemia is hypercholesterolemia.

46. A method of decreasing HMGCR expression in a cell, comprising contacting the cell with the RNAi agent of claim 1.

47. The method of claim 46, wherein the method further comprises incubating the cell for a time sufficient for decreasing the level of HMGCR mRNA by at least 50% as compared to an untreated or control treated cell.
